SEQUENCE LISTING (106 sequences)

SEQ ID NO: 1 - protein, 19 aa, Artificial Sequence
  Synthetic: Signal peptide amino acid sequence
MEWSWVFLFFLSVTTGVHS

SEQ ID NO: 2 - protein, 10 aa, Artificial Sequence
  Synthetic: Myc-tag amino acid sequence
EQKLISEEDL

SEQ ID NO: 3 - protein, 245 aa, Artificial Sequence
  Synthetic: Murine 38C2 scFv amino acid sequence
DVVMTQTPLSLPVRLGDQASISCRSSQSLLHTYGSPYLNWYLQKPGQSPKLLIYKVSNRFSGVPDRFSGSGSGTDFTLRISRVEAEDLGVYFCSQGTHLPYTFGGGTKLEIKGGGGSGGGGSGGGGSEVKLVESGGGLVQPGGTMKLSCEISGLTFRNYWMSWVRQSPEKGLEWVAEIRLRSDNYATHYAESVKGKFTISRDDSKSRLYLQMNSLRTEDTGIYYCKTYFYSFSYWGQGTLVTVSA

SEQ ID NO: 4 - protein, 245 aa, Artificial Sequence
  Synthetic: humanized 38C2 scFv amino acid sequence
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKGGGGSGGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 5 - protein, 46 aa, Homo sapiens
  misc_feature (1)..(46): CD8 hinge amino acid sequence
AKPTTTPAPRPPTPAPTIASQPLSLRPEACRPAAGGAVHTRGLDFA

SEQ ID NO: 6 - protein, 28 aa, Homo sapiens
  misc_feature (1)..(28): CD3-zeta transmembrane domain amino acid sequence
LDPKLCYLLDGILFIYGVILTALFLRVK

SEQ ID NO: 7 - protein, 41 aa, Homo sapiens
  misc_feature (1)..(41): CD28 intracellular domain amino acid sequence
RSKRSRLLHSDYMNMTPRRPGPTRKHYQPYAPPRDFAAYRS

SEQ ID NO: 8 - protein, 113 aa, Homo sapiens
  misc_feature (1)..(113): CD3-zeta intracellular domain amino acid sequence
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 502 aa, Artificial Sequence
  Synthetic: Murine PUCR with Myc-tag amino acid sequence
MEWSWVFLFFLSVTTGVHSDVVMTQTPLSLPVRLGDQASISCRSSQSLLHTYGSPYLNWYLQKPGQSPKLLIYKVSNRFSGVPDRFSGSGSGTDFTLRISRVEAEDLGVYFCSQGTHLPYTFGGGTKLEIKGGGGSGGGGSGGGGSEVKLVESGGGLVQPGGTMKLSCEISGLTFRNYWMSWVRQSPEKGLEWVAEIRLRSDNYATHYAESVKGKFTISRDDSKSRLYLQMNSLRTEDTGIYYCKTYFYSFSYWGQGTLVTVSAEQKLISEEDLAKPTTTPAPRPPTPAPTIASQPLSLRPEACRPAAGGAVHTRGLDFALDPKLCYLLDGILFIYGVILTALFLRVKRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 502 aa, Artificial Sequence
  Synthetic: Humanized PUCR with Myc-tag with signal peptide amino acid sequence
MEWSWVFLFFLSVTTGVHSELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKGGGGSGGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSEQKLISEEDLAKPTTTPAPRPPTPAPTIASQPLSLRPEACRPAAGGAVHTRGLDFALDPKLCYLLDGILFIYGVILTALFLRVKRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - DNA, 54 bp, Artificial Sequence
  Synthetic: Signal peptide nucleic acid sequence
atggagtggtcctgggtgttcctgttctttctgtccgtgaccaccggtgtccac

SEQ ID NO: 12 - DNA, 30 bp, Artificial Sequence
  Synthetic: Myc-tag nucleic acid sequence
gagcagaaactcatttctgaagaggacctt

SEQ ID NO: 13 - DNA, 735 bp, Artificial Sequence
  Synthetic: murine 38C2 scFv nucleic acid sequence
gatgtagttatgacccagacgcctctttctctccccgtccggctcggagaccaagcctccatctcttgccgaagttcacaatcattgttgcacacgtatggatccccatatctgaattggtatctccaaaagcctggacagtcccccaagctgttgatctataaagtaagtaatagattttccggcgttcctgaccgcttcagtggctcaggaagcggtacggattttactcttcggatttcccgcgtcgaagctgaagatcttggtgtctatttctgttctcagggaacgcacctgccatacacattcggagggggcactaagctcgaaatcaagggcgggggcgggtcaggtggtgggggcagcggcgggggtggcagcgaggttaagcttgtggaaagtggaggcgggcttgtgcagccgggcgggaccatgaaactgtcctgcgagataagtggactcacttttaggaactattggatgagctgggtgcgacagtcccccgagaagggccttgaatgggttgccgaaatacggcttcgatcagacaactatgcgacgcactacgctgaaagcgtcaaaggaaaattcactatcagccgggacgacagcaagagtagactttatttgcagatgaatagtttgaggacggaagatacgggaatatattattgcaaaacatacttctattcattttcatactggggtcagggcacgttggttacggtttcagcc

SEQ ID NO: 14 - DNA, 735 bp, Artificial Sequence
  Synthetic: humanized 38C2 scFv nucleic acid sequence
gagcttcagatgacccaaagtcccagctctctctccgcctctgtcggagacagggtcacgataacctgtcgaagtagccagagtcttctccatacttacggaagcccatatcttaactggtatcttcagaaacccggtcaatcacccaagctgctgatatataaagtgtctaaccggttttctggtgtgccgagtcgattttcaggatcagggagcggcacggatttcactcttacgatctctagtttgcaacctgaggattttgctgtatacttttgcagccaaggtactcatcttccttatacgttcggagggggtaccaaagtagaaattaaaggaggaggagggtccggaggagggggcagcggaggaggaggctcagaagtacaactcgtggaatctggcggggggctggtgcaacctgggggttctctccgcctgagctgtgctgcatccggcttcaccttttctaattattggatgagctgggtacggcagtcaccggagaaaggtctggagtgggtgtctgagatacgacttagatcagacaactacgcgacgcattacgccgagagcgtgaaaggaagatttaccataagcagagacaattcaaaaaacaccctgtacctccaaatgaatagcctcagggcggaagatactgggatatattactgtaaaacctacttttacagttttagttattggggccagggaacgcttgtaactgttagctct

SEQ ID NO: 15 - DNA, 138 bp, Artificial Sequence
  Synthetic: CD8 hinge nucleic acid sequence
gctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcc

SEQ ID NO: 16 - DNA, 84 bp, Artificial Sequence
  Synthetic: CD3-zeta transmembrane domain nucleic acid sequence
ctcgatccgaagttgtgctacctgttggacggcattctctttatatacggtgtcatcctgacagcgttgtttctccgagtgaag

SEQ ID NO: 17 - DNA, 123 bp, Artificial Sequence
  Synthetic: CD28 intracellular domain nucleic acid sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 18 - DNA, 339 bp, Artificial Sequence
  Synthetic: CD3-zeta intracellular domain nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 19 - DNA, 1449 bp, Artificial Sequence
  Synthetic: Murine PUCR with Myc-tag nucleic acid sequence
gatgtagttatgacccagacgcctctttctctccccgtccggctcggagaccaagcctccatctcttgccgaagttcacaatcattgttgcacacgtatggatccccatatctgaattggtatctccaaaagcctggacagtcccccaagctgttgatctataaagtaagtaatagattttccggcgttcctgaccgcttcagtggctcaggaagcggtacggattttactcttcggatttcccgcgtcgaagctgaagatcttggtgtctatttctgttctcagggaacgcacctgccatacacattcggagggggcactaagctcgaaatcaagggcgggggcgggtcaggtggtgggggcagcggcgggggtggcagcgaggttaagcttgtggaaagtggaggcgggcttgtgcagccgggcgggaccatgaaactgtcctgcgagataagtggactcacttttaggaactattggatgagctgggtgcgacagtcccccgagaagggccttgaatgggttgccgaaatacggcttcgatcagacaactatgcgacgcactacgctgaaagcgtcaaaggaaaattcactatcagccgggacgacagcaagagtagactttatttgcagatgaatagtttgaggacggaagatacgggaatatattattgcaaaacatacttctattcattttcatactggggtcagggcacgttggttacggtttcagccgagcagaagctcatttccgaagaagatctcgcaaagccgacaacgacgccggcaccccggcctcccacccccgcccccactatagctagtcaacctctttcactgcgccctgaagcgtgtagacctgcagccgggggagcagtccatacgcgcggacttgatttcgccctcgaccccaagttgtgttaccttttggacgggatcctcttcatttacggtgtcattcttactgccttgtttctcagggtaaaaaggtctaagagatcccgactcctccattctgactacatgaatatgacaccgaggagaccgggaccaactcggaagcattatcagccatacgcgcccccccgcgatttcgcggcatacaggtcaagagtcaagttctcccgcagcgcagacgcgcccgcttatcagcaaggtcaaaatcaactctacaatgagctcaatctgggacgacgggaggagtacgatgtcctcgacaagaggagaggtcgggatcctgaaatgggtggcaaaccccagcgacgcaagaatcctcaggagggtctctacaacgagctgcaaaaagataaaatggcggaggcgtatagtgaaatagggatgaaaggggaaagacgccggggaaaaggacatgatggtctgtatcagggtctgtcaacagctactaaagacacatacgatgcgctgcacatgcaagcgttgccgccgagg

SEQ ID NO: 20 - DNA, 1449 bp, Artificial Sequence
  Synthetic: Humanized PUCR with Myc-tag nucleic acid sequence
gagcttcagatgacccaaagtcccagctctctctccgcctctgtcggagacagggtcacgataacctgtcgaagtagccagagtcttctccatacttacggaagcccatatcttaactggtatcttcagaaacccggtcaatcacccaagctgctgatatataaagtgtctaaccggttttctggtgtgccgagtcgattttcaggatcagggagcggcacggatttcactcttacgatctctagtttgcaacctgaggattttgctgtatacttttgcagccaaggtactcatcttccttatacgttcggagggggtaccaaagtagaaattaaaggaggaggagggtccggaggagggggcagcggaggaggaggctcagaagtacaactcgtggaatctggcggggggctggtgcaacctgggggttctctccgcctgagctgtgctgcatccggcttcaccttttctaattattggatgagctgggtacggcagtcaccggagaaaggtctggagtgggtgtctgagatacgacttagatcagacaactacgcgacgcattacgccgagagcgtgaaaggaagatttaccataagcagagacaattcaaaaaacaccctgtacctccaaatgaatagcctcagggcggaagatactgggatatattactgtaaaacctacttttacagttttagttattggggccagggaacgcttgtaactgttagctctgagcagaagctcatttccgaagaagatctcgcaaagccgacaacgacgccggcaccccggcctcccacccccgcccccactatagctagtcaacctctttcactgcgccctgaagcgtgtagacctgcagccgggggagcagtccatacgcgcggacttgatttcgccctcgaccccaagttgtgttaccttttggacgggatcctcttcatttacggtgtcattcttactgccttgtttctcagggtaaaaaggtctaagagatcccgactcctccattctgactacatgaatatgacaccgaggagaccgggaccaactcggaagcattatcagccatacgcgcccccccgcgatttcgcggcatacaggtcaagagtcaagttctcccgcagcgcagacgcgcccgcttatcagcaaggtcaaaatcaactctacaatgagctcaatctgggacgacgggaggagtacgatgtcctcgacaagaggagaggtcgggatcctgaaatgggtggcaaaccccagcgacgcaagaatcctcaggagggtctctacaacgagctgcaaaaagataaaatggcggaggcgtatagtgaaatagggatgaaaggggaaagacgccggggaaaaggacatgatggtctgtatcagggtctgtcaacagctactaaagacacatacgatgcgctgcacatgcaagcgttgccgccgagg

SEQ ID NO: 21 - protein, 5 aa, Artificial Sequence
  Synthetic: linker
  misc_feature (1)..(5): “Gly Gly Gly Gly Ser” can repeat n amount of times, where n is a positive integer equal to or greater than 1
GGGGS

SEQ ID NO: 22 - protein, 20 aa, Artificial Sequence
  Synthetic: linker
GGGGSGGGGSGGGGSGGGGS

SEQ ID NO: 23 - protein, 15 aa, Artificial Sequence
  Synthetic: linker
GGGGSGGGGSGGGGS

SEQ ID NO: 24 - protein, 27 aa, Homo sapiens
  misc_feature (1)..(27): transmembrane domain of human CD28
FWVLVVVGGVLACYSLLVTVAFIIFWV

SEQ ID NO: 25 - protein, 66 aa, Homo sapiens
  misc_feature (1)..(66): transmembrane domain of human CD28
IEVMYPPPYLDNEKSNGTIIHVKGKHLCPSPLFPGPSKPFWVLVVVGGVLACYSLLVTVAFIIFWV

SEQ ID NO: 26 - protein, 42 aa, Homo sapiens
  misc_feature (1)..(42): 4-1BB intracellular domain
KRGRKKLLYIFKQPFMRPVQTTQEEDGCSCRFPEEEEGGCEL

SEQ ID NO: 27 - DNA, 126 bp, Artificial Sequence
  Synthetic: 4-1BB intracellular domain nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 28 - protein, 46 aa, Artificial Sequence
  Synthetic: CD8 hinge amino acid sequence
  misc_feature (30)..(30): Xaa can be any amino acid except cysteine
AKPTTTPAPRPPTPAPTIASQPLSLRPEAXRPAAGGAVHTRGLDFA

SEQ ID NO: 29 - protein, 45 aa, Homo sapiens
  misc_feature (1)..(45): CD8 hinge
TTTPAPRPPTPAPTIASQPLSLRPEACRPAAGGAVHTRGLDFACD

SEQ ID NO: 30 - DNA, 135 bp, Artificial Sequence
  Synthetic: CD8 hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 31 - protein, 30 aa, Artificial Sequence
  Synthetic: Linker
GGGGSGGGGSGGGGSGGGGSGGGGSGGGGS

SEQ ID NO: 32 - protein, 45 aa, Artificial Sequence
  Synthetic: Linker
GGGGSGGGGSGGGGSGGGGSGGGGSGGGGSGGGGSGGGGSGGGGS

SEQ ID NO: 33 - protein, 60 aa, Artificial Sequence
  Synthetic: Linker
GGGGSGGGGSGGGGSGGGGSGGGGSGGGGSGGGGSGGGGSGGGGSGGGGSGGGGSGGGGS

SEQ ID NO: 34 - protein, 75 aa, Artificial Sequence
  Synthetic: Linker
GGGGSGGGGSGGGGSGGGGSGGGGSGGGGSGGGGSGGGGSGGGGSGGGGSGGGGSGGGGSGGGGSGGGGSGGGGS

SEQ ID NO: 35 - protein, 150 aa, Artificial Sequence
  Synthetic: Linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 36 - protein, 225 aa, Artificial Sequence
  Synthetic: Linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 37 - protein, 300 aa, Artificial Sequence
  Synthetic: Linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 38 - protein, 21 aa, Artificial Sequence
  Synthetic: hydrophobic stretch of the CD3-zeta transmembrane domain sequence
LCYLLDGILFIYGVILTALFL

SEQ ID NO: 39 - DNA, 30 bp, Artificial Sequence
  Synthetic: Myc-tag nucleic acid sequence
gagcagaagctgattagcgaagaggacctg

SEQ ID NO: 40 - protein, 112 aa, Artificial Sequence
  Synthetic: Humanized 38C2 variable kappa heavy chain
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIK

SEQ ID NO: 41 - protein, 107 aa, Homo sapiens [sequence omitted]

SEQ ID NO: 42 - protein, 118 aa, Artificial Sequence
  Synthetic: Humanized 38C2 variable heavy chain
EVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSS

SEQ ID NO: 43 - protein, 108 aa, Homo sapiens
  misc_feature (1)..(108): Human gamma 1 heavy chain constant domain 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 44 - protein, 505 aa, Artificial Sequence
  Synthetic: Full length humanized 38C2 scFab with signal peptide
MEWSWVFLFFLSVTTGVHSELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGGSGGGSGGGGSGGGSGGGGSGGGGSGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 45 - protein, 773 aa, Artificial Sequence
  Synthetic: Full length PUCR comprising 38C2 scFab amino acid sequence
MEWSWVFLFFLSVTTGVHSELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGGSGGGSGGGGSGGGSGGGGSGGGGSGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 54 bp, Artificial Sequence
  Synthetic: Signal peptide nucleic acid sequence
atggaatggagttgggtgttccttttctttctgagtgtcaccaccggagtgcac

SEQ ID NO: 47 - DNA, 1461 bp, Artificial Sequence
  Synthetic: humanized 38C2 scFab nucleic acid sequence
agcgaactgcagatgacccagtccccatccagtctgagcgctagcgttggtgacagagttactatcacctgccgctcttcacagagcctgttgcacacttacggctctccttacctgaactggtatcttcagaagcctggccaaagccctaagctgctcatctacaaggtgtctaacaggttctccggggttccgtcccgcttttcagggagcgggtcaggaacagacttcaccttgacaatctcaagcctccagcccgaggattttgccgtctatttctgctcacaaggcacacatctgccgtatacctttgggggcgggacaaaagtcgagatcaaaaggaccgtcgctgcaccatccgtgtttatcttcccaccaagtgacgaacagctcaagagcggtactgcctccgttgtttgtctgctgaacaacttctatccaagggaagcaaaggtgcaatggaaagtagacaacgctctgcagtcaggcaactcccaggagtcagtgaccgagcaggatagcaaagattcaacatacagcctgagcagcaccctcaccctgagtaaggccgattacgagaagcacaaggtttacgcctgcgaggtgacccaccagggcctttcatccccagtcaccaaatcttttaaccgcggcgaatgcgggggaggctctggtggaggcggttctggagggggctcaggaggaggcggtagcggcggtggtagtgggggtggcggatctggcggaggtggctcaggaggaggtagcggcggcgggggcagcgaggtccagctggtagagtcaggtggaggattggtgcagcccggcggcagtcttagactcagctgtgcggccagcggatttactttctcaaattattggatgtcttgggtcaggcagagcccagagaaaggcctggaatgggtgtcagagatccgactgagaagcgataattacgcgactcattatgcggaaagcgttaaaggtcggttcactatttcacgagataattctaagaataccctttatctgcagatgaacagcttgcgcgccgaggacacaggcatctactactgtaaaacttacttctattctttttcctactggggacaggggactctcgttacagtcagtagcgcctccaccaagggtcctagtgtctttcccctggccccctcatccaagtccacgtcaggaggcaccgcggctctgggctgtctggtcaaagactactttcctgagccagtcaccgtgtcctggaattccggcgcgcttacttctggcgtgcacactttccccgccgtcctccagagcagtgggctgtattccctgtcttccgtagtcactgtgccaagctccagtctgggaacccagacctatatttgtaatgtgaatcataagccgagcaacaccaaggtggacaagaaggtggaaccgaagtcatgtgacaaaacccacact

SEQ ID NO: 48 - DNA, 2322 bp, Artificial Sequence
  Synthetic: Full length PUCR comprising 38C2 scFab nucleic acid sequence
atggaatggagttgggtgttccttttctttctgagtgtcaccaccggagtgcacagcgaactgcagatgacccagtccccatccagtctgagcgctagcgttggtgacagagttactatcacctgccgctcttcacagagcctgttgcacacttacggctctccttacctgaactggtatcttcagaagcctggccaaagccctaagctgctcatctacaaggtgtctaacaggttctccggggttccgtcccgcttttcagggagcgggtcaggaacagacttcaccttgacaatctcaagcctccagcccgaggattttgccgtctatttctgctcacaaggcacacatctgccgtatacctttgggggcgggacaaaagtcgagatcaaaaggaccgtcgctgcaccatccgtgtttatcttcccaccaagtgacgaacagctcaagagcggtactgcctccgttgtttgtctgctgaacaacttctatccaagggaagcaaaggtgcaatggaaagtagacaacgctctgcagtcaggcaactcccaggagtcagtgaccgagcaggatagcaaagattcaacatacagcctgagcagcaccctcaccctgagtaaggccgattacgagaagcacaaggtttacgcctgcgaggtgacccaccagggcctttcatccccagtcaccaaatcttttaaccgcggcgaatgcgggggaggctctggtggaggcggttctggagggggctcaggaggaggcggtagcggcggtggtagtgggggtggcggatctggcggaggtggctcaggaggaggtagcggcggcgggggcagcgaggtccagctggtagagtcaggtggaggattggtgcagcccggcggcagtcttagactcagctgtgcggccagcggatttactttctcaaattattggatgtcttgggtcaggcagagcccagagaaaggcctggaatgggtgtcagagatccgactgagaagcgataattacgcgactcattatgcggaaagcgttaaaggtcggttcactatttcacgagataattctaagaataccctttatctgcagatgaacagcttgcgcgccgaggacacaggcatctactactgtaaaacttacttctattctttttcctactggggacaggggactctcgttacagtcagtagcgcctccaccaagggtcctagtgtctttcccctggccccctcatccaagtccacgtcaggaggcaccgcggctctgggctgtctggtcaaagactactttcctgagccagtcaccgtgtcctggaattccggcgcgcttacttctggcgtgcacactttccccgccgtcctccagagcagtgggctgtattccctgtcttccgtagtcactgtgccaagctccagtctgggaacccagacctatatttgtaatgtgaatcataagccgagcaacaccaaggtggacaagaaggtggaaccgaagtcatgtgacaaaacccacactgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - protein, 124 aa, Artificial Sequence
  Synthetic: Anti-PSMA Clone A11 Heavy Chain Variable Domain
QVQLVQSGGGLVQPGGSLRLSCAASGFTFSSYWMSWVRQAPGKGLEWVANIKQDGSEKYYVDSVKGRFTISRDNAKNSLYLQMNSLRAEDTAVYYCARVWDYYYDSSGDAFDIWGQGTMVTVSS

SEQ ID NO: 50 - protein, 107 aa, Artificial Sequence
  Synthetic: Anti-PSMA Clone A11 Light Chain Variable Domain
VIWMTQSPSSVSASVGDRVTITCRASQGISSWLAWYQQKPGKAPKLLIYAASNLQSGVPSRFSGSGSGTDFTLTISSLQPEDFATYYCQQANSFPLTFGGGTKVDIK

SEQ ID NO: 51 - protein, 380 aa, Homo sapiens
  misc_feature (1)..(380): IL13R amino acid sequence
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 52 - protein, 107 aa, Artificial Sequence
  Synthetic: Anti-VEGFR2 VK-B8 Light Chain Variable Domain
ETTLTQSPATLSVSPGERATVSCRASQSLGSNLGWFQQKPGQAPRLLIYGASTRATGIPARFSGSGSGTEFTLTISSLQSEDFAVYFCQQYNDWPITFGQGTRLEIK

SEQ ID NO: 53 - protein, 126 aa, Artificial Sequence
  Synthetic: Anti-VEGFR2 VK-B8 Heavy Chain Variable Domain
MAQVQLVQSGAEVKKPGSSVKVSCKAYGGTFGSYGVSWVRRAPGQGLEWMGRLIPIFGTRDYAQKFQGRVTLTADESTNTAYMELSSLRSEDTAVYYCARDGDYYGSGSYYGMDVWGQGTLVTVSS

SEQ ID NO: 54 - protein, 41 aa, Artificial Sequence
  Synthetic: PolyGlySer linker
GGGSGGGGSGGGSGGGGSGGGSGGGGSGGGGSGGGSGGGGS

SEQ ID NO: 55 - protein, 88 aa, Artificial Sequence
  Synthetic: Hybrid CD8 and CD28 hinge amino acid sequence
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 56 - protein, 46 aa, Artificial Sequence; identical to SEQ ID NO: 5
  Synthetic: CD8 portion of hybrid CD8 and CD28 hinge amino acid sequence

SEQ ID NO: 57 - protein, 2 aa, Artificial Sequence
  Synthetic: Hinge linker amino acid sequence
PR

SEQ ID NO: 58 - protein, 40 aa, Artificial Sequence
  Synthetic: CD28 portion of hybrid CD8 and CD28 hinge amino acid sequence
KIEVMYPPPYLDNEKSNGTIIHVKGKHLCPSPLFPGPSKP

SEQ ID NO: 59 - protein, 112 aa, Artificial Sequence
  Synthetic: CD3-zeta intracellular domain amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 264 bp, Artificial Sequence
  Synthetic: Hybrid CD8 and CD28 hinge nucleic acid sequence
gctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagccc

SEQ ID NO: 61 - DNA, 81 bp, Artificial Sequence
  Synthetic: CD28 transmembrane domain nucleic acid sequence
ttttgggtgctggtggtggttggtggagtcctggcttgctatagcttgctagtaacagtggcctttattattttctgggtg

SEQ ID NO: 62 - DNA, 339 bp, Artificial Sequence; identical to SEQ ID NO: 18
  Synthetic: CD3 intracellular domain nucleic acid sequence

SEQ ID NO: 63 - DNA, 30 bp, Artificial Sequence; identical to SEQ ID NO: 39
  Synthetic: Myc-tag nucleic acid sequence

SEQ ID NO: 64 - protein, 14 aa, Artificial Sequence
  Synthetic peptide
  misc_feature (3)..(14): Cyclic
AGCKNFFWKTFTSC

SEQ ID NO: 65 - protein, 8 aa, Artificial Sequence
  Synthetic peptide
  MOD_RES (1)..(1): D-amino acid
  misc_feature (2)..(7): Cyclic
  MOD_RES (4)..(4): D-amino acid
  MOD_RES (8)..(8): Thr(ol)
FCFWKTCT

SEQ ID NO: 66 - protein, 8 aa, Artificial Sequence
  Synthetic peptide
  MOD_RES (1)..(1): D-amino acid
  misc_feature (2)..(7): Cyclic
  MOD_RES (4)..(4): D-amino acid
  MOD_RES (8)..(8): Thr(ol)
FCYWKTCT

SEQ ID NO: 67 - protein, 8 aa, Artificial Sequence
  Synthetic peptide
  MOD_RES (1)..(1): D-amino acid
  misc_feature (2)..(7): Cyclic
  MOD_RES (4)..(4): D-amino acid
FCYWKTCT

SEQ ID NO: 68 - protein, 8 aa, Artificial Sequence
  Synthetic peptide
  MOD_RES (1)..(1): D-amino acid
  misc_feature (2)..(7): Cyclic
  MOD_RES (3)..(3): 1-Nal
  MOD_RES (4)..(4): D-amino acid
  MOD_RES (8)..(8): Thr(ol)
FCAWKTCT

SEQ ID NO: 69 - protein, 8 aa, Artificial Sequence
  Synthetic peptide
  MOD_RES (1)..(1): D-amino acid
  misc_feature (2)..(7): Cyclic
  MOD_RES (3)..(3): 1-Nal
  MOD_RES (4)..(4): D-amino acid
FCAWKTCT

SEQ ID NO: 70 - protein, 8 aa, Artificial Sequence
  Synthetic peptide
  MOD_RES (1)..(1): D-amino acid
  misc_feature (2)..(7): Cyclic
  MOD_RES (3)..(3): BzThi
  MOD_RES (4)..(4): D-amino acid
  MOD_RES (8)..(8): Thr(ol)
FCXWKTCT

SEQ ID NO: 71 - protein, 8 aa, Artificial Sequence
  Synthetic peptide
  MOD_RES (1)..(1): D-amino acid
  misc_feature (2)..(7): Cyclic
  MOD_RES (3)..(3): BzThi
  MOD_RES (4)..(4): D-amino acid
FCXWKTCT

SEQ ID NO: 72 - protein, 9 aa, Artificial Sequence
  Synthetic peptide
  MOD_RES (2)..(2): DAB
  misc_feature (2)..(9): Cyclic
  MOD_RES (6)..(6): D-amino acid
YXRFFWKTF

SEQ ID NO: 73 - protein, 8 aa, Artificial Sequence
  Synthetic peptide
  misc_feature: C-term NH2
  MOD_RES (1)..(1): p-Cl-Phe
  MOD_RES (2)..(2): D-amino acid
  misc_feature (2)..(7): Cyclic
  MOD_RES (4)..(4): D-Aph(Cbm)
  MOD_RES (8)..(8): D-amino acid
FCYXKTCY

SEQ ID NO: 74 - protein, 14 aa, Artificial Sequence
  Synthetic peptide
  misc_feature: C-term NH2
  MOD_RES (1)..(1): pGlu
EQRLGNQWAVGHLM

SEQ ID NO: 75 - protein, 13 aa, Artificial Sequence
  Synthetic peptide
  misc_feature: C-term NH2
  MOD_RES (1)..(1): N,N-dimethyl-Gly
  misc_feature (1)..(3): N3S
  MOD_RES (3)..(3): Cys(acm)
  MOD_RES (5)..(5): Ava
GSCGXQWAVGHLM

SEQ ID NO: 76 - protein, 8 aa, Artificial Sequence
  Synthetic peptide
  misc_feature: N-term N40-1-bzlg0
  misc_feature: C-term NHEt13
  MOD_RES (1)..(1): D-amino acid
FQWAVGHL

SEQ ID NO: 77 - protein, 14 aa, Artificial Sequence
  Synthetic peptide
  misc_feature: N-term N4
  misc_feature: C-term NH2
  MOD_RES (14)..(14): Nle
PQRYGNQWAVGHLL

SEQ ID NO: 78 - protein, 11 aa, Artificial Sequence
  Synthetic peptide
  misc_feature: C-term NH2
  MOD_RES (1)..(1): (N-alphaHis)Ac
  MOD_RES (2)..(3): Beta-Ala
  MOD_RES (10)..(10): Cha
  MOD_RES (11)..(11): Nle
HAAQWAVGHAL

SEQ ID NO: 79 - protein, 11 aa, Artificial Sequence
  Synthetic peptide
  misc_feature: N-term 3-cyano-4-trimethylammonium-benzoyl
  misc_feature: C-term NH2
  MOD_RES (1)..(2): Ala(SO3H)
  MOD_RES (3)..(3): Ava
  MOD_RES (8)..(8): NMeGly
  MOD_RES (10)..(10): Sta
AAXQWAVGHXL

SEQ ID NO: 80 - protein, 13 aa, Artificial Sequence
  Synthetic peptide
  misc_feature: C-term NH2
LEEEEEAYGWMDF

SEQ ID NO: 81 - protein, 13 aa, Artificial Sequence
  Synthetic peptide
  misc_feature: C-term NH2
  MOD_RES (1)..(1): D-amino acid
EEEEEEAYGWMDF

SEQ ID NO: 82 - protein, 8 aa, Artificial Sequence
  Synthetic peptide
  misc_feature: C-term NH2
  MOD_RES (1)..(1): D-amino acid
EAYGWMDF

SEQ ID NO: 83 - protein, 10 aa, Artificial Sequence
  Synthetic peptide
  misc_feature: C-term NH2
HHEAYGWMDF

SEQ ID NO: 84 - protein, 10 aa, Artificial Sequence
  Synthetic peptide
  misc_feature: C-term NH2
  MOD_RES (8)..(8): Nle
HHEAYGWLDF

SEQ ID NO: 85 - protein, 13 aa, Artificial Sequence
  Synthetic peptide
  misc_feature: N-term N4
  misc_feature: C-term NH2
  MOD_RES (1)..(1): D-amino acid
EEEEEEAYGWMDF

SEQ ID NO: 86 - protein, 8 aa, Artificial Sequence
  Synthetic peptide
  misc_feature: C-term NH2
  MOD_RES (1)..(1): gamma-D-Glu
  misc_feature (1)..(4): Cyclic
  MOD_RES (4)..(4): D-amino acid
EAYKWMDF

SEQ ID NO: 87 - protein, 11 aa, Artificial Sequence
  Synthetic peptide
  misc_feature: C-term NH2
  misc_feature: see specification as filed for detailed description of substitutions and preferred embodiments
  misc_feature (3)..(3): Cys modified with a unique side chain
  MOD_RES (9)..(9): Nle
GSCEAYGWLDF

SEQ ID NO: 88 - protein, 9 aa, Artificial Sequence
  Synthetic peptide
  misc_feature: C-term NHC2H5
  MOD_RES (1)..(1): pGlu
  MOD_RES (6)..(6): D-Ser(tBu)
EHWSYSLRP

SEQ ID NO: 89 - protein, 10 aa, Artificial Sequence
  Synthetic peptide
  misc_feature: C-term NH2
  MOD_RES (1)..(1): pGlu
  MOD_RES (6)..(6): D-Ser(tBu)
  MOD_RES (10)..(10): AzGly
EHWSYSLRPG

SEQ ID NO: 90 - protein, 9 aa, Artificial Sequence
  Synthetic peptide
  misc_feature: C-term NHC2H5
  MOD_RES (1)..(1): pGlu
  MOD_RES (6)..(6): D-amino acid
EHWSYLLRP

SEQ ID NO: 91 - protein, 9 aa, Artificial Sequence
  Synthetic peptide
  misc_feature: C-term NHC2H5
  MOD_RES (1)..(1): pGlu
  MOD_RES (6)..(6): D-Nal(2)
EHWSYALRP

SEQ ID NO: 92 - protein, 10 aa, Artificial Sequence
  Synthetic peptide
  misc_feature: C-term NH2
  MOD_RES (1)..(1): pGlu
  MOD_RES (6)..(6): D-amino acid
EHWSYWLRPG

SEQ ID NO: 93 - protein, 10 aa, Artificial Sequence
  Synthetic peptide
  misc_feature: N-term Ac
  misc_feature: C-term NH2
  MOD_RES (1)..(1): D-amino acid
  MOD_RES (2)..(2): D-Cpa
  MOD_RES (3)..(3): D-amino acid
  MOD_RES (6)..(6): D-amino acid
  MOD_RES (8)..(8): I-Lys
  MOD_RES (10)..(10): D-amino acid
AXASYDLKPA

SEQ ID NO: 94 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (5)..(5): Aph(Ac)
  MOD_RES (6)..(6): D-Aph(Ac)
  MOD_RES (8)..(8): I-Lys
  MOD_RES (10)..(10): D-amino acid
AXXSFFLKPA

SEQ ID NO: 95 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (6)..(6): D-Hci
  MOD_RES (8)..(8): I-Lys
  MOD_RES (10)..(10): D-amino acid
AXXSYXLKPA

SEQ ID NO: 96 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (5)..(5): Lys(Nic)
  MOD_RES (6)..(6): D-Lys(Nic)
  MOD_RES (8)..(8): I-Lys
  MOD_RES (10)..(10): D-amino acid
AXXSKKLKPA

SEQ ID NO: 97 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (5)..(5): Aph(Atz)
  MOD_RES (6)..(6): D-Aph(Atz)
  MOD_RES (8)..(8): I-Lys
  MOD_RES (10)..(10): D-amino acid
AXXSFFLKPA

SEQ ID NO: 98 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (6)..(6): D-Cit
  MOD_RES (10)..(10): D-amino acid
AXXSYXLRPA

SEQ ID NO: 99 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (5)..(5): Aph(L-hydroorotyl)
  MOD_RES (6)..(6): D-Aph(carbamoyl)
  MOD_RES (8)..(8): I-Lys
  MOD_RES (10)..(10): D-amino acid
AXXSFFLKPA

SEQ ID NO: 100 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (6)..(6): D-hArg(Et2)
  MOD_RES (8)..(8): hArg(Et2)
  MOD_RES (10)..(10): D-amino acid
AXXSYRLRPA

SEQ ID NO: 101 - protein, 10 aa, Artificial Sequence
  Synthetic peptide
  misc_feature: N-term Ac
  misc_feature: C-term NH2
  MOD_RES (1)..(1): D-Nal
  MOD_RES (2)..(2): D-Cpa
  MOD_RES (3)..(3): D-Pal
  MOD_RES (5)..(5): N-MeTyr
  MOD_RES (6)..(6): D-hCit
  MOD_RES (7)..(7): Nle
  MOD_RES (10)..(10): D-amino acid
AXXSYXLRPA

SEQ ID NO: 102 - protein, 483 aa, Artificial Sequence
  Synthetic: Murine PUCR with Myc-tag without signal peptide amino acid sequence
DVVMTQTPLSLPVRLGDQASISCRSSQSLLHTYGSPYLNWYLQKPGQSPKLLIYKVSNRFSGVPDRFSGSGSGTDFTLRISRVEAEDLGVYFCSQGTHLPYTFGGGTKLEIKGGGGSGGGGSGGGGSEVKLVESGGGLVQPGGTMKLSCEISGLTFRNYWMSWVRQSPEKGLEWVAEIRLRSDNYATHYAESVKGKFTISRDDSKSRLYLQMNSLRTEDTGIYYCKTYFYSFSYWGQGTLVTVSAEQKLISEEDLAKPTTTPAPRPPTPAPTIASQPLSLRPEACRPAAGGAVHTRGLDFALDPKLCYLLDGILFIYGVILTALFLRVKRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 103 - protein, 483 aa, Artificial Sequence
  Synthetic: Humanized PUCR with Myc-tag without signal peptide amino acid sequence
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKGGGGSGGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSEQKLISEEDLAKPTTTPAPRPPTPAPTIASQPLSLRPEACRPAAGGAVHTRGLDFALDPKLCYLLDGILFIYGVILTALFLRVKRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 104 - protein, 486 aa, Artificial Sequence
  Synthetic: Full length humanized 38C2 scFab without signal peptide amino acid sequence
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGGSGGGSGGGGSGGGSGGGGSGGGGSGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 105 - protein, 754 aa, Artificial Sequence
  Synthetic: Full length PUCR comprising 38C2 scFab without signal peptide amino acid sequence
ELQMTQSPSSLSASVGDRVTITCRSSQSLLHTYGSPYLNWYLQKPGQSPKLLIYKVSNRFSGVPSRFSGSGSGTDFTLTISSLQPEDFAVYFCSQGTHLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGGSGGGSGGGGSGGGSGGGGSGGGGSGGGSGGGGSEVQLVESGGGLVQPGGSLRLSCAASGFTFSNYWMSWVRQSPEKGLEWVSEIRLRSDNYATHYAESVKGRFTISRDNSKNTLYLQMNSLRAEDTGIYYCKTYFYSFSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - DNA, 2268 bp, Artificial Sequence
  Synthetic: Full length PUCR comprising 38C2 scFab without signal peptide nucleic acid sequence
agcgaactgcagatgacccagtccccatccagtctgagcgctagcgttggtgacagagttactatcacctgccgctcttcacagagcctgttgcacacttacggctctccttacctgaactggtatcttcagaagcctggccaaagccctaagctgctcatctacaaggtgtctaacaggttctccggggttccgtcccgcttttcagggagcgggtcaggaacagacttcaccttgacaatctcaagcctccagcccgaggattttgccgtctatttctgctcacaaggcacacatctgccgtatacctttgggggcgggacaaaagtcgagatcaaaaggaccgtcgctgcaccatccgtgtttatcttcccaccaagtgacgaacagctcaagagcggtactgcctccgttgtttgtctgctgaacaacttctatccaagggaagcaaaggtgcaatggaaagtagacaacgctctgcagtcaggcaactcccaggagtcagtgaccgagcaggatagcaaagattcaacatacagcctgagcagcaccctcaccctgagtaaggccgattacgagaagcacaaggtttacgcctgcgaggtgacccaccagggcctttcatccccagtcaccaaatcttttaaccgcggcgaatgcgggggaggctctggtggaggcggttctggagggggctcaggaggaggcggtagcggcggtggtagtgggggtggcggatctggcggaggtggctcaggaggaggtagcggcggcgggggcagcgaggtccagctggtagagtcaggtggaggattggtgcagcccggcggcagtcttagactcagctgtgcggccagcggatttactttctcaaattattggatgtcttgggtcaggcagagcccagagaaaggcctggaatgggtgtcagagatccgactgagaagcgataattacgcgactcattatgcggaaagcgttaaaggtcggttcactatttcacgagataattctaagaataccctttatctgcagatgaacagcttgcgcgccgaggacacaggcatctactactgtaaaacttacttctattctttttcctactggggacaggggactctcgttacagtcagtagcgcctccaccaagggtcctagtgtctttcccctggccccctcatccaagtccacgtcaggaggcaccgcggctctgggctgtctggtcaaagactactttcctgagccagtcaccgtgtcctggaattccggcgcgcttacttctggcgtgcacactttccccgccgtcctccagagcagtgggctgtattccctgtcttccgtagtcactgtgccaagctccagtctgggaacccagacctatatttgtaatgtgaatcataagccgagcaacaccaaggtggacaagaaggtggaaccgaagtcatgtgacaaaacccacactgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2017/0112878 A1
(43) Pub. Date: Apr. 27, 2017

(54) PROGRAMMABLE UNIVERSAL CELL RECEPTORS AND METHOD OF USING THE SAME

(71) Applicant: Sorrento Therapeutics, Inc., San Diego, CA (US)

(72) Inventors: Gunnar Jörg Floris Kaufmann, San Diego, CA (US); Yanwen Fu, San Diego, CA (US); Yan-liang Zhang, San Diego, CA (US); James T. Patterson, San Diego, CA (US)

(21) Appl. No.: 15/333,115

(22) Filed: Oct. 24, 2016

Related U.S. Application Data
(60) Provisional application No. 62/382,691, filed on Sep. 1, 2016.
(60) Provisional application No. 62/245,978, filed on Oct. 23, 2015.

(51) Int. Cl. A61K 35/17; C12N 9/00; C07K 14/705; C12N 15/85; C07K 14/725

(52) U.S. Cl. CPC A61K 35/17; C12N 15/85; C07K 14/7051; C07K 14/70521; C07K 14/70517; C12N 9/0002; C07K 2319/03; C07K 2319/70; A61K 2035/124

(57) ABSTRACT

The present invention provides programmable universal cell receptors (PUCRs) comprising a catalytic antibody region, a transmembrane domain and a cytoplasmic domain. The PUCRs disclosed herein may be conjugated to a specificity agent in order to program the receptor for specificity to any molecule of interest. Also provided are nucleic acids encoding such PUCRs, and cells expressing the PUCRs. Such cells may be used in treating a variety of medical conditions and diseases including cancer and infectious diseases.

### RELATED APPLICATIONS

This application claims priority to U.S. Provisional Patent Application No. 62/245,978, filed on Oct. 23, 2015, and to U.S. Provisional Patent Application No. 62/382,691, filed Sep. 1, 2016, the entire contents of each of which are expressly incorporated herein by reference.

### SEQUENCE LISTING

The instant application contains a Sequence Listing which has been submitted electronically in ASCII format and is hereby incorporated by reference in its entirety. Said ASCII copy, created on Oct. 24, 2016, is named 126591-00103_ST25.txt and is 112 kilobytes in size.

### BACKGROUND OF THE INVENTION

The limited availability of effective treatments for complex diseases, such as cancer and infectious diseases, is a global health concern. Conventionally-developed pharmaceutical drugs and biological effector molecules for treating complex diseases are often of limited use due to high toxicity. For example, cancer treatments involving chemotherapy are often non-specific and result in non-desirable side effects.

In recent developments, cell-based therapies have been developed which utilize a patient's own cells (e.g., immune cells) to attack a diseased cell (e.g., a cancer cell), or a disease-causing organism. Efforts to develop specific cell-based therapies are impeded by our technical inability to rapidly develop personalized cell-based therapies to target specific diseased cell populations or disease-causing organisms in a subject. The problem is further exacerbated by the heterogeneous antigen profile of complex diseases, such as cancer.

Accordingly, there remains a need for improved customized cell-based therapies that can be used for treating complex diseases.

### SUMMARY OF THE INVENTION

The present invention provides nucleic acids, host cells, pharmaceutical compositions thereof, kits thereof, and methods of using the compositions disclosed herein.

In one aspect, the invention provides an isolated nucleic acid sequence encoding a programmable universal cell receptor (also referred to herein as a PUCR), wherein said programmable universal cell receptor comprises a catalytic antibody, or a catalytic portion thereof, comprising a reactive amino acid residue; a transmembrane domain; and an intracellular domain.

In some embodiments, the catalytic antibody, or a catalytic portion thereof, is selected from the group consisting of an aldolase catalytic antibody, a beta lactamase catalytic antibody, an amidase catalytic antibody, a thioesterase catalytic antibody, and catalytic portions thereof. In some embodiments, the catalytic antibody, or a catalytic portion thereof, is an aldolase catalytic antibody, or a catalytic portion thereof.

In some embodiments, the reactive amino acid residue of the catalytic antibody or a catalytic portion thereof, is selected from the group consisting of a reactive cysteine residue, a reactive tyrosine residue, a reactive lysine residue, and a reactive tyrosine residue. In some embodiments, the reactive amino acid residue is a reactive lysine residue.

In some embodiments, the catalytic antibody, or a catalytic portion thereof, is a humanized monoclonal antibody 38C2, or a catalytic portion thereof. In some embodiments, the catalytic antibody, or a catalytic portion thereof, comprises the amino acid sequence of SEQ ID NO: 4, or a catalytic portion thereof. In some embodiments, the catalytic antibody, or a catalytic portion thereof, comprises the amino acid sequence of SEQ ID NO: 3, or a catalytic portion thereof. In some embodiments, the catalytic antibody, or a catalytic portion thereof, comprises the amino acid sequence of SEQ ID NO: 40. In some embodiments, the catalytic antibody, or a catalytic portion thereof, comprises the amino acid sequence of SEQ ID NO: 41. In some embodiments, the catalytic antibody, or a catalytic portion thereof, comprises the amino acid sequence of SEQ ID NO: 42. In some embodiments, the catalytic antibody, or a catalytic portion thereof, comprises the amino acid sequence of SEQ ID NO: 43. In some embodiments, the catalytic antibody, or a catalytic portion thereof, comprises the amino acid sequence of SEQ ID NO: 44. In some embodiments, the catalytic antibody, or a catalytic portion thereof, comprises the amino acid sequence of SEQ ID NO: 104. In some embodiments, the catalytic antibody, or a catalytic portion thereof, comprises the amino acid sequence of SEQ ID NO: 44. In some embodiments, the catalytic antibody, or a catalytic portion thereof, is encoded by the nucleic acid sequence of SEQ ID NO: 13. In some embodiments, the catalytic antibody, or a catalytic portion thereof, is encoded by the nucleic acid sequence of SEQ ID NO: 14. In some embodiments, the catalytic antibody, or a catalytic portion thereof, is encoded by the nucleic acid sequence of SEQ ID NO: 47. In some embodiments, the catalytic antibody, or a catalytic portion thereof, is a humanized monoclonal antibody 33F12, or a catalytic portion thereof. In some embodiments, the catalytic antibody, or a catalytic portion thereof, is murine monoclonal antibody 38C2 or 33F12, or a catalytic portion thereof.

In some embodiments, the catalytic portion is a single chain variable fragment (scFv). In some embodiments, the catalytic portion is a Fab fragment. In some embodiments, the catalytic portion is a scFab. In further embodiments, the catalytic portion is selected from the group consisting of a scFab, a diabody, a F(ab′)₂ fragment, a Fd fragment consisting of the VH and CH1 domains, and a dAb fragment.

In some embodiments, the intracellular domain comprises a signaling domain. In some embodiments, the signaling domain is a CD3-ζ signaling domain. In some embodiments, the CD3-ζ signaling domain comprises the amino acid sequence of SEQ ID NO: 8. In some embodiments, the CD3-ζ signaling domain comprises the amino acid sequence of SEQ ID NO: 59. In some embodiments, the CD3-ζ signaling domain is encoded by the nucleic acid sequence of SEQ ID NO: 18. In some embodiments, the CD3-ζ signaling domain is encoded by the nucleic acid sequence of SEQ ID NO: 62. In other embodiments, the signaling domain is a CD28 signaling domain. In some embodiments, the CD28 signaling domain comprises the amino acid sequence of SEQ ID NO: 7. In some embodiments, the CD28 signaling domain is encoded by the nucleic acid sequence of SEQ ID NO: 17.

In some embodiments, the intracellular domain comprises a co-stimulatory signaling domain. In some embodiments, the co-stimulatory signaling domain comprises an intracellular domain of a protein selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand, and any combination thereof.

In some embodiments, the transmembrane domain comprises the transmembrane domain of a protein selected from the group consisting of: the alpha chain of the T-cell receptor, the beta chain of the T-cell receptor, the zeta chain of the T-cell receptor, CD28, CD3 epsilon, CD45, CD4, CD5, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and fragments thereof. In some embodiments, the transmembrane domain is a CD3-ζ transmembrane domain. In some embodiments, the CD3-ζ transmembrane domain comprises the amino acid sequence of SEQ ID NO: 6. In some embodiments, the CD3-ζ transmembrane domain is encoded by the nucleic acid sequence of SEQ ID NO: 16. In some embodiments, the transmembrane domain is a CD28 transmembrane domain. In some embodiments, the CD28 transmembrane domain comprises the amino acid sequence of SEQ ID NO: 24. In some embodiments, the CD28 transmembrane domain is encoded by the nucleic acid sequence of SEQ ID NO: 61.

In even further embodiments, the programmable universal cell receptor further comprises a hinge region. In some embodiments, the hinge region is a CD8 hinge region. In some embodiments, the CD8 hinge region comprises the amino acid sequence of SEQ ID NO: 5. In some embodiments, the hinge region is a hybrid CD8 and CD28 hinge region. In some embodiments, the hinge region comprises the amino acid sequence of SEQ ID NO: 55. In In some embodiments, the hinge region comprises the amino acid sequence of SEQ ID NO: 56. In some embodiments, the hinge region comprises the amino acid sequence of SEQ ID NO: 57. In some embodiments, the hinge region comprises the amino acid sequence of SEQ ID NO: 58. In some embodiments, the hinge region is encoded by the nucleic acid sequence of SEQ ID NO: 15. In some embodiments, the hinge region is encoded by the nucleic acid sequence of SEQ ID NO: 60.

In some embodiments, the programmable universal cell receptor further comprises a detectable moiety. In some embodiments, the detectable moiety is a polypeptide. In some embodiments, the detectable moiety is selected from the group consisting of a GST-tag, a HIS-tag, a myc-tag, and a HA-tag. In some embodiments, the myc-tag comprises the amino acid sequence of SEQ ID NO: 2. In some embodiments, the myc-tag is encoded by the nucleic acid sequence of SEQ ID NO: 12. In some embodiments, the myc-tag is encoded by the nucleic acid sequence of SEQ ID NO: 39.

In one aspect, the present invention provides an isolated nucleic acid sequence encoding a programmable universal cell receptor, wherein the programmable universal cell receptor comprises an amino acid sequence as set forth in SEQ ID NO: 10. Also provided is an isolated nucleic acid sequence encoding a programmable universal cell receptor, wherein the programmable universal cell receptor comprises an amino acid sequence as set forth in SEQ ID NO: 9. Also provided is an isolated nucleic acid sequence encoding a programmable universal cell receptor, wherein the programmable universal cell receptor comprises an amino acid sequence as set forth in SEQ ID NO: 102. Also provided is an isolated nucleic acid sequence encoding a programmable universal cell receptor, wherein the programmable universal cell receptor comprises an amino acid sequence as set forth in SEQ ID NO: 103. Also provided is an isolated nucleic acid sequence encoding a programmable universal cell receptor, wherein the programmable universal cell receptor comprises an amino acid sequence as set forth in SEQ ID NO: 105. Also provided is an isolated nucleic acid sequence encoding a programmable universal cell receptor, wherein the programmable universal cell receptor comprises an amino acid sequence as set forth in SEQ ID NO: 45. In some embodiments, the nucleic acid sequence encoding a programmable universal cell receptor comprises the nucleic acid sequence of SEQ ID NO: 19. In some embodiments, the nucleic acid sequence encoding a programmable universal cell receptor comprises the nucleic acid sequence of SEQ ID NO: 20. In some embodiments, the nucleic acid sequence encoding a programmable universal cell receptor comprises the nucleic acid sequence of SEQ ID NO: 48. In some embodiments, the nucleic acid sequence encoding a programmable universal cell receptor comprises the nucleic acid sequence of SEQ ID NO: 106.

In another aspect, the present invention provides a vector comprising a nucleic acid sequence disclosed herein. In some embodiments, the vector is a viral vector. In some embodiments, the viral vector is selected from the group consisting of a retroviral vector, a lentiviral vector, an adenovirus vector, and an adeno-associated virus vector. In some embodiments, the viral vector is a murine leukemia virus (MLV)-based retroviral vector. In some embodiments, the viral vector is a Moloney murine leukemia virus (MoMuLV)-based retroviral vector.

In one aspect, the present invention provides an isolated host cell comprising the isolated nucleic acids disclosed herein.

In some embodiments, the programmable universal cell receptor provided herein is conjugated to a specificity agent via a reactive moiety, wherein the reactive moiety is bound to the reactive amino acid residue of the catalytic antibody, or catalytic portion thereof. In some embodiments, the programmable universal cell receptor is covalently bound to the specificity agent via the reactive moiety. In some embodiments, the reactive moiety is selected from the group consisting of a diketone, a N-sulfonyl-beta-lactam, and an azetidinone. In some embodiments, the specificity agent comprises a reactive moiety that is conjugated via a linker. In even further embodiments, the linker is selected from the group consisting of a peptide, a small molecule, an alkyl linker, and a PEG linker.

In some embodiments, the specificity agent binds to a protein associated with cancer. In some embodiments, the protein associated with cancer is selected from the group consisting of CD19, an integrin, VEGFR2, PSMA, CEA, GM2, GD2, GD3, EGFR, EGFRvIII, HER2, IL13R, folate receptor, and MUC-1. In some embodiments, the protein associated with cancer is selected from the group consisting of cholecystokinin B receptor, gonadotropin-releasing hormone receptor, somatostatin receptor 2, gastrin-releasing peptide receptor, neurokinin 1 receptor, melanocortin 1 receptor, a neurotensin receptor, neuropeptide Y receptor, and C-type lectin like molecule 1. In some embodiments, the specificity agent comprises a targeting molecule listed in Table 4.

In other embodiments, the specificity agent binds to a viral protein. In some embodiments, the viral protein is selected from the group consisting of an HIV protein, a hepatitis virus protein, an influenza virus protein, a herpes virus protein, a rotavirus protein, a respiratory syncytial virus protein, a poliovirus protein, a rhinovirus protein, a cytomegalovirus protein, a simian immunodeficiency virus protein, an encephalitis virus protein, a varicella zoster virus protein, and an Epstein-Barr virus protein.

In some embodiments, the specificity agent binds to a protein expressed by a disease-causing organism. In some embodiments, the disease-causing organism is a unicellular. In other embodiments, the disease-causing organism is multicellular. In some embodiments, the disease-causing organism is selected from the group consisting of a virus, a prion, a bacterium, a fungus, a protozoan, and a parasite.

In some embodiments, the specificity agent comprises a binding protein, small molecule, a peptide, a peptidomimetic, a therapeutic agent, a targeting agent, a protein agonist, a protein antagonist, a metabolic regulator, a hormone, a toxin, or a growth factor. In some embodiments, the small molecule is folic acid or DUPA. In some embodiments, the binding protein is an antibody, an antigen-binding portion of an antibody (e.g., an scFv), a ligand, a cytokine, or a receptor. In some embodiments, the binding protein is an antibody or an antigen binding fragment thereof. In some embodiments, the antigen binding fragment is a scFv or an Fab fragment. In some embodiments, the antigen binding fragment is a single chain Fab fragment (scFab). In some embodiments, the antibody or antibody binding fragment thereof comprises a kappa light chain (e.g., a humanized kappa light chain or a human kappa light chain). In some embodiments, the antibody or antibody binding fragment thereof comprises a variable kappa light chain (e.g., a humanized variable kappa light chain or a human variable kappa light chain).

In some embodiments, the host cell comprises a programmable universal cell receptor which is conjugated to a specificity agent specific for a first antigen, and a programmable universal cell receptor which is conjugated to a specificity agent specific for a second antigen, which is different than the first antigen.

In some embodiments, the host cell comprises a programmable universal cell receptor which is conjugated to a linker.

In some embodiments, the host cell is an immune cell. In some embodiments, the immune cell is selected from the group consisting of a dendritic cell, a monocyte, a mast cell, an eosinophil, a T cell, a B cell, a cytotoxic T lymphocyte, a macrophage, a Natural Killer (NK) cell, a monocyte, and a Natural Killer T (NKT) cell. In some embodiments, the NK cell is a NK-92 cell or a modified NK-92 cell. In some embodiments, the immune cell is a modified NK-92 cell (ATCC Deposit No. PTA-6672). In some embodiments, the host cell is isolated from a human subject having cancer.

In one aspect, the present invention provides a population of host cells wherein the population of comprises: a) a subpopulation of host cells comprising a programmable universal cell receptor linked to a specificity agent that binds to a first antigen; and b) a subpopulation of host cells comprising a programmable universal cell receptor linked to a specificity agent that binds to a second antigen, which is different than the first antigen.

In one aspect, the present invention provides a method for treating a cancer or inhibiting tumor growth in a subject in need thereof, the method comprising administering to the subject a host cell or a population of host cells disclosed herein, thereby treating the cancer or inhibiting tumor growth in the subject. In some embodiments, the present invention provides a method for treating cancer associated with VEGFR2. In some embodiments, the present invention provides a method for treating cancer associated with PSMA, e.g., prostate cancer. In some embodiments, the present invention provides a method for treating cancer associated with CD19, e.g., acute lymphoblastic lymphoma (ALL), non-Hodgkin's lymphoma, lung cancer, and chronic lymphocytic leukemia (CLL). In some embodiments, the present invention provides a method for treating cancer associated with HER2, e.g., ovarian cancer, stomach cancer, uterine cancer and breast cancer. In some embodiments, the present invention provides a method for treating cancer associated with EGFR, e.g., non small cell lung cancer (NSCLC), colon cancer, rectal cancer, head and neck squamous cell carcinoma (HNSCC), breast cancer and pancreatic cancer. In some embodiments, the present invention provides a method for treating cancer associated with IL13R, e.g., breast cancer or malignant glioma.

In another aspect, the present invention provides a method of treating a medical condition caused by a disease-causing organism in a subject in need thereof, the method comprising administering to the subject a host cell or a population of host cells disclosed herein, thereby treating the medical condition caused by the disease-causing organism in the subject.

In one aspect, the present invention provides a method of making a customized therapeutic host cell for use in the treatment of cancer in a subject in need thereof, the method comprising contacting an immune cell with a specificity agent that binds to a programmable universal cell receptor that is expressed on the cell membrane of the immune cell, wherein the specificity agent binds to a cancer-associated antigen corresponding to a cancer antigen profile of the subject in need thereof. In some embodiments, the immune cell is selected from the group consisting of a dendritic cell, a mast cell, a monocyte, an eosinophil, a T cell, a B cell, a cytotoxic T lymphocyte, a macrophage, a Natural Killer (NK) cell, a monocyte, and a Natural Killer T (NKT) cell. In some embodiments, the immune cell is a T cell or NK cell. In some embodiments, the NK cell is a NK-92 cell or a modified NK-92 cell. In some embodiments, the NK cell is a modified NK-92 cell (ATCC Deposit No. PTA-6672). In some embodiments, the cancer-associated antigen is selected from the group consisting of CD19, an integrin, VEGFR2, PSMA, CEA, GM2, GD2, GD3, sialyl Tn (STn), EGFR, EGFRvIII, HER2, IL13R, folate receptor, and MUC-1. In some embodiments, the protein associated with cancer is selected from the group consisting of cholecystokinin B receptor, gonadotropin-releasing hormone receptor, somatostatin receptor 2, gastrin-releasing peptide receptor, neurokinin 1 receptor, melanocortin 1 receptor, a neurotensin receptor, neuropeptide Y receptor, and C-type lectin like molecule 1. In yet more embodiments, the specificity agent comprises a binding protein, small molecule, a peptide, a peptidomimetic, a therapeutic agent, a targeting agent, a protein agonist, a protein antagonist, a metabolic regulator, a hormone, a toxin, or a growth factor.

In one aspect, the present invention provides a method for treating a cancer in a subject in need thereof, said method comprising determining a cancer antigen profile of the subject; selecting a specificity agent that binds to the antigen previously identified in the cancer antigen profile; and administering an immune cell comprising a programmable universal cell receptor bound to the specificity agent previously identified.

In one aspect, the present invention provides a kit comprising a container comprising a population of host cells comprising a programmable universal cell receptor, wherein the programmable universal cell receptor comprises a catalytic antibody, or a catalytic portion thereof, comprising a reactive amino acid residue; wherein the reactive amino acid residue is not bound to a substrate; a transmembrane domain; and an intracellular domain. In some embodiments, the host cell is an immune cell. In some embodiments, the immune cell is a modified NK-92 cell (ATCC Deposit No. PTA-6672). In some embodiments, the kit further comprises a specificity agent. In some embodiments, the kit comprises from about 1×10² to about 1×10¹⁶ immune cells.

In another aspect, the present invention provides a kit comprising a container comprising a nucleic acid disclosed herein.

In yet a further aspect, the present invention provides a kit comprising a container comprising a vector disclosed herein.

### BRIEF DESCRIPTION OF THE DRAWINGS

FIG. 1 depicts a schematic graph for programming of a host cell (e.g., a NK cell or a T cell) comprising a programmable universal cell receptor conjugated to a specificity agent.

FIG. 2 depicts a schematic reaction for site-specific conjugation of a small molecule onto the reactive Lys93 residue in the variable domain of the catalytic antibody h38C2 (humanized 38C2). The lysine residue is located in a hydrophobic core of the antibody. The side chain NH₂ group of Lys93 remains unprotonated under physiological conditions, where it can attack a reactive moiety to form a covalent bond.

FIG. 3 depicts an SDS-PAGE analysis for the purification of the humanized and murine 38C2 scFv-Fc under both non-reducing and reducing conditions.

FIG. 4 depicts the mass spectrometry analysis of the humanized 38C2 scFv-Fc reactivity with azetidinone-PEG5-methyl ester.

FIG. 5 depicts the mass spectrometry analysis of the murine 38c2 scFv-Fc reactivity with azetidinone-PEG5-methyl ester.

FIG. 6 depicts the peptide mapping data of humanized 38C2 scFv-Fc conjugated to azetidinone-PEG5-methyl ester. The mass of the peptide fragment was shown to contain Lys93 of humanized 38C2 scFv-Fc, indicating that the conjugation reaction occurred on Lys 93 of the heavy chain.

FIG. 7 depicts the chemical structure for the exemplary specificity agent folic acid-diketone (2-[[4-[(2-amino-4-oxo-3H-pteridin-6-yl)methylamino]benzoyl]amino]-5-[2-[2-[2-[[5-[4-(3,5-dioxohexyl)anilino]-5-oxo-pentanoyl]amino]ethoxy]ethoxy]ethylamino]-5-oxo-pentanoic acid).

FIG. 8 depicts the chemical structure for the exemplary specificity agent folic acid-azetidinone (2-[[4-[(2-amino-4-oxo-3H-pteridin-6-yl)methylamino]benzoyl]amino]-5-oxo-5-[2-[2-[3-oxo-3-[4-[3-oxo-3-(2-oxoazetidin-1-yl)propyl]anilino]propoxy]ethoxy]ethylamino]pentanoic acid).

FIG. 9 depicts the chemical structure for the exemplary specificity agent diketone-PEG5-DUPA ((2S)-2-[[(1S)-4-[[8-[[(1S)-1-benzyl-2-[[(1S)-1-benzyl-2-[2-[2-[3-[2-[2-[2-[2-[3-[4-(3,5-dioxohexyl)anilino]-3-oxo-propoxy]ethoxy]ethoxy]ethoxy]ethoxy]propanoylamino]ethoxy]ethylamino]-2-oxo-ethyl]amino]-2-oxo-ethyl]amino]-8-oxo-octyl]amino]-1-carboxy-4-oxo-butyl]carbamoylamino]pentanedioic acid).

FIG. 10 depicts the chemical structure for the exemplary specificity agent DUPA-azetidinone ((2S)-2-[[(1S)-4-[[8-[[(1S)-1-benzyl-2-[[(1S)-1-benzyl-2-oxo-2-[2-[2-[3-[2-[2-[2-[2-[3-oxo-3-[4-[3-oxo-3-(2-oxoazetidin-1-yl)propyl]anilino]propoxy]ethoxy]ethoxy]ethoxy]ethoxy]propanoylamino]ethoxy]ethylam ino]ethyl]amino]-2-oxo-ethyl]amino]-8-oxo-octyl]amino]-1-carboxy-4-oxo-butyl]carbamoylamino]pentanedioic acid).

FIG. 11 depicts the chemical structure for exemplary specificity agent azetidinone-PEG8-Biotin (5-[(3aS,4S,6aR)-2-oxo-1,3,3a,4,6,6a-hexahydrothieno[3,4-d]imidazol-4-yl]-N-[2-[2-[2-[2-[2-[2-[2-[3-[2-[3-oxo-3-[4-[3-oxo-3-(2-oxoazetidin-1-yl) propyl]anilino]propoxy]ethoxy]propanoylamino]ethoxy]ethoxy]ethoxy]ethoxy]ethoxy]eth oxy]ethyl]pentanamide).

FIG. 12 depicts flow cytometry data of wild-type NKL cells (“−PUCR”; middle row) or NKL cells expressing a PUCR comprising 38C2 scFab (“+PUCR”; lower row) that were reacted with either 1 μM or 10 μM of the specificity agent AZD-PEG8-Biotin. Conjugation of the AZD-PEG8-Biotin to the PUCR was detected using DTAF-conjugated streptavidin and analyzed by FACS. Background fluorescence control is shown in the left graph, upper row. DTAF-conjugated streptavidin exposed cells (secondary control) is shown in the right graph, right graph.

FIG. 13 depicts flow cytometry data of wild-type NKL cells (“−PUCR”) or NKL cells expressing a PUCR comprising 38C2 scFab (“+PUCR”) that were reacted with either 1 μM or 10 μM of the specificity agent AZD-PEG8-Biotin. Conjugation of the AZD-PEG8-Biotin to the PUCR was detected using 1 μM DTAF-conjugated streptavidin (“DTAF-Streptavidin”) and analyzed by FACS. Background fluorescence was subtracted.

FIG. 14 shows fluorescent detection images of a non-reducing SDS-PAGE analysis of a conjugation reaction to program recombinant 38C2 scFv-Fc with anti-VEGFR2 VK-B8 Fab fragment conjugated to the AZD-PEG13-PFP ester linker. The left panel shows a fluorescent image of the unstained gel. Anti-VEGFR2 VK-B8 Fab fragment conjugated to the AZD-PEG13-PFP ester linker was fluorescently labeled with AlexaFluor®488 NHS ester (“VKB8 Fab AZD 488”). As control, anti-VEGFR2 VKB8 Fab fragment not conjugated to the AZD-PEG13-PFP ester linker that was either fluorescently labeled with AlexaFluor®488 NHS ester (“VKB8 Fab 488”), or non-fluorescently labeled (“VKB8 Fab”) was used. Fluorescently labelled anti-VEGFR2 VKB8 Fab fragment conjugated to the AZD-PEG13-PFP ester linker, or fluorescently labeled anti-VEGFR2 VKB8 Fab fragment not conjugated to the AZD-PEG13-PFP ester linker, were reacted with murine catalytic 38C2 scFv-Fc. No fluorescence was detected with the murine catalytic 38C2 scFv-Fc was run on the gel (“m38C2”). Reaction of the anti-VEGFR2 VKB8 Fab fragment conjugated to the AZD-PEG13-PFP ester linker with the murine catalytic 38C2 scFv-Fc (“VKB8 Fab AZD 488+m38C2”) resulted the detection of fluorescent high molecular weight complexes of VK-B8 Fab fragment-conjugated 38C2 scFv (indicated with an “*”). In contrast, no fluorescent high molecular weight complexes were observed using anti-VEGFR2 VKB8 Fab fragment that was not conjugated to the AZD-PEG13-PFP ester linker (“VKB8 Fab 488+m38C2”). Right panel shows the same gel following Sypro® Ruby protein staining to detect gel loading.

FIG. 15 shows a binding curve for recombinant PSMA binding to wild-type KHYG-1 natural killer cells (“KHYG-1”; circles) or KHYG-1 natural killer cells expressing PUCR comprising 38C2 scFab programmed with 0.1 nM, 1 nM, 10 nM, or 100 nM of DK-PEG5-DUPA (“KHYG-1/Fab38C2”; squares).

FIG. 16A shows the cytotoxicity (% killing) of PSMA-positive LNCaP cells by either wild-type KHYG-1 NK cells (“KHYG-1”; circles) or KHYG-1 NK cells expressing PUCR comprising 38C2 scFab programmed with either 3.2 nM, 10 nM, 32 nM, 100 nM, 320 nM, or 1000 nM of DK-PEG5-DUPA (“KHYG-1/Fab38C2”; squares).

FIG. 16B shows the cytotoxicity (% killing) of PSMA-negative PC-3 cells by either wild-type KHYG-1 NK cells (“KHYG-1”; circles) or KHYG-1 NK cells expressing PUCR comprising 38C2 scFab programmed with either 3.2 nM, 10 nM, 32 nM, 100 nM, 320 nM, or 1000 nM of DK-PEG5-DUPA (“KHYG-1/Fab38C2”; squares).

FIG. 17 depicts the chemical structure for exemplary linker diketone-PEG5-PFP ester (2,3,4,5,6-pentafluorophenyl) 3-[2-[2-[2-[2-[3-[4-(3,5-dioxohexyl)anilino]-3-oxo-propoxy]ethoxy]ethoxy]ethoxy]ethoxy]propanoate).

FIG. 18A depicts the mass spectrometry analysis of the anti-PSMA Clone A11 Fab fragment.

FIG. 18B depicts the mass spectrometry analysis of the products resulting from reacting anti-PSMA Clone A11 Fab fragment with the diketone-PEG5-PFP ester linker.

FIG. 19 depicts the chemical structure for exemplary linker azetidinone-PEG13-PFP ((2,3,4,5,6-pentafluorophenyl) 3-[2-[2-[2-[2-[2-[2-[2-[2-[2-[2-[2-[2-[3-oxo-3-[4-[3-oxo-3-(2-oxoazetidin-1-yl)propyl]anilino]propoxy]ethoxy]ethoxy]ethoxy]ethoxy]ethoxy]ethoxy]ethoxy]ethoxy]ethoxy]ethoxy]ethoxy]ethoxy]propanoate).

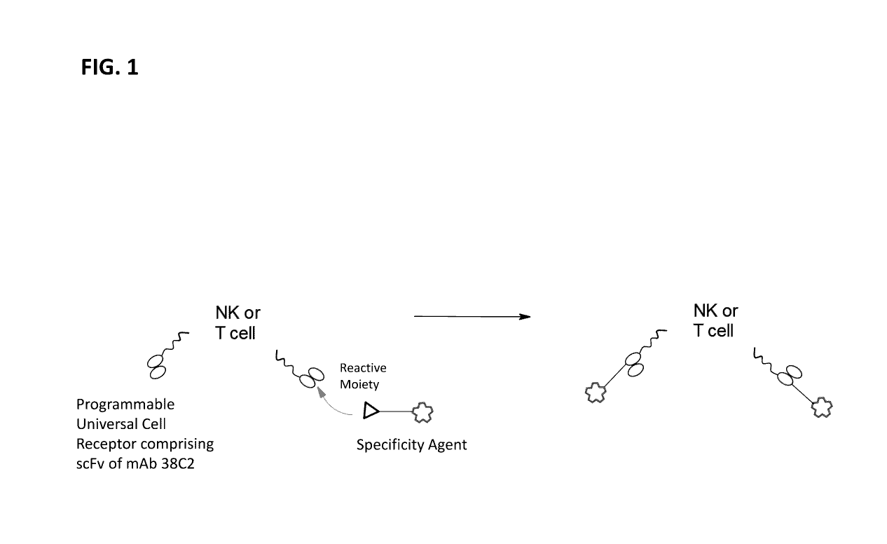

### DETAILED DESCRIPTION OF THE INVENTION

### I. Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

The terms “high expression levels” or “high levels of expression”, as used interchangeably herein, refer to a level of a molecular marker (e.g., a protein and/or an RNA (e.g., a mRNA)) which is increased relative to a normal level, i.e., that of a healthy subject who does not have cancer. In some preferred embodiments, the high level of expression refers to a level which is associated with cancer in a subject.

The term “programmable universal cell receptor” or “PUCR”, used interchangeably herein, refers to a recombinant molecule that contains an extracellular domain (also referred to herein as a catalytic antibody region) comprising a catalytic antibody, or a catalytic portion thereof, a transmembrane domain, and an intracellular domain. In some embodiments, the programmable universal cell receptor is encoded by a nucleic acid molecule that has been codon-optimized for the specific host cell expressing the receptor.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like. In some embodiments, the antibody is a donkey antibody. In some embodiments, the antibody is a rat antibody. In some embodiments, the antibody is a horse antibody. In some embodiments, the antibody is a camel antibody. In some embodiments, the antibody is a shark antibody.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and Winter et al., PCT Publication No. WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. The term antigen binding portion of an antibody includes a “single chain Fab fragment” otherwise known as an “scFab.”

A “single chain Fab fragment” or “scFab” is a polypeptide comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments a) VH-CH1-linker-VL-CL, b) VL-CL-linker-VH-CH1, c) VH-CL-linker-VL-CH1 and d) VL-CH1-linker-VH-CL, may be stabilized via the natural disulfide bond between the CL domain and the CH1 domain. In addition, these single chain Fab fragments may be further stabilized by generation of interchain disulfide bonds via insertion of cysteine residues (e.g., position 44 in the variable heavy chain and position 100 in the variable light chain according to Kabat numbering). The term “N-terminus” denotes the last amino acid of the N-terminus. The term “C-terminus” denotes the last amino acid of the C-terminus.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “catalytic antibody” refers to an immunoglobulin molecule capable of catalyzing a biochemical reaction with a reactive moiety. Catalytic antibodies may be produced by reactive immunization, whereby an animal is immunized with a reactive hapten as the immunogen. The catalytic antibody may be produced in any animal, including but not limited to, a mouse, a rat, a cow, a dog, a sheep, a goat, a donkey, a horse, a human, a primate, a pig, and a chicken. In some embodiments, the catalytic antibody is a full-length antibody. In some embodiments, the catalytic antibody is a murine antibody. In some embodiments, the catalytic antibody is a human antibody. In some embodiments, the catalytic antibody is a humanized antibody. In some embodiments, the catalytic antibody is a chimeric antibody. Many catalytic antibodies, and methods of generating catalytic antibodies, that may be used in accordance with the present invention, are known in the art (see, e.g., Zhu et al., (2004) J. MOL. BIOL. 343: 1269-80; Rader et al. (1998) PROC. NAT'L. ACAD. SCI. USA 95: 8910-8915; U.S. Pat. Nos. 6,210,938; 6,368,839; 6,326,176; 6,589,766; and U.S. Pat. Nos. 5,985,626, 5,733,757; 5,500,358; 5,126,258; 5,030,717; and 4,659,567; the contents of which are herein incorporated by reference, and in particular, the disclosure regarding catalytic antibodies and methods of generating catalytic antibodies). In some embodiments, the catalytic antibody is an aldolase antibody. In other embodiments the catalytic antibody is the murine antibody 38C2, or a chimeric or humanized version of said antibody (see, e.g., Karlstrom et al. (2000) PROC. NAT'L. ACAD. SCI. USA 97(8): 3878-3883; and Rader et al. (2003) J. MOL. BIOL. 332: 889-99). Murine antibody 38C2 has a reactive lysine near to, but outside, HCDR3, and is a catalytic antibody generated by reactive immunization that mechanistically mimics natural aldolase enzymes (see, e.g., Barbas et al. (1997) SCIENCE 278: 2085-2092). In some embodiments, the catalytic antibody is the murine antibody 33F12, or a chimeric or humanized version of said antibody (see, e.g., Goswami et al. (2009) BIOORG. MED. CHEM. LETT. 19(14): 3821-4). In some embodiments, the catalytic antibody is the antibody produced by the hybridoma 40F12 (Zhu et al., (2004) J. MOL. BIOL. 343: 1269-80; Rader et al., (1998)) or a chimeric or humanized version of said antibody. In some embodiments, the catalytic antibody is the antibody produced by the hybridoma 42F1 (Zhu et al., (2004); Rader et al., (1998)) or a chimeric or humanized version of said antibody. In other embodiments, the catalytic antibody is the antibody produced by the hybridoma 85A2 (ATCC accession number PTA-1015), or a chimeric or humanized version of said antibody. In some embodiments, the catalytic antibody is the antibody produced by the hybridoma 85C7 (ATCC accession number PTA-1014) or a chimeric or humanized version of said antibody. In other embodiments, the catalytic antibody is the antibody produced by the hybridoma 92F9 (ATCC accession number PTA-1017), or a chimeric or humanized version of said antibody. In some embodiments, the catalytic antibody is the antibody produced by the hybridoma 93F3 (ATCC accession number PTA-823), or a chimeric or humanized version of said antibody. In other embodiments, the catalytic antibody is the antibody produced by the hybridoma 84G3 (ATCC accession number PTA-824), or a chimeric or humanized version of said antibody. In some embodiments, the catalytic antibody is the antibody produced by the hybridoma 84G11 (ATCC accession number PTA-1018), or a chimeric or humanized version of said antibody. In other embodiments, the catalytic antibody is the antibody produced by the hybridoma 84H9 (ATCC accession number PTA-1019), or a chimeric or humanized version of said antibody. In some embodiments, the catalytic antibody is the antibody produced by the hybridoma 85H6 (ATCC accession number PTA-825), or a chimeric or humanized version of said antibody. In other embodiments, the catalytic antibody is the antibody produced by the hybridoma 90G8 (ATCC accession number PTA-1016), or a chimeric or humanized version of said antibody. In some embodiments, the catalytic antibody is a beta lactamase antibody. In other embodiments, the catalytic antibody is an esterase antibody. In some embodiments, the catalytic antibody is an amidase antibody. In other embodiments, the catalytic antibody is an thioesterase antibody. In some embodiments, the catalytic antibody is a donkey antibody. In some embodiments, the catalytic antibody is a rat antibody. In some embodiments, the catalytic antibody is a horse antibody. In some embodiments, the catalytic antibody is a camel antibody. In some embodiments, the catalytic antibody is a shark antibody.

As used herein, the terms “catalytic portion” or “catalytic fragment” refer to a fragment of a catalytic antibody that retains the ability to catalyze a biochemical reaction with a reactive moiety. In some embodiments, the catalytic portion of a catalytic antibody retains a reactive amino acid residue, e.g., a reactive lysine residue, which enables the amino acid residue to catalyze a biochemical reaction. For example, a catalytic portion of an aldolase antibody may comprise a reactive lysine and the microenvironment necessary to catalyze aldol and/or retro-aldol reactions using the enamine mechanism of natural aldolases. Various forms of catalytic portions of catalytic antibodies are contemplated in some embodiments, as long as the catalytic portion retains a reactive amino acid residue. In some embodiments, the catalytic portion is (i) a Fab fragment of a catalytic antibody, a monovalent fragment consisting of the VL, VH, CL and CH1 domains; (ii) a F(ab′)₂ fragment of a catalytic antibody, a bivalent fragment comprising two Fab fragments linked by a disulfide bridge at the hinge region; (iii) a Fd fragment of a catalytic antibody, consisting of the VH and CH1 domains; (iv) a Fv fragment of a catalytic antibody, consisting of the VL and VH domains of a single arm of a catalytic antibody, (v) a dAb fragment of a catalytic antibody, which comprises a single variable domain; and (vi) an isolated complementarity determining region (CDR) of a catalytic antibody. In some embodiments, the catalytic portion is the CDR3 from the VH domain of a catalytic antibody (e.g., a catalytic antibody disclosed herein). In some embodiments, the catalytic portion is a single chain Fab (scFab). Furthermore, although the two domains of the Fv fragment, VL and VH of a catalytic antibody, are coded for by separate genes, they can be joined, using recombinant methods, by a synthetic linker that enables them to be made as a single protein chain in which the VL and VH regions pair to form monovalent molecules (known as single chain Fv (scFv)). In some embodiments, the catalytic portion of a catalytic antibody is a scFv. In other embodiments, the catalytic portion of a catalytic antibody is a scFab. Such single chain antibodies are also intended to be encompassed within the term “catalytic portion” of a catalytic antibody. Other forms of single chain antibodies, such as diabodies are also encompassed.

As used herein, the term “reactive amino acid residue” refers to an amino acid residue present in a catalytic antibody that is biochemically reactive, via a reactive side chain, with a reactive moiety. The reactive amino acid residue may be naturally-present in the catalytic antibody. Alternatively, the reactive amino acid residue may arise by purposely mutating the DNA encoding the catalytic antibody so as to encode the particular reactive amino acid residue of interest. In one embodiment, the reactive amino acid residue, or its reactive functional groups (e.g., a nucleophilic amino group or sulfhydryl group), may be attached to an amino acid residue of an antibody, to thereby form a catalytic antibody. In some embodiments, the reactive amino acid residue is a cysteine (e.g., a reactive cysteine residue of a thioesterase antibody). In other embodiments, the reactive amino acid residue is a serine. In some embodiments, the reactive amino acid residue is a tyrosine. In some embodiments, the reactive amino acid residue is a lysine (e.g., a reactive lysine residue of an aldolase antibody). In other embodiments, the reactive amino acid residue is Lys93 on the heavy chain of the murine antibody 38C2 according to Kabat numbering. In other embodiments, the reactive amino acid residue is Lys93 of humanized antibody 38C2 according to Kabat numbering. In some embodiments, the reactive amino acid residue is Lys93 of murine antibody 33F12 according to Kabat numbering. In other embodiments, the reactive amino acid residue is Lys93 of humanized antibody 33F12 according to Kabat numbering. In some embodiments, the reactive amino acid residue is Lys93 of murine antibody 40F12 according to Kabat numbering. In other embodiments, the reactive amino acid residue is Lys93 of humanized antibody 40F12 according to Kabat numbering. In some embodiments, the reactive amino acid residue is Lys93 of murine antibody 42F1 according to Kabat numbering. In other embodiments, the reactive amino acid residue is Lys93 of humanized antibody 42F1 according to Kabat numbering. In some embodiments, the reactive amino acid residue is Lys89 of murine antibody 84G3 according to Kabat numbering. In other embodiments, the reactive amino acid residue is Lys89 of humanized antibody 84G3 according to Kabat numbering. In some embodiments, the reactive amino acid residue is Lys89 of murine antibody 93F3 according to Kabat numbering. In other embodiments, the reactive amino acid residue is Lys89 of humanized antibody 93F3 according to Kabat numbering.

As used herein, the term “codon-optimized” refers to the alteration of codons in the gene or coding regions of a nucleic acid molecule to reflect the typical codon usage of the host organism without altering the polypeptide encoded by the nucleic acid molecule (e.g., a DNA molecule).

The term “humanized”, as used herein in reference to antibodies (e.g., catalytic antibodies) and portions thereof, refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; Presta (1992) CURR. OP. STRUCT. BIOL. 2: 593-596.

As used herein, the term “detectable moiety” refers to a moiety that is attached through covalent or non-covalent means to a programmable universal chimeric receptor and/or a specificity agent. In some embodiments, the detectable moiety provides a means for detection or quantitation of the programmable universal chimeric receptor and/or the specificity agent comprising the detectable moiety. In other embodiments, the detectable moiety provides a means for separating and/or purifying the programmable universal chimeric receptor and/or the specificity agent comprising the detectable moiety. In some embodiments, the detectable moiety comprises a polypeptide (e.g., a GST-tag, a His-tag, a myc-tag, or a HA-tag, a fluorescent protein (e.g., a GFP or a YFP)). In some embodiments, the detectable moiety comprises a radioactive moiety, a fluorescent moiety, a chemiluminescent moiety, a mass label, a charge label, or an enzyme (e.g., an enzyme for which substrate converting activity of the enzyme is observed to reveal the presence of the programmable universal chimeric receptor and/or the specificity agent). Detectable moieties for use in the present invention may be attached to any part of the programmable universal cell receptor and/or specificity agent. In some embodiments, the detectable moiety is attached to the N-terminus of the programmable universal cell receptor. In some embodiments, the detectable moiety is attached to the N-terminus of the specificity agent. In some embodiments, the detectable moiety is attached to the C-terminus of the programmable universal cell receptor. In some embodiments, the detectable moiety is attached to the C-terminus of the specificity agent. In some embodiments, the programmable universal cell receptor and/or specificity agent comprises one, two, three, four, five, six, seven, eight, nine, ten or more detectable moieties. In some embodiments the detectable moiety is cleavable. In other embodiments, the detectable moiety is non-cleavable. In some embodiments, the detectable moiety is attached to the programmable universal cell receptor and/or specificity agent via a linker. In some embodiments, the linker is cleavable. In other embodiments, the linker is non-cleavable.

As used herein, the term “specificity agent” refers to a molecule that can be bound (e.g., covalently or non-covalently conjugated) to the catalytic antibody region of the PUCR. Said specificity agent comprises a reactive moiety that is bound to the reactive amino acid residue present in the catalytic antibody region of the PUCR. When bound to the catalytic antibody region of the PUCR, the specificity agent confers specificity to the PUCR for a target molecule. In some embodiments the specificity agent comprises a binding protein (e.g., an antibody or antigen binding fragment thereof). In other embodiments, the specificity agent comprises a peptide. In some embodiments, the specificity agent comprises a peptidomimetic (e.g., RGD peptidomimetics). In other embodiments, the specificity agent comprises a small molecule (e.g., folic acid or 2-[3-(1, 3-dicarboxy propyl)-ureido] pentanedioic acid (DUPA)). In some embodiments, the specificity agent comprises a therapeutic agent. In other embodiments, the specificity agent comprises a targeting agent (e.g., a cell targeting molecule). In some embodiments, the specificity agent comprises a protein agonist. In other embodiments, the specificity agent comprises a metabolic regulator. In some embodiments, the specificity agent comprises a hormone. In other embodiments, the specificity agent comprises a toxin. In some embodiments, the specificity agent comprises a growth factor. In other embodiments, the specificity agent comprises a ligand. In some embodiments, the specificity agent comprises a protein. In other embodiments, the specificity agent comprises a peptoid. In some embodiments, the specificity agent comprises a DNA aptamer. In other embodiments, the specificity agent comprises a peptide nucleic acid. In some embodiments, the specificity agent comprises a vitamin. In other embodiments, the specificity agent comprises a substrate or a substrate analog. In some embodiments, the specificity agent comprises a cyclic arginine-glycine-aspartic acid peptide (cRGD).

In some embodiments, the specificity agent comprises a linker. In some embodiments, the linker is a flexible linker. In some embodiments, the linker is a non-flexible linker. In some embodiments, the linker is cleavable. In some embodiments, the linker is hydrolysable. In some embodiments, the linker is non-cleavable. In some embodiments, the linker is a polyethylene glycol (PEG) linker.

In other embodiments, the specificity agent is covalently linked to the catalytic antibody, or catalytic portion thereof, of the PUCR. In some embodiments, the specificity agent is non-covalently linked to the catalytic antibody, or catalytic portion thereof, of the PUCR. In other embodiments, the covalent bond between the specificity agent and the catalytic antibody, or catalytic portion thereof, of the PUCR is reversible. In some embodiments, the covalent bond between the specificity agent and the catalytic antibody, or catalytic portion thereof, of the PUCR is irreversible. In some embodiments, the specificity agent is a folic acid-diketone molecule (2-[[4-[(2-amino-4-oxo-3H-pteridin-6-yl)methylamino]benzoyl]amino]-5-[2-[2-[2-[[5-[4-(3,5-dioxohexyl)anilino]-5-oxo-pentanoyl]amino]ethoxy]ethoxy]ethylamino]-5-oxo-pentanoic acid). In other embodiments, the specificity agent is a folic acid-azetidinone molecule (2-[[4-[2-amino-4-oxo-3H-pteridin-6-yl)methylamino]benzoyl]amino]-5-oxo-5-[2-[2-[3-oxo-3-[4-[3-oxo-3-(2-oxoazetidin-1-yl)propyl]anilino]propoxy]ethoxy]ethylamino]pentanoic acid). In some embodiments, the specificity agent is a DUPA-diketone molecule ((2S)-2-[[(1S)-4-[[8-[[(1S)-1-benzyl-2-[[(1S)-1-benzyl-2-[2-[2-[3-[2-[2-[2-[2-[3-[4-(3,5-dioxohexyl)anilino]-3-oxo-propoxy]ethoxy]ethoxy]ethoxy]ethoxy]propanoylamino]ethoxy]ethylamino]-2-oxo-ethyl]amino]-2-oxo-ethyl]amino]-8-oxo-octyl]amino]-1-carboxy-4-oxo-butyl]carbamoylamino]pentanedioic acid; also referred to herein as DK-PEG5-DUPA and diketone-PEG5-DUPA). In other embodiments, the specificity agent is a DUPA-azetidinone molecule ((2S)-2-[[(1S)-4-[[8-[[(1S)-1-benzyl-2-[[1S)-1-benzyl-2-oxo-2-[2-[2-[3-[2-[2-[2-[2-[3-oxo-3-[4-[3-oxo-3-(2-oxoazetidin-1-yl)propyl]anilino]propoxy]ethoxy]ethoxy]ethoxy]ethoxy]propanoylamino]ethoxy]ethylam ino]ethyl]amino]-2-oxo-ethyl]amino]-8-oxo-octyl]amino]-1-carboxy-4-oxo-butyl]carbamoylamino]pentanedioic acid). In some embodiments, the specificity agent is AZD-PEG8-Biotin (5-[(3aS,4S,6aR)-2-oxo-1,3,3a,4,6,6a-hexahydrothieno[3,4-d]imidazol-4-yl]-N-[2-[2-[2-[2-[2-[2-[2-[3-[2-[3-oxo-3-[4-[3-oxo-3-(2-oxoazetidin-1-yl) propyl]anilino]propoxy]ethoxy]propanoylamino]ethoxy]ethoxy]ethoxy]ethoxy]ethoxy]eth oxy]ethyl]pentanamide; also referred to herein as AZD-PEG8-Biotin”). In some embodiments, the specificity agent is azetidinone-PEG5-methyl ester (also referred to herein as AZD-PEG5-methyl ester). In some embodiments, the specificity agent is SCS-873 (see, e.g., Popkov et al. (2009) PROC. NAT'L. ACAD. SCI. USA 106(11): 4378-83). In other embodiments, the specificity agent is cRGD-dk (see, e.g., Popkov et al. (2009)).

The term “binding protein”, as used herein, refers to a protein or polypeptide that can specifically bind to a target molecule. In some embodiments the binding protein is an antibody or antigen binding fragment thereof, and the target molecule is an antigen. In some embodiments, said antigen comprises one or more post-translational modifications. In some embodiments the binding protein is a protein or polypeptide that specifically binds to a target molecule (e.g., a protein complex binding-partner). In some embodiments the binding protein is a ligand. In some embodiments, the binding protein is a cytokine. In some embodiments, the binding protein is a receptor. In some embodiments, the target molecule is an antigen. In other embodiments, the target molecule is a protein. In some embodiments, the target molecule is a peptide. In some embodiments, the target molecule is a protein complex. In some embodiments, the target molecule is a lipid. In some embodiments, the target molecule is a carbohydrate. In some embodiments the target molecule is a protein comprising one or more post-translational modifications. In some embodiments, the target molecule is an extracellular matrix component.

The term “specifically binds”, as used herein, indicates that a binding protein forms a complex with a target molecule that is relatively stable under physiologic conditions. Specific binding can be characterized by an equilibrium dissociation constant of at least about 1×10⁶ M or less (e.g., a smaller equilibrium dissociation constant denotes tighter binding). Methods for determining whether two molecules specifically bind are well known in the art and include, for example, equilibrium dialysis, surface plasmon resonance, and the like.

The term “reactive moiety”, as used herein, refers to a moiety that is capable of participating in a reaction with the reactive amino acid residue of the catalytic antibody, or catalytic portion thereof, of a PUCR. In some embodiments, the reactive moiety is covalently bound to the reactive amino acid residue. In some embodiments, the reactive moiety is covalently bound to a side chain of the reactive amino acid residue. In some embodiments, the reactive moiety is non-covalently bound to the reactive amino acid residue. In some embodiments, the reactive moiety is a chemical group selected from the group consisting of a ketone, a diketone, a beta lactam, an active ester haloketone, a lactone, an anhydride, a maleimide, an epoxide, an aldehyde amidine, a guanidine, an imine, an eneamine, a phosphate, a phosphonate, an epoxide, an aziridine, a thioepoxide, a masked or protected diketone (e.g., a ketal), a lactam, a haloketone, an aldehyde, and the like. For example, when the catalytic antibody, or catalytic portion thereof, is an aldolase antibody, or a catalytic portion thereof, (e.g., murine or humanized 38C2), the specificity agent may be covalently linked to the reactive lysine (e.g., Lys93) via a diketone or a azetidinone reactive moiety. Further, when the catalytic antibody, or catalytic portion thereof, is a thioesterase antibody, or a catalytic portion thereof, the specificity agent may be covalently linked to the reactive cysteine via a reactive moiety comprising a maleimide-containing component or other thiol-reactive groups such as iodoacetamides, aryl halides, disulfhydryls and the like. In some embodiments, the reactive moiety is a diketone. In other embodiments, the reactive moiety is a azetidinone. In some embodiments, the reactive moiety is a N-sulfonyl-beta-lactam.

The term “conjugation functional group”, as used herein, refers to a moiety present on a linker described herein that is capable of participating in a reaction with a moiety present on a specificity agent. In some embodiments, the conjugation functional group is capable of participating in a click-chemistry reaction with a moiety present on a specificity agent. In some embodiments, the conjugation functional group comprises a orthogonal functional group. In some embodiments, the conjugation functional group is capable of forming a covalent bond with a moiety present on a specificity agent. In some embodiments, the conjugation functional group is capable of forming a non-covalent bond with a moiety present on a specificity agent.

As used herein, the term “host cell” refers to any cell that has been modified, transfected, transformed, and/or manipulated in any way to express a programmable universal cell receptor disclosed herein. For example, in some embodiments, the host cell has been modified to comprise an exogenous polynucleotide (e.g., a vector, linear DNA molecule, mRNA) encoding a programmable universal cell receptor disclosed herein. In some embodiments, the host cell is a eukaryotic cell. In some embodiments, the host cell is a mammalian cell. In some embodiments, the host cell is a primate cell. In some embodiments, the cell is a murine cell. In some embodiments, the cell is a rat cell. In some embodiments, the cell is a domestic animal cell (e.g., a dog or a cat cell). In some embodiments, the cell is an equine cell. In some embodiments, the cell is a cow cell. In some embodiments, the cell is a non-human primate cell. In some embodiments, the cell is a human cell. In some embodiments, the host cell is isolated from a subject. In some embodiments, the host cell is derived from a subject, whereby a cell is isolated from a subject, modified as described herein, and administered to the same subject from whom the host cell was derived. In some embodiments, the host cell is derived from a subject, whereby a cell is isolated from a subject, modified as described herein, and administered to a different subject from whom the host cell was derived. It should be understood that the term “host cell” is intended to refer not only to a particular subject cell, but to the progeny of such cell. Because certain modifications may occur in succeeding generations due to either mutation(s) or environmental influence(s), such progeny may not, in fact, be identical to the parent cell, but are still included within the scope of the term “host cell”, as used herein. In some embodiments, the host cell is an immune cell. In some embodiments, the immune cell is selected from the group consisting of a dendritic cell, a mast cell, an eosinophil, a T cell (e.g., a regulatory T cell), a B cell, a cytotoxic T lymphocyte, a macrophage, a Natural Killer cell, a monocyte, and a Natural Killer T (NKT) cell. In some embodiments, the host cell is a cell from an immortalized cell line. In some embodiments, the host cell is a cell from an established cell line. In some embodiments the host cell is a T cell. In some embodiments, the host cell is a CD8+ T cell. In some embodiments, the host cell is a CD4+ T cell. In some embodiments, the host cell is a NK cell. In some embodiments, the host cell is a NK-92 cell. In some embodiments, the host cell is a modified NK-92 cell (e.g., the modified NK-92 cell deposited as ATCC Deposit No. PTA-6672; also described, e.g., in U.S. Pat. No. 8,034,332). In some embodiments, the host cell is a KHYG-1 natural killer cell (DSMZ Accession No. ACC 725; see, e.g., Yagita et al. (2000) LEUKEMIA 14(5): 922-30). In some embodiments, the host cell is a NKL natural killer cell (see, e.g., Robertson et al. (1996) EXP. HEMATOL. 24(3): 406-15). In some embodiments, the host cell is a cytotoxic T lymphocyte.

As used herein, the term “nucleic acid” or “polynucleotide”, used interchangeably herein, refers to deoxyribonucleic acids (DNA) or ribonucleic acids (RNA), and polymers thereof, in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) NUCLEIC ACID RES. 19:5081; Ohtsuka et al. (1985) J. BIOL. CHEM. 260:2605-2608; and Rossolini et al. (1994) MOL. CELL PROBES 8:91-98).

As used herein, the term “subject” includes human and non-human animals. Non-human animals include all vertebrates (e g, mammals and non-mammals) such as, mice, rats, rabbits, humans, non-human primates, sheep, horses, dogs, cats, cows, chickens, amphibians, and reptiles. Except when noted, the terms “patient” or “subject” are used herein interchangeably. In particular embodiments, a subject having cancer, e.g., pancreatic cancer, prostate cancer, breast cancer, non-small cell lung cancer (NSCLC), or ovarian cancer, is a subject who has been previously diagnosed as having cancer, e.g., breast cancer, prostate cancer, ovarian cancer, cervical cancer, skin cancer, NSCLC, pancreatic cancer, colorectal cancer, renal cancer, liver cancer, brain cancer, lymphoma, leukemia, lung cancer and the like. In other embodiments, a subject having a medical condition caused by a disease-causing organism, e.g., a virus, a prion, a bacterium, a fungus, a protozoan, or a parasite, is a subject who has been diagnosed as having a medical condition caused by a disease-causing organism, e.g., a virus, a prion, a bacterium, a fungus, a protozoan, or a parasite. In some embodiments, the medical condition is an infectious disease. In some embodiments, the medical condition is an HIV infection. In some embodiments, the medical condition is hepatitis (e.g., hepatitis C). In some embodiments, the medical condition is malaria. In some embodiments, the medical condition is giardiasis.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that comprise a protein or peptide sequence. Polypeptides include any peptide or protein comprising two or more amino acids joined to each other by peptide bonds. As used herein, the term “polypeptide” refers to both short chains, which also commonly are referred to in the art as peptides, oligopeptides and oligomers, for example, and to longer chains, which generally are referred to in the art as proteins, of which there are many types. Polypeptides also include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, and unless otherwise specified, the terms “treat,” “treating” and “treatment” refer to the eradication or amelioration of a disease or disorder (e.g., a cancer or a disease caused by a disease causing organism (e.g., an infectious disease)) or of one or more symptoms associated with the disease or disorder. In certain embodiments, the terms refer to minimizing the spread or worsening of the disease or disorder (e.g., a cancer) resulting from the administration of one or more prophylactic or therapeutic agents to a subject with such a disease or disorder.

The terms “transfected” or “transformed” or “transduced”, as used herein, refer to a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

As used herein, and unless otherwise specified, the terms “cancer” and “cancerous” refer to or describe the physiological condition in mammals that is typically characterized by unregulated cell growth. Examples of cancer include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

### II. Compositions of the Invention

Provided herein are compositions and methods of use for the treatment of a disease, such as a cancer or an infectious disease, using a programmable universal cell receptor (PUCR).

In one aspect, the invention provides a number of programmable universal cell receptors (PUCRs) comprising a catalytic antibody, or a catalytic portion thereof, that may be engineered to target any molecule of interest (e.g., a host protein associated with cancer or a disease causing organism protein). In one aspect, the invention provides a cell (e.g., a T cell) engineered to express a PUCR, wherein the cell may be customized for therapeutic use (e.g., the cell exhibits an anti-tumor property). In some embodiments, the cell (e.g., a T cell) is transfected with a nucleic acid, e.g., mRNA, cDNA, DNA, encoding a PUCR. In some embodiments, the cell is transformed with a nucleic acid molecule encoding a PUCR and the PUCR is expressed on the cell surface of the cell. In some embodiments, the cell (e.g., a T cell) is transduced with a viral vector encoding a PUCR. In some embodiments, the viral vector is a retroviral vector. In some embodiments, the viral vector is a lentiviral vector. In some embodiments, the cell stably expresses the PUCR. In other embodiments, the cell transiently expresses the PUCR. In some embodiments, the cell inducibly expresses the PUCR.

In one aspect, the catalytic antibody region of the PUCR comprises a full length catalytic antibody, or a catalytic portion of said catalytic antibody. In one aspect, the catalytic antibody, or a catalytic portion thereof, is a non-human (e g, a murine) antibody, or catalytic portion thereof.

In one aspect, the catalytic antibody, or catalytic portion thereof, is a humanized catalytic antibody, or a catalytic portion thereof. Humanization of a non-human catalytic antibody, or of a catalytic portion thereof, may be desired in the clinical setting, where non-human-specific residues may induce an anti-non-human antibody response in patients who receive treatment comprising administration of a PUCR.

In one aspect, the catalytic antibody region of the PUCR comprises a catalytic scFv antibody fragment. In one aspect, the catalytic antibody region of the PUCR comprises a catalytic scFv antibody fragment that is humanized, as compared to the murine sequence of the scFv from which it is derived. The parental murine scFv amino acid sequence is the murine 38C2 scFv amino acid sequence provided herein as SEQ ID NO: 3. In one embodiment, the parental murine scFv sequence is encoded by the nucleic acid sequence provided herein as SEQ ID NO: 13. In one embodiment, the catalytic antibody region of the PUCR comprises the humanized 38C2 scFv construct provided herein as SEQ ID NO: 4. In one embodiment, the catalytic antibody region of the PUCR is encoded by the nucleic acid sequence provided herein as SEQ ID NO: 14.

In another aspect the catalytic antibody region of the PUCR comprises a catalytic scFab. In some embodiments, the catalytic scFab is derived from murine 38C2 catalytic antibody. In some embodiments, the catalytic scFab is derived from humanized 38C2 catalytic antibody. In some embodiments, the catalytic scFab comprises the amino acid sequence of SEQ ID NO: 40. In some embodiments, the catalytic scFab comprises the amino acid sequence of SEQ ID NO: 41. In some embodiments, the catalytic scFab comprises the amino acid sequence of SEQ ID NO: 54. In some embodiments, the catalytic scFab comprises the amino acid sequence of SEQ ID NO: 42. In some embodiments, the catalytic scFab comprises the amino acid sequence of SEQ ID NO: 43. In some embodiments, the catalytic scFab comprises the amino acid sequence of SEQ ID NO: 44. In one embodiment, the catalytic antibody region of the PUCR is encoded by the nucleic acid sequence provided herein as SEQ ID NO: 47.

TABLE 1
Exemplary Catalytic Antibody Region Sequences

| scFv | Sequence |
|---|---|
| murine 38C2 scFv | DVVMTQTPLSLPVRLGDQASISCRSSQSLLHTYGSPYLNWYLQK |
| amino acid sequence | PGQSPKLLIYKVSNRFSGVPDRFSGSGSGTDFTLRISRVEAEDL |
| The scFv is in a VL- | GVYFCSQGTHLPYTFGGGTKLEIKGGGGSGGGGSGGGGSEVKLV |
| linker-VH configuration. | ESGGGLVQPGGTMKLSCEISGLTFRNYWMSWVRQSPEKGLEWVA |
| The underlined sequence | EIRLRSDNYATHYAESVKGKFTISRDDSKSRLYLQMNSLRTEDT |
| is a poly Gly4Ser linker. | GIYYCKTYFYSFSYWGQGTLVTVSA |
| | (SEQ ID NO: 3) |
| murine 38C2 scFv | GATGTAGTTATGACCCAGACGCCTCTTTCTCTCCCCGTCCGGCT |
| nucleic acid sequence | CGGAGACCAAGCCTCCATCTCTTGCCGAAGTTCACAATCATTGT |
| | TGCACACGTATGGATCCCCATATCTGAATTGGTATCTCCAAAAG |
| | CCTGGACAGTCCCCCAAGCTGTTGATCTATAAAGTAAGTAATAG |
| | ATTTTCCGGCGTTCCTGACCGCTTCAGTGGCTCAGGAAGCGGTA |
| | CGGATTTTACTCTTCGGATTTCCCGCGTCGAAGCTGAAGATCTT |
| | GGTGTCTATTTCTGTTCTCAGGGAACGCACCTGCCATACACATT |
| | CGGAGGGGGCACTAAGCTCGAAATCAAGGGCGGGGGCGGGTCAG |
| | GTGGTGGGGGCAGCGGCGGGGGTGGCAGCGAGGTTAAGCTTGTG |
| | GAAAGTGGAGGCGGGCTTGTGCAGCCGGGCGGGACCATGAAACT |
| | GTCCTGCGAGATAAGTGGACTCACTTTTAGGAACTATTGGATGA |
| | GCTGGGTGCGACAGTCCCCCGAGAAGGGCCTTGAATGGGTTGCC |
| | GAAATACGGCTTCGATCAGACAACTATGCGACGCACTACGCTGA |
| | AAGCGTCAAAGGAAAATTCACTATCAGCCGGGACGACAGCAAGA |
| | GTAGACTTTATTTGCAGATGAATAGTTTGAGGACGGAAGATACG |
| | GGAATATATTATTGCAAAACATACTTCTATTCATTTTCATACTG |
| | GGGTCAGGGCACGTTGGTTACGGTTTCAGCC |
| | (SEQ ID NO: 13) |
| humanized 38C2 scFv | ELQMTQSPSSLSASVGDRVTITCRSSQSLLHTYGSPYLNWYLQK |
| The scFv is in a VL- | PGQSPKLLIYKVSNRFSGVPSRFSGSGSGTDFTLTISSLQPEDF |
| linker-VH configuration. | AVYFCSQGTHLPYTFGGGTKVEIKGGGGSGGGGSGGGGSEVQLV |
| The underlined sequence | ESGGGLVQPGGSLRLSCAASGFTFSNYWMSWVRQSPEKGLEWVS |
| is the poly Gly4Ser | EIRLRSDNYATHYAESVKGRFTISRDNSKNTLYLQMNSLRAEDT |
| linker. | GIYYCKTYFYSFSYWGQGTLVTVSS |
| | (SEQ ID NO: 4) |
| humanized 38C2 scFv | GAGCTTCAGATGACCCAAAGTCCCAGCTCTCTCTCCGCCTCTGT |
| nucleic acid sequence | CGGAGACAGGGTCACGATAACCTGTCGAAGTAGCCAGAGTCTTC |
| | TCCATACTTACGGAAGCCCATATCTTAACTGGTATCTTCAGAAA |
| | CCCGGTCAATCACCCAAGCTGCTGATATATAAAGTGTCTAACCG |
| | GTTTTCTGGTGTGCCGAGTCGATTTTCAGGATCAGGGAGCGGCA |
| | CGGATTTCACTCTTACGATCTCTAGTTTGCAACCTGAGGATTTT |
| | GCTGTATACTTTTGCAGCCAAGGTACTCATCTTCCTTATACGTT |
| | CGGAGGGGGTACCAAAGTAGAAATTAAAGGAGGAGGAGGGTCCG |
| | GAGGAGGGGGCAGCGGAGGAGGAGGCTCAGAAGTACAACTCGTG |
| | GAATCTGGCGGGGGGCTGGTGCAACCTGGGGGTTCTCTCCGCCT |
| | GAGCTGTGCTGCATCCGGCTTCACCTTTTCTAATTATTGGATGA |
| | GCTGGGTACGGCAGTCACCGGAGAAAGGTCTGGAGTGGGTGTCT |
| | GAGATACGACTTAGATCAGACAACTACGCGACGCATTACGCCGA |
| | GAGCGTGAAAGGAAGATTTACCATAAGCAGAGACAATTCAAAAA |
| | ACACCCTGTACCTCCAAATGAATAGCCTCAGGGCGGAAGATACT |
| | GGGATATATTACTGTAAAACCTACTTTTACAGTTTTAGTTATTG |
| | GGGCCAGGGAACGCTTGTAACTGTTAGCTCT |
| | (SEQ ID NO: 14) |
| Full length humanized | MEWSWVFLFFLSVTTGVHSELQMTQSPSSLSASVGDRVTITCRS |
| 38C2 scFab with signal | SQSLLHTYGSPYLNWYLQKPGQSPKLLIYKVSNRFSGVPSRFSG |
| peptide | SGSGTDFTLTISSLQPEDFAVYFCSQGTHLPYTFGGGTKVEIKR |
| | TVAAPSVFIFPPSDEQLKSGTASVVCLLNNFYPREAKVQWKVDN |
| | ALQSGNSQESVTEQDSKDSTYSLSSTLTLSKADYEKHKVYACEV |
| | THQGLSSPVTKSFNRGECGGGSGGGGSGGGSGGGGSGGGSGGGG |
| | SGGGGSGGGSGGGGSEVQLVESGGGLVQPGGSLRLSCAASGFTF |
| | SNYWMSWVRQSPEKGLEWVSEIRLRSDNYATHYAESVKGRFTIS |
| | RDNSKNTLYLQMNSLRAEDTGIYYCKTYFYSFSYWGQGTLVTVS |
| | SASTKGPSVFPLAPSSKSTSGGTAALGCLVKDYFPEPVTVSWNS |
| | GALTSGVHTFPAVLQSSGLYSLSSVVTVPSSSLGTQTYICNVNH |
| | KPSNTKVDKKVEPKSCDKTHT |
| | (SEQ ID NO: 44) |
| Full length humanized | ELQMTQSPSSLSASVGDRVTITCRSSQSLLHTYGSPYLNWYLQK |
| 38C2 scFab without | PGQSPKLLIYKVSNRFSGVPSRFSGSGSGTDFTLTISSLQPEDF |
| signal peptide | AVYFCSQGTHLPYTFGGGTKVEIKRTVAAPSVFIFPPSDEQLKS |
| | GTASVVCLLNNFYPREAKVQWKVDNALQSGNSQESVTEQDSKDS |
| | TYSLSSTLTLSKADYEKHKVYACEVTHQGLSSPVTKSFNRGECG |
| | GGSGGGGSGGGSGGGGSGGGSGGGGSGGGGSGGGSGGGGSEVQL |
| | VESGGGLVQPGGSLRLSCAASGFTFSNYWMSWVRQSPEKGLEWV |
| | SEIRLRSDNYATHYAESVKGRFTISRDNSKNTLYLQMNSLRAED |
| | TGIYYCKTYFYSFSYWGQGTLVTVSSASTKGPSVFPLAPSSKST |
| | SGGTAALGCLVKDYFPEPVTVSWNSGALTSGVHTFPAVLQSSGL |
| | YSLSSVVTVPSSSLGTQTYICNVNHKPSNTKVDKKVEPKSCDKT |
| | HT (SEQ ID NO: 104) |
| Humanized 38C2 scFab | AGCGAACTGCAGATGACCCAGTCCCCATCCAGTCTGAGCGCTAG |
| nucleic acid sequence | CGTTGGTGACAGAGTTACTATCACCTGCCGCTCTTCACAGAGCC |
| | TGTTGCACACTTACGGCTCTCCTTACCTGAACTGGTATCTTCAG |
| | AAGCCTGGCCAAAGCCCTAAGCTGCTCATCTACAAGGTGTCTAA |
| | CAGGTTCTCCGGGGTTCCGTCCCGCTTTTCAGGGAGCGGGTCAG |
| | GAACAGACTTCACCTTGACAATCTCAAGCCTCCAGCCCGAGGAT |
| | TTTGCCGTCTATTTCTGCTCACAAGGCACACATCTGCCGTATAC |
| | CTTTGGGGGCGGGACAAAAGTCGAGATCAAAAGGACCGTCGCTG |
| | CACCATCCGTGTTTATCTTCCCACCAAGTGACGAACAGCTCAAG |
| | AGCGGTACTGCCTCCGTTGTTTGTCTGCTGAACAACTTCTATCC |
| | AAGGGAAGCAAAGGTGCAATGGAAAGTAGACAACGCTCTGCAGT |
| | CAGGCAACTCCCAGGAGTCAGTGACCGAGCAGGATAGCAAAGAT |
| | TCAACATACAGCCTGAGCAGCACCCTCACCCTGAGTAAGGCCGA |
| | TTACGAGAAGCACAAGGTTTACGCCTGCGAGGTGACCCACCAGG |
| | GCCTTTCATCCCCAGTCACCAAATCTTTTAACCGCGGCGAATGC |
| | GGGGGAGGCTCTGGTGGAGGCGGTTCTGGAGGGGGCTCAGGAGG |
| | AGGCGGTAGCGGCGGTGGTAGTGGGGGTGGCGGATCTGGCGGAG |
| | GTGGCTCAGGAGGAGGTAGCGGCGGCGGGGGCAGCGAGGTCCAG |
| | CTGGTAGAGTCAGGTGGAGGATTGGTGCAGCCCGGCGGCAGTCT |
| | TAGACTCAGCTGTGCGGCCAGCGGATTTACTTTCTCAAATTATT |
| | GGATGTCTTGGGTCAGGCAGAGCCCAGAGAAAGGCCTGGAATGG |
| | GTGTCAGAGATCCGACTGAGAAGCGATAATTACGCGACTCATTA |
| | TGCGGAAAGCGTTAAAGGTCGGTTCACTATTTCACGAGATAATT |
| | CTAAGAATACCCTTTATCTGCAGATGAACAGCTTGCGCGCCGAG |
| | GACACAGGCATCTACTACTGTAAAACTTACTTCTATTCTTTTTC |
| | CTACTGGGGACAGGGGACTCTCGTTACAGTCAGTAGCGCCTCCA |
| | CCAAGGGTCCTAGTGTCTTTCCCCTGGCCCCCTCATCCAAGTCC |
| | ACGTCAGGAGGCACCGCGGCTCTGGGCTGTCTGGTCAAAGACTA |
| | CTTTCCTGAGCCAGTCACCGTGTCCTGGAATTCCGGCGCGCTTA |
| | CTTCTGGCGTGCACACTTTCCCCGCCGTCCTCCAGAGCAGTGGG |
| | CTGTATTCCCTGTCTTCCGTAGTCACTGTGCCAAGCTCCAGTCT |
| | GGGAACCCAGACCTATATTTGTAATGTGAATCATAAGCCGAGCA |
| | ACACCAAGGTGGACAAGAAGGTGGAACCGAAGTCATGTGACAAA |
| | ACCCACACT (SEQ ID NO: 47) |

In one embodiment, said antibody fragments are functional in that they retain the ability to catalyze a biochemical reaction, e.g., they mimic natural aldolase enzymes, as the full length catalytic antibody from which they are derived. In one embodiment, said antibody fragments are functional in that they provide a programmable moiety that may be bound to a specificity agent of interest. In one embodiment, said antibody fragments are functional in that they provide a programmable moiety that may be bound to a linker of interest.

In one aspect, the PUCR of the invention is encoded by a transgene whose sequence has been codon optimized for expression in a mammalian cell (e.g., a human cell). In some embodiments, the entire PUCR construct of the invention is encoded by a transgene whose entire sequence has been codon-optimized for expression in a mammalian cell (e.g., a human cell). In other embodiments, regions of the PUCR construct of the invention are encoded by a transgene comprising non-codon-optimized sequence regions and codon-optimized sequence regions. Codon-optimization refers to the discovery that the frequency of occurrence of synonymous codons (i.e., codons that code for the same amino acid) in coding DNA is biased in different organisms. Such codon degeneracy allows an identical polypeptide to be encoded by a variety of nucleotide sequences. A variety of codon-optimization methods are known in the art (see, e.g., U.S. Pat. Nos. 5,786,464 and 6,114,148). In one aspect, the PUCR of the invention comprises an intracellular domain. In some embodiments, the intracellular domain comprises a signaling domain. For example, in some embodiments, the signaling domain comprises a signaling domain, or fragments thereof, of, but not limited to, the following proteins: a CD3-zeta chain, 4-1BB and CD28 signaling modules, and any combination thereof. In some embodiments the intracellular domain comprises a co-stimulatory signaling domain. For example, in some embodiments, the co-stimulatory signaling domain comprises an intracellular domain, or fragment thereof, of, but not limited to, the following proteins: CD27, CD28, 4-1BB, OX40, CD30, CD40, PD-1, ICOS, lymphocyte function-associated antigen-1 (LFA-1), CD2, CD7, LIGHT, NKG2C, B7-H3, a CD83 ligand, and any combination thereof.

In one aspect, the PUCR of the invention comprises a transmembrane domain. In some embodiments, the transmembrane domain comprises the transmembrane domain, or fragments thereof, of the following proteins: the alpha chain of the T-cell receptor, the beta chain of the T-cell receptor, the zeta chain of the T-cell receptor, CD28, CD3 epsilon, CD45, CD4, CD5, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and any combination thereof.

In one aspect of the invention, the PUCR comprises a hinge region. In some embodiments, said hinge region is a CD8 hinge region. In some embodiments, said hinge region is a CD28 hinge region. In some embodiments, said hinge region is a hybrid CD8 and CD28 hinge region.

In one aspect of the invention, the PUCR is conjugated (i.e., bound) to a specificity agent. In some embodiments, the specificity agent one or more of a binding protein (e.g., an antigen or antigen binding fragment thereof), a peptide, a peptidomimetic, a small molecule, a therapeutic agent, a targeting agent, a protein agonist, a protein antagonist, a metabolic regulator, a hormone, a toxin, or a growth factor.

Furthermore, in one aspect, the present invention provides PUCR compositions and their use in medicaments or methods for treating, among other diseases, cancer and diseases caused by disease-causing organisms. In one aspect of the invention, the PUCR can be used to inhibit tumor growth. In another aspect of the invention, the PUCR can be used to kill an infectious agent (e.g., a disease causing organism, such as a bacterium, a protozoan, a fungus, or a parasite).

In one aspect, the invention provides a cell (e.g., a T cell) engineered to express a PUCR, wherein the PUCR can be programmed to target any molecule of interest (e.g., an antigen). In some embodiments, the molecule of interest is a protein associated with cancer. In some embodiments, the protein associated with cancer is present on the cell membrane of a cancerous cell. In some embodiments, the molecule of interest is an antigen from a disease-causing organism. In some embodiments, the molecule of interest is an antigen from a disease-causing organism that is present on the cell membrane of the disease-causing organism. In some embodiments, the molecule of interest is an antigen present on the cell membrane of a cell of the disease-causing organism. In some embodiments, the molecule of interest is an antigen from a disease-causing organism that is present on the cell membrane of a host cell infected with the disease-causing organism. In some embodiments, the molecule of interest is an extracellular matrix component. In some embodiments, the molecule of interest is a complex carbohydrate-containing molecule (e.g., a glycoprotein). In some embodiments, the molecule of interest is a viral protein. In some embodiments, the molecule of interest is a protein complex.

In one aspect, the invention provides methods of making a customized therapeutic host cell for use in the treatment of a disease (e.g., cancer or an infectious disease). In another aspect, the invention provides methods of treating a cancer or inhibiting tumor growth in a subject in need thereof. In one aspect, the invention further provides methods of treating a medical condition caused by a disease-causing organism (e.g., a bacterium, a virus, a prion, a fungus, a parasite, or a protozoan).

In one aspect, the invention provides kits comprising host cell expressing a PUCR described herein.

### A. Programmable Universal Cell Receptors (PUCR)

The present invention encompasses isolated nucleic acid molecules comprising sequences encoding a programmable universal cell receptor (PUCR), wherein the PUCR comprises a catalytic antibody, or a catalytic portion thereof, wherein the sequence of the catalytic antibody, or portion thereof, is contiguous with and in the same reading frame as a nucleic acid sequence encoding a transmembrane domain and an intracellular domain. PUCRs are particularly advantageous because they can be programmed by attaching one or more specificity agents to the PUCR which enables a cell expressing the now programmed PUCR to target a ligand to which the specificity agent specifically binds. Thus, PUCRs can be customized, as desired, to target any ligand of interest which makes them particularly advantageous for immunotherapy.

In one embodiment of the invention, a PUCR comprises a catalytic antibody (e.g., a catalytic 38C2 antibody) or a catalytic portion thereof (e.g., an scFv or an scFab); a hinge region (e.g., a CD8 hinge region or a hybrid CD8 and CD28 hinge region); a transmembrane domain (e.g., a CD3ζ transmembrane domain or a CD28 transmembrane domain); an intracellular domain (e.g., a CD28 intracellular domain and/or a CD3ζ intracellular domain). In some embodiments, the PUCR further comprises a signal peptide. In some embodiments, the PUCR further comprises a detectable moiety (e.g., a myc tag).

In one embodiment of the invention, the PUCR comprises a murine 38C2 scFv or Fab fragment or scFab, a CD8 hinge region; a CD3ζ transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a murine 38C2 scFv or Fab fragment; a CD8 hinge region; a CD3ζ transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a humanized 38C2 scFv, Fab fragment, or scFab; a CD8 hinge region; a CD3ζ transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a humanized 38C2 scFv or Fab fragment, a CD8 hinge region; a CD3ζ transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a murine 33F12 scFv or Fab fragment or scFab, a CD8 hinge region; a CD3ζ transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a murine 33F12 scFv or Fab fragment, a CD8 hinge region; a CD3ζ transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a humanized 33F12 scFv or Fab fragment or scFab, a CD8 hinge region; a CD3ζ transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a humanized 33F12 scFv or Fab fragment, a CD8 hinge region; a CD3ζ transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a murine 38C2 scFv or Fab fragment or scFab; a hybrid CD8 and CD28 hinge region; a CD28 transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a murine 38C2 scFv or Fab fragment or scFab, a hybrid CD8 and CD28 hinge region; a CD28 transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a humanized 38C2 scFv or Fab fragment or scFab; a hybrid CD8 and CD28 hinge region; a CD28 transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a humanized 38C2 scFv or Fab fragment or scFab, a hybrid CD8 and CD28 hinge region; a CD28 transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a murine 33F12 scFv or Fab fragment or scFab, a hybrid CD8 and CD28 hinge region; a CD28 transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a murine 33F12 scFv or Fab fragment or scFab, a hybrid CD8 and CD28 hinge region; a CD28 transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B. Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

In one embodiment of the invention, the PUCR comprises a humanized 33F12 scFv or Fab fragment or scFab; a hybrid CD8 and CD28 hinge region; a CD28 transmembrane domain; a CD28 intracellular domain; and a CD3ζ intracellular domain. Optionally, the PUCR may include an N-terminal signal peptide. Alternative intracellular domains that may be included in the PUCR include, but are not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD7 intracellular domain, a LIGHT intracellular domain, a LIGHT intracellular domain, a NKG2C intracellular domain, a CD83 ligand intracellular domain. Thus, in some embodiments, the PUCR comprises a humanized 33F12 scFv or Fab fragment or scFab, a hybrid CD8 and CD28 hinge region; a CD28 transmembrane domain; and one or more intracellular domains selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand intracellular domains. Alternative transmembrane domains that may be included in the PUCR include, but are not limited to, a transmembrane domain derived from CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, or FGFR2B.

Alternative hinge regions that may be included in the PUCR include, but are not limited to, the hinge region of an antibody (e.g., IgG, IgG1, IgG2, IgG3, IgG4, IgA, IgM, IgE, IgD), a (Gly4Ser)ₙ linker, or an XTEN peptide.

1. Catalytic Antibodies

In one aspect of the invention, a PUCR comprises a catalytic antibody, or a catalytic portion thereof, referred to herein as the “catalytic antibody region”. Catalytic antibodies are immunoglobulins that comprise a reactive amino acid residue which enables them to react with a variety of molecular entities in a self-assembly process and become linked with the molecular entity (see, e.g., Guo et al. (2006) Proc. Nat'l. Acad. Sci. USA 103(29): 11009-14; U.S. Pat. No. 5,733,757). Many catalytic antibodies, and methods of generating them, are known in the art (see, e.g., U.S. Pat. Nos. 6,210,938; 6,368,839; 6,326,176; 6,589,766; and U.S. Pat. Nos. 5,985,626, 5,733,757; 5,500,358; 5,126,258; 5,030,717; and 4,659,567; the entire contents of which are herein incorporated by reference in their entirety). Catalytic antibodies suitable for use in the PUCRs of the present invention may be obtained by conventional immunization, reactive immunization in vivo, or by reactive selection in vitro, such as with phage display.

In some embodiments, the catalytic antibody is an aldolase catalytic antibody. Aldolase catalytic antibodies comprise a reactive lysine residue having an ε-amino group (e.g., Lys93 of murine or humanized 38C2). Through the reactive lysine residue, these antibodies catalyze aldol and retro-aldol reactions using the enamine mechanism of natural aldolases (Wagner et al. (1995) SCIENCE 270, 1797-1800; Barbas et al. (1997) SCIENCE 278, 2085-2092; Zhong et al. (1999) ANGEW. CHEM. INT. ED. 38, 3738-3741; Karlstrom et al. (2000) PROC. NAT'L. ACAD. SCI. USA, 97: 3878-3883). Thus, aldolase catalytic antibodies may be covalently linked to a reactivity moiety comprising a ketone, diketone, beta lactam, active ester haloketone, lactone, anhydride, maleimide, epoxide, aldehyde amidine, guanidine, imines, eneamines, phosphates, phosphonates, epoxides, aziridines, thioepoxides, masked or protected diketones (ketals for example), lactams, haloketones, aldehydes, and the like, that is associated with a specificity agent of interest. In some embodiments the catalytic antibody is the murine antibody 38C2, or a chimeric or humanized version of said antibody. In some embodiments, the catalytic antibody is the murine antibody 33F12, or a chimeric or humanized version of said antibody (see, e.g., Goswami et al. (2009) BIOORG. MED. CHEM. LETT. 19(14): 3821-4). In some embodiments, the catalytic antibody is the antibody produced by the hybridoma 40F12 (Zhu et al., (2004) J. MOL. BIOL. 343: 1269-80; Rader et al., (1998)) or a chimeric or humanized version of said antibody. In some embodiments, the catalytic antibody is the antibody produced by the hybridoma 42F1 (Zhu et al., (2004); Rader et al., (1998)) or a chimeric or humanized version of said antibody. In other embodiments, the catalytic antibody is the antibody produced by the hybridoma 85A2 (ATCC accession number PTA-1015), or a chimeric or humanized version of said antibody. In some embodiments, the catalytic antibody is the antibody produced by the hybridoma 85C7 (ATCC accession number PTA-1014) or a chimeric or humanized version of said antibody. In other embodiments, the catalytic antibody is the antibody produced by the hybridoma 92F9 (ATCC accession number PTA-1017), or a chimeric or humanized version of said antibody. In some embodiments, the catalytic antibody is the antibody produced by the hybridoma 93F3 (ATCC accession number PTA-823), or a chimeric or humanized version of said antibody. In other embodiments, the catalytic antibody is the antibody produced by the hybridoma 84G3 (ATCC accession number PTA-824), or a chimeric or humanized version of said antibody. In some embodiments, the catalytic antibody is the antibody produced by the hybridoma 84G11 (ATCC accession number PTA-1018), or a chimeric or humanized version of said antibody. In other embodiments, the catalytic antibody is the antibody produced by the hybridoma 84H9 (ATCC accession number PTA-1019), or a chimeric or humanized version of said antibody. In some embodiments, the catalytic antibody is the antibody produced by the hybridoma 85H6 (ATCC accession number PTA-825), or a chimeric or humanized version of said antibody. In other embodiments, the catalytic antibody is the antibody produced by the hybridoma 90G8 (ATCC accession number PTA-1016), or a chimeric or humanized version of said antibody. Additional aldolase catalytic antibodies are known in the art (see, e.g., Kumar et al. (2009) BIOORG. MED. CHEM. LETT. 19(14): 3821-4).

Other catalytic antibodies may also be used in the PUCRs of the present invention. For example, in some embodiments, the catalytic antibody is a beta lactamase catalytic antibody. In other embodiments, the catalytic antibody is an esterase catalytic antibody (see, e.g., Wirsching et al. (1995) SCIENCE 270: 1775-82). In some embodiments, the catalytic antibody is an amidase catalytic antibody. In other embodiments, the catalytic antibody is an thioesterase catalytic antibody (see, e.g., Janda et al. (1994) PROC. NAT'L. ACAD. SCI. USA 91: 2532-2536).

In some embodiments, the catalytic antibody, or catalytic portion thereof, comprises a reactive amino acid residue selected from the group consisting of a reactive cysteine residue, a reactive tyrosine residue, a reactive lysine residue, and a reactive serine residue. For example, thioesterase catalytic antibodies contain a reactive cysteine residue. Thioesterase catalytic antibodies may be covalently linked with maleimide-containing moieties or other thiol-reactive groups such as iodoacetamides, aryl halides, disulfhydryls, and the like.

In some embodiments, the catalytic antibody, or catalytic portion thereof, for use in the PUCRs of the present invention is a catalytic antibody that forms reversible covalent linkages. In other embodiments, the catalytic antibody, or catalytic portion thereof, for use in the PUCRs of the present invention is a catalytic antibody that forms non-reversible covalent linkages. For example, catalytic antibodies derived from reactive immunization with 1,3-diketones form reversible covalent linkages. Due to this reversibility, a reactive moiety comprising a diketone derivative compound that is bound to an aldolase antibody (e.g., 38C2) can be released from the antibody through competition with the covalent binding hapten JW (Wagner et al. (1995) SCIENCE 270, 1797-800), or related compounds. This allows for immediate neutralization of the conjugation of a specificity agent to a PUCR, as necessary. Alternatively, the catalytic antibody forms non-reversible covalent linkages. The use of a catalytic antibody that forms a non-reversible covalent linkage may be particularly advantageous when the PUCR is programmed with a specificity agent comprising a reactive moiety that is a diketone. Without wishing to be being bound by any particular theory, it is believed that said non-reversible covalent linkages are stable regardless of the pH of the surrounding environment (e.g., from pH 3.0 to pH 11.0). This stability is particularly advantageous when targeting tumors, since some tumor environments exhibit reduced pH as compared to normal tissue environments. This stability is also advantageous in formulating, delivering and storing the PUCRs of the present invention.

In some embodiments, the catalytic portion of the catalytic antibody used in the PUCRs of the present invention is a scFv. In some embodiments, the scFv is an scFv derived from murine aldolase catalytic antibody 38C2. In other embodiments, the scFv is an scFv derived from humanized aldolase catalytic antibody 38C2. In some embodiments, the scFv is an scFv derived from murine aldolase catalytic antibody 33F12. In other embodiments, the scFv is an scFv derived from humanized aldolase catalytic antibody 33F12.

ScFvs can be prepared according to methods known in the art (see, for example, Bird et al. (1988) SCIENCE 242:423-426, and Huston et al. (1988) PROC. NATL. ACAD. SCI. USA 85:5879-5883). ScFv molecules can be produced by linking VH and VL regions together using flexible polypeptide linkers. In some embodiments, the scFvs for use in the present invention comprise a linker (e.g., a Ser-Gly linker) with an optimized length and/or amino acid composition. The linker length can greatly affect how the variable regions of a scFv fold and interact. For examples of linker orientation and size see, for example, Hollinger et al. (1993) PROC. NAT'L. ACAD. SCI. USA 90: 6444-6448, U.S. Pat. Appl. Publ. Nos. 2005/0100543, 2005/0175606, 2007/0014794, and PCT Publication Nos. WO 2006/020258 and WO 2007/024715, the contents of which are herein incorporated by reference, and in particular, the disclosure regarding linkers).

In some embodiments, the scFv for use in the PUCRs of the present invention comprises a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally-occurring amino acid. In some embodiments, the linker sequence comprises amino acids glycine and serine. In other embodiments, the linker sequence comprises glycine and serine repeats, such as (Gly₄Ser)ₙ, where n is a positive integer equal to or greater than 1 (SEQ ID NO: 21). In other embodiments, the linker is (Gly₄Ser)₄ (SEQ ID NO: 22) or (Gly₄Ser)₃ (SEQ ID NO: 23). Variation in the linker length may retain or enhance activity, giving rise to superior efficacy in activity studies.

In some embodiments, the catalytic portion of the catalytic antibody used in the PUCRs of the present invention is a scFab. In some embodiments, the scFab is an scFab derived from murine aldolase catalytic antibody 38C2. In other embodiments, the scFab is an scFab derived from humanized aldolase catalytic antibody 38C2. In some embodiments, the scFab is an scFab derived from murine aldolase catalytic antibody 33F12. In other embodiments, the scFab is an scFab derived from humanized aldolase catalytic antibody 33F12.

scFabs can be prepared according to methods known in the art (see, for example, Hust et al. (2007) BMC BIOTECHNOL. 7:14; and Koerber et al. (2015) J. MOL. BIOL. 427(2): 576-86). In some embodiments, the scFab comprises a polypeptide linker of at least 30 amino acids, preferably between 32 and 50 amino acids. In some embodiments, the polypeptide linker is a poly-GlySer linker (e.g., the linker of SEQ ID NO: 54). It will be understood by one of ordinary skill in the art that the catalytic antibody, or a catalytic portion thereof, for use in the PUCR of the present invention may be modified to vary its amino acid sequence (as compared to a wild-type catalytic antibody or catalytic portion thereof), to increase or decrease its catalytic activity, but not eliminate its catalytic activity. In some embodiments, the catalytic antibody, or catalytic portion thereof (e.g., a scFv), is substantially identical to a catalytic antibody, or catalytic portion thereof, disclosed herein.

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. In some embodiments, the identity exists over a region that is at least about 30 nucleotides (or 10 amino acids) in length, or more preferably over a region that is 60 to 150 or 600 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman (1970) ADV. APPL. MATH. 2: 482c, by the homology alignment algorithm of Needleman and Wunsch (1970) J. MOL. BIOL. 48: 443-53, by the search for similarity method of Pearson and Lipman (1988) PROC. NAT'L. ACAD. SCI. USA 85: 2444, by computerized implementations of these algorithms (GAP, BESTFIT, FASTA, and TFASTA in the Wisconsin Genetics Software Package), or by manual alignment and visual inspection. Two examples of algorithms that are suitable for determining percent sequence identity and sequence similarity are the BLAST and BLAST 2.0 algorithms, which are described in Altschul et al., (1977) NUC. ACIDS RES. 25: 3389-3402; and Altschul et al. (1990) J. MOL. BIOL. 215: 403-410, respectively. Software for performing BLAST analyses is publicly available through the National Center for Biotechnology Information. Percent identity between two amino acid sequences can also be determined using the algorithm of E. Meyers and W. Miller (1988) COMPUT. APPL. BIOSCI. 4:11-17, which has been incorporated into the ALIGN program (version 2.0), using a PAM 120 weight residue table, a gap length penalty of 12 and a gap penalty of 4. In addition, the percent identity between two amino acid sequences can be determined using the algorithm disclosed in Needleman and Wunsch (1970) J. MOL. BIOL. 48:444-453, which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

2. Transmembrane Domains

The transmembrane domain of the PUCRs of the present invention can be in any form known in the art. As used herein, the term “transmembrane domain” refers to any polypeptide structure that is thermodynamically stable in a cell membrane, preferably a eukaryotic cell membrane (e.g., a mammalian cell membrane). Transmembrane domains compatible for use in the PUCRs disclosed herein may be obtained from any naturally occurring transmembrane protein, or a fragment thereof. Alternatively, the transmembrane domain can be a synthetic, non-naturally occurring transmembrane protein, or a fragment thereof, e.g., a hydrophobic protein segment that is thermodynamically stable in a cell membrane (e.g., a mammalian cell membrane). Typical transmembrane domains comprise from about 15 to about 35 hydrophobic amino acid residues that form a helix which spans about 30 angstroms of the cellular membrane bilayer.

In some embodiments, the transmembrane domain is derived from a type I membrane protein, i.e., a membrane protein having a single membrane-spanning region that is oriented such that the N-terminus of the protein is present on the extracellular side of the lipid bilayer of the cell and the C-terminus of the protein is present on the cytoplasmic side. In some embodiments, the transmembrane protein may be derived from a type II membrane protein, i.e., a membrane protein having single membrane-spanning region that is oriented such that the C-terminus of the protein is present on the extracellular side of the lipid bilayer of the cell and the N-terminus of the protein is present on the cytoplasmic side. In yet other embodiments, the transmembrane domain is derived from a type III membrane protein, i.e., a membrane protein having multiple membrane-spanning segments.

In some embodiments, the transmembrane domain of the PUCRs of the present invention is derived from a Type I single-pass membrane protein. Single-pass membrane proteins include, but are not limited to, CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B. In some embodiments, the transmembrane domain is derived from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B. In some embodiments, the transmembrane domain is derived from CD8α. In some embodiments, the transmembrane domain is derived from 4-1BB/CD137. In other embodiments, the transmembrane domain is derived from CD28 or CD34. In some embodiments the transmembrane domain is synthetic. In some embodiments, the synthetic transmembrane domain comprises predominantly hydrophobic residues such as leucine and valine. In some embodiments, a triplet of phenylalanine, tryptophan and valine will be found at each end of a synthetic transmembrane domain. Optionally, a polypeptide linker, e.g., between 2 and 10 amino acids in length may form a linkage between the transmembrane domain and an intracellular domain of the PUCR. In some embodiments, the polypeptide linker is a glycine-serine doublet.

Transmembrane domains for use in the PUCRs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids in length. Examples of synthetic transmembrane domains are known in the art, for example in U.S. Pat. No. 7,052,906 B1 and PCT Publication No. WO 2000/032776 A2, the contents of which are herein incorporated by reference, and in particular, the disclosure regarding synthetic transmembrane domains).

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5 or more).

In some embodiments, the transmembrane domain of the PUCR comprises a transmembrane domain of CD3; or a functional portion thereof, such as a transmembrane domain that comprises the amino acid sequence LDPKLCYLLDGILFIYGVILT ALFLRVK(SEQ ID NO: 6), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 6. In some embodiments, the transmembrane domain of the PUCR comprises a transmembrane domain of CD3 encoded by the nucleic acid sequence of SEQ ID NO: 16, or a nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 16. The amino acid sequence LCYLLDGILFIYGVILTALFL (SEQ ID NO: 38) is the defined hydrophobic stretch of the CD3ζ transmembrane domain sequence.

In some embodiments, the transmembrane domain of the PUCR, comprises a transmembrane domain of human CD28 (e.g., Accession No. P01747.1) or a functional portion thereof, such as a transmembrane domain that comprises the amino acid sequence FWVLVVVGGVLACYSLLVTVAFIIFWV (SEQ ID NO: 24), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 24. In some embodiments, the transmembrane domain of CD28 comprises the amino acid sequence IEVMYPPPYLDNEKSNGTIIHVKGKHLCPSPLFPGPSKPFWVL VVVGGVLACYSLLVTVAFIIFWV (SEQ ID NO: 25), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 25. In some embodiments, the transmembrane domain of the PUCR comprises a transmembrane domain of CD28 encoded by the nucleic acid sequence of SEQ ID NO: 61, or a nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 61.

3. Intracellular Domain

The PUCRs disclosed herein comprise an intracellular domain or region. In some embodiments, the intracellular domain of the PUCRs comprise a signaling domain. A signaling domain is generally responsible for activation of at least one of the normal effector functions of the cell (e.g., an immune cell (e.g., a T cell) in which the PUCR is being expressed. The term “effector function” refers to a specialized function of a cell. For example, the effector function of a T cell may include a cytolytic activity or helper activity, including, for example, the secretion of cytokines. Thus, the term “signaling domain” refers to the portion of a protein which transduces the effector function signal and directs the cell to perform a specialized function. While usually the entire intracellular signaling domain can be employed, in many cases it is not necessary to use the entire chain or domain. Thus, to the extent that a truncated portion of the intracellular signaling domain is used, such truncated portion may be used in place of the intact domain as long as it transduces the effector function signal. The term “signaling domain” therefore also includes any truncated portion of a signaling domain sufficient to transduce an effector function signal. However, in some embodiments, the PUCR comprises a signaling domain that does not transduce an effect function signal in the cell in which the PUCR is expressed. Examples of intracellular signaling domains suitable for use in the PUCRs disclosed herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary signaling domains that act in a stimulatory manner may contain signaling motifs which are known as immunoreceptor tyrosine-based activation motifs (ITAMs). Primary signaling domains containing ITAMs for use in the PUCRs of the present invention include, but are not limited to, the signaling domains of TCR zeta, FcR gamma, FcR beta, CD3 gamma, CD3 delta, CD3 epsilon, CD5, CD22, CD79a, CD79b, and CD66d. In some embodiments, the PUCR of the present invention comprises a signaling domain of CD3ζ. In other embodiments, the PUCR of the present invention comprises a signaling domain of CD28. In some embodiments of the invention, the PUCR comprises a signaling domain of 4-1BB (also known as CD137). In some embodiments of the invention, the PUCR comprises a combination of two or more of the signaling domains described herein. In some embodiments of the invention, the PUCR comprises both a signaling domain of CD28 and a signaling domain of CD3ζ. In some embodiments of the invention, the PUCR comprises both a signaling domain of CD28 and a signaling domain of 4-1BB. In some embodiments of the invention, the PUCR comprises both a signaling domain of 4-1BB and a signaling domain of CD3ζ.

In some embodiments of the invention, the PUCR comprises an intracellular domain of CD28. In some embodiments, the CD28 intracellular domain comprises the amino acid sequence of SEQ ID NO: 7, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 7. In some embodiments, the CD28 intracellular domain is encoded by the nucleic acid sequence of SEQ ID NO: 17, or a nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 17.

In some embodiments of the invention, the PUCR comprises an intracellular domain of CD3ζ. In some embodiments, the intracellular domain of CD3ζ comprises the amino acid sequence of SEQ ID NO: 8, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 8. In some embodiments, the CD3ζ intracellular domain is encoded by the nucleic acid sequence of SEQ ID NO: 18, or an nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 18.

In some embodiments of the invention, the PUCR comprises an intracellular domain of CD3ζ. In some embodiments, the intracellular domain of CD3ζ comprises the amino acid sequence of SEQ ID NO: 59, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 59. In some embodiments, the CD3ζ intracellular domain is encoded by the nucleic acid sequence of SEQ ID NO: 62, or an nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 62.

In some embodiments of the invention, the PUCR comprises an intracellular domain of 4-1BB. 4-1BB is a tumor necrosis factor-receptor family member expressed following CD28 activation. In some embodiments, the 4-1BB intracellular domain comprises the amino acid sequence KRGRKKLLYIFKQPFMRPVQ TTQEEDGCSCRFPEEEEGGCEL (SEQ ID NO: 26), a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 26. In some embodiments, the 4-1BB intracellular domain is encoded by the nucleic acid sequence of SEQ ID NO: 27, or an nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 27.

TABLE 2
Exemplary Intracellular Domain Sequences

| | |
|---|---|
| CD28 intracellular | RSKRSRLLHSDYMNMTPRRPGPTRKHYQPYAPPRDFAAYRS |
| domain amino acid | (SEQ ID NO: 7) |
| sequence | |
| CD28 intracellular | AGGAGTAAGAGGAGCAGGCTCCTGCACAGTGACTACATGAACATGACTC |
| domain nucleic acid | CCCGCCGCCCCGGGCCCACCCGCAAGCATTACCAGCCCTATGCCCCACC |
| sequence | ACGCGACTTCGCAGCCTATCGCTCC |
| | (SEQ ID NO: 17) |
| CD3ζ intracellular | RVKFSRSADAPAYQQGQNQLYNELNLGRREEYDVLDKRRGRDPEMGGKP |
| domain amino acid | QRRKNPQEGLYNELQKDKMAEAYSEIGMKGERRRGKGHDGLYQGLSTAT |
| sequence | KDTYDALHMQALPPR |
| | (SEQ ID NO: 8) |
| CD3ζ intracellular | AGAGTGAAGTTCAGCAGGAGCGCAGACGCCCCCGCGTACCAGCAGGGCC |
| domain nucleic acid | AGAACCAGCTCTATAACGAGCTCAATCTAGGACGAAGAGAGGAGTACGA |
| sequence | TGTTTTGGACAAGAGACGTGGCCGGGACCCTGAGATGGGGGGAAAGCCG |
| | AGAAGGAAGAACCCTCAGGAAGGCCTGTACAATGAACTGCAGAAAGATA |
| | AGATGGCGGAGGCCTACAGTGAGATTGGGATGAAAGGCGAGCGCCGGAG |
| | GGGCAAGGGGCACGATGGCCTTTACCAGGGTCTCAGTACAGCCACCAAG |
| | GACACCTACGACGCCCTTCACATGCAGGCCCTGCCCCCTCGCTAA |
| | (SEQ ID NO: 18) |
| CD3ζ intracellular | RVKFSRSADAPAYQQGQNQLYNELNLGRREEYDVLDKRRGRDPEMGGKP |
| domain amino acid | RRKNPQEGLYNELQKDKMAEAYSEIGMKGERRRGKGHDGLYQGLSTATK |
| sequence | DTYDALHMQALPPR |
| | (SEQ ID NO: 59) |
| CD3ζ intracellular | AGAGTGAAGTTCAGCAGGAGCGCAGACGCCCCCGCGTACCAGCAGGGCC |
| domain nucleic acid | AGAACCAGCTCTATAACGAGCTCAATCTAGGACGAAGAGAGGAGTACGA |
| sequence | TGTTTTGGACAAGAGACGTGGCCGGGACCCTGAGATGGGGGGAAAGCCG |
| | AGAAGGAAGAACCCTCAGGAAGGCCTGTACAATGAACTGCAGAAAGATA |
| | AGATGGCGGAGGCCTACAGTGAGATTGGGATGAAAGGCGAGCGCCGGAG |
| | GGGCAAGGGGCACGATGGCCTTTACCAGGGTCTCAGTACAGCCACCAAG |
| | GACACCTACGACGCCCTTCACATGCAGGCCCTGCCCCCTCGCTAA |
| | (SEQ ID NO: 62) |
| 4-1BB intracellular | KRGRKKLLYIFKQPFMRPVQTTQEEDGCSCRFPEEEEGGCEL |
| domain amino acid | (SEQ ID NO: 26) |
| sequence | |
| 4-1BB intracellular | AAACGGGGCAGAAAGAAACTCCTGTATATATTCAAACAACCATTTATGA |
| domain nucleic acid | GACCAGTACAAACTACTCAAGAGGAAGATGGCTGTAGCTGCCGATTTCC |
| sequence | AGAAGAAGAAGAAGGAGGATGTGAACTG |
| | (SEQ ID NO: 27) |

In some embodiments, a signaling domain used in a PUCR of the present invention comprises a modified ITAM which has been altered (e.g., mutated or truncated) as compared to the native ITAM. In some embodiments, said modified ITAM has increased activity as compared to the native ITAM. In some embodiments, said modified ITAM has decreased activity as compared to the native ITAM. In some embodiments, the signaling domain comprises one ITAM. In some embodiments, the signaling domain comprises multiple (e.g., one, two, three, four or more) ITAMs.

In some embodiments, the intracellular domain of a PUCR of the present invention comprises a co-stimulatory signaling domain. In some embodiments, the intracellular domain of the PUCR of the present invention comprises a signaling domain and a co-stimulatory domain. The term “co-stimulatory signaling domain,” as used herein, refers to a portion of a protein that mediates signal transduction within a cell to induce a response, e.g., an effector function. The co-stimulatory signaling domain of a PUCR of the present invention can be a cytoplasmic signaling domain from a co-stimulatory protein, which transduces a signal and modulates responses mediated by immune cells (e.g., T cells or NK cells).

Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB ligand/TNFSF9, BAFF/BLyS/TNFSF13B, BAFF R/TNFRSF13C, CD27/TNFRSF7, CD27 ligand/TNFSF7, CD30/TNFRSF8, CD30 ligand/TNFSF8, CD40/TNFRSF5, CD40/TNFSF5, CD40 ligand/TNFSF5, DR3/TNFRSF25, GITR/TNFRSF18, GITR ligand/TNFSF18, HVEM/TNFRSF14, LIGHT/TNFSF14, lymphotoxin-alpha/TNF-beta, OX40/TNFRSF4, OX40 ligand/TNFSF4, RELT/TNFRSF19L, TACI/TNFRSF13B, TL1A/TNFSF15, TNF-α, and TNF RII/TNFRSF1B); members of the interleukin-1 receptor/toll-like receptor (TLR) superfamily (e.g., TLR1, TLR2, TLR3, TLR4, TLR5, TLR6, TLR7, TLR8, TLR9, and TLR10); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, ikaros, integrin alpha 4/CD49d, integrin alpha 4 beta 1, integrin alpha 4 beta 7/LPAM-1, LAG-3, TCL1A, TCL1B, CRTAM, DAP10, DAP12, MYD88, TRIF, TIRAP, TRAF, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory domain comprises an intracellular domain of an activating receptor protein selected from the group consisting of α₄β₁ integrin, β₂ integrins (CD11a-CD18, CD11b-CD18, CD11b-CD18), CD226, CRTAM, CD27, NKp46, CD16, NKp30, NKp44, NKp80, NKG2D, KIR-S, CD100, CD94/NKG2C, CD94/NKG2E, NKG2D, PENS, CEACAM1, BY55, CRACC, Ly9, CD84, NTBA, 2B4, SAP, DAP10, DAP12, EAT2, FcRγ, CD3ζ, and ERT. In some embodiments, the co-stimulatory domain comprises an intracellular domain of an inhibitory receptor protein selected from the group consisting of KIR-L, LILRB1, CD94/NKG2A, KLRG-1, NKR-P1A, TIGIT, CEACAM, SIGLEC 3, SIGLEC 7, SIGLEC9, and LAIR-1. In some embodiments, the co-stimulatory domain comprises an intracellular domain of a protein selected from the group consisting of CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like.

In some embodiments, a co-stimulatory signaling domain used in a PUCR of the present invention comprises a modified co-stimulatory signaling domain which has been altered (e.g., mutated or truncated) as compared to the native co-stimulatory signaling domain. In some embodiments, the co-stimulatory signaling domain comprises up to 10 amino acid residue variations (e.g., 1, 2, 3, 4, 5, 6, 7, 8, 9, or 10) as compared to a wild-type co-stimulatory signaling domain. Co-stimulatory signaling domains comprising one or more amino acid variations may be referred to as variant co-stimulatory signaling domains. Mutation of one or more amino acid residues of a co-stimulatory signaling domain may result in an increase in signaling transduction and enhanced stimulation of a cellular responses relative to co-stimulatory signaling domains that does not comprise the mutation. Mutation of one or more amino acid residues of the co-stimulatory signaling domain may alternatively result in a decrease in signaling transduction and reduced stimulation of a cellular responses relative to co-stimulatory signaling domains that does not comprise the mutation. For example, mutation of residues 186 and 187 of the native CD28 amino acid sequence may result in an increase in co-stimulatory activity and induction of immune responses by the co-stimulatory signaling domain of the PUCR. In some embodiments, the mutations are substitution of a lysine at each of positions 186 and 187 with a glycine residue of the CD28 co-stimulatory signaling domain, referred to as a CD28_{LL→GG} variant. Additional mutations that can be made in co-stimulatory signaling domains that may enhance or reduce co-stimulatory activity of the domain will be evident to one of ordinary skill in the art.

In some embodiments, a PUCR of the present invention may comprise more than one co-stimulatory signaling domain (e.g., 2, 3, 4, 5, 6, 7, 8, or more co-stimulatory signaling domains). In some embodiments, the PUCR comprises two or more co-stimulatory signaling domains from different co-stimulatory proteins, such as any two or more co-stimulatory proteins described herein. In some embodiments, the PUCR comprises two or more co-stimulatory signaling domains from the same co-stimulatory protein (i.e., repeats).

Selection of the type(s) of co-stimulatory signaling domain(s) may be based on factors such as the type of host cell that will be expressing the PUCR (e.g., T cells, NK cells, macrophages, neutrophils, or eosinophils), and the desired cellular effector function (e.g., an immune effector function).

The signaling sequences (i.e., a signaling domain and/or a co-stimulatory signaling domain) in the intracellular domain may be linked to each other in a random or specified order. The intracellular domain of the PUCR may comprise one or more linkers disposed between the signaling sequences. In some embodiments, the linker may be a short oligo- or a polypeptide linker, e.g., between 2 and 10 amino acids (e.g., 2, 3, 4, 5, 6, 7, 8, 9, or 10 amino acids) in length. In some embodiments, the linker may be more than 10 amino acids in length. Any linker disclosed herein, or apparent to those of skill in the art, may be used in the intracellular domain of a PUCR of the present invention.

4. Hinge Regions

In some embodiments, the PUCR further comprises a hinge region. In some embodiments, the hinge region is located between the catalytic antibody region and the transmembrane domain A hinge region is an amino acid segment that is generally found between two domains of a protein and may allow for flexibility of the PUCR and movement of one or both of the domains relative to one another. Any amino acid sequence that provides such flexibility and movement of the catalytic antibody region relative to the transmembrane domain of the PUCR can be used.

In some embodiments, the hinge region comprises from about 10 to about 100 amino acids, e.g., from about 15 to about 75 amino acids, from about 20 to about 50 amino acids, or from about 30 to about 60 amino acids. In some embodiments, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. In some embodiments the hinge region is more than 100 amino acids in length. In some embodiments, the hinge region is a hinge region of a naturally-occurring protein. Hinge regions of any protein known in the art to comprise a hinge region may be used in the PUCRs described herein. In some embodiments, the hinge region is at least a portion of a hinge region of a naturally occurring protein and confers flexibility to the extracellular region of the PUCR.

In some embodiments, the hinge region is a CD8 hinge region. In some embodiments, the hinge region is a CD8αhinge region. In some embodiments, the hinge region is a portion of a CD8 hinge region, e.g., a fragment containing at least 15 (e.g., 20, 25, 30, 35, or 40) consecutive amino acids of the CD8 hinge region. In some embodiments, the hinge region is a portion of a CD8αhinge region, e.g., a fragment containing at least 15 (e.g., 20, 25, 30, 35, or 40) consecutive amino acids of the CD8αhinge region. The CD8 hinge region may comprise the amino acid sequence of SEQ ID NO: 5, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 5. Alternatively, the CD8 hinge region may comprise the amino acid sequence comprises the amino acid sequence of SEQ ID NO: 28, SEQ ID NO: 29, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 28 or SEQ ID NO: 29. In some embodiments, the CD8 hinge region is encoded by the nucleic acid sequence of SEQ ID NO: 30, or an nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 30.

In some embodiments, the hinge region is a hybrid CD8 and CD28 hinge region. In some embodiments, the hybrid CD8 and CD28 hinge region may comprise the amino acid sequence of SEQ ID NO: 55, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 55. In some embodiments, the hybrid CD8 and CD28 hinge region may comprise the amino acid sequence comprises the amino acid sequence of SEQ ID NO: 56, or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 56. In some embodiments, the hybrid CD8 and CD28 hinge region may comprise the amino acid sequence of SEQ ID NO: 58 or a functional portion thereof, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 58. In some embodiments, the hybrid CD8 and CD28 hinge region may comprise a linker sequence (e.g., the linker sequence of SEQ ID NO: 57). In some embodiments, the CD8 and CD28 hinge region is encoded by the nucleic acid sequence of SEQ ID NO: 60, or an nucleic acid sequence having at least 85%, 86%, 87%, 88%, 89%, 90%, 91%, 92%, 93%, 94%, 95%, 96%, 97%, 98%, 99% or more sequence identity to the nucleic acid sequence of SEQ ID NO: 60.

TABLE 3
Exemplary Hinge Region Sequences

| | |
|---|---|
| CD8 | AKPTTTPAPRPPTPAPTIASQPLSLRPEACRPAAGGAVHTRG |
| hinge | LDFA (SEQ ID NO: 5) |
| amino | |
| acid | |
| sequence | |
| CD8 | GCTAAGCCCACCACGACGCCAGCGCCGCGACCACCAACACCG |
| hinge | GCGCCCACCATCGCGTCGCAGCCCCTGTCCCTGCGCCCAGAG |
| nucleic | GCGTGCCGGCCAGCGGCGGGGGGCGCAGTGCACACGAGGGG |
| acid | GCTGGACTTCGCC (SEQ ID NO: 15) |
| sequence | |
| CD8 | AKPTTTPAPRPPTPAPTIASQPLSLRPEAXRPAAGGAVHTRG |
| hinge | LDFA |
| amino | wherein X is any amino acid except |
| acid | cysteine (SEQ ID NO: 28) |
| sequence | |
| CD8 | TTTPAPRPPTPAPTIASQPLSLRPEACRPAAGGAVHTRGLDF |
| hinge | ACD (SEQ ID NO: 29) |
| CD8 | ACCACGACGCCAGCGCCGCGACCACCAACACCGGCGCCCACC |
| hinge | ATCGCGTCGCAGCCCCTGTCCCTGCGCCCAGAGGCGTGCCGG |
| nucleic | CCAGCGGCGGGGGGCGCAGTGCACACGAGGGGGCTGGACTTC |
| acid | GCCTGTGAT (SEQ ID NO: 30) |
| sequence | |
| Hybrid | AKPTTTPAPRPPTPAPTIASQPLSLRPEACRPAAGGAVHTRG |
| CD8 and | LDFAPRKIEVMYPPPYLDNEKSNGTIIHVKGKHLCPSPLFPG |
| CD28 | PSKP (SEQ ID NO: 55) |
| hinge | |
| amino | |
| acid | |
| sequence | |
| CD8 | AKPTTTPAPRPPTPAPTIASQPLSLRPEACRPAAGGAVHTRG |
| portion | LDFA (SEQ ID NO: 56) |
| of | |
| hybrid | |
| CD8 and | |
| CD28 | |
| hinge | |
| amino | |
| acid | |
| sequence | |
| Hinge | PR (SEQ ID NO: 57) |
| linker | |
| amino | |
| acid | |
| sequence | |
| CD28 | KIEVMYPPPYLDNEKSNGTIIHVKGKHLCPSPLFPGPSKP |
| portion | (SEQ ID NO: 58) |
| of | |
| hybrid | |
| CD8 and | |
| CD28 | |
| hinge | |
| amino | |
| acid | |
| sequence | |
| Hybrid | GCTAAGCCCACCACGACGCCAGCGCCGCGACCACCAACACCG |
| CD8 and | GCGCCCACCATCGCGTCGCAGCCCCTGTCCCTGCGCCCAGAG |
| CD28 | GCGTGCCGGCCAGCGGCGGGGGGCGCAGTGCACACGAGGGG |
| hinge | GCTGGACTTCGCCCCTAGGAAAATTGAAGTTATGTATCCTCC |
| nucleic | TCCTTACCTAGACAATGAGAAGAGCAATGGAACCATTATCCA |
| acid | TGTGAAAGGGAAACACCTTTGTCCAAGTCCCCTATTTCCCGG |
| sequence | ACCTTCTAAGCCC (SEQ ID NO: 60) |

In some embodiments, the hinge region is a hinge region of an antibody (e.g., IgG, IgA, IgM, IgE, or IgD antibodies). In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody.

In some embodiments, the hinge region is a non-naturally occurring peptide. In some embodiments, the hinge region is disposed between the C-terminus of the catalytic domain and the N-terminus of the transmembrane domain of the PUCR. In some embodiments, the hinge region is a (GlyₓSer)ₙ linker, wherein x and n, independently can be an integer between 3 and 12, including 3, 4, 5, 6, 7, 8, 9, 10, 11, 12, or more. In some embodiments, the hinge region is (Gly₄Ser)ₙ, wherein n can be an integer between 3 and 60, or more, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. In some embodiments, the hinge region is (Gly₄Ser)₃ (SEQ ID NO: 23). In some embodiments, the hinge region is (Gly₄Ser)₆ (SEQ ID NO: 31). In some embodiments, the hinge region is (Gly₄Ser)₉ (SEQ ID NO: 32). In some embodiments, the hinge region is (Gly₄Ser)₁₂ (SEQ ID NO: 33). In some embodiments, the hinge region is (Gly₄Ser)₁₅ (SEQ ID NO: 34). In some embodiments, the hinge region is (Gly₄Ser)₃₀ (SEQ ID NO: 35). In some embodiments, the hinge region is (Gly₄Ser)₄₅ (SEQ ID NO: 36). In some embodiments, the hinge region is (Gly₄Ser)₆₀ (SEQ ID NO: 37). In some embodiments, the hinge region is a poly-GlySer linker (SEQ ID NO: 54).

In some embodiments, the hinge region is an extended recombinant polypeptide (XTEN), which is an unstructured polypeptide consisting of hydrophilic residues of varying lengths (e.g., 10-80 amino acid residues). Amino acid sequences of XTEN peptides are known in the art (see, e.g., U.S. Pat. No. 8,673,860, the contents of which are herein incorporated by reference). In some embodiments, the hinge region is an XTEN peptide and comprises 60 amino acids. In some embodiments, the hinge region is an XTEN peptide and comprises 30 amino acids. In some embodiments, the hinge region is an XTEN peptide and comprises 45 amino acids. In some embodiments, the hinge region is an XTEN peptide and comprises 15 amino acids.

5. Signal Peptides

In some embodiments, the PUCRs disclosed herein further comprises a signal peptide (also known as a signal sequence) at the N-terminus of the polypeptide. In general, signal sequences are peptide sequences that target a polypeptide to the desired site in a cell. In some embodiments, the signal sequence targets the PUCR to the secretory pathway of the cell and will allow for integration and anchoring of the PUCR into the lipid bilayer of the cellular membrane. Signal sequences, including signal sequences of naturally occurring proteins or synthetic, non-naturally occurring signal sequences, that are compatible for use in the PUCRs described herein will be evident to those of skill in the art. In some embodiments, the signal sequence for use in the PUCRs of the present invention is the signal sequence of CD8α. In other embodiments, the signal sequence is the signal sequence of CD28. In some embodiments, the signal sequence is the signal sequence of the murine kappa chain. In yet other embodiments, the signal sequence is the signal sequence of CD16. In some embodiments, the signal sequence is the signal sequence of murine immunoglobulin heavy chain. In some embodiments, the signal peptide comprises the amino acid sequence MEWSWVFLFFLSVTTGVHS (SEQ ID NO: 1). In some embodiments, the signal peptide is encoded by the nucleic acid sequence of SEQ ID NO: 11. In some embodiments, the signal peptide is encoded by the nucleic acid sequence of SEQ ID NO: 46.

### B. Specificity Agents

One advantage of the PUCRs described herein is that they may be programmed to confer specificity to the PUCR to any target molecule (e.g., an antigen). Thus, a specificity agent may be conjugated and/or attached to the PUCR and program the PUCR to target any molecule of interest (e.g., an antigen). In some embodiments, the specificity agent comprises a binding protein (e.g., an antibody or antigen binding fragment thereof). Thus, in some embodiments of the invention, the PUCR may be conjugated to a specificity agent comprising an antibody, or antigen-binding portion thereof, to create a programmed PUCR having specificity for an antigen of interest. In some embodiments, said binding protein is an antibody or antigen binding fragment thereof. In some embodiments, said binding protein is a ligand. In some embodiments, said binding protein is a cytokine. In some embodiments, said binding protein is a receptor.

In some embodiments, the specificity agent comprises a peptide (e.g., a peptide comprising one or more Arg-Gly-Asp (RGD) motifs). In some embodiments, the specificity agent comprises a peptidomimetic (e.g., RGD peptidomimetics). In other embodiments, the specificity agent comprises a small molecule (e.g., folic acid or 2-[3-(1, 3-dicarboxy propyl)-ureido] pentanedioic acid (DUPA)). In some embodiments, the specificity agent comprises a therapeutic agent. In other embodiments, the specificity agent comprises a targeting agent. In some embodiments, the specificity agent comprises a protein agonist. In other embodiments, the specificity agent comprises a metabolic regulator. In some embodiments, the specificity agent comprises a hormone. In other embodiments, the specificity agent comprises a toxin. In some embodiments, the specificity agent comprises a growth factor. In some embodiments, the specificity agent comprises a detectable moiety, such as, but not limited to, biotin. In other embodiments, the specificity agent comprises a ligand. In some embodiments, the specificity agent comprises a protein. In other embodiments, the specificity agent comprises a peptoid. In some embodiments, the specificity agent comprises a DNA aptamer. In other embodiments, the specificity agent comprises a peptide nucleic acid. In some embodiments, the specificity agent comprises a vitamin. In other embodiments, the specificity agent comprises a substrate or a substrate analog. In some embodiments, the specificity agent comprises a cyclic arginine-glycine-aspartic acid peptide (cRGD).

In some embodiments, the specificity agent binds to a protein associated with cancer. In some embodiments, the specificity agent comprises an antibody, or antigen-binding fragment thereof, that specifically binds a protein associated with cancer. Examples of an antigen-binding fragment include, but are not limited to, a Fab fragment or an scFv.

In some embodiments, the protein associated with cancer is a protein that is highly expressed in a cancerous cell (e.g., a tumor cell). In some embodiments, the protein associated with cancer is a protein that is highly expressed on the surface of a cancerous cell (e.g., a tumor cell). In some embodiments, the protein associated with cancer is a cancer biomarker. In some embodiments, the protein associated with cancer is a protein selected from the group consisting of CD19, VEGFR2, PSMA, CEA, GM2, GD2, GD3, EGFR, EGFRvIII, HER2, IL13R, folate receptor, and MUC-1. In some embodiments, the protein associated with cancer is an integrin (e.g., αᵥβ₃). In some embodiments, the protein associated with cancer is selected from the group consisting of cholecystokinin B receptor, gonadotropin-releasing hormone receptor, somatostatin receptor 2, gastrin-releasing peptide receptor, neurokinin 1 receptor, melanocortin 1 receptor, a neurotensin receptor, neuropeptide Y receptor, and C-type lectin like molecule 1. In some embodiments, the specificity agent comprises a targeting molecule listed in Table 4. In some embodiments, the specificity agent binds to a carbohydrate antigen associated with cancer. In some embodiments, the carbohydrate antigen associated with cancer is Tn antigen (GalNAcα-Ser/Thr; see Ju et al. (2008) CANCER RES. 68(6): 1636-46). In some embodiments, the carbohydrate antigen associated with cancer is the STn antigen (NeuAcα6GalNAcα-Ser/Thr; Ju et al. (2008)).

TABLE 4
Exemplary Targeting Molecules

| | | |
|---|---|---|
| SS-14 | Ala-Gly-cyclo(Cys-Lys- | SEQ ID |
| (somatostatin | Asn-Phe-Phe-Trp-Lys-Thr- | NO: 64 |
| analog) | Phe-Thr-Ser-Cys) | |
| OC | D-Phe1-cyclo(Cys2-Phe3- | SEQ ID |
| (somatostatin | D-Trp4-Lys5-Thr6- | NO: 65 |
| analog) | Cys7)Thr(ol)8 | |
| TOC | D-Phe1-cyclo(Cys2-Tyr3- | SEQ ID |
| (somatostatin | D-Trp4-Lys5-Thr6- | NO: 66 |
| analog) | Cys7)Thr(ol)8 | |
| TATE | D-Phe1-cyclo(Cys2-Tyr3- | SEQ ID |
| (somatostatin | D-Trp4-Lys5-Thr6- | NO: 67 |
| analog) | Cys7)Thr8 | |
| NOC | D-Phe1-cyclo(Cys2-1-NaI3- | SEQ ID |
| (somatostatin | D-Trp4-Lys5-Thr6- | NO: 68 |
| analog) | Cys7)Thr(ol)8 | |
| NOC-ATE | D-Phe1-cyclo(Cys2-1-NaI3- | SEQ ID |
| (somatostatin | D-Trp4-Lys5-Thr6- | NO: 69 |
| analog) | Cys7)Thr8 | |
| BOC | D-Phe1-cyclo(Cys2- | SEQ ID |
| (somatostatin | BzThi3-D-Trp4-Lys5-Thr6- | NO: 70 |
| analog) | Cys7)Thr(ol)8 | |
| BOC-ATE | D-Phe1-cyclo(Cys2- | SEQ ID |
| (somatostatin | BzThi3-D-Trp4-Lys5-Thr6- | NO: 71 |
| analog) | Cys7)Thr8 | |
| KE108 | Tyr-cyclo(DAB-Arg-Phe- | SEQ ID |
| (somatostatin | Phe-D-Trp-Lys-Thr-Phe) | NO: 72 |
| analog) | | |
| LM3 | p-Cl-Phe-cyclo(D-Cys-Tyr- | SEQ ID |
| (somatostatin | D-Aph(Cbm)-Lys-Thr- | NO: 73 |
| analog) | Cys)D-Tyr-NH2 | |
| BN | pGlu1-Gln2-Arg3-Leu4- | SEQ ID |
| (bombesin | Gly5-Asn6-Gln7-Trp8- | NO: 74 |
| analog) | Ala9-Val10-Gly11-His12- | |
| | Leu13-Met14-NH2 | |
| RP527 | N3S-Gly-5-Ava-[Gln7- | SEQ ID |
| (bombesin | Trp8-Ala9-Val10-Gly11- | NO: 75 |
| analog) | His12-Leu13-Met14-NH2] | |
| Demobesin 1 | N40-1-bzlg0[D-Phe6- | SEQ ID |
| (bombesin | Gln7-Trp8-Ala9-Val10- | NO: 76 |
| analog) | Gly11-His12-Leu-NHEt13] | |
| Demobesin 4 | N4-[Pro1-Gln2-Arg3-Tyr4- | SEQ ID |
| (bombesin | Gly5-Asn6-Gln7-Trp8- | NO: 77 |
| analog) | Ala9-Val10-Gly11-His12- | |
| | Leu13-Nle14-NH2] | |
| BBS-38 | (NαHis)Ac-β-Ala-β-Ala- | SEQ ID |
| (bombesin | [Gln7-Trp8-Ala9-Val10- | NO: 78 |
| analog) | Gly11-His12-Cha13-Nle14- | |
| | NH2] | |
| BAY 86-4367 | 3-cyano-4- | SEQ ID |
| (bombesin | trimethylammonium- | NO: 79 |
| analog) | benzoyl-Ala(SO3H)- | |
| | Ala(SO3H)-Ava[Gln7- | |
| | Trp8-Ala9-Val10- | |
| | NMeGly11-His12-Sta13- | |
| | Leu14-NH2] | |
| MG | Leu1-Glu2-Glu3-Glu4- | SEQ ID |
| (minigastrin | Glu5-Glu6-Ala7-Tyr8- | NO: 80 |
| analog) | Gly9-Trp10-Met11-Asp12- | |
| | Phe13-NH2 | |
| MGO | D-Glu1-Glu2-Glu3-Glu4- | SEQ ID |
| (minigastrin | Glu5-Glu6-Ala7-Tyr8- | NO: 81 |
| analog) | Gly9-Trp10-Met11-Asp12- | |
| | Phe13-NH2 | |
| MG11 | D-Glu-Ala-Tyr-Gly-Trp- | SEQ ID |
| (minigastrin | Met-Asp-Phe-NH2 | NO: 82 |
| analog) | | |
| H2-Met | His-His-Glu-Ala-Tyr-Gly- | SEQ ID |
| (minigastrin | Trp-Met-Asp-Phe-NH2 | NO: 83 |
| analog) | | |
| H2-Nle | His-His-Glu-Ala-Tyr-Gly- | SEQ ID |
| (minigastrin | Trp-Nle-Asp-Phe-NH2 | NO: 84 |
| analog) | | |
| Demogastrin | N4-D-Glu-(Glu)5-Ala-Tyr- | SEQ ID |
| (minigastrin | Gly-Trp-Met-Asp-Phe-NH2 | NO: 85 |
| analog) | | |
| Cyclo-MG1 | c(γ-D-Glu-Ala-Tyr-D-Lys)- | SEQ ID |
| (minigastrin | Trp-Met-Asp-Phe-NH2 | NO: 86 |
| analog) | | |
| MGD5 | Gly-Ser- | SEQ ID |
| (minigastrin | Cys(succinimidopropionyl- | NO: 87 |
| analog) | Glu-Ala-Tyr-Gly-Trp-Nle- | |
| | Asp-Phe-NH2)-Glu-Ala- | |
| | Tyr-Gly-Trp-Nle-Asp-Phe- | |
| | NH2 | |
| Buserelin | pGlu1-His2-Trp3-Ser4- | SEQ ID |
| (GnRH | Tyr5-D-Ser(tBu)6-Leu7- | NO: 88 |
| analog) | Arg8-Pro9-NHC2H5 | |
| Goserelin | pGlu1-His2-Trp3-Ser4- | SEQ ID |
| (GnRH | Tyr5-D-Ser(tBu)6-Leu7- | NO: 89 |
| analog) | Arg8-Pro9-AzGly10-NH2 | |
| Leuprolide | pGlu1-His2-Trp3-Ser4- | SEQ ID |
| (GnRH | Tyr5-D-Leu6-Leu7-Arg8- | NO: 90 |
| analog) | Pro9-NHC2H5 | |
| Nafarelin | pGlu1-His2-Trp3-Ser4- | SEQ ID |
| (GnRH | Tyr5-D-Nal(2)6-Leu7- | NO: 91 |
| analog) | Arg8-Pro9-NHC2H5 | |
| Triptorelin | pGlu1-His2-Trp3-Ser4- | SEQ ID |
| (GnRH | Tyr5-D-Trp6-Leu7-Arg8- | NO: 92 |
| analog) | Pro9-Gly10-NH2 | |
| Abarelix | Ac-D-Ala1-D-Cpa2-D- | SEQ ID |
| (GnRH | Ala3-Ser4-Tyr5-D-Asp6- | NO: 93 |
| analog) | Leu7-Ilys8-Pro9-D-Ala10- | |
| | NH2 | |
| Acyline | Ac-D-Nal1-D-Cpa2-D- | SEQ ID |
| (GnRH | Pal3-Ser4-Aph(Ac)5-D- | NO: 94 |
| analog) | Aph(Ac)6-Leu7-Ilys8-Pro9- | |
| | D-Ala10-NH2 | |
| Antarelix | Ac-D-Nal1-D-Cpa2-D- | SEQ ID |
| (GnRH | Pal3-Ser4-Tyr5-D-Hci6- | NO: 95 |
| analog) | Leu7-Ilys8-Pro9-D-Ala10- | |
| | NH2 | |
| Antide | Ac-D-Nal1-D-Cpa2-D- | SEQ ID |
| (GnRH | Pal3-Ser4-Lys(Nic)5-D- | NO: 96 |
| analog) | Lys(Nic)6-Leu7-Ilys8-Pro9- | |
| | D-Ala10-NH2 | |
| Azaline B | Ac-D-Nal1-D-Cpa2-D- | SEQ ID |
| (GnRH | Pal3-Ser4-Aph(Atz)5-D- | NO: 97 |
| analog) | Aph(Atz)6-Leu7-Ilys8- | |
| | Pro9-D-Ala10-NH2 | |
| Cetrorelix | Ac-D-Nal1-D-Cpa2-D- | SEQ ID |
| (GnRH | Pal3-Ser4-Tyr5-D-Cit6- | NO: 98 |
| analog) | Leu7-Arg8-Pro9-D-Ala10- | |
| | NH2 | |
| Degarelix | Ac-D-Nal1-D-Cpa2-D- | SEQ ID |
| (GnRH | Pal3-Ser4-Aph(L- | NO: 99 |
| analog) | hydroorotyl)5-D-Aph | |
| | (carbamoyl)6-Leu7-Ilys8- | |
| | Pro9-D-Ala10-NH2 | |
| Ganirelix | Ac-D-Nal1-D-Cpa2-D- | SEQ ID |
| (GnRH | Pal3-Ser4-Tyr5-D- | NO: 100 |
| analog) | hArg(Et2)6-Leu7-hArg(Et2) | |
| | 8-Pro9-D-Ala10-NH2 | |
| Ozarelix | Ac-D-Nal1-D-Cpa2-D- | SEQ ID |
| (GnRH | Pal3-Ser4-N-MeTyr5-D- | NO: 101 |
| analog) | hCit6-Nle7-Arg8-Pro9-D- | |
| | Ala10-NH2 | |

In some embodiments, the protein associated with cancer is carcinoembryonic antigen (CEA). In some embodiments, the specificity agent comprises an anti-CEA antibody or antigen binding fragment thereof, e.g., an scFv or a Fab fragment, comprising heavy and light chain variable regions corresponding to anti-CEA humanized MN14 (hMN14) antibody (see Sharkey et al. (1995) CANCER RES. 55 (23 Suppl.): 5935 and variable sequences described in U.S. Patent Application Publication No. 2002/0165360, the contents of each of which are incorporated by reference herein). In some embodiments, the protein associated with cancer is CEA, and the cancer is selected from the group consisting of colon cancer, rectal cancer, pancreatic cancer, breast cancer, ovary cancer and lung cancer.

In some embodiments, the protein associated with cancer is prostate-specific membrane antigen (PSMA). In some embodiments, the specificity agent comprises an anti-PSMA antibody or antigen binding fragment thereof, e.g., an scFv or a Fab fragment, comprising light and heavy chain variable domain amino acid sequences as described in PCT Publication No. WO 2016/145139, the contents of which are incorporated by reference herein. In some embodiments, the specificity agent comprises an anti-PSMA antibody or antigen binding fragment thereof, e.g., an scFv or a Fab fragment, comprising a light chain variable amino acid sequence as set forth in SEQ ID NO: 50 and heavy chain variable domain amino acid sequence as set forth in SEQ ID NO: 49. In some embodiments, the specificity agent comprises DUPA. In some embodiments, the specificity agent comprises a PSMA binding ligand as disclosed in U.S. Patent Application Publication No. US 2010/0324008, which is incorporated herein by reference.

| Anti-PSMA Heavy Chain | Anti-PSMA Light Chain |
|---|---|
| Variable Domain | Variable Domain |
| QVQLVQSGGGLVQPGGSLRLSC | VIWMTQSPSSVSASVGDRVTIT |
| AASGFTFSSYWMSWVRQAPGKG | CRASQGISSWLAWYQQKPGKAP |
| LEWVANIKQDGSEKYYVDSVKG | KLLIYAASNLQSGVPSRFSGSG |
| RFTISRDNAKNSLYLQMNSLRA | SGTDFTLTISSLQPEDFATYYC |
| EDTAVYYCARVWDYYYDSSGDA | QQANSFPLTFGGGTKVDIK |
| FDIWGQGTMVTVSS | SEQ ID NO: 50 |
| SEQ ID NO: 49 | |

In some embodiments, the protein associated with cancer is PSMA, and the cancer is selected from the group consisting of prostate cancer, endometrial cancer, breast cancer, kidney cancer, and colon cancer.

In some embodiments, the protein associated with cancer is interleukin 13 receptor (IL-13R). In some embodiments, the specificity agent comprises an anti-IL-13R antibody or antigen binding fragment thereof, e.g., an scFv or a Fab fragment. In some embodiments, the specificity agent comprises an agent which binds to IL13R, such as an 1L13 ligand domain that binds to IL13R (SEQ ID NO: 51). In some embodiments, the protein associated with cancer is IL13R, and the cancer is breast cancer or malignant glioma.

| | |
|---|---|
| IL13R | MAFVCLAIGCLYTFLISTTFGCTSSSDTEIKVNPPQD |
| amino | FEIVDPGYLGYLYLQWQPPLSLDHFKECTVEYELKYR |
| acid | NIGSETWKTIITKNLHYKDGFDLNKGIEAKIHTLLPW |
| sequence | QCTNGSEVQSSWAETTYWISPQGIPETKVQDMDCVYY |
| | NWQYLLCSWKPGIGVLLDTNYNLFYWYEGLDHALQCV |
| | DYIKADGQNIGCRFPYLEASDYKDFYICVNGSSENKP |
| | IRSSYFTFQLQNIVKPLPPVYLTFTRESSCEIKLKWS |
| | IPLGPIPARCFDYEIEIREDDTTLVTATVENETYTLK |
| | TTNETRQLCFVVRSKVNIYCSDDGIWSEWSDKQCWEG |
| | EDLSKKTLLRFWLPFGFILILVIFVTGLLLRKPNTYP |
| | KMIPEFFCDT (SEQ ID NO: 51) |

In some embodiments, the protein associated with cancer is Cluster of Differentiation 19 (CD19). In some embodiments, the specificity agent comprises an anti-CD19 antibody or antigen binding fragment thereof, e.g., an scFv or a Fab fragment. In some embodiments, the protein associated with cancer is CD19, and the cancer is selected from the group consisting of acute lymphoblastic lymphoma (ALL), non-Hodgkin's lymphoma, lung cancer, and chronic lymphocytic leukemia (CLL).

In some embodiments, the protein associated with cancer is human epidermal growth factor receptor 2(HER2; also known as ErbB-2). In some embodiments, the specificity agent comprises an anti-HER2 antibody or antigen binding fragment thereof, e.g., an scFv or a Fab fragment. In some embodiments, the protein associated with cancer is HER2, and the cancer is selected from the group consisting of ovarian cancer, stomach cancer, uterine cancer and breast cancer.

In some embodiments, the protein associated with cancer is epidermal growth factor receptor (EGFR). In some embodiments, the specificity agent comprises an anti-EGFR antibody or antigen binding fragment thereof, e.g., an scFv or a Fab fragment. In some embodiments, the protein associated with cancer is EGFR, and the cancer is selected from the group consisting of non small cell lung cancer (NSCLC), colon cancer, rectal cancer, head and neck squamous cell carcinoma (HNSCC), breast cancer and pancreatic cancer.

In some embodiments, the protein associated with cancer is IL13R, e.g., breast cancer or malignant glioma.

In some embodiments, the protein associated with cancer is vascular endothelial growth factor receptor 2 (VEGFR2). In some embodiments, the specificity agent comprises an anti-VEGFR2 antibody or antigen binding fragment thereof, e.g., an scFv or a Fab fragment, comprising heavy and light chain variable regions corresponding to anti-VEGFR2 human VK-B8 antibody (see PCT Publication No. WO 2013/149219, the contents of which are incorporated by reference herein. In some embodiments, the specificity agent comprises an anti-VEGFR2 antibody or antigen binding fragment thereof, e.g., an scFv or a Fab fragment, comprising a light chain variable amino acid sequence as set forth in SEQ ID NO: 52 and heavy chain variable domain amino acid sequence as set forth in SEQ ID NO: 53.

| Anti-VEGFR2 VK-B8 | Anti-VEGFR2 VK-B8 |
|---|---|
| Heavy Chain Variable | Light Chain Variable |
| Domain | Domain |
| MAQVQLVQSGAEVKKPGSSVK | ETTLTQSPATLSVSPGERATV |
| VSCKAYGGTFGSYGVSWVRRA | SCRASQSLGSNLGWFQQKPGQ |
| PGQGLEWMGRLIPIFGTRDYA | APRLLIYGASTRATGIPARFS |
| QKFQGRVTLTADESTNTAYME | GSGSGTEFTLTISSLQSEDFA |
| LSSLRSEDTAVYYCARDGDYY | VYFCQQYNDWPITFGQGTRLE |
| GSGSYYGMDVWGQGTLVTVSS | IK |
| (SEQ ID NO: 53) | (SEQ ID NO: 52) |

In some embodiments, the protein associated with cancer is VEGFR2, and the cancer is selected from the group consisting of renal cell carcinoma, ovarian cancer, melanoma, non small cell lung cancer (NSCLC), colon cancer, rectal cancer, head and neck squamous cell carcinoma (HNSCC), breast cancer, myeloma, leukemia, lymphoma, and pancreatic cancer.

In some embodiments, the protein associated with cancer is ganglioside GD3 (GD3). In some embodiments, the specificity agent comprises an anti-ganglioside GD3 antibody or antigen binding fragment thereof, e.g., an scFv or a Fab fragment, comprising heavy and light chain variable regions corresponding to anti-GD3 antibody MB3.6 (see U.S. Patent Application Publication No. 2007/0031438 for variable amino acid sequences, which is incorporated by reference herein).

In some embodiments, the protein associated with cancer is c-type lectin-like molecule 1 (CLL1). In some embodiments, the specificity agent comprises an anti-CLL1 antibody or antigen binding fragment thereof, e.g., an scFv or a Fab fragment. In some embodiments, the specificity agent comprises an agent that binds to CLL1.

In some embodiments, the protein associated with cancer is cholecytoskinin B receptor (CCKBR). In some embodiments, the specificity agent comprises an anti-CCKBR antibody or antigen binding fragment thereof, e.g., an scFv or a Fab fragment. In some embodiments, the specificity agent comprises an agent that binds to CCKBR. In some embodiments, the specificity agent comprises a CCKBR antagonist. In some embodiments, the specificity agent comprises pentagastrin. In some embodiments, the specificity agent comprises a minigastrin. In some embodiments, the specificity agent comprises a minigastrin analog. In some embodiments, the minigastrin analog is selected from the group consisting of MG (SEQ ID NO: 80), MGO (SEQ ID NO: 81), MG11 (SEQ ID NO: 82), H2-Met (SEQ ID NO: 83), H2 Nle (SEQ ID NO: 84), Demogastrin (SEQ ID NO: 85), Cyclo-MG-1 (SEQ ID NO: 86), and MGD5 (SEQ ID NO: 87).

In some embodiments, the protein associated with cancer is gonadotropin releasing hormone receptor (GnRHR). In some embodiments, the specificity agent comprises an anti-GnRHR antibody or antigen binding fragment thereof, e.g., an scFv or a Fab fragment. In some embodiments, the specificity agent comprises gonadotropin releasing hormone (GnRH). In some embodiments, the specificity agent comprises a GnRH analog. In some embodiments, the GnRH analog is selected from the group consisting of Buserelin (SEQ ID NO: 88), Goserelin (SEQ ID NO: 89), Leuprolide (SEQ ID NO: 90), Nafarelin (SEQ ID NO: 91), Triptorelin (SEQ ID NO: 92), Abarelix (SEQ ID NO: 93), Acyline (SEQ ID NO: 94), Antarelix (SEQ ID NO: 95), Antide (SEQ ID NO: 96), Azaline B (SEQ ID NO: 97), Cetrorelix (SEQ ID NO: 98), Degarelix (SEQ ID NO: 99), Ganirelix (SEQ ID NO: 100), and Ozarelix (SEQ ID NO: 101). In some embodiments, the specificity agent comprises triptorelin. In some embodiments, the protein associated with cancer is GnRHR, and the cancer is selected from the group consisting of ovarian cancer, prostate cancer, breast cancer, endometrial cancer, melanoma, glioblastoma, lung cancer, and pancreatic cancer.

In some embodiments, the protein associated with cancer is somatostatin receptor 2 (SSRT2). In some embodiments, the specificity agent comprises an anti-SSRT2 antibody or antigen binding fragment thereof, e.g., an scFv or a Fab fragment. In some embodiments, the specificity agent comprises octreotate. In some embodiments, the specificity agent comprises octreotide. In some embodiments, the specificity agent comprises a somatostatin analog. In some embodiments, the somatostatin analog is selected from the group consisting of SS-14 (SEQ ID NO: 64), OC (SEQ ID NO: 65), TOC (SEQ ID NO: 66), TATE (SEQ ID NO: 67), NOC (SEQ ID NO: 68), NOC-ATE (SEQ ID NO: 69) BOC (SEQ ID NO: 70), BOC-ATE (SEQ ID NO: 71), KE108 (SEQ ID NO: 72), and LM3 (SEQ ID NO: 73). In some embodiments, the specificity agent comprises [Tyr3]-octreotate. In some embodiments, the specificity agent comprises a SSRT2-binding peptide as disclosed in U.S. Patent Application Publication No. 2004/0044177, which is incorporated herein by reference. In some embodiments, the protein associated with cancer is SSRT2, and the cancer is selected from the group consisting of neuroendocrine cancer, gastroenteropancreatic cancer, pancreatic cancer, lung cancer, carcinoid cancer, colorectal cancer, head and neck cancer, liver cancer, melanoma, stomach cancer, thyroid cancer, urothelial cancer, endometrial cancer, and breast cancer.

In some embodiments, the protein associated with cancer is aᵥβ₃ integrin. In some embodiments, the specificity agent comprises an anti-aᵥβ₃ antibody or antigen binding fragment thereof, e.g., an scFv or a Fab fragment. In some embodiments, the specificity agent comprises a cyclic arginine-glycine-aspartic acid peptide (cRGD).

In some embodiments, the protein associated with cancer is gastrin-releasing peptide receptor (GRPR). In some embodiments, the specificity agent comprises an anti-GRPR antibody or antigen binding fragment thereof, e.g., an scFv or a Fab fragment. In some embodiments, the specificity agent comprises bombesin. In some embodiments, the specificity agent comprises a bombesin analog. In some embodiments, the bombesin analog is selected from the group consisting of BN (SEQ ID NO: 74), RP527 (SEQ ID NO: 75), Demobesin 1 (SEQ ID NO: 76), Demobesin 4 (SEQ ID NO: 77), BBS-38 (SEQ ID NO: 78), and BAY 86-4367 (SEQ ID NO: 79).

In some embodiments, the protein associated with cancer is neurokinin 1 receptor (NK1R). In some embodiments, the specificity agent comprises an anti-NK1R antibody or antigen binding fragment thereof, e.g., an scFv or a Fab fragment.

In some embodiments, the protein associated with cancer is melanocortin 1 receptor (MC1R). In some embodiments, the specificity agent comprises an anti-MC1R antibody or antigen binding fragment thereof, e.g., an scFv or a Fab fragment.

In some embodiments, the protein associated with cancer is neurotensin receptor 1 (NTSR1). In some embodiments, the specificity agent comprises an anti-NTSR1 antibody or antigen binding fragment thereof, e.g., an scFv or a Fab fragment.

In some embodiments, the protein associated with cancer is a neuropeptide Y receptor (e.g., Y₁, Y₂, Y₄ and Y₅). In some embodiments, the specificity agent comprises an anti-neuropeptide Y receptor antibody or antigen binding fragment thereof, e.g., an scFv or a Fab fragment (e.g., an anti-Y₁, anti-Y₂, anti-Y₄, or anti-Y₅ antibody or antigen binding fragment thereof).

In some embodiments, the protein associated with cancer is folate receptor. In some embodiments, the specificity agent comprises an anti-folate receptor antibody or antigen binding fragment thereof, e.g., an scFv or a Fab fragment. In some embodiments, the specificity agent comprises folate. In some embodiments, the specificity agent comprises a folate receptor binding antifolate as described in International Publication No. WO 2010/033733, which is incorporated herein by reference. In some embodiments, the protein associated with cancer is folate receptor, and the cancer is selected from the group consisting of non small cell lung cancer (NSCLC), colorectal cancer, colon cancer, rectal cancer, ovarian cancer, renal cancer, gastric cancer, and breast cancer.

In some embodiments, the specificity agent binds to a protein from a disease-causing organisms (e.g., a prion, a virus, a protozoan, a parasite, a fungus, and a bacterium). In some embodiments, the specificity agent comprises an antibody, or an antigen-binding fragment thereof, that specifically binds to a protein from a disease-causing organism. In some embodiments, the specificity agent binds to a viral protein. In some embodiments, the specificity agent binds to an HIV protein. In some embodiments, the specificity agent binds to a bacterial protein. In some embodiments, the specificity agent binds to a fungal protein. In some embodiments, the specificity agent binds to a parasite protein. In some embodiments, the specificity agent binds to a protozoan protein.

The specificity agents for use in the present invention are conjugated to the catalytic antibody of the PUCRs disclosed herein via a reactive moiety. In some embodiments, the specificity agent comprises a reactive moiety that reacts with the reactive amino acid residue of the catalytic antibody region of the PUCR of the present invention. Reactive moieties for use in the present invention will be readily apparent to one of ordinary skill in the art. In some embodiments, the reactive moiety is a chemical group selected from the group consisting of a ketone, a diketone, a beta lactam, an active ester haloketone, a lactone, an anhydride, a maleimide, an epoxide, an aldehyde amidine, a guanidine, an imine, an eneamine, a phosphate, a phosphonate, an epoxide, an aziridine, a thioepoxide, a masked or protected diketone (e.g., a ketal), a lactam, a haloketone, an aldehyde, and the like. In some embodiments, the reactive moiety comprises a maleimide-containing component or other thiol-reactive groups such as iodoacetamides, aryl halides, disulfhydryls and the like. In some embodiments, the reactive moiety is a diketone. In other embodiments, the reactive moiety is a azetidinone. In some embodiments, the reactive moiety is a N-sulfonyl-beta-lactam.

In some embodiments, the specificity agent comprises a linker. Without wishing to be bound by any particular theory, in some embodiments, the specify agent comprises a linker that does not interfere with the activation of the host cell comprising the PUCR to which the specificity agent is attached. In some embodiments, the linker is a flexible linker. In some embodiments, the linker is a non-flexible linker. In some embodiments, the linker is a cleavable linker. In some embodiments, the linker is a hydrolysable linker.

In some embodiments, the linker is a non-cleavable linker. In some embodiments, the linker comprises a small molecule. In some embodiments, the linker comprises a peptide. In some embodiments, the linker comprises a non-peptide linker. In some embodiments, the non-peptide linker is an alkyl linker. An exemplary non-peptide linker is a polyethylene glycol (PEG) linker. In some embodiments, the linker comprises a hydrocarbon, peptidic, glycan, polyethylene glycol, or other linkage and/or polymer spacer. In some embodiments, the linker comprises (PEG)ₙ, wherein n can be an integer between 1 and 50, including 1, 2, 3, 4, 5, 6, 7, 8, 9, 10, 11, 12, 13, 14, 15, 16, 17, 18, 19, 20, 21, 22, 23, 24, 25, 27, 27, 28, 29, 30, 31, 32, 33, 34, 35, 36, 37, 38, 39, 40, 41, 42, 43, 44, 45, 46, 47, 48, 49, 50 or more. In some embodiments, the linker comprises (PEG)ₙ, wherein n is 5 or 13. In some embodiments, the linker comprises (PEG)ₙ, wherein n is 24 or 48. In some embodiments, the linker has a molecular weight of 100 to 5000 kDa, preferably 100 to 500 kDa. Peptide linkers may be altered to form derivatives. Any linker disclosed herein may be used to conjugate a reactive moiety to a specificity agent. Other linkers for use in the present invention are known in the art and will be readily apparent to those of skill in the art (see, e.g., U.S. Pat. Nos. 5,122,368; 5,824,805; and 8,309,093; and U.S. Pat. Appl. Publ. Nos. 2006/0024317; 2003/0083263; 2005/0238649; and 2005/0009751; the contents of which are herein incorporated by reference, and in particular the disclosure regarding linkers).

### D. Linkers

In an additional aspect of the invention, the PUCRs described herein may be conjugated to a linker comprising at least one reactive moiety. In some embodiments, the linker further comprises a conjugation functional group that may be reacted with a specificity agent in order to attach the specificity agent to the PUCR, thus programming the PUCR. In some embodiments, the specificity agent is conjugated to a linker disclosed herein via a conjugation functional group. In some embodiments, the PUCR is conjugated to a linker comprising a reactive moiety via a reactive amino acid residue.

The linker may comprise any reactive moiety described herein. In some embodiments, the reactive moiety is covalently bound to the reactive amino acid residue of the PUCR. In some embodiments, the reactive moiety is covalently bound to a side chain of the reactive amino acid residue of the PUCR. In some embodiments, the reactive moiety is non-covalently bound to the reactive amino acid residue of the PUCR. In some embodiments, the reactive moiety is a chemical group selected from the group consisting of a ketone, a diketone, a beta lactam, an active ester haloketone, a lactone, an anhydride, a maleimide, an epoxide, an aldehyde amidine, a guanidine, an imine, an eneamine, a phosphate, a phosphonate, an epoxide, an aziridine, a thioepoxide, a masked or protected diketone (e.g., a ketal), a lactam, a haloketone, an aldehyde, and the like. For example, when the PUCR comprises an aldolase antibody, or a catalytic portion thereof, (e.g., murine or humanized 38C2), the linker may be conjugated to the reactive lysine (e.g., Lys93) via a diketone or a azetidinone reactive moiety. Further, when the PUCR comprises a thioesterase antibody, or a catalytic portion thereof, the linker may be conjugated to the reactive cysteine via a reactive moiety comprising a maleimide-containing component or other thiol-reactive groups such as iodoacetamides, aryl halides, disulfhydryls and the like. In some embodiments, the reactive moiety of the linker is a diketone. In other embodiments, the reactive moiety of the linker is a azetidinone. In some embodiments, the reactive moiety of the linker is a N-sulfonyl-beta-lactam.

In some embodiments, the linker comprises a conjugation functional group. In some embodiments, the linker comprises at least one, two, three, four, five, six, seven, eight, nine, ten or more conjugation functional groups. In some embodiments, the conjugation functional group comprises a first chemical moiety capable of reacting with a second chemical moiety present on a specificity agent via a click-chemistry reaction. Click chemistry reactions are chemical reaction occurring between a pair of terminal reactive moieties that rapidly and selectively react (“click”) with each other to form a targeting or effector moiety conjugated binding polypeptide. In some embodiments, the click chemistry reaction is catalyzed by copper (Cu(I)). In some embodiments, the click chemistry reaction does not require a copper catalyst. In some embodiments, the conjugation functional group comprises a orthogonal reactive functional group. In these embodiments, the conjugation functional group of the linker is capable of reacting with a compatible orthogonal functional group present on a specificity agent. Multiple orthogonal reactive functional groups, and the orthogonal functional groups that they are capable of reacting with, are known in the art and can be used in the methods described herein (see, e.g., Lang and Chin (2014) CHEM. REV. 114: 4764-4806; and Lang and Chin (2014) ACS CHEM. BIOL. 9: 16-20). Orthogonal functional groups include, but are not limited to: aldehyde, ketone, aminooxy, hydrazine, seleno-substitution, dibenzocyclooctyl, trans-cyclooctene, alkyne, azide, tetrazine, olefins, etc. Such reactions of suitable orthogonal functional groups are represented by, but are not limited to: ketone/alkoxyamine condensation, aldehyde/alkoxyamine condensation, Diels-Alder cycloaddition, Staudinger ligation, cross-metathesis, Pd-catalyzed cross coupling, strain-promoted alkyne-azide cycloadditions, strain-promoted alkyne-nitrone cyclcoaddition, copper-catalyzed alkyne-azide cycloaddition, photo-click cycloaddition, and 1,2-aminothiol-CBT condensations. Orthogonal groups also include enzyme substrates.

In some embodiments, the linker is a flexible linker. In some embodiments, the linker is a non-flexible linker. In some embodiments, the linker is a cleavable linker. In some embodiments, the linker is a hydrolysable linker. In some embodiments, the linker is a non-cleavable linker. In some embodiments, the linker comprises a small molecule. In some embodiments, the linker comprises a peptide. In some embodiments, the linker comprises a non-peptide linker. In some embodiments, the linker comprises a hydrocarbon, peptidic, glycan, polyethylene glycol, or other linkage and/or polymer spacer. An exemplary non-peptide linker is a polyethylene glycol (PEG) linker. In some embodiments, the linker comprises (PEG)ₙ, wherein n can be an integer between 1 and 50, including 1, 2, 3, 4, 5, 6, 7,8, 9, 10, 11, 12, 13, 14, 15, 16, 17, 18, 19, 20, 21, 22, 23, 24, 25, 27, 27, 28, 29, 30, 31, 32, 33, 34, 35, 36, 37, 38, 39, 40, 41, 42, 43, 44, 45, 46, 47, 48, 49, 50 or more. In some embodiments, the linker comprises (PEG)ₙ, wherein n is 5 or 13. In some embodiments, the linker comprises (PEG)ₙ, wherein n is 24 or 48. In some embodiments, the linker has a molecular weight of 100 to 5000 kDa, preferably 100 to 500 kDa.

In some embodiments, the linker employs “C-Lock” conjugation methods and linker chemistry. This chemistry re-connects polypeptides previously bound by disulfide bonds (e.g., antibody heavy and light chains) following the reduction of the disulfide bonds. The crosslinking introduces one linker per broken disulfide bond. In some embodiments, conjugation is accomplished using a maleimido or vinyl moiety which can react with individual sulfhydryl group on an antibody via Michael addition reaction. The free sulfhydryl group can be formed by reducing a disulfide bond in an antibody. Suitable compositions and methods that provide conjugation through cysteine without decreased structural stability are disclosed in WO 2013/173391, incorporated in its entirety by this reference.

In some embodiments, the linker employs “K-Lock” site-selective conjugation technology targeting lysine residues present in a polypeptide. For example, when the specificity agent is an antibody, the “K-Lock” site-selective conjugation technology targets two native Lys sites out of 80-90 Lys present in an antibody without the need for antibody modification using cell engineering or enzymatic modification steps. In some embodiments, conjugation is accomplished by forming an amide bond with a lysine side chain as disclosed, e.g., in WO 2013/173392 and WO 2013/173393, incorporated in their entirety by this reference. In some embodiments, the linker is attached to a specificity agent (e.g., an antibody or antigen-binding fragment thereof) comprising a variable kappa light chain. In some embodiments, the linker is attached to a lysine of the variable kappa light chain (e.g., the lysine corresponding to Lys188 according to Kabat numbering).

In some embodiments the linker is diketone-PEG5-PFP ester ((2,3,4,5,6-pentafluorophenyl) 3-[2-[2-[2-[2-[3-[4-(3,5-dioxohexyl)anilino]-3-oxo-propoxy]ethoxy]ethoxy]ethoxy]ethoxy]propanoate, also referred to herein as DK-PEG5-PFP ester). In some embodiments, the linker is azetidinone-PEG13-PFP ester ((2,3,4,5,6-pentafluorophenyl) 3-[2-[2-[2-[2-[2-[2-[2-[2-[2-[2-[2-[2-[3-oxo-3-[4-[3-oxo-3-(2-oxoazetidin-1-yl)propyl]anilino]propoxy]ethoxy]ethoxy]ethoxy]ethoxy]ethoxy]ethoxy]ethoxy]ethoxy]ethoxy]ethoxy]ethoxy]ethoxy]propanoate, also referred to herein as AZD-PEG5-PFP ester.

### C. Detectable Moieties

In some embodiments, the PUCR and/or the specificity agent and/or the linker of the present invention comprises a detectable moiety. In some embodiments, the detectable moiety is covalently attached to the PUCR. In some embodiments, the detectable moiety is covalently attached to the specificity agent. In some embodiments, the detectable moiety is non-covalently attached to the PUCR. In some embodiments, the detectable moiety provides a means for detection or quantitation of the PUCR and/or the specificity agent comprising the detectable moiety. In some embodiments, the detectable moiety provides a mean for determining the efficiency of conjugation of a specificity agent to a PUCR of the present invention.

In some embodiments, the detectable moiety is a polypeptide (e.g., a GST-tag, a His-tag, a myc-tag, or a HA-tag, a fluorescent protein (e.g., a GFP or a YFP)). In some embodiments, the detectable moiety is a radioactive moiety, a fluorescent moiety, a chemiluminescent moiety, a mass label, a charge label, or an enzyme (e.g., for which substrate converting activity of the enzyme is observed to reveal the presence of the programmable universal chimeric receptor and/or the specificity agent). In some embodiments, the detectable moiety is biotin.

In some embodiments, the detectable moiety is attached to the N-terminus of the programmable universal cell receptor. In some embodiments, the detectable moiety is attached to the N-terminus of the specificity agent. In some embodiments, the detectable moiety is attached to the C-terminus of the programmable universal cell receptor. In some embodiments, the detectable moiety is attached to the C-terminus of the specificity agent.

In some embodiments, the programmable universal cell receptor and/or specificity agent comprises one, two, three, four, five, six, seven, eight, nine, ten or more detectable moieties.

In some embodiments the detectable moiety is cleavable. In other embodiments, the detectable moiety is non-cleavable. In some embodiments, the detectable moiety is attached to the programmable universal cell receptor and/or specificity agent via a linker. In some embodiments, the linker is cleavable. In other embodiments, the linker is non-cleavable. Linkers for use with the detectable moieties can be any linker disclosed herein, or any linker readily apparent to one of skill in the art.

Any of the nucleic acids encoding a PUCR described herein can be prepared by a routine method, such as recombinant technology. Methods for preparing a PUCR described herein involve generation of a nucleic acid that encodes a polypeptide comprising each of the domains of the PUCRs, including the catalytic antibody region, the transmembrane domain, and the intracellular domain. In some embodiments, the nucleic acid encodes an intracellular domain comprising a signaling domain. In some embodiments, the nucleic acid encodes an intracellular domain comprising a co-stimulatory signaling domain. In some embodiments, the nucleic acid encodes a hinge region between the catalytic antibody region of the PUCR and the transmembrane domain. The nucleic acid encoding the chimeric receptor may also encode a signal sequence.

Sequences of each of the components of the PUCRs disclosed herein may be obtained via routine technology, e.g., PCR amplification from any one of a variety of sources known in the art. In some embodiments, sequences of one or more of the components of the PUCRs are obtained from a mammalian cell (e.g., a murine cell or a human cell). Alternatively, the sequences of one or more components of the PUCRs can be synthesized. Sequences of each of the components (e.g., domains) can be joined directly or indirectly (e.g., using a nucleic acid sequence encoding a peptide linker) to form a nucleic acid sequence encoding the PUCR, using methods such as PCR amplification or ligation. Alternatively, the nucleic acid encoding the PUCR may be synthesized. In some embodiments, the nucleic acid is DNA. In other embodiments, the nucleic acid is RNA (e.g., mRNA).

In further embodiments, isolated isolated polypeptide molecule encoded by any of the nucleic acid molecules disclosed herein are also contemplated. Methods of purifying and isolated said polypeptides are well known in the art (see, e.g., Sambrook et al. (2012) MOLECULAR CLONING: A LABORATORY MANUAL, volumes 1-4, Cold Spring Harbor Press, NY).

### D. Host Cells

Isolated host cells expressing the PUCRs described herein are also contemplated in the present invention. In some embodiments, the host cells are immune cells (e.g., T cells, NK cells, macrophages, monocytes, neutrophils, eosinophils, cytotoxic T lymphocytes, regulatory T cells, or any combination thereof). In some embodiments, the isolated host cells are T cells. In some embodiments, the isolated host cells are NK cells. In other embodiments, the isolated host cells are established cell lines, for example, NK-92 cells. In some embodiments, the isolated host cells are modified NK-92 cells (ATCC Deposit No. PTA-6672). In some embodiments, the host cell is a KHYG-1 natural killer cell. In some embodiments, the host cell is a NKL natural killer cell. In one embodiment, the host cell is a placental NK cell.

In some embodiments, the isolated host cells are immune cells. A population of immune cells can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs.

The methods of preparing host cells expressing a PUCR of the present invention may comprise expanding the isolated host cells ex vivo. Expanding host cells may involve any method that results in an increase in the number of cells expressing a PUCR, for example, by allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing a PUCR of the present invention are expanded ex vivo prior to administration to a subject.

Methods for preparing host cells expressing any of the PUCRs described herein may also comprise activating the isolated host cells (e.g., T cells) ex vivo. Activating a host cell means stimulating a host cell into an active state in which the cell may be able to perform effector functions (e.g., cytotoxic function). Methods of activating a host cell will depend on the type of host cell used for expression of the PUCR. For example, T cells may be activated ex vivo in the presence of one or more molecule such as an anti-CD3 antibody, an anti-CD28 antibody, IL-2, or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, 1L12, IL-21, and K562 cells. In some embodiments, the host cells expressing any of the PUCRs described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

To create the isolated host cells that express a PUCR disclosed herein, expression vectors for stable or transient expression of the PUCR may be constructed via conventional methods and introduced into the isolated host cells. For example, nucleic acids (e.g., DNA or mRNA) encoding the PUCR may be cloned into a suitable expression vector, such as a viral vector in operable linkage to a suitable promoter. In some embodiments, the promoter is an inducible promoter. In some embodiments, the promoter is a constitutive promoter. In some embodiments, the promoter is tissue-specific. In some embodiments, the promoter is cell-specific. The expression vector may be provided to a cell in the form of a viral vector. Viral vector technology is well known in the art and is described, for example, in Sambrook et al. (2012) MOLECULAR CLONING: A LABORATORY MANUAL, volumes 1-4, Cold Spring Harbor Press, NY, and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., as disclosed in PCT Application Nos. WO 01/96584; WO 01/29058; and U.S. Pat. No. 6,326,193). Suitable vectors and methods for producing vectors containing transgenes are well known and available in the art. In some embodiments, the vector is a viral vector. In some embodiments the viral vector is selected from the group consisting of a retroviral vector, a lentiviral vector, an adenovirus vector, and an adeno-associated vector. In some embodiments, the vector is a murine leukemia virus (MLV)-based retroviral vector (see, e.g., Kim et al. (1998) J VIROL. 72(2): 994-1004, which is incorporated by reference herein). In some embodiments, the vector is a Moloney murine leukemia virus (MoMuLV)-based retroviral vector.

A variety of promoters can be used for expression of a PUCR described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, herpes simplex tk virus promoter. Additional promoters for expression of a PUCR include any constitutively active promoter in a mammalian cell (e.g., an immune cell). Alternatively, any regulatable promoter may be used, such that its expression can be modulated within a host cell.

Vectors for use in the present invention may contain, for example, one or more of the following: a selectable marker gene (e.g., a neomycin gene for selection of stable or transient transfectants); an enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the PUCR.

Methods of delivering nucleic acids encoding a PUCR (e.g., a vector) to a host cell are well known in the art. Nucleic acids encoding a PUCR (e.g., DNA or mRNA) can be introduced into host cells using any of a number of different methods, for instance, commercially available methods which include, but are not limited to, electroporation (Amaxa Nucleofector-II (Amaxa Biosystems), ECM 830 (BTX) (Harvard Instruments), or the Gene Pulser II (BioRad), Multiporator (Eppendorf), cationic liposome mediated transfection using lipofection, polymer encapsulation, peptide mediated transfection, or biolistic particle delivery systems such as “gene guns” (see, for example, Nishikawa et al. (2001) HUM GENE THER. 12(8): 861-70.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Also contemplated are lipofectamine-nucleic acid complexes. In some embodiments, vectors encoding a PUCR of the present invention are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655).

In some aspects, non-viral methods can be used to deliver a nucleic acid encoding a PUCR described herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. (2011) HUM. MOL. GENET. 20:R14-R20; Singh et al. (2008) CANCER RES. 15: 2961-2971; Huang et al. (2008) MOL. THER. 16: 580-589; Grabundzija et al. (2010) MOL. THER. 18: 1200-1209; Kebriaei et al. (2013) BLOOD. 122: 166; Williams (2008) Molecular Therapy 16: 1515-16; Bell et al. (2007) NAT. PROTOC. 2: 3153-65; and Ding et al. (2005) CELL 122: 473-83, the contents of each of which are incorporated herein by reference. The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. (2011). Use of the SBTS permits efficient integration and expression of a transgene, e.g., a nucleic acid encoding a PUCR described herein. Provided herein are methods of generating a cell, e.g., T cell or NK cell, that stably expresses a PUCR described herein, e.g., using a transposon system such as SBTS.

Exemplary transposons include a pT2-based transposon. See, e.g., Grabundzija et al. (2013) NUCLEIC ACIDS RES. 41: 1829-47; and Singh et al. (2008) CANCER RES. 68: 2961-71, the contents of each of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter).

In some embodiments, cells, e.g., T cells or NK cells, are generated that express a PUCR described herein by using a combination of gene insertion using the SBTS and genetic editing using a nuclease (e.g., zinc finger nucleases (ZFNs), Transcription Activator-Like Effector Nucleases (TALENs), the CRISPR/Cas system, or engineered meganuclease re-engineered homing endonucleases).

The isolated host cells included in the present invention may express more than one type of PUCR (e.g., two, three, four, five, six, seven, eight, nine, ten, or more types of PUCR). Thus, in some embodiments of the invention, the isolated host cells may express one type of PUCR. In some embodiments, the isolated host cells of the present invention may express two types of PUCRs. In some embodiments of the invention, the isolated host cells may express three types of PUCRs. In some embodiments of the invention, the isolated host cells may express four types of PUCRs. In some embodiments of the invention, the isolated host cells may express five types of PUCRs. In some embodiments of the invention, the isolated host cells may express six types of PUCRs. The expression of more than one type of PUCR may be particularly advantageous for therapeutic purposes. For example, in one embodiment, the host cell of the present invention may express a PUCR comprising a co-stimulatory domain from an activating receptor protein and a PUCR comprising a co-stimulatory domain from an inhibitory receptor protein. Each of said PUCR may be further programmed (e.g., conjugated) to different ligands. For example, in one embodiment, a host cell may comprise a PUCR comprising a co-stimulatory signaling domain from an activating receptor (e.g., DAP10) that has been programmed (i.e., conjugated) with a specificity agent that binds to a protein associated with cancer, and a second PUCR comprising a co-stimulatory signaling domain from an inhibitory receptor (e.g., CD94/NKG2A) that has been programmed (i.e., conjugated) with a specificity agent that binds a ligand that is not present, or minimally present, on the surface of normal cells (e.g., non-cancerous cells). When both of said PUCRs are expressed in the host cell, the activation of the host cell (e.g., a T cell) can be regulated such that the T cell is not activated, or exhibits reduced activation when it binds to a normal host cell.

In some embodiments of the invention, the host cell comprising a PUCR can be used for non-therapeutic purposes. For example, a host cell comprising a PUCR can be used for diagnostic purposes and/or can be used to determine whether a particular cell (e.g., a cancer cell) expresses a biomarker on its surface.

The isolated host cells of the present invention expressing a PUCR disclosed herein can be programmed using one or more of the specificity agents. One advantage of the present invention is that a host cell expressing a PUCR disclosed herein can be programmed to target one or more ligands of interest. Thus, a single host cell of the present invention may have multiple specificities. For example, in one embodiment, a host cell comprising a PUCR of the present invention comprises a PUCR which is conjugated to a specificity agent specific for a first ligand, and further comprises a PUCR which is conjugated to a specificity agent specific for a second ligand which is different from the first ligand. In one embodiment, said first ligand and said second ligand may be different epitopes of the same protein. In some embodiments, said first and second ligand may be different proteins.

In one embodiment, a host cell comprising a PUCR of the present invention comprises a PUCR which is conjugated to a specificity agent specific for a first antigen, and a PUCR which is conjugated to a specificity agent specific for a second antigen which is different from the first antigen. In some embodiments, the host cell expressing a PUCR disclosed herein may be programmed with multiple specificity agents (e.g., 2, 3, 4, 5, 6, 7, or 8 specificity agents). Thus, a single host cell may comprise two, three, four, five, six, seven, or more PUCRs, wherein each PUCR has been conjugated to a different specificity agent. Said specificity agents may all be the same type of specificity agent or different types of specificity agents. For example, a host cell expressing a PUCR disclosed herein can be programmed with a first specificity agent, wherein said first specificity agent comprises a binding protein (e.g., an antibody or antigen binding fragment thereof), and with a second specificity agent, wherein said second specificity agent comprises a small molecule (e.g., folic acid or 2-[3-(1, 3-dicarboxy propyl)-ureido] pentanedioic acid (DUPA). The ability to program the host cells expressing PUCR disclosed herein with two or more specificity agents may be particularly advantage for the treatment of complex diseases and/or medical conditions, such as cancer, where it may be desirable to target multiple ligands using the same host cell (e.g., an immune cell) expressing a PUCR disclosed herein.

The isolated host cells of the present invention expressing a PUCR disclosed herein can be conjugated to a linker comprising a reactive moiety via the reactive amino acid residue of the PUCR. In some embodiments, the PUCR is conjugated to the linker in vitro. In some embodiments, the PUCR is conjugated to the linker in vivo. The PUCR can then be programmed by reacting the a conjugation functional group present on the linker (e.g., a first orthogonal functional group) with a chemical moiety present on the specificity agent (e.g., a second orthogonal functional group). In some embodiments, specificity agent is reacted with a linker conjugated to the PUCR in vitro. In some embodiments, specificity agent is reacted with a linker conjugated to the PUCR in vivo.

Also provided in the present invention is a population of host cells (e.g., immune cells), wherein the population of host cells comprises a) a subpopulation of host cells comprising a PUCR linked to a specificity agent that binds to a first ligand, and b) a subpopulation of host cells comprising a PUCR linked to a second ligand, which is different that the first ligand. In some embodiments, the present invention provides populations of host cells (e.g., immune cells), wherein the population of host cells comprises a) a subpopulation of host cells comprising a PUCR linked to a specificity agent that binds to a first antigen, and b) a subpopulation of host cells comprising a PUCR linked to a second antigen, which is different that the first antigen. In some embodiments, the present invention provides a population of host cells, wherein the population of host cells comprises two, three, four, five, six, seven, or more subpopulation of host cells comprising a PUCR, wherein each subpopulation of host cells comprises a PUCR linked to a specificity agent that is different form the specificity agent of each of the other subpopulations of host cells.

### E. Kits

The invention also provides kits comprising one or more compositions disclosed herein. Kits of the invention include one or more containers comprising a population of host cells comprising a PUCR disclosed herein, and in some embodiments, further comprise instructions for use in accordance with any of the methods described herein. The kit may further comprise a description of selection an individual suitable or treatment (e.g., a specificity agent). Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising a population of host cells comprising a PUCR, wherein the PUCR comprises a catalytic antibody, or a catalytic portion thereof, comprising a reactive amino acid residue, wherein the reactive amino acid residue is not bound to a specificity agent; a transmembrane domain; and an intracellular domain, and b) instructions for administering the population of host cells to a subject for the effective treatment of a disease. In some embodiments, said disease is a cancer. In other embodiments, said disease is a medical condition caused by a disease-causing organism (e.g., a prion, a virus, a bacterium, a fungus, a protozoan, and a parasite). In some embodiments, the kit further comprises one or more specificity agent(s). The population of host cells comprising a PUCR and the specificity agent(s) can be present in separate containers or in a single container. In some embodiments, the population of host cells comprising a PUCR is comprised of from about 1×10¹ host cells to about 1×10¹² host cells. In some embodiments, the population of host cells comprising a PUCR is comprised of about 1×10¹ host cells. In some embodiments, the population of host cells comprising a PUCR is comprised of about 1×10² host cells. In some embodiments, the population of host cells comprising a PUCR is comprised of about 1×10³ host cells. In some embodiments, the population of host cells comprising a PUCR is comprised of about 1×10⁴ host cells. In some embodiments, the population of host cells comprising a PUCR is comprised of about 1×10⁵ host cells. In some embodiments, the population of host cells comprising a PUCR is comprised of about 1×10⁶ host cells. In some embodiments, the population of host cells comprising a PUCR is comprised of about 1×10⁷ host cells. In some embodiments, the population of host cells comprising a PUCR is comprised of about 1×10⁸ host cells. In some embodiments, the population of host cells comprising a PUCR is comprised of about 1×10⁹ host cells. In some embodiments, the population of host cells comprising a PUCR is comprised of about 1×10¹⁰ host cells. In some embodiments, the population of host cells comprising a PUCR is comprised of about 1×10¹¹ host cells. In some embodiments, the population of host cells comprising a PUCR is comprised of about 1×10¹² host cells. In other embodiments, the kit comprises a) a composition comprising a nucleic acid molecule encoding a PUCR, wherein the PUCR comprises a catalytic antibody, or a catalytic portion thereof, comprising a reactive amino acid residue, a transmembrane domain, and an intracellular domain; and b) instructions for introducing the nucleic acid molecule encoding a PUCR into an isolated host cell.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information.

The instructions relating to the use of the compositions disclosed herein include information as to dosage, dosing schedule, and route of administration for the intended treatment. The containers may be unit doses, bulk packages (e.g., multi-dose packages) or sub-unit doses.

### II. Methods for Use of the Compositions of the Invention

The compositions of the present invention are suitable for treating a variety of medical conditions and diseases due to the versatility of the PUCRs disclosed herein. As disclosed above, the nucleic acids encoding a PUCR of the present invention can be used to generate isolated host cells that can be programmed to target any ligand of interest. For example, in some embodiments, the host cell is an immune cell (e.g., a T cell or a NK cell). In said embodiments, the present invention advantageously provides programmable immunotherapy methods that may be customized, as the need may arise, to treat a disease. Said programmable immunotherapy methods are particularly advantageous in treating complex diseases, such as cancer and infectious diseases.

One particular advantage of the methods of the present invention is that a population of host cells comprising a PUCR can be readily created using the methods described herein, and stored (e.g., cryopreserved) or administered to a subject without first being programmed (i.e., without first being conjugated to a specificity agent). The population of host cells can then be retrieved (e.g., isolated from the subject), programmed at-will (e.g., conjugated with a specificity agent of interest), and administered to the subject, as the need may arise. Thus, for example, if the host cell is, e.g., a T-cell, a population of T cells comprising a PUCR of the present invention can be generated and administered to a human subject. Without wishing to be bound by any particular theory, said population of T cells comprising a PUCR can be caused to multiply, expand, and/or establish in the subject, thus providing a potentially unlimited supply of T cells comprising a PUCR which may be retrieved (e.g., isolated from the subject), and programmed at-will, as the need may arise.

A potential issue that can arise in patients being treated with host cells expressing chimeric antigen receptors (i.e., CAR T cells) is that anaphylaxis may develop after multiple treatments with the cells, particularly in chimeric antigen receptors comprising non-human derived protein sequence or regions (e.g., a murine scFv). It is believed that such an anaphylactic response is caused by a humoral anti-CAR response, i.e., anti-CAR antibodies having an anti-IgE isotype. Without wishing to be bound by any particular theory, a particular advantage of the methods of the present invention is that the host cells comprising the PUCRs of the present invention may not induce a humoral response in the subject to whom they are administered. For instance, in some embodiments, the PUCR can be designed to solely comprise humanized and/or human sequences. Therefore, when administered to a subject, the host cells comprising the PUCR may be designed be antigenically-dormant. In some embodiments, even if said host cells comprise a PUCR that has been programmed (i.e., conjugated) to a specificity agent comprising a non-human derived protein sequence or region, said programmed PUCR are only be exposed to the subject's immune system for a limited amount of time after administration of the host cell to the subject, such that a deleterious immune reaction does not develop in the subject. This may be either because the PUCR is internalized during normal plasma membrane recycling processes (e.g., endocytosis) or because the host cell comprising the programmed PUCR dies.

In another aspect of the present invention, a subject may be administered a population of host cells comprising a PUCR that has been conjugated with a linker comprising a conjugation functional group, as described herein. The subject can then be administered a specificity agent comprising a chemical moiety that is capable of reacting with the conjugation functional group in order to program the PUCR at-will. Thus, in some embodiments, the PUCR is programmed in vivo or in situ. In other embodiments, the population of host cells comprising a PUCR that has been conjugated with a linker can be removed from the subject and programmed with a specificity agent comprising a chemical moiety that is capable of reacting with the conjugation functional group ex vivo.

In one aspect, the present invention provides for a method of making a customized therapeutic host cell for use in the treatment of a disease in a subject in need thereof, the method comprising contacting an immune cell with a specificity agent that binds to a PUCR that is expressed on the cell membrane of the immune cell, wherein the specificity agent binds to a disease-associated antigen corresponding to a disease antigen profile of the subject in need thereof. In some embodiments, the customized therapeutic host cell is contacted with the specificity agent that binds to a PUCR in vivo. In some embodiments, the customized therapeutic host cell is contacted with the specificity agent that binds to a PUCR in vitro. In some embodiments, the customized therapeutic host cell is contacted with the specificity agent that binds to a PUCR in situ. Also provided is a method of making a customized therapeutic host cell for use in the treatment of a cancer in a subject in need thereof, the method comprising contacting an immune cell with a specificity agent that binds to a PUCR that is expressed on the cell membrane of the immune cell, wherein the specificity agent binds to a cancer-associated antigen corresponding to a cancer antigen profile of the subject in need thereof. The present invention also provides a method of making a customized therapeutic host cell for use in the treatment of an infectious disease in a subject in need thereof, the method comprising contacting an immune cell with a specificity agent that binds to a PUCR that is expressed on the cell membrane of the immune cell, wherein the specificity agent binds to a disease-causing organism antigen corresponding to a disease-causing organism antigen profile of the subject in need thereof.

In one aspect, the present invention provides methods for treating a cancer or inhibiting tumor growth in a subject in need thereof, the method comprising administering to the subject an isolated host cell comprising a PUCR of the present invention, or a population of said host cells. In some embodiments, the isolated host cell is an immune cell (e.g., a T cell or a NK cell). In some embodiments, the isolated host cell comprising a PUCR of the present invention is derived from the subject. In some embodiments, the isolated host cell comprising a PUCR of the present invention is not derived from the subject. In some embodiments, the isolated host cell is a cell from an established cell line (e.g., an NK-92 cell).

Any cancer known in the art may be treated with the methods of the present invention, including, but not limited to, prostate cancer, biliary tract cancer, brain cancer (including glioblastomas and medelloblastomas), breast cancer, cervical cancer, choriocarcinoma, colon cancer, endometrial cancer, esophageal cancer, gastric cancer, hematological neoplasms (including, e.g., acute lymphocytic and myelogeneous leukemia, multiple myeloma, AIDS associated leukemias and adult T-cell leukemia lymphoma), intraepithelial neoplasms (including, e.g., Bowen's disease and Paget's disease), liver cancer, lung cancer, lymphomas (including, e.g., Hodgkin's disease and lymphozytic lymphomas) neuroblastomas, oral cancer (including squamous cell carcinoma), ovarian cancer (including those arising from epithelial cells, stromal cells, germ cells and mesenchymal cells), pancreatic cancer, rectal cancer, sarcomas (including e.g., leiomyosarcoma, rhabdomyosarcoma, liposarcoma, fibrosarcoma and osteosarcoma), skin cancer (including, e.g., melanoma, Kaposi's sarcoma, basocellular cancer and squamous cell cancer), testicular cancer (including, e.g., germinal tumors (seminoma, non-seminoma (teratomas, choriocarcinomas), stromal tumors and germ cell tumors), thyroid cancer (including, e.g., thyroid adenocarcinoma and medullar carcinoma), and renal cancer (including, e.g., adenocarcinoma and Wilms tumor).

In some embodiments, the cancer is associated with high expression levels of a protein. In said embodiments, the isolated host cells comprising the PUCRs of the present invention can be programmed (e.g., conjugated) with a specificity agent that targets (e.g., specifically binds to) the proteins whose high expression levels is associated with the cancer. In some embodiments, the protein whose high expression levels is associated with a cancer is expressed on the surface of the cancerous cell. In some embodiments, the protein whose high expression levels is associated with a cancer, is not expressed on the surface of the cancerous cell.

In one aspect, the present invention provides a method for treating cancer in a subject in need thereof, said method comprising: (a) determining a cancer antigen profile of the subject; (b) selecting a specificity agent that binds to the antigen identified in (a); and (c) administering an immune cell comprising a PUCR bound to (e.g., conjugated) to the specificity agent identified in (b), thereby treating the cancer in the subject in need thereof.

In one aspect, the invention provides a method of inhibiting growth of a tumor expressing a cancer associated antigen, comprising contacting a cancer cell of the tumor with an immune cell comprising a PUCR conjugated to a specificity agent that binds to the cancer associated antigen, such that the immune cell is activated in response to the antigen and targets the cancer cell of the tumor, wherein the growth of the tumor is inhibited. In some embodiments, the immune cell is a T cell. In other embodiments, the immune cell is a NK cell. In some embodiments, the immune cell is a NK-92 cell. In some embodiments, the immune cell kills the tumor cell.

In one aspect, the present invention provides a method for inhibiting the proliferation or reducing the population of cancer cells expressing a cancer associated antigen, the method comprising contacting the cancer-associated antigen-expressing cell population with a host cell comprising a PUCR of the present invention conjugated to a specificity agent that binds to the cancer-associated antigen, thereby inhibiting the proliferation or reducing the population of cancer cells expressing a cancer associated antigen. In certain aspects, the method results in a reduction in the quantity, number, amount or percentage of malignant and/or cancer cells by at least 25%, at least 30%, at least 40%, at least 50%, at least 65%, at least 75%, at least 85%, at least 95%, or at least 99% in a subject, as compared to the quantity, number, amount or percentage of malignant and/or cancer cells in a subject prior to administering the host cell. In one embodiment, the subject is a human.

In another aspect, the present invention provides a method of treating a medical condition caused by a disease-causing organism in a subject, the method comprising administering to the subject an isolated host cell comprising a PUCR of the present invention, or a population of said host cells. In some embodiments, the isolated host cell is an immune cell (e.g., a T cell or a NK cell). In some embodiments, the isolated host cell comprising a PUCR of the present invention is derived from the subject. In some embodiments, the isolated host cell comprising a PUCR of the present invention is not derived from the subject. In some embodiments, the isolated host cell is a cell from an established cell line (e.g., an NK-92 cell).

In one aspect, the present invention provides a method for treating a medical condition caused by a disease-causing organism in a subject in need thereof, said method comprising: (a) determining a disease-causing organism antigen profile of the subject; (b) selecting a specificity agent that binds to the antigen identified in (a); and (c) administering an immune cell comprising a PUCR bound to (e.g., conjugated) to the specificity agent identified in (b), thereby treating the medical condition caused by a disease-causing organism in the subject in need thereof.

In one aspect, the present invention provides a method of resolving an infection caused by a disease-causing organism in a subject in need thereof, said method comprising: (a) determining a disease-causing organism antigen profile of the subject; (b) selecting a specificity agent that binds to the antigen identified in (a); and (c) administering an immune cell comprising a PUCR bound to (e.g., conjugated) to the specificity agent identified in (b), thereby resolving the infection caused by a disease-causing organism in the subject in need thereof.

In one aspect, the invention provides a method of killing a disease-causing organism in a subject in need thereof, comprising contacting a disease-causing organism with an immune cell comprising a PUCR conjugated to a specificity agent that binds to an antigen of the disease-causing organism, such that the immune cell is activated in response to the antigen and targets the disease-causing organism or a cell of the subject infected with the disease-causing organism, wherein the disease-causing organism is killed. In some embodiments, the immune cell is a T cell. In other embodiments, the immune cell is a NK cell. In some embodiments, the immune cell is a NK-92 cell. In some embodiments, the immune cell kills disease-causing organism or the cell of the subject infected with the disease causing organism.

In one aspect, the present invention provides a method for inhibiting the proliferation or reducing a population of a disease-causing organism, the method comprising contacting the population of disease-causing organisms in the subject with a host cell comprising a PUCR of the present invention conjugated to a specificity agent that binds to an antigen of the disease causing organism, thereby inhibiting the proliferation or reducing the population of a disease-causing organism. In certain aspects, the method results in a reduction in the quantity, number, amount or percentage of disease causing-organisms by at least 25%, at least 30%, at least 40%, at least 50%, at least 65%, at least 75%, at least 85%, at least 95%, or at least 99% in a subject, as compared to the quantity, number, amount or percentage of disease-causing organisms in a subject prior to administering the host cell. In one embodiment, the subject is a human.

In some embodiments, the disease-causing organism is selected from the group consisting of a prion, a virus, a protozoan, a bacterium, a fungus, or a parasite. Without wishing to be bound by any particular theory, the methods of the present invention are particularly advantageous for treating medical conditions caused by disease causing organism capable of undergoing antigenic variation as an immune evasion mechanism (e.g., Trypanosoma brucei; see, e.g., Horn (2014) MOL. BIOCHEM. PARASITOL. 195(2): 123-129). Thus, for example, by using the host cells comprising the PUCRs disclosed herein, therapies may be customized to target the antigenic variant being expressed by the disease-causing organism. In some embodiments, the disease-causing organism is a pathogenic virus or a pathogenic bacterium. In some embodiments, the virus is selected from the group consisting of HIV, an influenza virus, a herpes virus, a rotavirus, a respiratory syncytial virus, a poliovirus, a rhinovirus, a hepatitis virus (e.g., hepatitis viruses types A, B, C, D, E and/or G), a cytomegalovirus, a simian immunodeficiency virus, an encephalitis virus, a varicella zoster virus, an Epstein-Barr virus, and a virus belonging to a Coronaviridae, Birnaviridae or Filoviridae virus family. In some embodiments, the bacterium is selected from the group consisting of Mycobacterium (e.g., Mycobacterium tuberculosis), Chlamydia, Neisseria (e.g., Neisseria gonorrhoeae), Shigella, Salmonella, Moraxella (e.g., Moraxella catarrhalis), Vibrio (e.g., Vibrio cholerae), Treponema (e.g., Treponema pallidum), Pseudomonas, Bordetella (e.g., Bordetella pertussis), Brucella, Francisella (e.g., Francisella tularensis), Helicobacter (e.g., Helicobacter pylori), Leptospira (e.g., Leptospira interrogans), Legionella (e.g., Legionella pneumophila), Yersinia (e.g., Yersinia pestis), Streptococcus(e.g., Streptococcus pneumoniae), and Haemophilus (e.g., Haemophilus influenza). In some embodiments, the parasite is selected from the group consisting of Schistosoma (e.g., Schistosoma mansoni), Trypanosoma (e.g., Trypanosoma brucei), Fasciola (e.g., Fasciola hepatica), Trichuris (e.g., Trichuris trichiura), Plasmodium (e.g., Plasmodium vivax and Plasmodium falciparum). In some embodiments, the protozoan is selected from the group consisting of Entamoeba (e.g., Entamoeba histolytica), Cryptosporidium (e.g., Cryptosporidium parvum), Toxoplasma (e.g., Toxoplasma gondii) and Giardia (e.g., Giardia lamblia).

In some aspects of the invention, the host cells comprising a PUCR are administered to a subject, such that the host cells (or their progeny), persist in the subject for a given number of days, including, but not limited to, at least 0.5 days, one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, thirty-one days or more, after administration of the host cell to the subject. In some aspects of the invention, the host cells comprising a PUCR are administered to a subject, and the host cells (or their progeny), persist in the subject for at least one month, two months, three months, four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, two years, three years, four years, five years, or more, after administration of the host cell to the subject.

In some embodiments, the subject is administered a host cell comprising a PUCR that has been programmed (i.e., conjugated) with a specificity agent. Because some or all of the programmed PUCR may be internalized by said host cell during normal plasma membrane recycling processes, the host cell may exhibit reduced ability to bind to, or reduced specificity for, a target molecule. Without wishing to be bound by any theory, internalization of the programmed PUCR by the host cell may be particularly advantageous as it provides a means to regulate the activity (e.g., the cytotoxic activity) of the host cell comprising the programmed PUCR. In some embodiments, the subject must be re-administered a host cell comprising a PUCR that has been programmed with a specificity agent. In some embodiments, the source of a host cell comprising a PUCR that has not been programmed (i.e. is not conjugated) with a specificity agent, is the subject. In some embodiments, the source of a host cell comprising a PUCR that has not been programmed (i.e., is not conjugated) with a specificity agent, is not the subject.

In one aspect, the present invention provides a pharmaceutical composition comprising a host cell comprising a PUCR, as described herein, in combination with one or more pharmaceutically or physiologically acceptable carriers, diluents or excipients. Such compositions may comprise buffers (e.g., a buffered saline (e.g., phosphate buffered saline) and the like); carbohydrates such as glucose, mannose, sucrose, dextrans, sugar alcohols (e.g., mannitol); proteins (e.g., growth factors and cytokines); amino acids; antioxidants; chelating agents (e.g., EDTA or EGTA); adjuvants (e.g., aluminum hydroxide); and preservatives. In some embodiments, pharmaceutical compositions for use in the present invention are formulated for intravenous administration.

The compositions of the present invention may be administered by any means known in the art, including, e.g., by aerosol inhalation, injection, ingestion, transfusion, implantation or transplantation. The compositions described herein (e.g., a host cell comprising a PUCR, as described herein) may be administered to a subject trans arterially, subcutaneously, intradermally, intratumorally, intranodally, intramedullary, intramuscularly, by intravenous (i.v.) injection, or intraperitoneally. In one embodiment, the compositions of the present invention are administered to a subject by intradermal or subcutaneous injection. In another embodiment, the compositions of the present invention are administered by i.v. injection. In one embodiment, the compositions of the present invention are administered by injection directly into a tumor, lymph node, or site of infection. The precise amount or dosage of the compositions of the present invention to be administered to a subject can be determined by a physician with consideration of individual differences in age, weight, tumor size, metastasis, extent of an infection, pre-existing medical condition of a subject, and the current physiological condition of the subject.

In one embodiment, a pharmaceutical composition comprising the host cells described herein may be administered at a dosage of about 10¹ to about 10⁹ cells/kg body weight. Ranges intermediate to the above recited dosage, e.g., about 10² to about 10⁸ cells/kg body weight, about 10⁴ to about 10⁷ cells/kg body weight, about 10⁵ to about 10⁶ cells/kg body weight, are also intended to be part of this invention. In some embodiments, the host cells described herein may be administered at a dosage of about 10² to about 10¹¹ cells/m². Ranges intermediate to the above recited dosage, e.g., about 10³ to about 10⁹ cells/m², about 10⁴ to about 10⁷ cells/m², about 10⁵ to about 10⁶ cells/m², are also intended to be part of this invention. Furthermore, ranges of values using a combination of any of the above recited values as upper and/or lower limits are intended to be included. In some embodiments, about 10², 10³, 10⁴, 10⁵, 10⁶, 10⁷, 10⁸, 10⁹, 10¹⁰, 10¹¹, 10¹², or more, host cells described herein are administered to a subject. Host cell compositions may also be administered multiple times at these dosages.

### III. Exemplification

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example I. Construction and Characterization of Humanized and Murine 38C2 scFv-Fc

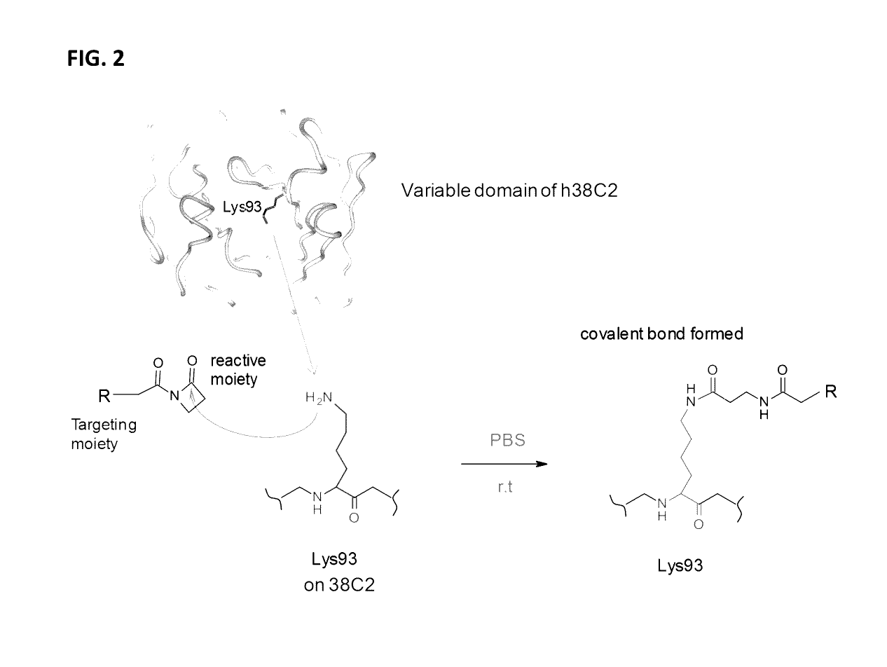

The murine monoclonal antibody 38C2 is a catalytic antibody discovered by Lerner/Barbas group at Scripps Research Institute in 1990s (Wagner et al. SCIENCE (1995) 270: 1797-1800). The variable domain contains a lysine residue located in a hydrophobic core. Due to the microchemical environment, the lysine side chain NH₂ group remains unprotonated under physiological conditions, feasible to attack a reactive moiety to form a covalent bond (FIG. 2). As illustrated in FIG. 2, the Lys93 residue in the variable domain of a 38C2 antibody (e.g., humanized 38C2 antibody) may serve as a nucleophile to interact with the reactive moiety of a specificity agent, resulting in the formation of a covalent bond between the Lys93 residue and the specificity agent.

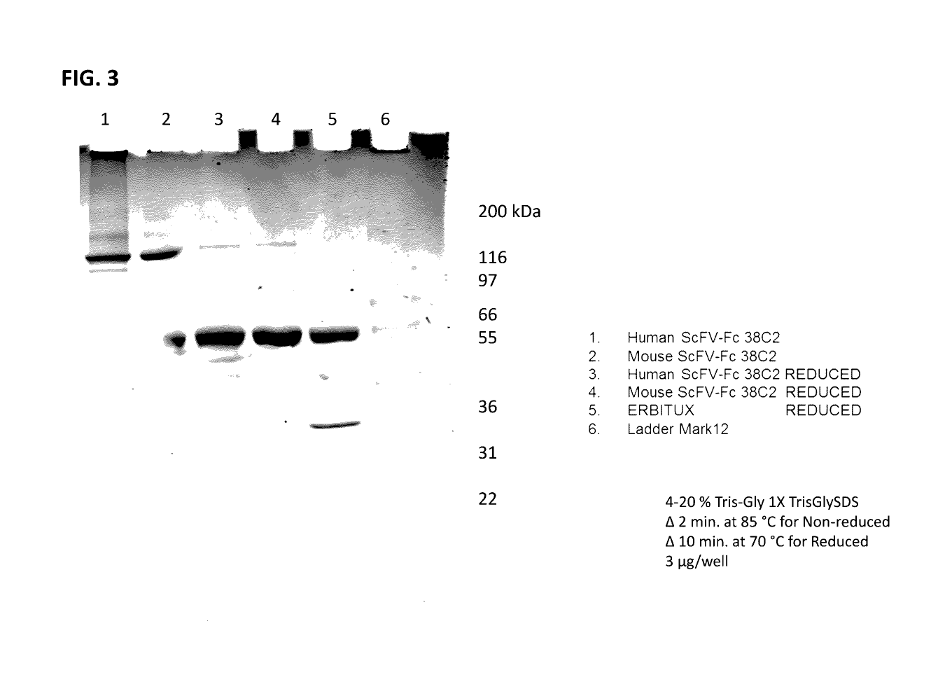

To generate humanized and murine 38C2 single chain variable fragment (scFv) of humanized or murine 38C2, the heavy chain and light chain variable domain sequences of the murine and humanized 38C2 IgG (Rader et al. J. MOL. BIOL. (2003) 332: 889-899) were codon optimized, synthesized, and reformatted as genes encoding the scFv, and cloned into a mammalian cell expression vector, so that the scFv fragment was fused in frame to Fc (fragment constant) portion of human IgG1 for expression and purification. Chinese Hamster Ovary (CHO) cells were transfected with either expression vector using the transfection reagent lipofectamine (ThermoFisher). Both murine and humanized 38C2 scFv-Fc were purified to homogeneity for catalytic activity tests. FIG. 3 shows an SDS-PAGE analysis for both the humanized and murine 38C2 scFv-Fc under non-reducing and reducing conditions. As shown in FIG. 3, both humanized and murine 38C2 scFv-Fc were purified using protein A affinity chromatography and the molecular weight for a single scFv-Fc was about 60 kDa under reducing conditions, whereas under non-reducing conditions, the molecular weight of scFv-Fc was about 120 kDa, indicating that dimers were formed.

To determine whether the purified murine and humanized 38C2 scFv-Fc retained catalytic activity, and hence were functional, a representative specificity agent containing a reactive moiety, i.e., azetidinone-PEG5-methyl ester, was conjugated with the molecules. Briefly, 1.6 μL of azetidinone-PEG5-methyl ester in DMSO (1.0 mg/mL) was mixed with 96 μL of 38C2 scFv-Fc (0.52 mg/mL) in PBS, pH 7.4 in a PCR tube. The PCR tube was constantly rotated overnight at room temperature using a tube rotator. Excess azetidinone-PEG5-methyl ester was removed from the reactions by centrifugal filtering using an Amicon Ultra-4 Centrifugal Filter Unit with Ultracel-10 membrane (EMD Millipore Cat. No. UFC801008).

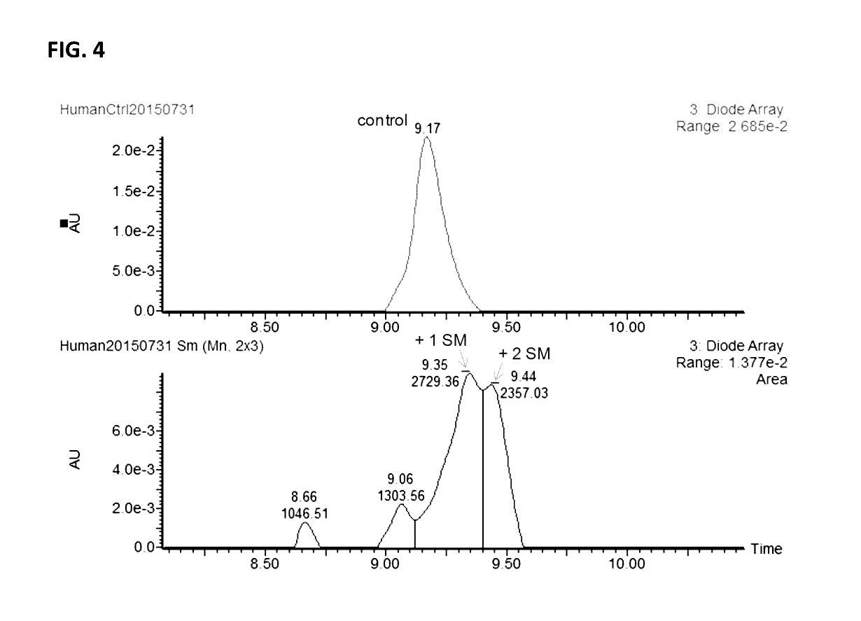

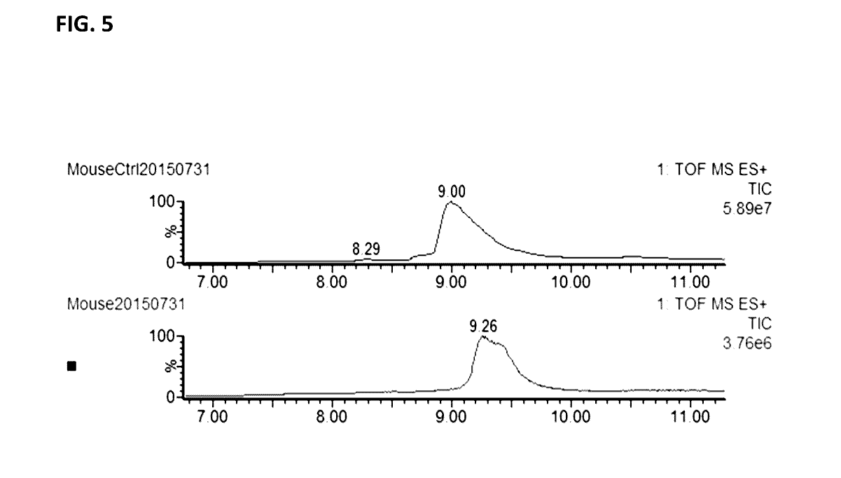

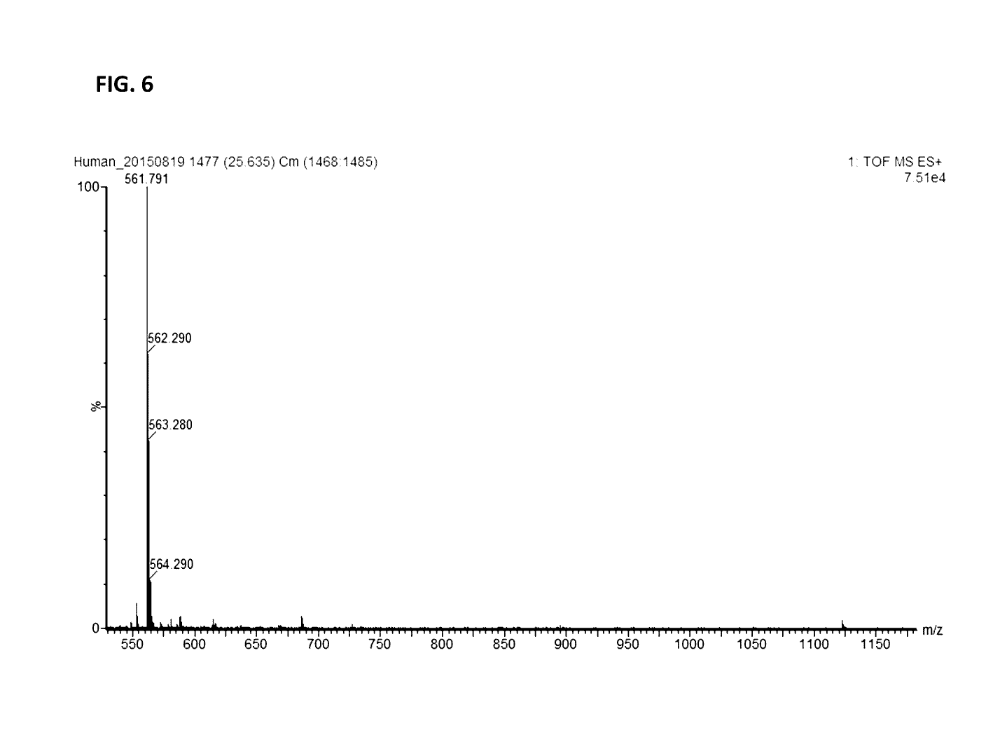

Each sample was then submitted for mass spectrometry analysis. The mass spectrometry data indicated that the majority of the 38C2 scFv-Fc was conjugated to 1 or 2 copies of azetidinone-PEG5-methyl ester, confirming that purified murine or humanized 38C2 scFv-Fc was functional in catalyzing the conjugation reaction with azetidinone-PEG5-methyl ester (see FIGS. 4 and 5). Peptide mapping was performed to confirm the conjugation site of azetidinone-PEG5-methyl ester on humanized 38C2 scFv-Fc as described, e.g., in Xie et al. (2009) WATERS APPLICATION NOTE 720002897EN (see FIG. 6). The mass of the peptide fragment containing azetidinone-PEG5-methyl ester was shown to contain a lysine residue, further suggesting that the conjugation occurred on Lys 93 of the heavy chain (FIG. 6, Table 5).

TABLE 5
Calculated and measured mass of conjugated
peptide fragment on heavy chain.

| | Theoretical | Measured | |
|---|---|---|---|
| | Mono | Mono | Error(ppm) |
| (M + H)⁺ | 1122.51659 | 1122.578 | 54.3 |
| (M + 2H)²⁺ | 561.76223 | 561.791 | 51.6 |

### Example 2. Generation of Programmable Universal Cell Receptors

In order to generate a programmable universal cell receptor (PUCR), the gene encoding the domains of the PUCR was codon optimized and custom synthesized (GenScript). The full length gene of the PUCR encodes in-frame sequences for: 1) a signal peptide for secretion or cell surface expression of the molecule; 2) a myc-tag for PUCR expression detection; 3) a catalytic antibody or catalytic portion thereof (e.g., scFv-Fc) as described in Example 1; ; 4) a hinge region (e.g., a CD8 hinge region); 5) a transmembrane domain (e.g., a CD3zeta transmembrane domain); 6) a cytoplasmic domain (e.g., a CD28 intracellular domain for T cell persistence and/or a CD3zeta intracellular domain for NK or T cell activation). The amino acid and nucleic acid sequences of each of the components are listed in Table 5 below.

TABLE 6
PUCR Component Sequences

| SEQ | | |
|---|---|---|
| ID | | |
| NO: | Description | Sequence |
| 1 | Signal | MEWSWVFLFFLSVTTGVHS |
| | peptide | |
| | amino acid | |
| | sequence | |
| 2 | Myc-tag | EQKLISEEDL |
| | amino acid | |
| | sequence | |
| 3 | Murine 38C2 | DVVMTQTPLSLPVRLGDQASISCRSSQSLLHTY |
| | scFv amino | GSPYLNWYLQKPGQSPKLLIYKVSNRFSGVPD |
| | acid | RFSGSGSGTDFTLRISRVEAEDLGVYFCSQGTH |
| | sequence | LPYTFGGGTKLEIKGGGGSGGGGSGGGGSEVK |
| | The scFv | LVESGGGLVQPGGTMKLSCEISGLTFRNYWMS |
| | is in a VL- | WVRQSPEKGLEWVAEIRLRSDNYATHYAESV |
| | linker-VH | KGKFTISRDDSKSRLYLQMNSLRTEDTGIYYCK |
| | configur- | TYFYSFSYWGQGTLVTVSA |
| | ation. The | |
| | underlined | |
| | sequence | |
| | is a poly | |
| | Gly₄Ser | |
| | linker. | |
| 4 | humanized | ELQMTQSPSSLSASVGDRVTITCRSSQSLLHTY |
| | 38C2 scFv | GSPYLNWYLQKPGQSPKLLIYKVSNRFSGVPSR |
| | amino acid | FSGSGSGTDFTLTISSLQPEDFAVYFCSQGTHLP |
| | sequence | YTFGGGTKVEIKGGGGSGGGGSGGGGSEVQLV |
| | The scFv | ESGGGLVQPGGSLRLSCAASGFTFSNYWMSW |
| | is in a VL- | VRQSPEKGLEWVSEIRLRSDNYATHYAESVKG |
| | linker-VH | RFTISRDNSKNTLYLQMNSLRAEDTGIYYCKTY |
| | configur- | FYSFSYWGQGTLVTVSS |
| | ation. The | |
| | underlined | |
| | sequence | |
| | is the | |
| | poly | |
| | Gly4Ser | |
| | linker. | |
| 5 | CD8 hinge | AKPTTTPAPRPPTPAPTIASQPLSLRPEACRPAA |
| | amino acid | GGAVHTRGLDFA |
| | sequence | |
| | This is a | |
| | fragment | |
| | of the CD8 | |
| | hinge | |
| | sequence. | |
| | The length | |
| | can be ex- | |
| | tended into | |
| | the N- | |
| | terminal | |
| | region of | |
| | CD8 | |
| | molecule. | |
| | Depending | |
| | on the | |
| | construct, | |
| | the only | |
| | cysteine | |
| | (under- | |
| | lined) in | |
| | the hinge | |
| | can be | |
| | mutated | |
| | for | |
| | increased | |
| | expression | |
| | of the | |
| | PUCR. | |
| 6 | CD3ζ | LDPKLCYLLDGILFIYGVILTALFLRVK |
| | transmem- | |
| | brane | |
| | domain | |
| | amino acid | |
| | sequence | |
| | The un- | |
| | derlined | |
| | is the | |
| | defined | |
| | hydrophobic | |
| | stretch of | |
| | the CD3ζ | |
| | transmem- | |
| | brane | |
| | domain | |
| | sequence. | |
| | Charged | |
| | residues | |
| | flanking | |
| | the trans- | |
| | membrane | |
| | domain on | |
| | both | |
| | sides are | |
| | also in- | |
| | cluded to | |
| | stop trans- | |
| | location. | |
| 7 | CD28 in- | RSKRSRLLHSDYMNMTPRRPGPTRKHYQPYAP |
| | tracellular | PRDFAAYRS |
| | domain | |
| | amino acid | |
| | sequence | |
| 8 | CD3ζ in- | RVKFSRSADAPAYQQGQNQLYNELNLGRREE |
| | tracellular | YDVLDKRRGRDPEMGGKPQRRKNPQEGLYNE |
| | domain | LQKDKMAEAYSEIGMKGERRRGKGHDGLYQG |
| | amino acid | LSTATKDTYDALHMQALPPR |
| | sequence | |
| 9 | Amino acid | MEWSWVFLFFLSVTTGVHSDVVMTQTPLSLPV |
| | sequence | RLGDQASISCRSSQSLLHTYGSPYLNWYLQKPG |
| | of murine | QSPKLLIYKVSNRFSGVPDRFSGSGSGTDFTLRI |
| | PUCR with | SRVEAEDLGVYFCSQGTHLPYTFGGGTKLEIKG |
| | Myc-tag | GGGSGGGGSGGGGSEVKLVESGGGLVQPGGT |
| | with signal | MKLSCEISGLTFRNYWMSWVRQSPEKGLEWV |
| | peptide | AEIRLRSDNYATHYAESVKGKFTISRDDSKSRL |
| | | YLQMNSLRTEDTGIYYCKTYFYSFSYWGQGTL |
| | | VTVSAEQKLISEEDLAKPTTTPAPRPPTPAPTIA |
| | | SQPLSLRPEACRPAAGGAVHTRGLDFALDPKL |
| | | CYLLDGILFIYGVILTALFLRVKRSKRSRLLHSD |
| | | YMNMTPRRPGPTRKHYQPYAPPRDFAAYRSR |
| | | VKFSRSADAPAYQQGQNQLYNELNLGRREEY |
| | | DVLDKRRGRDPEMGGKPQRRKNPQEGLYNEL |
| | | QKDKMAEAYSEIGMKGERRRGKGHDGLYQGL |
| | | STATKDTYDALHMQALPPR |
| 10 | Humanized | MEWSWVFLFFLSVTTGVHSELQMTQSPSSLSA |
| | PUCR with | SVGDRVTITCRSSQSLLHTYGSPYLNWYLQKP |
| | Myc-tag | GQSPKLLIYKVSNRFSGVPSRFSGSGSGTDFTLT |
| | with signal | ISSLQPEDFAVYFCSQGTHLPYTFGGGTKVEIK |
| | peptide | GGGGSGGGGSGGGGSEVQLVESGGGLVQPGG |
| | amino acid | SLRLSCAASGFTFSNYWMSWVRQSPEKGLEW |
| | sequence | VSEIRLRSDNYATHYAESVKGRFTISRDNSKNT |
| | | LYLQMNSLRAEDTGIYYCKTYFYSFSYWGQGT |
| | | LVTVSSEQKLISEEDLAKPTTTPAPRPPTPAPTIA |
| | | SQPLSLRPEACRPAAGGAVHTRGLDFALDPKL |
| | | CYLLDGILFIYGVILTALFLRVKRSKRSRLLHSD |
| | | YMNMTPRRPGPTRKHYQPYAPPRDFAAYRSR |
| | | VKFSRSADAPAYQQGQNQLYNELNLGRREEY |
| | | DVLDKRRGRDPEMGGKPQRRKNPQEGLYNEL |
| | | QKDKMAEAYSEIGMKGERRRGKGHDGLYQGL |
| | | STATKDTYDALHMQALPPR |
| 102 | Murine | DVVMTQTPLSLPVRLGDQASISCRSSQSLLHTY |
| | PUCR with | GSPYLNWYLQKPGQSPKLLIYKVSNRFSGVPD |
| | Myc-tag, | RFSGSGSGTDFTLRISRVEAEDLGVYFCSQGTH |
| | without | LPYTFGGGTKLEIKGGGGSGGGGSGGGGSEVK |
| | signal | LVESGGGLVQPGGTMKLSCEISGLTFRNYWMS |
| | peptide | WVRQSPEKGLEWVAElRLRSDNYATHYAESV |
| | (amino acid | KGKFTISRDDSKSRLYLQMNSLRTEDTGIYYCK |
| | sequence) | TYFYSFSYWGQGTLVTVSAEQKLISEEDLAKPT |
| | | TTPAPRPPTPAPTIASQPLSLRPEACRPAAGGAV |
| | | HTRGLDFALDPKLCYLLDGILFIYGVILTALFLR |
| | | VKRSKRSRLLHSDYMNMTPRRPGPTRKHYQP |
| | | YAPPRDFAAYRSRVKFSRSADAPAYQQGQNQL |
| | | YNELNLGRREEYDVLDKRRGRDPEMGGKPQR |
| | | RKNPQEGLYNELQKDKMAEAYSEIGMKGERR |
| | | RGKGHDGLYQGLSTATKDTYDALHMQALPPR |
| 103 | Humanized | ELQMTQSPSSLSASVGDRVTITCRSSQSLLHTY |
| | PUCR with | GSPYLNWYLQKPGQSPKLLIYKVSNRFSGVPSR |
| | Myc-tag, | FSGSGSGTDFTLTISSLQPEDFAVYFCSQGTHLP |
| | without | YTFGGGTKVEIKGGGGSGGGGSGGGGSEVQLV |
| | signal | ESGGGLVQPGGSLRLSCAASGFTFSNYWMSW |
| | peptide | VRQSPEKGLEWVSEIRLRSDNYATHYAESVKG |
| | (amino acid | RFTISRDNSKNTLYLQMNSLRAEDTGIYYCKTY |
| | sequence) | FYSFSYWGQGTLVTVSSEQKLISEEDLAKPTTT |
| | | PAPRPPTPAPTIASQPLSLRPEACRPAAGGAVHT |
| | | RGLDFALDPKLCYLLDGILFIYGVILTALFLRVK |
| | | RSKRSRLLHSDYMNMTPRRPGPTRKHYQPYAP |
| | | PRDFAAYRSRVKFSRSADAPAYQQGQNQLYN |
| | | ELNLGRREEYDVLDKRRGRDPEMGGKPQRRK |
| | | NPQEGLYNELQKDKMAEAYSEIGMKGERRRG |
| | | KGHDGLYQGLSTATKDTYDALHMQALPPR |
| 11 | Signal | ATGGAGTGGTCCTGGGTGTTCCTGTTCTTTCT |
| | peptide | GTCCGTGACCACCGGTGTCCAC |
| | (nucleic | |
| | acid | |
| | sequence) | |
| 12 | Myc-tag | GAGCAGAAACTCATTTCTGAAGAGGACCTT |
| | (nucleic | |
| | acid | |
| | sequence) | |
| 13 | Murine | GATGTAGTTATGACCCAGACGCCTCTTTCTCT |
| | 38C2 scFv | CCCCGTCCGGCTCGGAGACCAAGCCTCCATC |
| | (nucleic | TCTTGCCGAAGTTCACAATCATTGTTGCACA |
| | acid | CGTATGGATCCCCATATCTGAATTGGTATCTC |
| | sequence) | CAAAAGCCTGGACAGTCCCCCAAGCTGTTGA |
| | | TCTATAAAGTAAGTAATAGATTTTCCGGCGT |
| | | TCCTGACCGCTTCAGTGGCTCAGGAAGCGGT |
| | | ACGGATTTTACTCTTCGGATTTCCCGCGTCGA |
| | | AGCTGAAGATCTTGGTGTCTATTTCTGTTCTC |
| | | AGGGAACGCACCTGCCATACACATTCGGAGG |
| | | GGGCACTAAGCTCGAAATCAAGGGCGGGGG |
| | | CGGGTCAGGTGGTGGGGGCAGCGGCGGGGG |
| | | TGGCAGCGAGGTTAAGCTTGTGGAAAGTGGA |
| | | GGCGGGCTTGTGCAGCCGGGCGGGACCATG |
| | | AAACTGTCCTGCGAGATAAGTGGACTCACTT |
| | | TTAGGAACTATTGGATGAGCTGGGTGCGACA |
| | | GTCCCCCGAGAAGGGCCTTGAATGGGTTGCC |
| | | GAAATACGGCTTCGATCAGACAACTATGCGA |
| | | CGCACTACGCTGAAAGCGTCAAAGGAAAATT |
| | | CACTATCAGCCGGGACGACAGCAAGAGTAG |
| | | ACTTTATTTGCAGATGAATAGTTTGAGGACG |
| | | GAAGATACGGGAATATATTATTGCAAAACAT |
| | | ACTTCTATTCATTTTCATACTGGGGTCAGGGC |
| | | ACGTTGGTTACGGTTTCAGCC |
| 14 | Humanized | GAGCTTCAGATGACCCAAAGTCCCAGCTCTC |
| | 38C2 scFv | TCTCCGCCTCTGTCGGAGACAGGGTCACGAT |
| | (nucleic | AACCTGTCGAAGTAGCCAGAGTCTTCTCCAT |
| | acid | ACTTACGGAAGCCCATATCTTAACTGGTATC |
| | sequence) | TTCAGAAACCCGGTCAATCACCCAAGCTGCT |
| | | GATATATAAAGTGTCTAACCGGTTTTCTGGT |
| | | GTGCCGAGTCGATTTTCAGGATCAGGGAGCG |
| | | GCACGGATTTCACTCTTACGATCTCTAGTTTG |
| | | CAACCTGAGGATTTTGCTGTATACTTTTGCAG |
| | | CCAAGGTACTCATCTTCCTTATACGTTCGGA |
| | | GGGGGTACCAAAGTAGAAATTAAAGGAGGA |
| | | GGAGGGTCCGGAGGAGGGGGCAGCGGAGGA |
| | | GGAGGCTCAGAAGTACAACTCGTGGAATCTG |
| | | GCGGGGGGCTGGTGCAACCTGGGGGTTCTCT |
| | | CCGCCTGAGCTGTGCTGCATCCGGCTTCACC |
| | | TTTTCTAATTATTGGATGAGCTGGGTACGGC |
| | | AGTCACCGGAGAAAGGTCTGGAGTGGGTGTC |
| | | TGAGATACGACTTAGATCAGACAACTACGCG |
| | | ACGCATTACGCCGAGAGCGTGAAAGGAAGA |
| | | TTTACCATAAGCAGAGACAATTCAAAAAACA |
| | | CCCTGTACCTCCAAATGAATAGCCTCAGGGC |
| | | GGAAGATACTGGGATATATTACTGTAAAACC |
| | | TACTTTTACAGTTTTAGTTATTGGGGCCAGGG |
| | | AACGCTTGTAACTGTTAGCTCT |
| 15 | CD8 hinge | GCTAAGCCCACCACGACGCCAGCGCCGCGA |
| | (nucleic | CCACCAACACCGGCGCCCACCATCGCGTCGC |
| | acid | AGCCCCTGTCCCTGCGCCCAGAGGCGTGCCG |
| | sequence) | GCCAGCGGCGGGGGGCGCAGTGCACACGAG |
| | | GGGGCTGGACTTCGCC |
| 16 | CD3ζ | CTCGATCCGAAGTTGTGCTACCTGTTGGACG |
| | transmem- | GCATTCTCTTTATATACGGTGTCATCCTGACA |
| | brane | GCGTTGTTTCTCCGAGTGAAG |
| | (domain | |
| | nucleic | |
| | acid | |
| | sequence) | |
| 17 | CD28 in- | AGGAGTAAGAGGAGCAGGCTCCTGCACAGT |
| | tracellular | GACTACATGAACATGACTCCCCGCCGCCCCG |
| | domain | GGCCCACCCGCAAGCATTACCAGCCCTATGC |
| | (nucleic | CCCACCACGCGACTTCGCAGCCTATCGCTCC |
| | acid | |
| | sequence) | |
| 18 | CD3ζ in- | AGAGTGAAGTTCAGCAGGAGCGCAGACGCC |
| | tracellular | CCCGCGTACCAGCAGGGCCAGAACCAGCTCT |
| | domain | ATAACGAGCTCAATCTAGGACGAAGAGAGG |
| | (nucleic | AGTACGATGTTTTGGACAAGAGACGTGGCCG |
| | acid | GGACCCTGAGATGGGGGGAAAGCCGAGAAG |
| | sequence) | GAAGAACCCTCAGGAAGGCCTGTACAATGA |
| | | ACTGCAGAAAGATAAGATGGCGGAGGCCTA |
| | | CAGTGAGATTGGGATGAAAGGCGAGCGCCG |
| | | GAGGGGCAAGGGGCACGATGGCCTTTACCA |
| | | GGGTCTCAGTACAGCCACCAAGGACACCTAC |
| | | GACGCCCTTCACATGCAGGCCCTGCCCCCTC |
| | | GCTAA |
| 19 | Murine | GATGTAGTTATGACCCAGACGCCTCTTTCTCT |
| | PUCR with | CCCCGTCCGGCTCGGAGACCAAGCCTCCATC |
| | Myc-tag, | TCTTGCCGAAGTTCACAATCATTGTTGCACA |
| | without | CGTATGGATCCCCATATCTGAATTGGTATCTC |
| | signal | CAAAAGCCTGGACAGTCCCCCAAGCTGTTGA |
| | peptide | TCTATAAAGTAAGTAATAGATTTTCCGGCGT |
| | (nucleic | TCCTGACCGCTTCAGTGGCTCAGGAAGCGGT |
| | acid | ACGGATTTTACTCTTCGGATTTCCCGCGTCGA |
| | sequence) | AGCTGAAGATCTTGGTGTCTATTTCTGTTCTC |
| | | AGGGAACGCACCTGCCATACACATTCGGAGG |
| | | GGGCACTAAGCTCGAAATCAAGGGCGGGGG |
| | | CGGGTCAGGTGGTGGGGGCAGCGGCGGGGG |
| | | TGGCAGCGAGGTTAAGCTTGTGGAAAGTGGA |
| | | GGCGGGCTTGTGCAGCCGGGCGGGACCATG |
| | | AAACTGTCCTGCGAGATAAGTGGACTCACTT |
| | | TTAGGAACTATTGGATGAGCTGGGTGCGACA |
| | | GTCCCCCGAGAAGGGCCTTGAATGGGTTGCC |
| | | GAAATACGGCTTCGATCAGACAACTATGCGA |
| | | CGCACTACGCTGAAAGCGTCAAAGGAAAATT |
| | | CACTATCAGCCGGGACGACAGCAAGAGTAG |
| | | ACTTTATTTGCAGATGAATAGTTTGAGGACG |
| | | GAAGATACGGGAATATATTATTGCAAAACAT |
| | | ACTTCTATTCATTTTCATACTGGGGTCAGGGC |
| | | ACGTTGGTTACGGTTTCAGCCGAGCAGAAGC |
| | | TCATTTCCGAAGAAGATCTCGCAAAGCCGAC |
| | | AACGACGCCGGCACCCCGGCCTCCCACCCCC |
| | | GCCCCCACTATAGCTAGTCAACCTCTTTCACT |
| | | GCGCCCTGAAGCGTGTAGACCTGCAGCCGGG |
| | | GGAGCAGTCCATACGCGCGGACTTGATTTCG |
| | | CCCTCGACCCCAAGTTGTGTTACCTTTTGGAC |
| | | GGGATCCTCTTCATTTACGGTGTCATTCTTAC |
| | | TGCCTTGTTTCTCAGGGTAAAAAGGTCTAAG |
| | | AGATCCCGACTCCTCCATTCTGACTACATGA |
| | | ATATGACACCGAGGAGACCGGGACCAACTC |
| | | GGAAGCATTATCAGCCATACGCGCCCCCCCG |
| | | CGATTTCGCGGCATACAGGTCAAGAGTCAAG |
| | | TTCTCCCGCAGCGCAGACGCGCCCGCTTATC |
| | | AGCAAGGTCAAAATCAACTCTACAATGAGCT |
| | | CAATCTGGGACGACGGGAGGAGTACGATGT |
| | | CCTCGACAAGAGGAGAGGTCGGGATCCTGA |
| | | AATGGGTGGCAAACCCCAGCGACGCAAGAA |
| | | TCCTCAGGAGGGTCTCTACAACGAGCTGCAA |
| | | AAAGATAAAATGGCGGAGGCGTATAGTGAA |
| | | ATAGGGATGAAAGGGGAAAGACGCCGGGGA |
| | | AAAGGACATGATGGTCTGTATCAGGGTCTGT |
| | | CAACAGCTACTAAAGACACATACGATGCGCT |
| | | GCACATGCAAGCGTTGCCGCCGAGG |
| 20 | Humanized | GAGCTTCAGATGACCCAAAGTCCCAGCTCTC |
| | PUCR with | TCTCCGCCTCTGTCGGAGACAGGGTCACGAT |
| | Myc-tag, | AACCTGTCGAAGTAGCCAGAGTCTTCTCCAT |
| | without | ACTTACGGAAGCCCATATCTTAACTGGTATC |
| | signal | TTCAGAAACCCGGTCAATCACCCAAGCTGCT |
| | peptide | GATATATAAAGTGTCTAACCGGTTTTCTGGT |
| | (nucleic | GTGCCGAGTCGATTTTCAGGATCAGGGAGCG |
| | acid | GCACGGATTTCACTCTTACGATCTCTAGTTTG |
| | sequence) | CAACCTGAGGATTTTGCTGTATACTTTTGCAG |
| | | CCAAGGTACTCATCTTCCTTATACGTTCGGA |
| | | GGGGGTACCAAAGTAGAAATTAAAGGAGGA |
| | | GGAGGGTCCGGAGGAGGGGGCAGCGGAGGA |
| | | GGAGGCTCAGAAGTACAACTCGTGGAATCTG |
| | | GCGGGGGGCTGGTGCAACCTGGGGGTTCTCT |
| | | CCGCCTGAGCTGTGCTGCATCCGGCTTCACC |
| | | TTTTCTAATTATTGGATGAGCTGGGTACGGC |
| | | AGTCACCGGAGAAAGGTCTGGAGTGGGTGTC |
| | | TGAGATACGACTTAGATCAGACAACTACGCG |
| | | ACGCATTACGCCGAGAGCGTGAAAGGAAGA |
| | | TTTACCATAAGCAGAGACAATTCAAAAAACA |
| | | CCCTGTACCTCCAAATGAATAGCCTCAGGGC |
| | | GGAAGATACTGGGATATATTACTGTAAAACC |
| | | TACTTTTACAGTTTTAGTTATTGGGGCCAGGG |
| | | AACGCTTGTAACTGTTAGCTCTGAGCAGAAG |
| | | CTCATTTCCGAAGAAGATCTCGCAAAGCCGA |
| | | CAACGACGCCGGCACCCCGGCCTCCCACCCC |
| | | CGCCCCCACTATAGCTAGTCAACCTCTTTCA |
| | | CTGCGCCCTGAAGCGTGTAGACCTGCAGCCG |
| | | GGGGAGCAGTCCATACGCGCGGACTTGATTT |
| | | CGCCCTCGACCCCAAGTTGTGTTACCTTTTGG |
| | | ACGGGATCCTCTTCATTTACGGTGTCATTCTT |
| | | ACTGCCTTGTTTCTCAGGGTAAAAAGGTCTA |
| | | AGAGATCCCGACTCCTCCATTCTGACTACAT |
| | | GAATATGACACCGAGGAGACCGGGACCAAC |
| | | TCGGAAGCATTATCAGCCATACGCGCCCCCC |
| | | CGCGATTTCGCGGCATACAGGTCAAGAGTCA |
| | | AGTTCTCCCGCAGCGCAGACGCGCCCGCTTA |
| | | TCAGCAAGGTCAAAATCAACTCTACAATGAG |
| | | CTCAATCTGGGACGACGGGAGGAGTACGAT |
| | | GTCCTCGACAAGAGGAGAGGTCGGGATCCTG |
| | | AAATGGGTGGCAAACCCCAGCGACGCAAGA |
| | | ATCCTCAGGAGGGTCTCTACAACGAGCTGCA |
| | | AAAAGATAAAATGGCGGAGGCGTATAGTGA |
| | | AATAGGGATGAAAGGGGAAAGACGCCGGGG |
| | | AAAAGGACATGATGGTCTGTATCAGGGTCTG |
| | | TCAACAGCTACTAAAGACACATACGATGCGC |
| | | TGCACATGCAAGCGTTGCCGCCGAGG |

Any of the foregoing exemplary sequences may be included in the PUCRs described herein.

### Example 3. Labeling of T Cells or NK Cells Expressing Programmable Universal Cell Receptor (PUCR-T or PUCR-NK Cells) with Specificity Agents

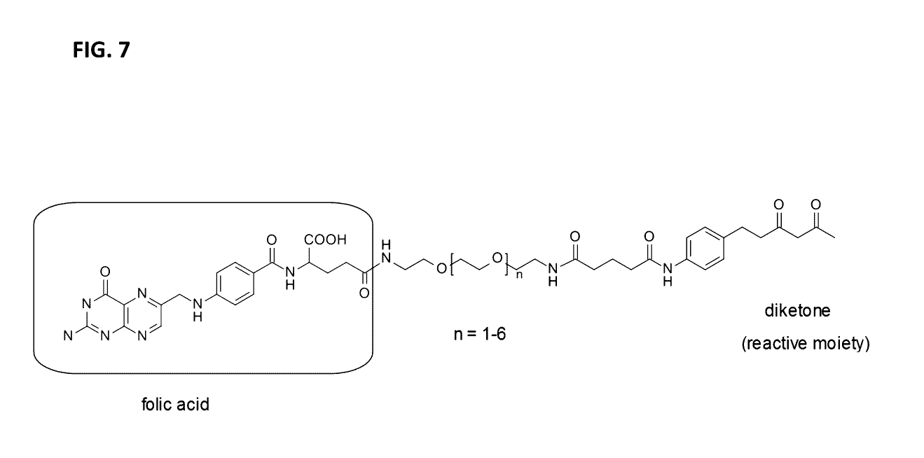

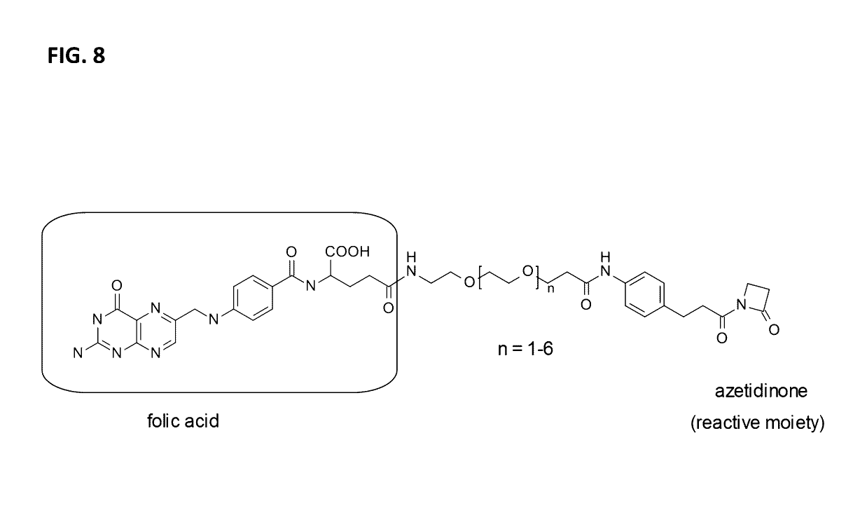

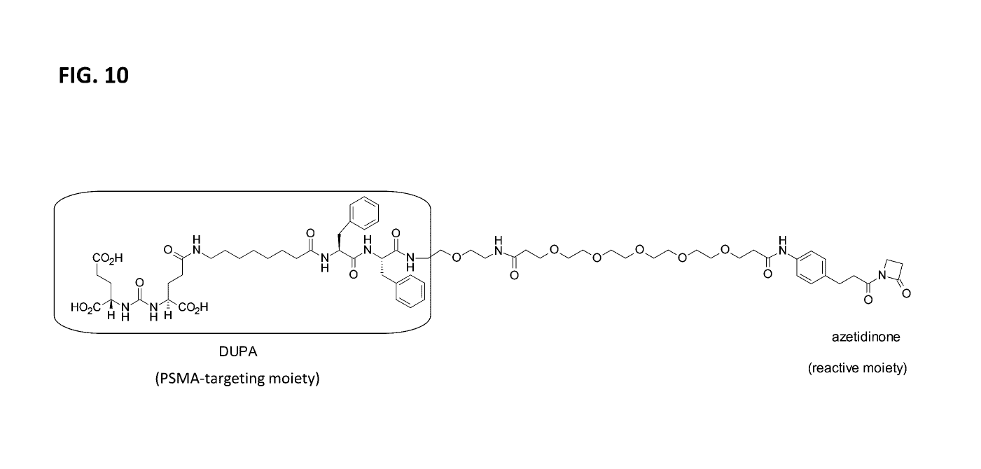

PUCR-T cells and PUCR-NK cells are generated. In order to label PUCR-T cells or PUCR-NK cells which express PUCR, cells are contacted with specificity agents (e.g., antigen binding molecules) which contain a targeting moiety (e.g., a tumor-specific protein-binding moiety) and a reactive moiety. The reactive moiety and the targeting moiety may be connected via a linker (e.g., a polyethylene glycol (PEG) fragment). For example, folic acid-diketone, folic acid-azetidinone, DUPA-diketone, and DUPA-azetidinone are used as specificity agents. The chemical structures of these four exemplary specificity agents are illustrated in FIGS. 7-11. Folic acid acts as a targeting moiety that targets folate receptors, which are highly overexpressed on the surface of many tumor types, and 2-[3-(1, 3-dicarboxy propyl)-ureido] pentanedioic acid (DUPA) acts as a targeting moiety that targets prostate specific membrane antigen (PSMA). The diketone or the azetidinone group is the reactive moiety that interacts with the reactive Lys residue in the catalytic antibody, e.g., a 38C2 antibody, or a catalytic portion thereof, within the PUCR.

For example, PUCR-T cells (1×10⁵) and PUCR-NK cells (1×10⁵) are incubated with 50 nM of folate-diketone in PBS for 2 hours at 4° C. After washing the cells three times, the cells are subjected to binding and cytotoxicity assays, as described in the below examples.

### Example 4. Binding Specificity of T Cells Comprising a PUCR Programmed with a Specificity Agents and a Targeted Antigen

To determine the binding specificity of a PUCR programmed (i.e., conjugated) with a specificity agent (e.g., folic acid-diketone), competitive binding assays are performed. Specifically, PUCR-T cells, or the folate-receptor-expressing KB cells (1×10⁵ cells) are incubated with 50 nM of folic acid-diketone and varied concentrations of free diketone or free folic acid, respectively. After a 2-hour incubation period at 4° C., cells are washed three times with FACS buffer. For KB cells, the cells are incubated with phycoerythrin (PE)-labeled anti-diketone antibody for 30 minutes at 4° C., and further washed twice with FACS buffer. The cells are immediately analyzed using Intellicyt HTFC, and binding of folic acid-diketone is determined by PE emission. For PUCR-T cells, the cells are incubated with phycoerythrin (PE)-labeled anti-folic acid antibody for 30 minutes at 4° C., and further washed twice with FACS buffer. The cells are immediately analyzed using Intellicyt HTFC, and binding of folic acid-diketone determined by PE emission.

### Example 5. Cytotoxicity of PUCR-T Cells

In order to determine whether the PUCR-T cells that have been programmed with a folic acid-diketone specificity agent can effectively target folate receptor expressing cells, cytotoxicity assays are performed. Specifically, PUCR-T cells programmed (i.e., conjugated) with folic acid-diketone are mixed at 10:1 effector cell (PUCR T-cell):target cell (KB cell) E:T ratio with folate receptor-expressing KB cells in 100 μl of folic acid-deficient RPMI media with 10% fetal bovine serum (FBS), and incubated with varied concentrations of folic acid-diketone for 24 hours at 37° C. Cytotoxic activity is calculated by quantitating the amount of lactate dehydrogenase released into the culture media using the CytoTox 96® non-radioactive cytotoxicity assay (Promega Cat. No. G1780).

### Example 6. Generation and Characterization of NKL Natural Killer Cells Expressing PUCR Programmed with a Specificity Agent Comprising a Detectable Moiety

To generate constructs encoding a PUCR comprising humanized 38C2 scFab, a nucleic acid encoding the humanized 38C2 scFab was designed by fusing a nucleic acid encoding humanized 38C2 VH domain, a nucleic acid encoding human kappa LC domain, a nucleic acid encoding a poly-GlySer linker, a nucleic acid encoding the humanized 38C2 VH domain, and a nucleic acid encoding the human gamma 1 HC constant domain 1. The resulting nucleic acid fragment was cloned into a retroviral vector to encode a PUCR comprising an N-terminal leader peptide, followed by the 38C2 scFab, followed by a hybrid CD8 and CD28 hinge, a CD28 transmembrane domain, a CD28 intracellular domain, and a CD3ζ intracellular domain. The nucleic acid and amino acid sequences of the PUCR are shown below. NKL cells were transduced with the viral vector encoding the PUCR.

TABLE 7
PUCR Component Sequences

| SEQ | | |
|---|---|---|
| ID | Descrip- | |
| NO: | tion | Sequence |
| 1 | Signal | MEWSWVFLFFLSVTTGVHS |
| | peptide | |
| | amino | |
| | acid | |
| | sequence | |
| 40 | Human- | ELQMTQSPSSLSASVGDRVTITCRSSQSLLHTYGSPYLN |
| | ized | WYLQKPGQSPKLLIYKVSNRFSGVPSRFSGSGSGTDFTL |
| | 38C2 | TISSLQPEDFAVYFCSQGTHLPYTFGGGTKVEIK |
| | variable | |
| | kappa | |
| | heavy | |
| | chain | |
| 41 | Human | RTVAAPSVFIFPPSDEQLKSGTASVVCLLNNFYPREAKV |
| | kappa | QWKVDNALQSGNSQESVTEQDSKDSTYSLSSTLTLSKA |
| | light | DYEKHKVYACEVTHQGLSSPVTKSFNRGEC |
| | chain | |
| | constant | |
| 54 | PolyGly | GGGSGGGGSGGGSGGGGSGGGSGGGGSGGGGSGGGS |
| | Ser | GGGGS |
| | linker | |
| 42 | Human- | EVQLVESGGGLVQPGGSLRLSCAASGFTFSNYWMSWV |
| | ized | RQSPEKGLEWVSEIRLRSDNYATHYAESVKGRFTISRD |
| | 38C2 | NSKNTLYLQMNSLRAEDTGIYYCKTYFYSFSYWGQGT |
| | variable | LVTVSS |
| | heavy | |
| | chain | |
| 43 | Human | ASTKGPSVFPLAPSSKSTSGGTAALGCLVKDYFPEPVTV |
| | gamma 1 | SWNSGALTSGVHTFPAVLQSSGLYSLSSVVTVPSSSLGT |
| | heavy | QTYICNVNHKPSNTKVDKKVEPKSCDKTHT |
| | chain | |
| | constant | |
| | domain 1 | |
| 44 | Full | MEWSWVFLFFLSVTTGVHSELQMTQSPSSLSASVGDR |
| | length | VTITCRSSQSLLHTYGSPYLNWYLQKPGQSPKLLIYKVS |
| | human- | NRFSGVPSRFSGSGSGTDFTLTISSLQPEDFAVYFCSQG |
| | ized | THLPYTFGGGTKVEIKRTVAAPSVFIFPPSDEQLKSGTA |
| | 38C2 | SVVCLLNNFYPREAKVQWKVDNALQSGNSQESVTEQDS |
| | scFab | KDSTYSLSSTLTLSKADYEKHKVYACEVTHQGLSSPVT |
| | with | KSFNRGECGGGSGGGGSGGGSGGGGSGGGSGGGGSG |
| | signal | GGGSGGGSGGGGSEVQLVESGGGLVQPGGSLRLSCAA |
| | peptide | SGFTFSNYWMSWVRQSPEKGLEWVSEIRLRSDNYATH |
| | | YAESVKGRFTISRDNSKNTLYLQMNSLRAEDTGIYYCK |
| | | TYFYSFSYWGQGTLVTVSSASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVDK |
| | | KVEPKSCDKTHT |
| 104 | Full | ELQMTQSPSSLSASVGDRVTITCRSSQSLLHTYGSPYLN |
| | length | WYLQKPGQSPKLLIYKVSNRFSGVPSRFSGSGSGTDFTL |
| | human- | TISSLQPEDFAVYFCSQGTHLPYTFGGGTKVEIKRTVAA |
| | ized | PSVFIFPPSDEQLKSGTASVVCLLNNFYPREAKVQWKV |
| | 38C2 | DNALQSGNSQESVTEQDSKDSTYSLSSTLTLSKADYEK |
| | scFab | HKVYACEVTHQGLSSPVTKSFNRGECGGGSGGGGSGG |
| | without | GSGGGGSGGGSGGGGSGGGGSGGGSGGGGSEVQLVE |
| | signal | SGGGLVQPGGSLRLSCAASGFTFSNYWMSWVRQSPEK |
| | peptide | GLEWVSEIRLRSDNYATHYAESVKGRFTISRDNSKNTL |
| | | YLQMNSLRAEDTGIYYCKTYFYSFSYWGQGTLVTVSS |
| | | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHT |
| 55 | Hybrid | AKPTTTPAPRPPTPAPTIASQPLSLRPEACRPAAGGAVH |
| | CD8 and | TRGLDFAPRKIEVMYPPPYLDNEKSNGTIIHVKGKHLCP |
| | CD28 | SPLFPGPSKP |
| | hinge | |
| | amino | |
| | acid | |
| | sequence | |
| 56 | CD8 | AKPTTTPAPRPPTPAPTIASQPLSLRPEACRPAAGGAVH |
| | portion | TRGLDFA |
| | of | |
| | hybrid | |
| | CD8 and | |
| | CD28 | |
| | hinge | |
| | amino | |
| | acid | |
| | sequence | |
| 57 | Hinge | PR |
| | linker | |
| | amino | |
| | acid | |
| | sequence | |
| 58 | CD28 | KIEVMYPPPYLDNEKSNGTIIHVKGKHLCPSPLFPGPSK |
| | portion | P |
| | of | |
| | hybrid | |
| | CD8 and | |
| | CD28 | |
| | hinge | |
| | amino | |
| | acid | |
| | sequence | |
| 24 | CD28 | FWVLVVVGGVLACYSLLVTVAFIIFWV |
| | trans- | |
| | membrane | |
| | domain | |
| | amino | |
| | acid | |
| | sequence | |
| 7 | CD28 | RSKRSRLLHSDYMNMTPRRPGPTRKHYQPYAPPRDFA |
| | intra- | AYRS |
| | cellular | |
| | domain | |
| | amino | |
| | acid | |
| | sequence | |
| 59 | CD3ζ | RVKFSRSADAPAYQQGQNQLYNELNLGRREEYDVLDK |
| | intra- | RRGRDPEMGGKPRRKNPQEGLYNELQKDKMAEAYSEI |
| | cellular | GMKGERRRGKGHDGLYQGLSTATKDTYDALHMQALP |
| | domain | PR |
| | amino | |
| | acid | |
| | sequence | |
| 45 | Full | MEWSWVFLFFLSVTTGVHSELQMTQSPSSLSASVGDR |
| | length | VTITCRSSQSLLHTYGSPYLNWYLQKPGQSPKLLIYKVS |
| | PUCR | NRFSGVPSRFSGSGSGTDFTLTISSLQPEDFAVYFCSQG |
| | com- | THLPYTFGGGTKVEIKRTVAAPSVFIFPPSDEQLKSGTA |
| | prising | SVVCLLNNFYPREAKVQWKVDNALQSGNSQESVTEQDS |
| | 38C2 | KDSTYSLSSTLTLSKADYEKHKVYACEVTHQGLSSPVT |
| | scFab | KSFNRGECGGGSGGGGSGGGSGGGGSGGGSGGGGSG |
| | amino | GGGSGGGSGGGGSEVQLVESGGGLVQPGGSLRLSCAA |
| | acid | SGFTFSNYWMSWVRQSPEKGLEWVSEIRLRSDNYATH |
| | sequence | YAESVKGRFTISRDNSKNTLYLQMNSLRAEDTGIYYCK |
| | with | TYFYSFSYWGQGTLVTVSSASTKGPSVFPLAPSSKSTSG |
| | signal | GTAALGCLVKDYFPEPVTVSWNSGALTSGVHTFPAVL |
| | peptide | QSSGLYSLSSVVTVPSSSLGTQTYICNVNHKPSNTKVDK |
| | | KVEPKSCDKTHTAKPTTTPAPRPPTPAPTIASQPLSLRP |
| | | EACRPAAGGAVHTRGLDFAPRKIEVMYPPPYLDNEKSN |
| | | GTIIHVKGKHLCPSPLFPGPSKPFWVLVVVGGVLACYS |
| | | LLVTVAFIIFWVRSKRSRLLHSDYMNMTPRRPGPTRKH |
| | | YQPYAPPRDFAAYRSRVKFSRSADAPAYQQGQNQLYN |
| | | ELNLGRREEYDVLDKRRGRDPEMGGKPRRKNPQEGLY |
| | | NELQKDKMAEAYSEIGMKGERRRGKGHDGLYQGLST |
| | | ATKDTYDALHMQALPPR |
| 105 | Full | ELQMTQSPSSLSASVGDRVTITCRSSQSLLHTYGSPYLN |
| | length | WYLQKPGQSPKLLIYKVSNRFSGVPSRFSGSGSGTDFTL |
| | PUCR | TISSLQPEDFAVYFCSQGTHLPYTFGGGTKVEIKRTVAA |
| | com- | PSVFIFPPSDEQLKSGTASVVCLLNNFYPREAKVQWKV |
| | prising | DNALQSGNSQESVTEQDSKDSTYSLSSTLTLSKADYEK |
| | 38C2 | HKVYACEVTHQGLSSPVTKSFNRGECGGGSGGGGSGG |
| | scFab | GSGGGGSGGGSGGGGSGGGGSGGGSGGGGSEVQLVE |
| | amino | SGGGLVQPGGSLRLSCAASGFTFSNYWMSWVRQSPEK |
| | acid | GLEWVSEIRLRSDNYATHYAESVKGRFTISRDNSKNTL |
| | sequence | YLQMNSLRAEDTGIYYCKTYFYSFSYWGQGTLVTVSS |
| | without | ASTKGPSVFPLAPSSKSTSGGTAALGCLVKDYFPEPVTV |
| | signal | SWNSGALTSGVHTFPAVLQSSGLYSLSSVVTVPSSSLGT |
| | peptide | QTYICNVNHKPSNTKVDKKVEPKSCDKTHTAKPTTTPA |
| | | PRPPTPAPTIASQPLSLRPEACRPAAGGAVHTRGLDFAP |
| | | RKIEVMYPPPYLDNEKSNGTIIHVKGKHLCPSPLFPGPS |
| | | KPFWVLVVVGGVLACYSLLVTVAFIIFWVRSKRSRLLH |
| | | SDYMNMTPRRPGPTRKHYQPYAPPRDFAAYRSRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 46 | Signal | ATGGAATGGAGTTGGGTGTTCCTTTTCTTTCTGAGTG |
| | peptide | TCACCACCGGAGTGCAC |
| | nucleic | |
| | acid | |
| | sequence | |
| 47 | Human- | AGCGAACTGCAGATGACCCAGTCCCCATCCAGTCTG |
| | ized | AGCGCTAGCGTTGGTGACAGAGTTACTATCACCTGC |
| | 38C2 | CGCTCTTCACAGAGCCTGTTGCACACTTACGGCTCTC |
| | scFab | CTTACCTGAACTGGTATCTTCAGAAGCCTGGCCAAA |
| | nucleic | GCCCTAAGCTGCTCATCTACAAGGTGTCTAACAGGT |
| | acid | TCTCCGGGGTTCCGTCCCGCTTTTCAGGGAGCGGGT |
| | sequence | CAGGAACAGACTTCACCTTGACAATCTCAAGCCTCC |
| | | AGCCCGAGGATTTTGCCGTCTATTTCTGCTCACAAG |
| | | GCACACATCTGCCGTATACCTTTGGGGGCGGGACAA |
| | | AAGTCGAGATCAAAAGGACCGTCGCTGCACCATCCG |
| | | TGTTTATCTTCCCACCAAGTGACGAACAGCTCAAGA |
| | | GCGGTACTGCCTCCGTTGTTTGTCTGCTGAACAACTT |
| | | CTATCCAAGGGAAGCAAAGGTGCAATGGAAAGTAG |
| | | ACAACGCTCTGCAGTCAGGCAACTCCCAGGAGTCAG |
| | | TGACCGAGCAGGATAGCAAAGATTCAACATACAGCC |
| | | TGAGCAGCACCCTCACCCTGAGTAAGGCCGATTACG |
| | | AGAAGCACAAGGTTTACGCCTGCGAGGTGACCCACC |
| | | AGGGCCTTTCATCCCCAGTCACCAAATCTTTTAACCG |
| | | CGGCGAATGCGGGGGAGGCTCTGGTGGAGGCGGTTC |
| | | TGGAGGGGGCTCAGGAGGAGGCGGTAGCGGCGGTG |
| | | GTAGTGGGGGTGGCGGATCTGGCGGAGGTGGCTCAG |
| | | GAGGAGGTAGCGGCGGCGGGGGCAGCGAGGTCCAG |
| | | CTGGTAGAGTCAGGTGGAGGATTGGTGCAGCCCGGC |
| | | GGCAGTCTTAGACTCAGCTGTGCGGCCAGCGGATTT |
| | | ACTTTCTCAAATTATTGGATGTCTTGGGTCAGGCAGA |
| | | GCCCAGAGAAAGGCCTGGAATGGGTGTCAGAGATC |
| | | CGACTGAGAAGCGATAATTACGCGACTCATTATGCG |
| | | GAAAGCGTTAAAGGTCGGTTCACTATTTCACGAGAT |
| | | AATTCTAAGAATACCCTTTATCTGCAGATGAACAGC |
| | | TTGCGCGCCGAGGACACAGGCATCTACTACTGTAAA |
| | | ACTTACTTCTATTCTTTTTCCTACTGGGGACAGGGGA |
| | | CTCTCGTTACAGTCAGTAGCGCCTCCACCAAGGGTC |
| | | CTAGTGTCTTTCCCCTGGCCCCCTCATCCAAGTCCAC |
| | | GTCAGGAGGCACCGCGGCTCTGGGCTGTCTGGTCAA |
| | | AGACTACTTTCCTGAGCCAGTCACCGTGTCCTGGAA |
| | | TTCCGGCGCGCTTACTTCTGGCGTGCACACTTTCCCC |
| | | GCCGTCCTCCAGAGCAGTGGGCTGTATTCCCTGTCTT |
| | | CCGTAGTCACTGTGCCAAGCTCCAGTCTGGGAACCC |
| | | AGACCTATATTTGTAATGTGAATCATAAGCCGAGCA |
| | | ACACCAAGGTGGACAAGAAGGTGGAACCGAAGTCA |
| | | TGTGACAAAACCCACACT |
| 60 | Hybrid | GCTAAGCCCACCACGACGCCAGCGCCGCGACCACCA |
| | CD8 and | ACACCGGCGCCCACCATCGCGTCGCAGCCCCTGTCC |
| | CD28 | CTGCGCCCAGAGGCGTGCCGGCCAGCGGCGGGGGG |
| | hinge | CGCAGTGCACACGAGGGGGCTGGACTTCGCCCCTAG |
| | nucleic | GAAAATTGAAGTTATGTATCCTCCTCCTTACCTAGAC |
| | acid | AATGAGAAGAGCAATGGAACCATTATCCATGTGAAA |
| | sequence | GGGAAACACCTTTGTCCAAGTCCCCTATTTCCCGGA |
| | | CCTTCTAAGCCC |
| 61 | CD28 | TTTTGGGTGCTGGTGGTGGTTGGTGGAGTCCTGGCTT |
| | trans- | GCTATAGCTTGCTAGTAACAGTGGCCTTTATTATTTT |
| | membrane | CTGGGTG |
| | domain | |
| | nucleic | |
| | acid | |
| | sequence | |
| 17 | CD28 | AGGAGTAAGAGGAGCAGGCTCCTGCACAGTGACTA |
| | intra- | CATGAACATGACTCCCCGCCGCCCCGGGCCCACCCG |
| | cellular | CAAGCATTACCAGCCCTATGCCCCACCACGCGACTT |
| | domain | CGCAGCCTATCGCTCC |
| | nucleic | |
| | acid | |
| | sequence | |
| 62 | CD3ζ | AGAGTGAAGTTCAGCAGGAGCGCAGACGCCCCCGC |
| | intra- | GTACCAGCAGGGCCAGAACCAGCTCTATAACGAGCT |
| | cellular | CAATCTAGGACGAAGAGAGGAGTACGATGTTTTGGA |
| | domain | CAAGAGACGTGGCCGGGACCCTGAGATGGGGGGAA |
| | nucleic | AGCCGAGAAGGAAGAACCCTCAGGAAGGCCTGTAC |
| | acid | AATGAACTGCAGAAAGATAAGATGGCGGAGGCCTA |
| | sequence | CAGTGAGATTGGGATGAAAGGCGAGCGCCGGAGGG |
| | | GCAAGGGGCACGATGGCCTTTACCAGGGTCTCAGTA |
| | | CAGCCACCAAGGACACCTACGACGCCCTTCACATGC |
| | | AGGCCCTGCCCCCTCGCTAA |
| 48 | Full | ATGGAATGGAGTTGGGTGTTCCTTTTCTTTCTGAGTG |
| | length | TCACCACCGGAGTGCACAGCGAACTGCAGATGACCC |
| | PUCR | AGTCCCCATCCAGTCTGAGCGCTAGCGTTGGTGACA |
| | com- | GAGTTACTATCACCTGCCGCTCTTCACAGAGCCTGTT |
| | prising | GCACACTTACGGCTCTCCTTACCTGAACTGGTATCTT |
| | 38C2 | CAGAAGCCTGGCCAAAGCCCTAAGCTGCTCATCTAC |
| | scFab | AAGGTGTCTAACAGGTTCTCCGGGGTTCCGTCCCGC |
| | nucleic | TTTTCAGGGAGCGGGTCAGGAACAGACTTCACCTTG |
| | acid | ACAATCTCAAGCCTCCAGCCCGAGGATTTTGCCGTC |
| | sequence | TATTTCTGCTCACAAGGCACACATCTGCCGTATACCT |
| | with | TTGGGGGCGGGACAAAAGTCGAGATCAAAAGGACC |
| | signal | GTCGCTGCACCATCCGTGTTTATCTTCCCACCAAGTG |
| | peptide | ACGAACAGCTCAAGAGCGGTACTGCCTCCGTTGTTT |
| | | GTCTGCTGAACAACTTCTATCCAAGGGAAGCAAAGG |
| | | TGCAATGGAAAGTAGACAACGCTCTGCAGTCAGGCA |
| | | ACTCCCAGGAGTCAGTGACCGAGCAGGATAGCAAA |
| | | GATTCAACATACAGCCTGAGCAGCACCCTCACCCTG |
| | | AGTAAGGCCGATTACGAGAAGCACAAGGTTTACGCC |
| | | TGCGAGGTGACCCACCAGGGCCTTTCATCCCCAGTC |
| | | ACCAAATCTTTTAACCGCGGCGAATGCGGGGGAGGC |
| | | TCTGGTGGAGGCGGTTCTGGAGGGGGCTCAGGAGGA |
| | | GGCGGTAGCGGCGGTGGTAGTGGGGGTGGCGGATCT |
| | | GGCGGAGGTGGCTCAGGAGGAGGTAGCGGCGGCGG |
| | | GGGCAGCGAGGTCCAGCTGGTAGAGTCAGGTGGAG |
| | | GATTGGTGCAGCCCGGCGGCAGTCTTAGACTCAGCT |
| | | GTGCGGCCAGCGGATTTACTTTCTCAAATTATTGGAT |
| | | GTCTTGGGTCAGGCAGAGCCCAGAGAAAGGCCTGG |
| | | AATGGGTGTCAGAGATCCGACTGAGAAGCGATAATT |
| | | ACGCGACTCATTATGCGGAAAGCGTTAAAGGTCGGT |
| | | TCACTATTTCACGAGATAATTCTAAGAATACCCTTTA |
| | | TCTGCAGATGAACAGCTTGCGCGCCGAGGACACAGG |
| | | CATCTACTACTGTAAAACTTACTTCTATTCTTTTTCCT |
| | | ACTGGGGACAGGGGACTCTCGTTACAGTCAGTAGCG |
| | | CCTCCACCAAGGGTCCTAGTGTCTTTCCCCTGGCCCC |
| | | CTCATCCAAGTCCACGTCAGGAGGCACCGCGGCTCT |
| | | GGGCTGTCTGGTCAAAGACTACTTTCCTGAGCCAGT |
| | | CACCGTGTCCTGGAATTCCGGCGCGCTTACTTCTGGC |
| | | GTGCACACTTTCCCCGCCGTCCTCCAGAGCAGTGGG |
| | | CTGTATTCCCTGTCTTCCGTAGTCACTGTGCCAAGCT |
| | | CCAGTCTGGGAACCCAGACCTATATTTGTAATGTGA |
| | | ATCATAAGCCGAGCAACACCAAGGTGGACAAGAAG |
| | | GTGGAACCGAAGTCATGTGACAAAACCCACACTGCT |
| | | AAGCCCACCACGACGCCAGCGCCGCGACCACCAAC |
| | | ACCGGCGCCCACCATCGCGTCGCAGCCCCTGTCCCT |
| | | GCGCCCAGAGGCGTGCCGGCCAGCGGCGGGGGGCG |
| | | CAGTGCACACGAGGGGGCTGGACTTCGCCCCTAGGA |
| | | AAATTGAAGTTATGTATCCTCCTCCTTACCTAGACAA |
| | | TGAGAAGAGCAATGGAACCATTATCCATGTGAAAGG |
| | | GAAACACCTTTGTCCAAGTCCCCTATTTCCCGGACCT |
| | | TCTAAGCCCTTTTGGGTGCTGGTGGTGGTTGGTGGA |
| | | GTCCTGGCTTGCTATAGCTTGCTAGTAACAGTGGCCT |
| | | TTATTATTTTCTGGGTGAGGAGTAAGAGGAGCAGGC |
| | | TCCTGCACAGTGACTACATGAACATGACTCCCCGCC |
| | | GCCCCGGGCCCACCCGCAAGCATTACCAGCCCTATG |
| | | CCCCACCACGCGACTTCGCAGCCTATCGCTCCAGAG |
| | | TGAAGTTCAGCAGGAGCGCAGACGCCCCCGCGTACC |
| | | AGCAGGGCCAGAACCAGCTCTATAACGAGCTCAATC |
| | | TAGGACGAAGAGAGGAGTACGATGTTTTGGACAAG |
| | | AGACGTGGCCGGGACCCTGAGATGGGGGGAAAGCC |
| | | GAGAAGGAAGAACCCTCAGGAAGGCCTGTACAATG |
| | | AACTGCAGAAAGATAAGATGGCGGAGGCCTACAGT |
| | | GAGATTGGGATGAAAGGCGAGCGCCGGAGGGGCAA |
| | | GGGGCACGATGGCCTTTACCAGGGTCTCAGTACAGC |
| | | CACCAAGGACACCTACGACGCCCTTCACATGCAGGC |
| | | CCTGCCCCCTCGCTAA |
| 106 | Full | AGCGAACTGCAGATGACCCAGTCCCCATCCAGTCTG |
| | length | AGCGCTAGCGTTGGTGACAGAGTTACTATCACCTGC |
| | PUCR | CGCTCTTCACAGAGCCTGTTGCACACTTACGGCTCTC |
| | com- | CTTACCTGAACTGGTATCTTCAGAAGCCTGGCCAAA |
| | prising | GCCCTAAGCTGCTCATCTACAAGGTGTCTAACAGGT |
| | 38C2 | TCTCCGGGGTTCCGTCCCGCTTTTCAGGGAGCGGGT |
| | scFab | CAGGAACAGACTTCACCTTGACAATCTCAAGCCTCC |
| | without | AGCCCGAGGATTTTGCCGTCTATTTCTGCTCACAAG |
| | signal | GCACACATCTGCCGTATACCTTTGGGGGCGGGACAA |
| | peptide | AAGTCGAGATCAAAAGGACCGTCGCTGCACCATCCG |
| | nucleic | TGTTTATCTTCCCACCAAGTGACGAACAGCTCAAGA |
| | acid | GCGGTACTGCCTCCGTTGTTTGTCTGCTGAACAACTT |
| | sequence | CTATCCAAGGGAAGCAAAGGTGCAATGGAAAGTAG |
| | | ACAACGCTCTGCAGTCAGGCAACTCCCAGGAGTCAG |
| | | TGACCGAGCAGGATAGCAAAGATTCAACATACAGCC |
| | | TGAGCAGCACCCTCACCCTGAGTAAGGCCGATTACG |
| | | AGAAGCACAAGGTTTACGCCTGCGAGGTGACCCACC |
| | | AGGGCCTTTCATCCCCAGTCACCAAATCTTTTAACCG |
| | | CGGCGAATGCGGGGGAGGCTCTGGTGGAGGCGGTTC |
| | | TGGAGGGGGCTCAGGAGGAGGCGGTAGCGGCGGTG |
| | | GTAGTGGGGGTGGCGGATCTGGCGGAGGTGGCTCAG |
| | | GAGGAGGTAGCGGCGGCGGGGGCAGCGAGGTCCAG |
| | | CTGGTAGAGTCAGGTGGAGGATTGGTGCAGCCCGGC |
| | | GGCAGTCTTAGACTCAGCTGTGCGGCCAGCGGATTT |
| | | ACTTTCTCAAATTATTGGATGTCTTGGGTCAGGCAGA |
| | | GCCCAGAGAAAGGCCTGGAATGGGTGTCAGAGATC |
| | | CGACTGAGAAGCGATAATTACGCGACTCATTATGCG |
| | | GAAAGCGTTAAAGGTCGGTTCACTATTTCACGAGAT |
| | | AATTCTAAGAATACCCTTTATCTGCAGATGAACAGC |
| | | TTGCGCGCCGAGGACACAGGCATCTACTACTGTAAA |
| | | ACTTACTTCTATTCTTTTTCCTACTGGGGACAGGGGA |
| | | CTCTCGTTACAGTCAGTAGCGCCTCCACCAAGGGTC |
| | | CTAGTGTCTTTCCCCTGGCCCCCTCATCCAAGTCCAC |
| | | GTCAGGAGGCACCGCGGCTCTGGGCTGTCTGGTCAA |
| | | AGACTACTTTCCTGAGCCAGTCACCGTGTCCTGGAA |
| | | TTCCGGCGCGCTTACTTCTGGCGTGCACACTTTCCCC |
| | | GCCGTCCTCCAGAGCAGTGGGCTGTATTCCCTGTCTT |
| | | CCGTAGTCACTGTGCCAAGCTCCAGTCTGGGAACCC |
| | | AGACCTATATTTGTAATGTGAATCATAAGCCGAGCA |
| | | ACACCAAGGTGGACAAGAAGGTGGAACCGAAGTCA |
| | | TGTGACAAAACCCACACTGCTAAGCCCACCACGACG |
| | | CCAGCGCCGCGACCACCAACACCGGCGCCCACCATC |
| | | GCGTCGCAGCCCCTGTCCCTGCGCCCAGAGGCGTGC |
| | | CGGCCAGCGGCGGGGGGCGCAGTGCACACGAGGGG |
| | | GCTGGACTTCGCCCCTAGGAAAATTGAAGTTATGTA |
| | | TCCTCCTCCTTACCTAGACAATGAGAAGAGCAATGG |
| | | AACCATTATCCATGTGAAAGGGAAACACCTTTGTCC |
| | | AAGTCCCCTATTTCCCGGACCTTCTAAGCCCTTTTGG |
| | | GTGCTGGTGGTGGTTGGTGGAGTCCTGGCTTGCTAT |
| | | AGCTTGCTAGTAACAGTGGCCTTTATTATTTTCTGGG |
| | | TGAGGAGTAAGAGGAGCAGGCTCCTGCACAGTGACT |
| | | ACATGAACATGACTCCCCGCCGCCCCGGGCCCACCC |
| | | GCAAGCATTACCAGCCCTATGCCCCACCACGCGACT |
| | | TCGCAGCCTATCGCTCCAGAGTGAAGTTCAGCAGGA |
| | | GCGCAGACGCCCCCGCGTACCAGCAGGGCCAGAAC |
| | | CAGCTCTATAACGAGCTCAATCTAGGACGAAGAGAG |
| | | GAGTACGATGTTTTGGACAAGAGACGTGGCCGGGAC |
| | | CCTGAGATGGGGGGAAAGCCGAGAAGGAAGAACCC |
| | | TCAGGAAGGCCTGTACAATGAACTGCAGAAAGATA |
| | | AGATGGCGGAGGCCTACAGTGAGATTGGGATGAAA |
| | | GGCGAGCGCCGGAGGGGCAAGGGGCACGATGGCCT |
| | | TTACCAGGGTCTCAGTACAGCCACCAAGGACACCTA |
| | | CGACGCCCTTCACATGCAGGCCCTGCCCCCTCGCTA |
| | | A |

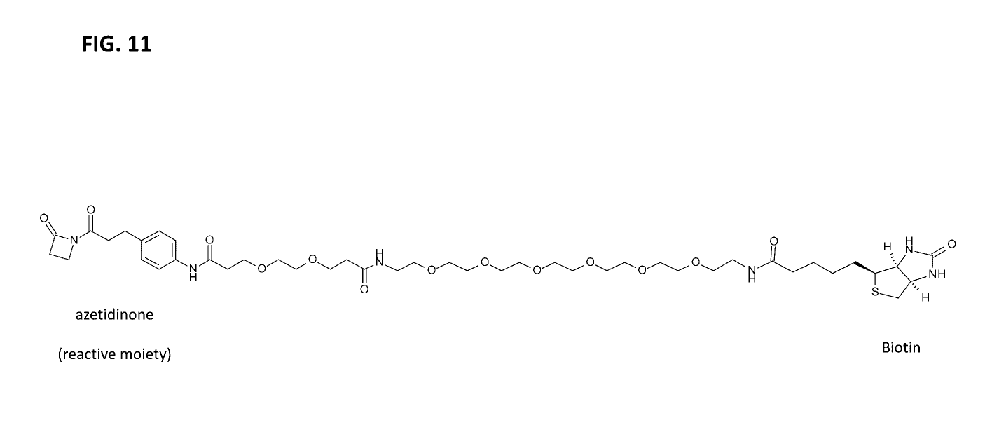

The PUCR comprising 38C2 scFab expressed on the membrane of the NKL cells was conjugated (i.e., programmed) with the specificity agent AZD-PEG8-biotin (see FIG. 11). Wild-type NKL that did not express PUCR were used as control. AZD-PEG8-biotin comprises the reactivity moiety azetidinone which reacts with and forms a stable covalent bond with the reactive lysine of the 38C2 scFab within the PUCR. Briefly, 1×10⁵ cells were washed twice with FluoroBrite™ DMEM and incubated with either 1 μM, or 10 μM DK-PEG8-Biotin at 4° C. for 1 hour. After incubation, cells were washed 3 times with FluoroBrite™ DMEM. DTAF-conjugated streptavidin secondary in FluoroBrite™ DMEM with 1% BSA was added to the cells over a 30 min incubation period at room temperature in the dark. Then, the cells were washed 3 times prior to FACS analysis. Fluorescence was measured using a ACEA Biosciences NovoCyte flow cytometer. Data was analyzed with FlowJo software, and DTAF-conjugated streptavidin binding was quantified by increased mean fluorescence intensity. Data was plotted using GraphPad Prism software.

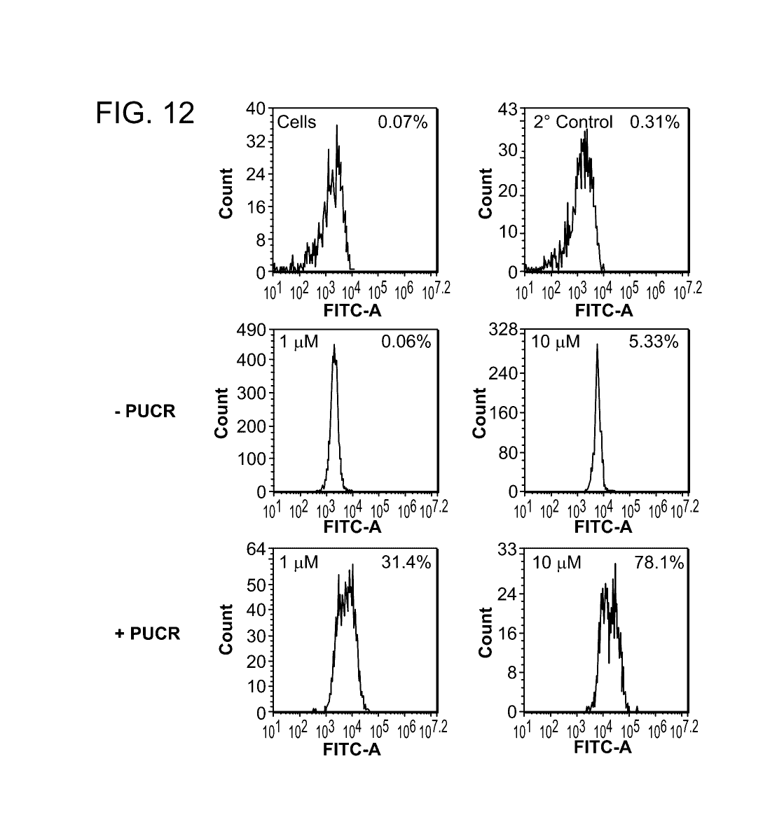

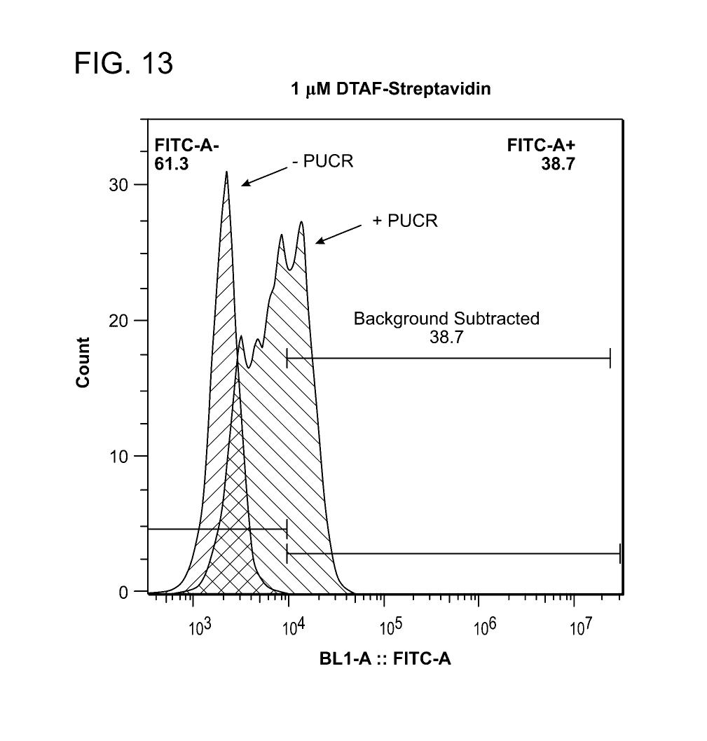

As shown in FIGS. 12 and 13, PUCR expressed in NKL cells was successfully programmed using either 1 μM or 10 μM of AZD-PEG8-Biotin. Although an increase in non-specific background staining was observed when the concentration of AZD-PEG8-Biotin was increased, specific conjugation (i.e., programming) of the expressed PUCR was observed and readily ascertained by comparing the degree of labeling in non-PUCR expressing NKL cells vs. PUCR-expressing NKL cells (see FIG. 12).

### Example 7. Conjugation of a Linker Comprising the Diketone Reactive Moiety and a Conjugation Functional Group to an Anti-PSMA Fab Fragment

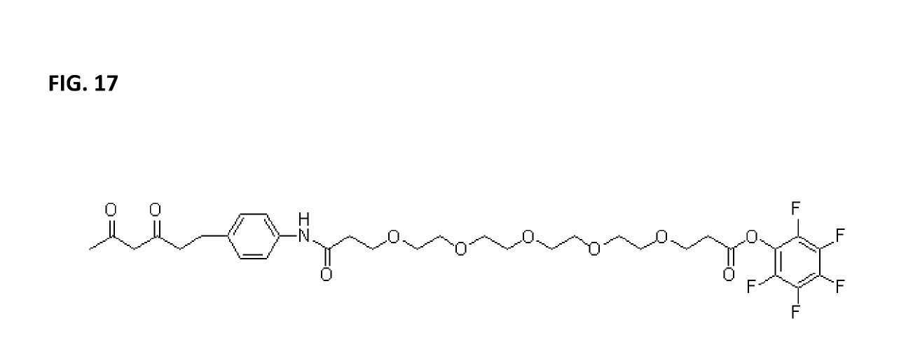

To demonstrate that Fab fragments can be conjugated with a linker comprising a reactive moiety via a conjugation functional group, which will allow for programming of a PUCR using the linker-conjugated Fab, recombinant anti-PSMA Clone A11 Fab fragment comprising a light chain variable domain amino acid sequence as set forth in SEQ ID NO: 50 and heavy chain variable domain amino acid sequence as set forth in SEQ ID NO: 49 was conjugated to the diketone-PEG5-PFP ester linker (see FIG. 17).

Diketone-PEG5-PFP (DK-PEG5-PFP) ester linker comprises the reactive moiety diketone. Briefly, recombinant anti-PSMA Clone A11 Fab fragment (5 mg/mL) was reacted with either 1.2, 2.5, 5, or 10 eq of the DK-PEG5-PFP ester linker. Conjugation reactions were performed in DPBS buffer with mixing for approximately 16-18 h at 4° C. Free linker was removed by centrifugal filtration.

To detect conjugation of the DK-PEG5-PFP ester linker to the anti-PSMA Clone A11 Fab fragment, hydrophobic interaction chromatography (HIC) HPLC was performed. Briefly, the analysis by HIC HPLC used a TOSOH TSKgel Butyl-NPR (4.6 mm ID×10 cm, 2.5 μm) column at 40° C. on an Agilent 1260 Infinity system. Analytical runs were performed using 25 μg sample using a linear gradient of 0-60% B over 30 min: A=50 mM sodium phosphate with 1 M ammonium sulfate (pH 7), B=50 mM sodium phosphate with 10% isopropanol (pH 7). A11 data was analyzed using OpenLAB Software.

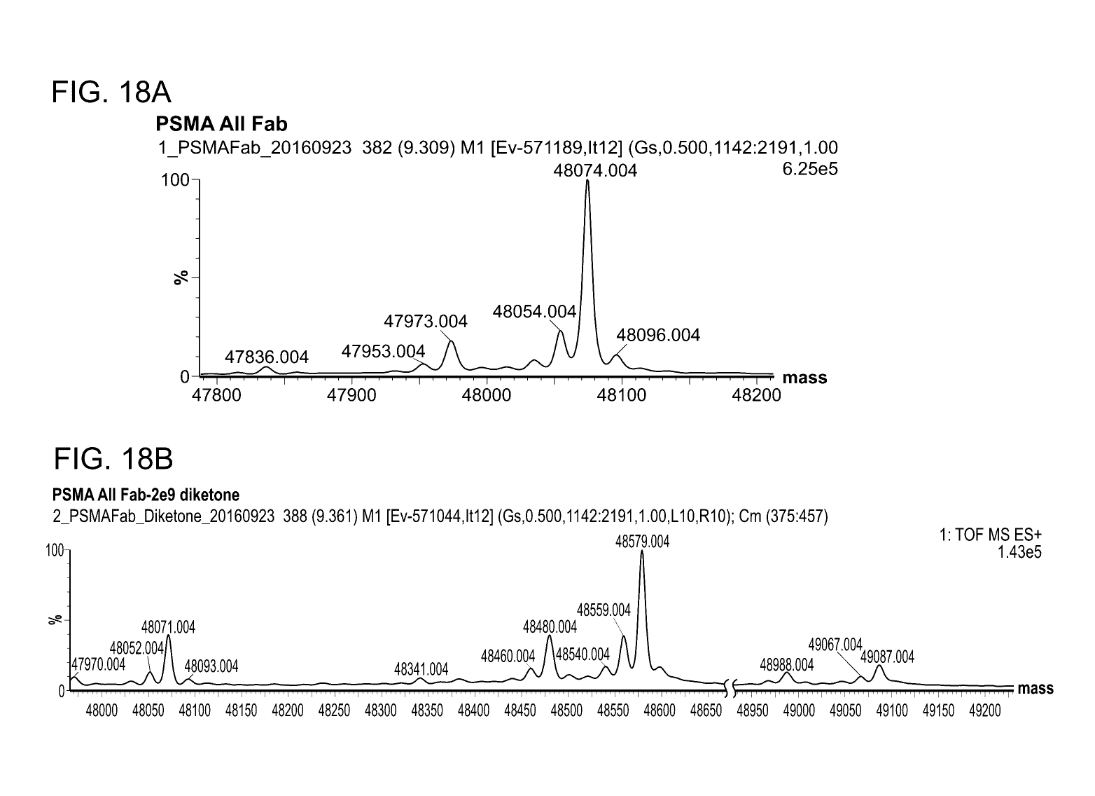

FIG. 18A shows the mass spectrum of unconjugated recombinant anti-PSMA Clone A11 Fab fragment. In contrast, FIG. 18B shows the mass spectrum of the conjugation reaction of recombinant anti-PSMA Clone A11 Fab fragment reacted with the DK-PEG5-PFP linker. A fairly homogenous peak corresponding to a single linker conjugation event was observed at a mass of about 48580. Minor peaks were also observed at a mass of about 49050 corresponding to the conjugation of two linker moieties per Fab fragment. These results demonstrate that a Fab fragment can be successfully conjugated to a linker comprising a reactive moiety via a conjugation functional group, which will allow for programming of a PUCR.

### Example 8. In Vitro Programming of Recombinant 38C2 scFv-Fc with a VEGFR2-Specific Fab Fragment Conjugated to a Linker Comprising the Reactive Moiety Azetidinone

To determine whether a molecule comprising an scFv-Fc derived from a catalytic antibody retains catalytic activity and can be successfully conjugated to a specificity agent conjugated to a liker comprising a reactive moiety, the following experiment was performed.

Conjugation of a Linker Comprising a Reactive Moiety and a Conjugation Functional Group to the Anti-VEGFR2 VK-B8 Fab Fragment.

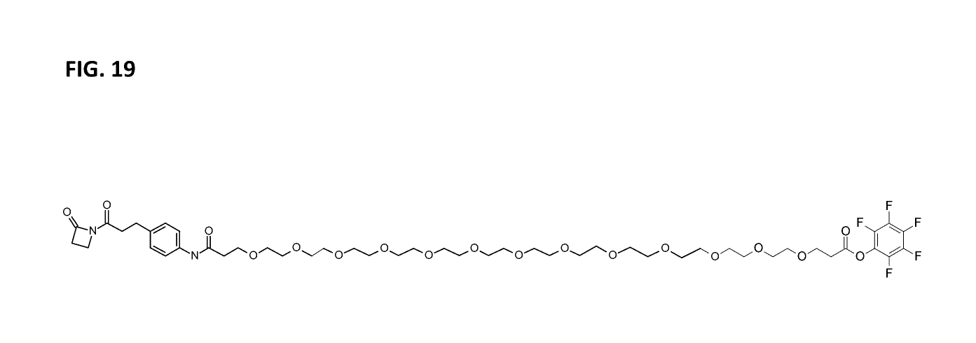

Recombinant anti-VEGFR2 VK-B8 Fab fragment comprising a light chain variable domain amino acid sequence as set forth in SEQ ID NO: 52 and a heavy chain variable domain amino acid sequence as set forth in SEQ ID NO: 53, was conjugated to the AZD-PEG13-PFP ester linker (see FIG. 19). AZD-PEG13-PFP ester comprises the reactive moiety azetidinone (AZD). AZD-PEG13-PFP ester was reacted with anti-VEGFR2 VK-B8 Fab fragment (5 mg/mL) using 2.5 eq of linker. Conjugation reactions were carried out in DPBS buffer with mixing for approximately 16-18 hours at 4° C. Free linker was removed by centrifugal filtration.

Analysis of Programming of Recombinant 38C2 scFv-Fc with Anti-VEGFR2 VKB8 Fab Fragment Conjugated to the AZD-PEG13-PFP Ester Linker.

For detection, the anti-VEGFR2 VKB8 Fab fragment conjugated to the AZD-PEG13-PFP ester linker was fluorescently labeled (“VKB8 Fab AZD 488”). As control, both anti-VEGFR2 VKB8 Fab fragment not conjugated to the AZD-PEG13-PFP ester linker that was either fluorescently labeled (“VKB8 Fab 488”) or non-fluorescently labeled (“VKB8 Fab”) were used. The Fab fragments were buffer exchanged into 100 mM sodium bicarbonate buffer, pH 8, and reacted with 10 eq AlexaFluor®488 NHS ester for 2 hours in the dark at room temperature. Fluorescently-labeled Fab fragments were then buffer exchanged to DPBS. To program the recombinant 38C2 scFv-Fc with the labeled Fab fragments, murine 38C2 scFv-Fc (“m38C2”) was incubated at 1 mg/mL with subsaturating 1.5 eq Fab fragment (i.e., 0.75 eq Fab per reactive 38C2 lysine) for 16-18 hours at room temperature to prevent overloading of SDS-PAGE gel during further analysis.

Sodium Dodecyl Sulfate Polyacrylamide Gel Electrophoresis (SDS-PAGE) Analysis.

SDS-PAGE analysis was performed using NuPAGE Novex 4-12% Bis-Tris Protein Gels and the NuPAGE MOPS SDS Running Buffer in a XCell SureLock Mini electrophoresis system. A11 samples (2.5 μg) included NuPAGE LDS Sample Buffer. Samples were heated to 95° C. for 5 min. prior to loading. PageRuler Prestained NIR protein ladder (10 μL) was used for analysis of fluorescent gel prior to SYPRO RUBY staining. Gels were fixed for 5 min. and stained with Sypro® Ruby Protein Gel Stain following the manufacturer instructions. Imaging was performed with a Bio-Rad ChemiDoc MP System and analyzed by Image Lab Software.

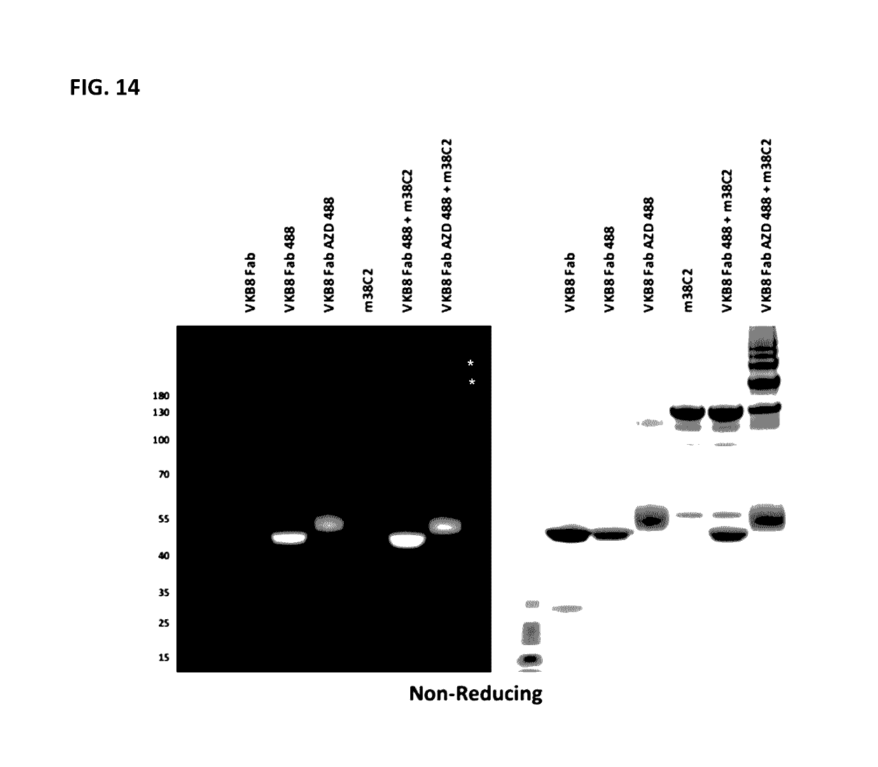

As shown in FIG. 14, recombinant murine 38C2 scFv-Fc was successfully conjugated (i.e., programmed) to the AZD-labeled VK-B8 Fab fragment as demonstrated by the detection of fluorescent high molecular weight complexes of VK-B8 Fab fragment-conjugated 38C2 scFv-Fc. Without wishing to be bound by any particularly theory, the retention of catalytic activity by the murine catalytic 38C2 scFv-Fc is particularly surprising given that scFvs lack the structural rigidity and stability which is provided by the constant regions present in full length antibodies and Fab fragments. These results are further supported by the successful conjugation of azetidinone-PEG5-methyl ester to humanized recombinant 38C2 scFv-Fc as demonstrated in Example 1 and FIG. 6. These results demonstrate that scFvs derived from catalytic antibodies retain catalytic activity, and may be successfully incorporated into a PUCR for programming with a specificity agent.

### Example 9. Generation and Characterization of KHYG-1 Natural Killer Cells Expressing PUCR Programmed with a PSMA-Targeting Specificity Agent

To determine whether PUCR expressed on the surface of KHYG-1 NK cells can be programmed to specifically bind to an antigen of interest, the following experiment was performed. KHYG-1 cells were transduced with constructs encoding a PUCR comprising 38C2 scFab (described in Example 6). A transduction efficiency of approximately 30% was achieved for this experiment.

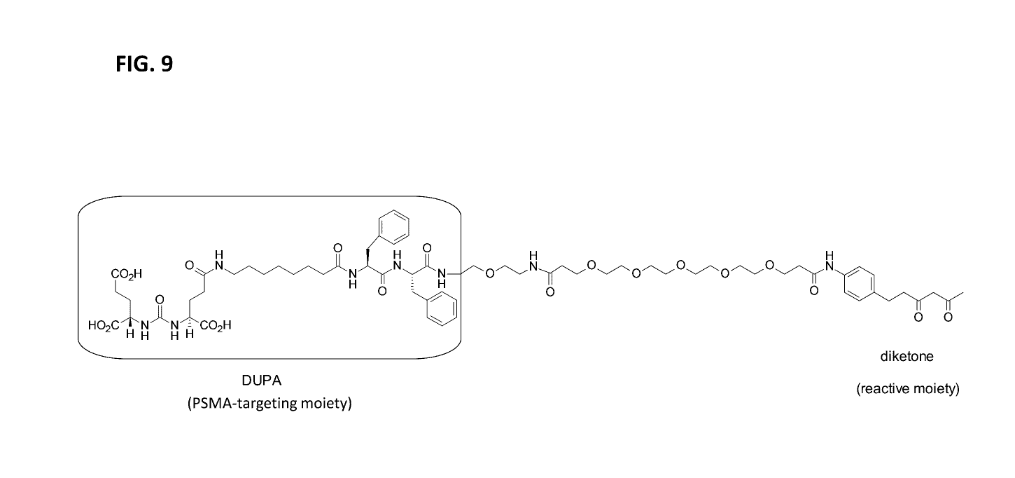

PUCRs expressed on the surface membrane of KHYG-1 NK cells were conjugated (i.e., programmed) to the specificity agent DK-PEG5-DUPA (see FIG. 9). DUPA is a targeting moiety specific for prostate specific membrane antigen (PSMA). The reactivity moiety diketone reacts with the reactive lysine of the 38C2 scFab, forming a reversible covalent bond. Wild-type KHYG-1 NK cells and NK cells transduced with a construct to express a PUCR comprising 38C2 scFab were adjusted to 0.3×10⁶ cells/mL (20×10⁶ cells total) in RPMI-1640 media with 10% heat-inactivated fetal bovine serum (FBS) and 100 IU/mL IL-2. After overnight incubation, cells were pelleted and resuspended at 3×10⁶ cells/mL in RPMI-1640 media with 10% heat-inactivated FBS. To program the PUCR expressed by the transduced KHYG-1 NK cells, the specificity agent DK-PEG5-DUPA at either 0.1 nM, 1 nM, 10 nM, and 100 nM, was prepared by performing ½ log serial dilutions with RPMI with 10% heat-inactivated FBS in 96-well deep well plates. Each concentration of DK-PEG5-DUPA was tested in triplicate using 50 μL, of the specificity agent per well in a 96-well v-bottom plate. After 1.5-2 hours incubation at 37° C. in humidified 5% CO₂ atmosphere, the KHYG-1 NK cells were pelleted and washed with RPMI with 10% heat-inactivated FBS to remove free specificity agent.

Programming of the PUCR was assessed by detecting the binding of recombinant PSMA to KHYG-1 NK cells comprising the programmed PUCR. Briefly, the treated NK cells were washed using FACS buffer (PBS, 0.5% BSA, 10% FBS, 0.05% sodium azide). Recombinant human PSMA (R&D Systems #4234-ZN) was fluorescently labeled with DyLight 488 (Abcam Cat. No. ab201799). Fluorescent-labeled PSMA protein was added to the either the wild type KHYG-1 NK cells or KHYG-1 NK cells expressing the PUCR programmed with the DK-PEG5-DUPA specificity agent, and allowed to incubate during 30-60 min at 4° C. The cells were then washed with FACS buffer. Fluorescence was measured using a ACEA Biosciences NovoCyte flow cytometer. Data was analyzed with FlowJo software, and PSMA binding to effector cells was quantified by increased mean fluorescence intensity. Data was plotted using GraphPad Prism software.

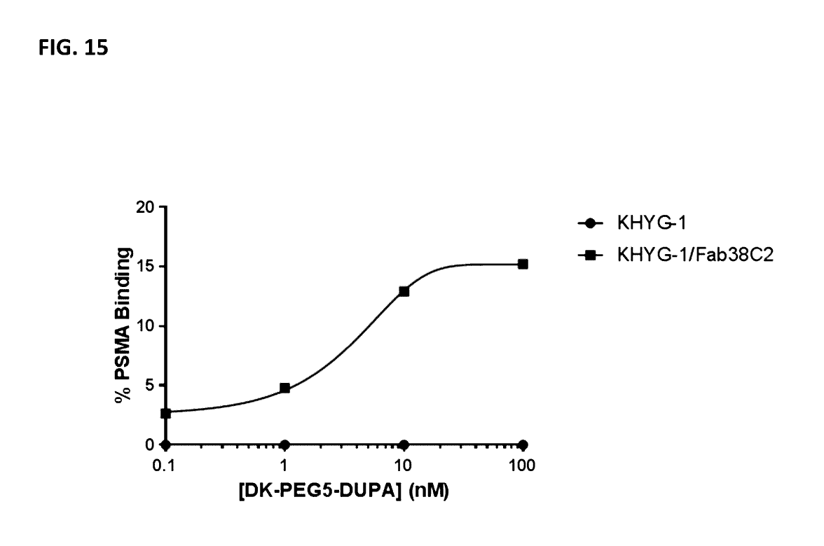

As shown in FIG. 15, cells expressing PUCR comprising 38C2 Fab fragment programmed with the DK-PEG5-DUPA specificity agent specifically bound to recombinant PSMA, demonstrating that the PUCR could be successfully programmed to target an antigen of interest (i.e., PSMA).

### Example 10. Cytotoxic Activity of KHYG-1 Natural Killer Cells Expressing PUCR Programmed with a PSMA-Targeting Specificity Agent

To determine whether KHYG-1 NK cells expressing PUCR programmed with the DK-PEG5-DUPA specificity agent are able to specifically kill PSMA-expressing cells, cytotoxicity assays were performed as described below. KHYG-1 NK cells were transduced with constructs encoding a PUCR comprising 38C2 scFab (described in Example 6). A transduction efficiency of approximately 70%-80% was achieved for this experiment.

Briefly, the ability of either wild-type KHYG-1 NK cells (control) or KHYG-1 cells expressing PUCR comprising 38C2 scFab programmed using increasing concentrations of DK-PEG5-DUPA to kill either PSMA-positive LNCaP cells (ATCC® CRL-1740™) or PSMA-negative PC-3 cells (ATCC® CRL-1435™) was determined by performing the cytotoxicity assay described below.

PUCRs expressed on the surface membrane of KHYG-1 NK cells were conjugated (i.e., programmed) to the specificity agent DK-PEG5-DUPA. Wild-type KHYG-1 NK cells and KHYG-1 NK cells transduced with a construct to express a PUCR comprising 38C2 scFab were adjusted to 0.3×10⁶ cells/mL (20×10⁶ cells total) in RPMI-1640 media with 10% heat-inactivated fetal bovine serum (FBS) and 100 IU/mL IL-2. After overnight incubation, cells were pelleted and resuspended at 3×10⁶ cells/mL in RPMI-1640 media with 10% heat-inactivated FBS. To program the PUCRs expressed by the transduced KHYG-1 NK cells, the specificity agent DK-PEG5-DUPA at either 3.2 nM, 10 nM, 32 nM, 100 nM, 320 nM, or 1000 nM was prepared by performing ½ log serial dilutions with RPMI with 10% heat-inactivated FBS in 96-well deep well plates. Each concentration of DK-PEG5-DUPA was tested in triplicate using 50 μL, of the specificity agent per well in a 96-well v-bottom plate. After 1.5-2 hours incubation at 37° C. in humidified 5% CO₂ atmosphere, the KHYG-1 NK cells were pelleted and washed with RPMI with 10% heat-inactivated FBS to remove free specificity agent and then 50 μL of KHYG-1 NK cells was added to the assay plates.

A KHYG-1 NK cell: target cell ratio of 10:1 with 10,000 target cells/well in 96-well plates was utilized for the assay. Briefly, firefly luciferase-transduced prostate cancer target cell lines LNCaP (ATCC® CRL-1740™), which are PSMA-positive (and were cultured in RPMI-1640 media having 10% non-heat-inactivated FBS and 0.5 μg/mL puromycin) or PC-3 (ATCC® CRL-1435™), which are PSMA-negative (and were cultured in RPMI-1640 media with 10% heat-inactivated FBS and 1.0 μg/mL puromycin) were used. Cells were resuspended in fresh RPMI-1640 media with 10% heat-inactivated FBS at a concentration of 0.2×10⁶ cells/mL and 50 μL of the cell suspension was added to the assay plate with gentle mixing. Target cells alone or target cells plus either wild-type KHYG-1 cells (control) or KHYG-1 NK cells expressing PUCR comprising 38C2 scFab programmed with DK-PEG5-DUPA were next incubated at 37° C. for 2 hours in a humidified 5% CO₂ incubator prior to the addition of 100 μL ONE Glo Luciferase Assay Reagent (Promega Cat. No. E6120). Samples were transferred to white 96-well flat bottom plates for luminescence measurements using a PerkinElmer EnSpire multimode plate reader. Data was analyzed using GraphPad Prism software.

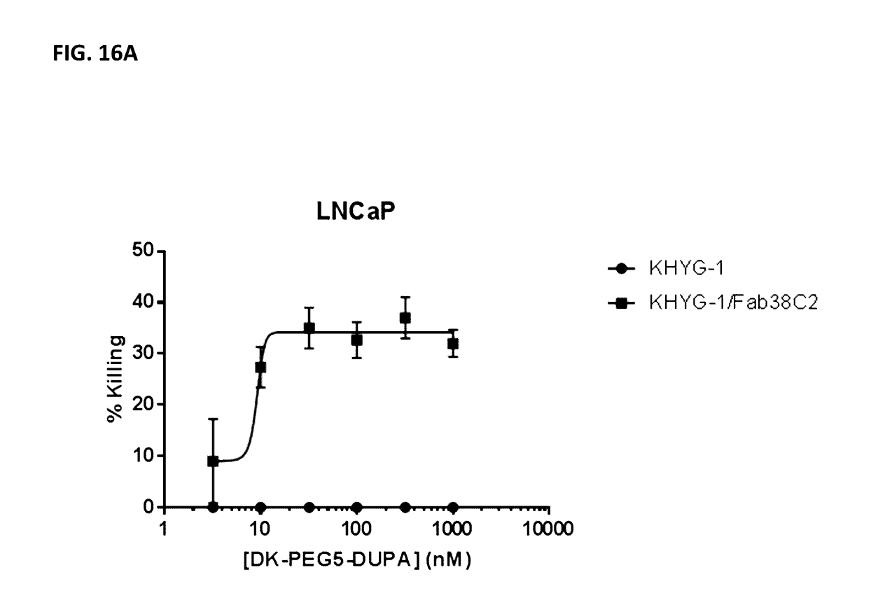

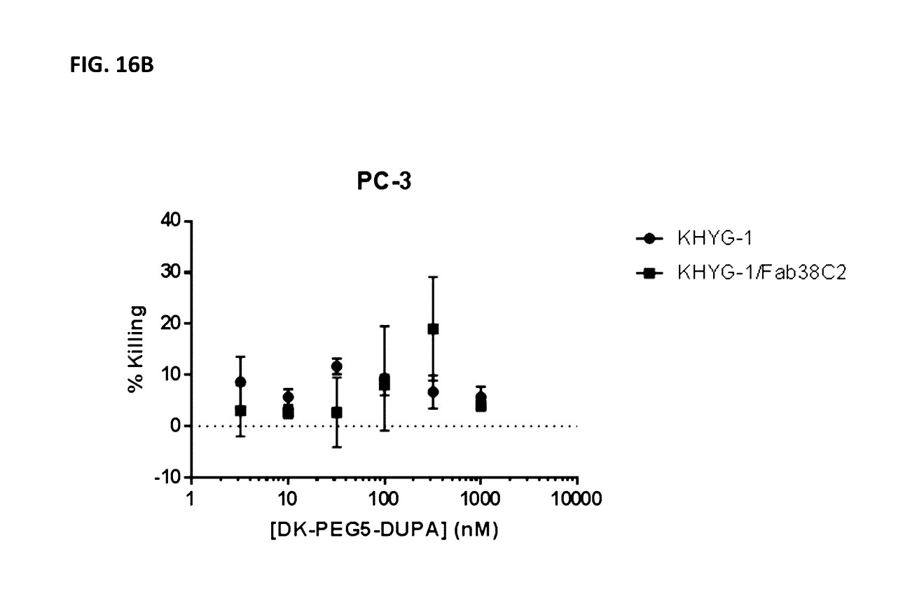

As shown in FIG. 16A, wild-type KHYG-1 NK cells were unable to kill PSMA-positive LNCaP cells. In contrast, KHYG-1 NK cells expressing PUCR programmed with DK-PEG5-DUPA specifically killed the PSMA-positive LNCaP cells. As shown in FIG. 16B, the specificity of the cytotoxicity of KHYG-1 NK cells expressing PUCR programmed with DK-PEG5-DUPA was further confirmed using PSMA-negative PC-3 cells. No significant difference was observed in the killing of PSMA-negative PC-3 cells by wild-type KHYG-1 NK cells and KHYG-1 NK cells expressing PUCR programmed with DK-PEG5-DUPA. Thus, this experiment demonstrates that NK cells expressing PUCR programmed with a PSMA-targeting specificity agent can successfully be used to specifically target and kill cells PSMA-positive cells.

TABLE 8
Sequence Summary

| SEQ | | |
|---|---|---|
| ID | | |
| NO: | Description | Sequence |
| 1 | Signal | MEWSWVFLFFLSVTTGVHS |
| | peptide | |
| | amino acid | |
| | sequence | |
| 2 | Myc-tag | EQKLISEEDL |
| | amino acid | |
| | sequence | |
| 3 | Murine | DVVMTQTPLSLPVRLGDQASISCRSSQSLLHTY |
| | 38C2 scFv | GSPYLNWYLQKPGQSPKLLIYKVSNRFSGVPDR |
| | amino acid | FSGSGSGTDFTLRISRVEAEDLGVYFCSQGTHL |
| | sequence | PYTFGGGTKLEIKGGGGSGGGGSGGGGSEVKL |
| | | VESGGGLVQPGGTMKLSCEISGLTFRNYWMS |
| | | WVRQSPEKGLEWVAEIRLRSDNYATHYAESV |
| | | KGKFTISRDDSKSRLYLQMNSLRTEDTGIYYCK |
| | | TYFYSFSYWGQGTLVTVSA |
| 4 | humanized | ELQMTQSPSSLSASVGDRVTITCRSSQSLLHTY |
| | 38C2 scFv | GSPYLNWYLQKPGQSPKLLIYKVSNRFSGVPSR |
| | amino acid | FSGSGSGTDFTLTISSLQPEDFAVYFCSQGTHLP |
| | sequence | YTFGGGTKVEIKGGGGSGGGGSGGGGSEVQLV |
| | | ESGGGLVQPGGSLRLSCAASGFTFSNYWMSWV |
| | | RQSPEKGLEWVSEIRLRSDNYATHYAESVKGR |
| | | FTISRDNSKNTLYLQMNSLRAEDTGIYYCKTYF |
| | | YSFSYWGQGTLVTVSS |
| 5 | CD8 hinge | AKPTTTPAPRPPTPAPTIASQPLSLRPEACRPAA |
| | amino acid | GGAVHTRGLDFA |
| | sequence | |
| 6 | CD3ζ | LDPKLCYLLDGILFIYGVILTALFLRVK |
| | trans- | |
| | membrane | |
| | domain | |
| | amino acid | |
| | sequence | |
| 7 | CD28 | RSKRSRLLHSDYMNMTPRRPGPTRKHYQPYAP |
| | intra- | PRDFAAYRS |
| | cellular | |
| | domain | |
| | amino acid | |
| | sequence | |
| 8 | CD3ζ | RVKFSRSADAPAYQQGQNQLYNELNLGRREEY |
| | intra- | DVLDKRRGRDPEMGGKPQRRKNPQEGLYNEL |
| | cellular | QKDKMAEAYSEIGMKGERRRGKGHDGLYQGL |
| | domain | STATKDTYDALHMQALPPR |
| | amino acid | |
| | sequence | |
| 9 | Murine | MEWSWVFLFFLSVTTGVHSDVVMTQTPLSLPV |
| | PUCR with | RLGDQASISCRSSQSLLHTYGSPYLNWYLQKPG |
| | Myc-tag | QSPKLLIYKVSNRFSGVPDRFSGSGSGTDFTLRI |
| | amino acid | SRVEAEDLGVYFCSQGTHLPYTFGGGTKLEIKG |
| | sequence | GGGSGGGGSGGGGSEVKLVESGGGLVQPGGT |
| | | MKLSCEISGLTFRNYWMSWVRQSPEKGLEWV |
| | | AEIRLRSDNYATHYAESVKGKFTISRDDSKSRL |
| | | YLQMNSLRTEDTGIYYCKTYFYSFSYWGQGTL |
| | | VTVSAEQKLISEEDLAKPTTTPAPRPPTPAPTIAS |
| | | QPLSLRPEACRPAAGGAVHTRGLDFALDPKLC |
| | | YLLDGILFIYGVILTALFLRVKRSKRSRLLHSDY |
| | | MNMTPRRPGPTRKHYQPYAPPRDFAAYRSRV |
| | | KFSRSADAPAYQQGQNQLYNELNLGRREEYD |
| | | VLDKRRGRDPEMGGKPQRRKNPQEGLYNELQ |
| | | KDKMAEAYSEIGMKGERRRGKGHDGLYQGLS |
| | | TATKDTYDALHMQALPPR |
| 10 | Humanized | MEWSWVFLFFLSVTTGVHSELQMTQSPSSLSA |
| | PUCR with | SVGDRVTITCRSSQSLLHTYGSPYLNWYLQKPG |
| | Myc-tag | QSPKLLIYKVSNRFSGVPSRFSGSGSGTDFTLTIS |
| | with signal | SLQPEDFAVYFCSQGTHLPYTFGGGTKVEIKGG |
| | peptide | GGSGGGGSGGGGSEVQLVESGGGLVQPGGSLR |
| | amino acid | LSCAASGFTFSNYWMSWVRQSPEKGLEWVSEI |
| | sequence | RLRSDNYATHYAESVKGRFTISRDNSKNTLYL |
| | | QMNSLRAEDTGIYYCKTYFYSFSYWGQGTLVT |
| | | VSSEQKLISEEDLAKPTTTPAPRPPTPAPTIASQP |
| | | LSLRPEACRPAAGGAVHTRGLDFALDPKLCYL |
| | | LDGILFIYGVILTALFLRVKRSKRSRLLHSDYMN |
| | | MTPRRPGPTRKHYQPYAPPRDFAAYRSRVKFS |
| | | RSADAPAYQQGQNQLYNELNLGRREEYDVLD |
| | | KRRGRDPEMGGKPQRRKNPQEGLYNELQKDK |
| | | MAEAYSEIGMKGERRRGKGHDGLYQGLSTAT |
| | | KDTYDALHMQALPPR |
| 11 | Signal | ATGGAGTGGTCCTGGGTGTTCCTGTTCTTTCT |
| | peptide | GTCCGTGACCACCGGTGTCCAC |
| | nucleic | |
| | acid | |
| | sequence | |
| 12 | Myc-tag | GAGCAGAAACTCATTTCTGAAGAGGACCTT |
| | nucleic | |
| | acid | |
| | sequence | |
| 13 | murine | GATGTAGTTATGACCCAGACGCCTCTTTCTCT |
| | 38C2 scFv | CCCCGTCCGGCTCGGAGACCAAGCCTCCATC |
| | nucleic | TCTTGCCGAAGTTCACAATCATTGTTGCACA |
| | acid | CGTATGGATCCCCATATCTGAATTGGTATCTC |
| | sequence | CAAAAGCCTGGACAGTCCCCCAAGCTGTTGA |
| | | TCTATAAAGTAAGTAATAGATTTTCCGGCGT |
| | | TCCTGACCGCTTCAGTGGCTCAGGAAGCGGT |
| | | ACGGATTTTACTCTTCGGATTTCCCGCGTCGA |
| | | AGCTGAAGATCTTGGTGTCTATTTCTGTTCTC |
| | | AGGGAACGCACCTGCCATACACATTCGGAGG |
| | | GGGCACTAAGCTCGAAATCAAGGGCGGGGG |
| | | CGGGTCAGGTGGTGGGGGCAGCGGCGGGGG |
| | | TGGCAGCGAGGTTAAGCTTGTGGAAAGTGGA |
| | | GGCGGGCTTGTGCAGCCGGGCGGGACCATG |
| | | AAACTGTCCTGCGAGATAAGTGGACTCACTT |
| | | TTAGGAACTATTGGATGAGCTGGGTGCGACA |
| | | GTCCCCCGAGAAGGGCCTTGAATGGGTTGCC |
| | | GAAATACGGCTTCGATCAGACAACTATGCGA |
| | | CGCACTACGCTGAAAGCGTCAAAGGAAAATT |
| | | CACTATCAGCCGGGACGACAGCAAGAGTAG |
| | | ACTTTATTTGCAGATGAATAGTTTGAGGACG |
| | | GAAGATACGGGAATATATTATTGCAAAACAT |
| | | ACTTCTATTCATTTTCATACTGGGGTCAGGGC |
| | | ACGTTGGTTACGGTTTCAGCC |
| 14 | humanized | GAGCTTCAGATGACCCAAAGTCCCAGCTCTC |
| | 38C2 scFv | TCTCCGCCTCTGTCGGAGACAGGGTCACGAT |
| | nucleic | AACCTGTCGAAGTAGCCAGAGTCTTCTCCAT |
| | acid | ACTTACGGAAGCCCATATCTTAACTGGTATC |
| | sequence | TTCAGAAACCCGGTCAATCACCCAAGCTGCT |
| | | GATATATAAAGTGTCTAACCGGTTTTCTGGT |
| | | GTGCCGAGTCGATTTTCAGGATCAGGGAGCG |
| | | GCACGGATTTCACTCTTACGATCTCTAGTTTG |
| | | CAACCTGAGGATTTTGCTGTATACTTTTGCAG |
| | | CCAAGGTACTCATCTTCCTTATACGTTCGGA |
| | | GGGGGTACCAAAGTAGAAATTAAAGGAGGA |
| | | GGAGGGTCCGGAGGAGGGGGCAGCGGAGGA |
| | | GGAGGCTCAGAAGTACAACTCGTGGAATCTG |
| | | GCGGGGGGCTGGTGCAACCTGGGGGTTCTCT |
| | | CCGCCTGAGCTGTGCTGCATCCGGCTTCACC |
| | | TTTTCTAATTATTGGATGAGCTGGGTACGGC |
| | | AGTCACCGGAGAAAGGTCTGGAGTGGGTGTC |
| | | TGAGATACGACTTAGATCAGACAACTACGCG |
| | | ACGCATTACGCCGAGAGCGTGAAAGGAAGA |
| | | TTTACCATAAGCAGAGACAATTCAAAAAACA |
| | | CCCTGTACCTCCAAATGAATAGCCTCAGGGC |
| | | GGAAGATACTGGGATATATTACTGTAAAACC |
| | | TACTTTTACAGTTTTAGTTATTGGGGCCAGGG |
| | | AACGCTTGTAACTGTTAGCTCT |
| 15 | CD8 | GCTAAGCCCACCACGACGCCAGCGCCGCGA |
| | hinge | CCACCAACACCGGCGCCCACCATCGCGTCGC |
| | nucleic | AGCCCCTGTCCCTGCGCCCAGAGGCGTGCCG |
| | acid | GCCAGCGGCGGGGGGCGCAGTGCACACGAG |
| | sequence | GGGGCTGGACTTCGCC |
| 16 | CD3ζ | CTCGATCCGAAGTTGTGCTACCTGTTGGACG |
| | trans- | GCATTCTCTTTATATACGGTGTCATCCTGACA |
| | membrane | GCGTTGTTTCTCCGAGTGAAG |
| | domain | |
| | nucleic | |
| | acid | |
| | sequence | |
| 17 | CD28 in- | AGGAGTAAGAGGAGCAGGCTCCTGCACAGT |
| | tracellular | GACTACATGAACATGACTCCCCGCCGCCCCG |
| | domain | GGCCCACCCGCAAGCATTACCAGCCCTATGC |
| | nucleic | CCCACCACGCGACTTCGCAGCCTATCGCTCC |
| | acid | |
| | sequence | |
| 18 | CD3ζ in- | AGAGTGAAGTTCAGCAGGAGCGCAGACGCC |
| | tracellular | CCCGCGTACCAGCAGGGCCAGAACCAGCTCT |
| | domain | ATAACGAGCTCAATCTAGGACGAAGAGAGG |
| | nucleic | AGTACGATGTTTTGGACAAGAGACGTGGCCG |
| | acid | GGACCCTGAGATGGGGGGAAAGCCGAGAAG |
| | sequence | GAAGAACCCTCAGGAAGGCCTGTACAATGA |
| | | ACTGCAGAAAGATAAGATGGCGGAGGCCTA |
| | | CAGTGAGATTGGGATGAAAGGCGAGCGCCG |
| | | GAGGGGCAAGGGGCACGATGGCCTTTACCA |
| | | GGGTCTCAGTACAGCCACCAAGGACACCTAC |
| | | GACGCCCTTCACATGCAGGCCCTGCCCCCTC |
| | | GCTAA |
| 19 | Murine | GATGTAGTTATGACCCAGACGCCTCTTTCTCT |
| | PUCR with | CCCCGTCCGGCTCGGAGACCAAGCCTCCATC |
| | Myc-tag | TCTTGCCGAAGTTCACAATCATTGTTGCACA |
| | nucleic | CGTATGGATCCCCATATCTGAATTGGTATCTC |
| | acid | CAAAAGCCTGGACAGTCCCCCAAGCTGTTGA |
| | sequence | TCTATAAAGTAAGTAATAGATTTTCCGGCGT |
| | | TCCTGACCGCTTCAGTGGCTCAGGAAGCGGT |
| | | ACGGATTTTACTCTTCGGATTTCCCGCGTCGA |
| | | AGCTGAAGATCTTGGTGTCTATTTCTGTTCTC |
| | | AGGGAACGCACCTGCCATACACATTCGGAGG |
| | | GGGCACTAAGCTCGAAATCAAGGGCGGGGG |
| | | CGGGTCAGGTGGTGGGGGCAGCGGCGGGGG |
| | | TGGCAGCGAGGTTAAGCTTGTGGAAAGTGGA |
| | | GGCGGGCTTGTGCAGCCGGGCGGGACCATG |
| | | AAACTGTCCTGCGAGATAAGTGGACTCACTT |
| | | TTAGGAACTATTGGATGAGCTGGGTGCGACA |
| | | GTCCCCCGAGAAGGGCCTTGAATGGGTTGCC |
| | | GAAATACGGCTTCGATCAGACAACTATGCGA |
| | | CGCACTACGCTGAAAGCGTCAAAGGAAAATT |
| | | CACTATCAGCCGGGACGACAGCAAGAGTAG |
| | | ACTTTATTTGCAGATGAATAGTTTGAGGACG |
| | | GAAGATACGGGAATATATTATTGCAAAACAT |
| | | ACTTCTATTCATTTTCATACTGGGGTCAGGGC |
| | | ACGTTGGTTACGGTTTCAGCCGAGCAGAAGC |
| | | TCATTTCCGAAGAAGATCTCGCAAAGCCGAC |
| | | AACGACGCCGGCACCCCGGCCTCCCACCCCC |
| | | GCCCCCACTATAGCTAGTCAACCTCTTTCACT |
| | | GCGCCCTGAAGCGTGTAGACCTGCAGCCGGG |
| | | GGAGCAGTCCATACGCGCGGACTTGATTTCG |
| | | CCCTCGACCCCAAGTTGTGTTACCTTTTGGAC |
| | | GGGATCCTCTTCATTTACGGTGTCATTCTTAC |
| | | TGCCTTGTTTCTCAGGGTAAAAAGGTCTAAG |
| | | AGATCCCGACTCCTCCATTCTGACTACATGA |
| | | ATATGACACCGAGGAGACCGGGACCAACTC |
| | | GGAAGCATTATCAGCCATACGCGCCCCCCCG |
| | | CGATTTCGCGGCATACAGGTCAAGAGTCAAG |
| | | TTCTCCCGCAGCGCAGACGCGCCCGCTTATC |
| | | AGCAAGGTCAAAATCAACTCTACAATGAGCT |
| | | CAATCTGGGACGACGGGAGGAGTACGATGT |
| | | CCTCGACAAGAGGAGAGGTCGGGATCCTGA |
| | | AATGGGTGGCAAACCCCAGCGACGCAAGAA |
| | | TCCTCAGGAGGGTCTCTACAACGAGCTGCAA |
| | | AAAGATAAAATGGCGGAGGCGTATAGTGAA |
| | | ATAGGGATGAAAGGGGAAAGACGCCGGGGA |
| | | AAAGGACATGATGGTCTGTATCAGGGTCTGT |
| | | CAACAGCTACTAAAGACACATACGATGCGCT |
| | | GCACATGCAAGCGTTGCCGCCGAGG |
| 20 | Humanized | GAGCTTCAGATGACCCAAAGTCCCAGCTCTC |
| | PUCR with | TCTCCGCCTCTGTCGGAGACAGGGTCACGAT |
| | Myc-tag | AACCTGTCGAAGTAGCCAGAGTCTTCTCCAT |
| | nucleic | ACTTACGGAAGCCCATATCTTAACTGGTATC |
| | acid | TTCAGAAACCCGGTCAATCACCCAAGCTGCT |
| | sequence | GATATATAAAGTGTCTAACCGGTTTTCTGGT |
| | | GTGCCGAGTCGATTTTCAGGATCAGGGAGCG |
| | | GCACGGATTTCACTCTTACGATCTCTAGTTTG |
| | | CAACCTGAGGATTTTGCTGTATACTTTTGCAG |
| | | CCAAGGTACTCATCTTCCTTATACGTTCGGA |
| | | GGGGGTACCAAAGTAGAAATTAAAGGAGGA |
| | | GGAGGGTCCGGAGGAGGGGGCAGCGGAGGA |
| | | GGAGGCTCAGAAGTACAACTCGTGGAATCTG |
| | | GCGGGGGGCTGGTGCAACCTGGGGGTTCTCT |
| | | CCGCCTGAGCTGTGCTGCATCCGGCTTCACC |
| | | TTTTCTAATTATTGGATGAGCTGGGTACGGC |
| | | AGTCACCGGAGAAAGGTCTGGAGTGGGTGTC |
| | | TGAGATACGACTTAGATCAGACAACTACGCG |
| | | ACGCATTACGCCGAGAGCGTGAAAGGAAGA |
| | | TTTACCATAAGCAGAGACAATTCAAAAAACA |
| | | CCCTGTACCTCCAAATGAATAGCCTCAGGGC |
| | | GGAAGATACTGGGATATATTACTGTAAAACC |
| | | TACTTTTACAGTTTTAGTTATTGGGGCCAGGG |
| | | AACGCTTGTAACTGTTAGCTCTGAGCAGAAG |
| | | CTCATTTCCGAAGAAGATCTCGCAAAGCCGA |
| | | CAACGACGCCGGCACCCCGGCCTCCCACCCC |
| | | CGCCCCCACTATAGCTAGTCAACCTCTTTCA |
| | | CTGCGCCCTGAAGCGTGTAGACCTGCAGCCG |
| | | GGGGAGCAGTCCATACGCGCGGACTTGATTT |
| | | CGCCCTCGACCCCAAGTTGTGTTACCTTTTGG |
| | | ACGGGATCCTCTTCATTTACGGTGTCATTCTT |
| | | ACTGCCTTGTTTCTCAGGGTAAAAAGGTCTA |
| | | AGAGATCCCGACTCCTCCATTCTGACTACAT |
| | | GAATATGACACCGAGGAGACCGGGACCAAC |
| | | TCGGAAGCATTATCAGCCATACGCGCCCCCC |
| | | CGCGATTTCGCGGCATACAGGTCAAGAGTCA |
| | | AGTTCTCCCGCAGCGCAGACGCGCCCGCTTA |
| | | TCAGCAAGGTCAAAATCAACTCTACAATGAG |
| | | CTCAATCTGGGACGACGGGAGGAGTACGAT |
| | | GTCCTCGACAAGAGGAGAGGTCGGGATCCTG |
| | | AAATGGGTGGCAAACCCCAGCGACGCAAGA |
| | | ATCCTCAGGAGGGTCTCTACAACGAGCTGCA |
| | | AAAAGATAAAATGGCGGAGGCGTATAGTGA |
| | | AATAGGGATGAAAGGGGAAAGACGCCGGGG |
| | | AAAAGGACATGATGGTCTGTATCAGGGTCTG |
| | | TCAACAGCTACTAAAGACACATACGATGCGC |
| | | TGCACATGCAAGCGTTGCCGCCGAGG |
| 21 | linker | (Gly4Ser)n, where n is a |
| | | positive integer equal to or |
| | | greater than 1 (SEQ ID NO: 21) |
| 22 | linker | (Gly₄Ser)₄ |
| 23 | linker | (Gly₄Ser)₃ |
| 24 | trans- | FWVLVVVGGVLACYSLLVTVAFIIFWV |
| | membrane | |
| | domain | |
| | of human | |
| | CD28 | |
| 25 | trans- | IEVMYPPPYLDNEKSNGTIIHVKGKHLCPSPLFP |
| | membrane | GPSKPFWVL VVVGGVLACYSLLVTVAFIIFWV |
| | domain | |
| | of human | |
| | CD28 | |
| 26 | 4-1BB in- | KRGRKKLLY FKQPFMRPVQ |
| | tracellular | TTQEEDGCSCRFPEEEEGGCEL |
| | domain | |
| 27 | 4-1BB in- | AAACGGGGCAGAAAGAAACTCCTGTATATAT |
| | tracellular | TCAAACAACCATTTATGAGACCAGTACAAAC |
| | domain | TACTCAAGAGGAAGATGGCTGTAGCTGCCGA |
| | nucleic | TTTCCAGAAGAAGAAGAAGGAGGATGTGAA |
| | acid | CTG |
| | sequence | |
| 28 | CD8 hinge | AKPTTTPAPRPPTPAPTIASQPLSLRPEAXRPAA |
| | amino acid | GGAVHTRGLDFA where X is any amino |
| | sequence | acid except cysteine |
| 29 | CD8 hinge | TTTPAPRPPTPAPTIASQPLSLRPEACRPAAGGA |
| | | VHTRGLDFACD |
| 30 | CD8 hinge | ACCACGACGCCAGCGCCGCGACCACCAACA |
| | nucleic | CCGGCGCCCACCATCGCGTCGCAGCCCCTGT |
| | acid | CCCTGCGCCCAGAGGCGTGCCGGCCAGCGGC |
| | sequence | GGGGGGCGCAGTGCACACGAGGGGGCTGGA |
| | | CTTCGCCTGTGAT |
| 31 | Linker | (Gly₄Ser)₆ |
| 32 | Linker | (Gly₄Ser)₉ |
| 33 | Linker | (Gly₄Ser)₁₂ |
| 34 | Linker | (Gly₄Ser)₁₅ |
| 35 | Linker | (G1y₄Ser)₃₀ |
| 36 | Linker | (G1y₄Ser)₄₅ |
| 37 | Linker | (G1y₄Ser)₆₀ |
| 38 | hydrophobic | LCYLLDGILFIYGVILTALFL |
| | stretch | |
| | of the CD3ζ | |
| | trans- | |
| | membrane | |
| | domain | |
| | sequence | |
| 39 | Myc-tag | GAGCAGAAGCTGATTAGCGAAGAGGACCTG |
| | nucleic | |
| | acid | |
| | sequence | |
| 40 | Humanized | ELQMTQSPSSLSASVGDRVTITCRSSQSLLHTY |
| | 38C2 | GSPYLNWYLQKPGQSPKLLIYKVSNRFSGVPSR |
| | variable | FSGSGSGTDFTLTISSLQPEDFAVYFCSQGTHLP |
| | kappa | YTFGGGTKVEIK |
| | heavy | |
| | chain | |
| 41 | Human | RTVAAPSVFIFPPSDEQLKSGTASVVCLLNNFYP |
| | kappa | REAKVQWKVDNALQSGNSQESVTEQDSKDST |
| | light | YSLSSTLTLSKADYEKHKVYACEVTHQGLSSPV |
| | chain | TKSFNRGEC |
| | constant | |
| 42 | Humanized | EVQLVESGGGLVQPGGSLRLSCAASGFTFSNY |
| | 38C2 | WMSWVRQSPEKGLEWVSEIRLRSDNYATHYA |
| | variable | ESVKGRFTISRDNSKNTLYLQMNSLRAEDTGIY |
| | heavy | YCKTYFYSFSYWGQGTLVTVSS |
| | chain | |
| 43 | Human | ASTKGPSVFPLAPSSKSTSGGTAALGCLVKDYF |
| | gamma 1 | PEPVTVSWNSGALTSGVHTFPAVLQSSGLYSLS |
| | heavy | SVVTVPSSSLGTQTYICNVNHKPSNTKVDKKVE |
| | chain | PKSCDKTHT |
| | constant | |
| | domain 1 | |
| 44 | Full | MEWSWVFLFFLSVTTGVHSELQMTQSPSSLSA |
| | length | SVGDRVTITCRSSQSLLHTYGSPYLNWYLQKPG |
| | humanized | QSPKLLIYKVSNRFSGVPSRFSGSGSGTDFTLTIS |
| | 38C2 scFab | SLQPEDFAVYFCSQGTHLPYTFGGGTKVEIKRT |
| | with | VAAPSVFIFPPSDEQLKSGTASVVCLLNNFYPRE |
| | signal | AKVQWKVDNALQSGNSQESVTEQDSKDSTYS |
| | peptide | LSSTLTLSKADYEKHKVYACEVTHQGLSSPVT |
| | | KSFNRGECGGGSGGGGSGGGSGGGGSGGGSG |
| | | GGGSGGGGSGGGSGGGGSEVQLVESGGGLVQ |
| | | PGGSLRLSCAASGFTFSNYWMSWVRQSPEKGL |
| | | EWVSEIRLRSDNYATHYAESVKGRFTISRDNSK |
| | | NTLYLQMNSLRAEDTGIYYCKTYFYSFSYWGQ |
| | | GTLVTVSSASTKGPSVFPLAPSSKSTSGGTAAL |
| | | GCLVKDYFPEPVTVSWNSGALTSGVHTFPAVL |
| | | QSSGLYSLSSVVTVPSSSLGTQTYICNVNHKPSN |
| | | TKVDKKVEPKSCDKTHT |
| 45 | Full | MEWSWVFLFFLSVTTGVHSELQMTQSPSSLSA |
| | length | SVGDRVTITCRSSQSLLHTYGSPYLNWYLQKPG |
| | PUCR com- | QSPKLLIYKVSNRFSGVPSRFSGSGSGTDFTLTIS |
| | prising | SLQPEDFAVYFCSQGTHLPYTFGGGTKVEIKRT |
| | 38C2 scFab | VAAPSVFIFPPSDEQLKSGTASVVCLLNNFYPRE |
| | amino acid | AKVQWKVDNALQSGNSQESVTEQDSKDSTYS |
| | sequence | LSSTLTLSKADYEKHKVYACEVTHQGLSSPVT |
| | | KSFNRGECGGGSGGGGSGGGSGGGGSGGGSG |
| | | GGGSGGGGSGGGSGGGGSEVQLVESGGGLVQ |
| | | PGGSLRLSCAASGFTFSNYWMSWVRQSPEKGL |
| | | EWVSEIRLRSDNYATHYAESVKGRFTISRDNSK |
| | | NTLYLQMNSLRAEDTGIYYCKTYFYSFSYWGQ |
| | | GTLVTVSSASTKGPSVFPLAPSSKSTSGGTAAL |
| | | GCLVKDYFPEPVTVSWNSGALTSGVHTFPAVL |
| | | QSSGLYSLSSVVTVPSSSLGTQTYICNVNHKPSN |
| | | TKVDKKVEPKSCDKTHTAKPTTTPAPRPPTPAP |
| | | TIASQPLSLRPEACRPAAGGAVHTRGLDFAPRK |
| | | IEVMYPPPYLDNEKSNGTIIHVKGKHLCPSPLFP |
| | | GPSKPFWVLVVVGGVLACYSLLVTVAFIIFWV |
| | | RSKRSRLLHSDYMNMTPRRPGPTRKHYQPYAP |
| | | PRDFAAYRSRVKFSRSADAPAYQQGQNQLYNE |
| | | LNLGRREEYDVLDKRRGRDPEMGGKPRRKNP |
| | | QEGLYNELQKDKMAEAYSEIGMKGERRRGKG |
| | | HDGLYQGLSTATKDTYDALHMQALPPR |
| 46 | Signal | ATGGAATGGAGTTGGGTGTTCCTTTTCTTTCT |
| | peptide | GAGTGTCACCACCGGAGTGCAC |
| | nucleic | |
| | acid | |
| | sequence | |
| 47 | humanized | AGCGAACTGCAGATGACCCAGTCCCCATCCA |
| | 38C2 scFab | GTCTGAGCGCTAGCGTTGGTGACAGAGTTAC |
| | nucleic | TATCACCTGCCGCTCTTCACAGAGCCTGTTG |
| | acid | CACACTTACGGCTCTCCTTACCTGAACTGGT |
| | sequence | ATCTTCAGAAGCCTGGCCAAAGCCCTAAGCT |
| | | GCTCATCTACAAGGTGTCTAACAGGTTCTCC |
| | | GGGGTTCCGTCCCGCTTTTCAGGGAGCGGGT |
| | | CAGGAACAGACTTCACCTTGACAATCTCAAG |
| | | CCTCCAGCCCGAGGATTTTGCCGTCTATTTCT |
| | | GCTCACAAGGCACACATCTGCCGTATACCTT |
| | | TGGGGGCGGGACAAAAGTCGAGATCAAAAG |
| | | GACCGTCGCTGCACCATCCGTGTTTATCTTCC |
| | | CACCAAGTGACGAACAGCTCAAGAGCGGTA |
| | | CTGCCTCCGTTGTTTGTCTGCTGAACAACTTC |
| | | TATCCAAGGGAAGCAAAGGTGCAATGGAAA |
| | | GTAGACAACGCTCTGCAGTCAGGCAACTCCC |
| | | AGGAGTCAGTGACCGAGCAGGATAGCAAAG |
| | | ATTCAACATACAGCCTGAGCAGCACCCTCAC |
| | | CCTGAGTAAGGCCGATTACGAGAAGCACAA |
| | | GGTTTACGCCTGCGAGGTGACCCACCAGGGC |
| | | CTTTCATCCCCAGTCACCAAATCTTTTAACCG |
| | | CGGCGAATGCGGGGGAGGCTCTGGTGGAGG |
| | | CGGTTCTGGAGGGGGCTCAGGAGGAGGCGG |
| | | TAGCGGCGGTGGTAGTGGGGGTGGCGGATCT |
| | | GGCGGAGGTGGCTCAGGAGGAGGTAGCGGC |
| | | GGCGGGGGCAGCGAGGTCCAGCTGGTAGAG |
| | | TCAGGTGGAGGATTGGTGCAGCCCGGCGGCA |
| | | GTCTTAGACTCAGCTGTGCGGCCAGCGGATT |
| | | TACTTTCTCAAATTATTGGATGTCTTGGGTCA |
| | | GGCAGAGCCCAGAGAAAGGCCTGGAATGGG |
| | | TGTCAGAGATCCGACTGAGAAGCGATAATTA |
| | | CGCGACTCATTATGCGGAAAGCGTTAAAGGT |
| | | CGGTTCACTATTTCACGAGATAATTCTAAGA |
| | | ATACCCTTTATCTGCAGATGAACAGCTTGCG |
| | | CGCCGAGGACACAGGCATCTACTACTGTAAA |
| | | ACTTACTTCTATTCTTTTTCCTACTGGGGACA |
| | | GGGGACTCTCGTTACAGTCAGTAGCGCCTCC |
| | | ACCAAGGGTCCTAGTGTCTTTCCCCTGGCCC |
| | | CCTCATCCAAGTCCACGTCAGGAGGCACCGC |
| | | GGCTCTGGGCTGTCTGGTCAAAGACTACTTT |
| | | CCTGAGCCAGTCACCGTGTCCTGGAATTCCG |
| | | GCGCGCTTACTTCTGGCGTGCACACTTTCCCC |
| | | GCCGTCCTCCAGAGCAGTGGGCTGTATTCCC |
| | | TGTCTTCCGTAGTCACTGTGCCAAGCTCCAGT |
| | | CTGGGAACCCAGACCTATATTTGTAATGTGA |
| | | ATCATAAGCCGAGCAACACCAAGGTGGACA |
| | | AGAAGGTGGAACCGAAGTCATGTGACAAAA |
| | | CCCACACT |
| 48 | Full | ATGGAATGGAGTTGGGTGTTCCTTTTCTTTCT |
| | length | GAGTGTCACCACCGGAGTGCACAGCGAACTG |
| | PUCR com- | CAGATGACCCAGTCCCCATCCAGTCTGAGCG |
| | prising | CTAGCGTTGGTGACAGAGTTACTATCACCTG |
| | 38C2 scFab | CCGCTCTTCACAGAGCCTGTTGCACACTTAC |
| | nucleic | GGCTCTCCTTACCTGAACTGGTATCTTCAGA |
| | acid | AGCCTGGCCAAAGCCCTAAGCTGCTCATCTA |
| | sequence | CAAGGTGTCTAACAGGTTCTCCGGGGTTCCG |
| | | TCCCGCTTTTCAGGGAGCGGGTCAGGAACAG |
| | | ACTTCACCTTGACAATCTCAAGCCTCCAGCC |
| | | CGAGGATTTTGCCGTCTATTTCTGCTCACAAG |
| | | GCACACATCTGCCGTATACCTTTGGGGGCGG |
| | | GACAAAAGTCGAGATCAAAAGGACCGTCGC |
| | | TGCACCATCCGTGTTTATCTTCCCACCAAGTG |
| | | ACGAACAGCTCAAGAGCGGTACTGCCTCCGT |
| | | TGTTTGTCTGCTGAACAACTTCTATCCAAGG |
| | | GAAGCAAAGGTGCAATGGAAAGTAGACAAC |
| | | GCTCTGCAGTCAGGCAACTCCCAGGAGTCAG |
| | | TGACCGAGCAGGATAGCAAAGATTCAACAT |
| | | ACAGCCTGAGCAGCACCCTCACCCTGAGTAA |
| | | GGCCGATTACGAGAAGCACAAGGTTTACGCC |
| | | TGCGAGGTGACCCACCAGGGCCTTTCATCCC |
| | | CAGTCACCAAATCTTTTAACCGCGGCGAATG |
| | | CGGGGGAGGCTCTGGTGGAGGCGGTTCTGGA |
| | | GGGGGCTCAGGAGGAGGCGGTAGCGGCGGT |
| | | GGTAGTGGGGGTGGCGGATCTGGCGGAGGT |
| | | GGCTCAGGAGGAGGTAGCGGCGGCGGGGGC |
| | | AGCGAGGTCCAGCTGGTAGAGTCAGGTGGA |
| | | GGATTGGTGCAGCCCGGCGGCAGTCTTAGAC |
| | | TCAGCTGTGCGGCCAGCGGATTTACTTTCTC |
| | | AAATTATTGGATGTCTTGGGTCAGGCAGAGC |
| | | CCAGAGAAAGGCCTGGAATGGGTGTCAGAG |
| | | ATCCGACTGAGAAGCGATAATTACGCGACTC |
| | | ATTATGCGGAAAGCGTTAAAGGTCGGTTCAC |
| | | TATTTCACGAGATAATTCTAAGAATACCCTTT |
| | | ATCTGCAGATGAACAGCTTGCGCGCCGAGGA |
| | | CACAGGCATCTACTACTGTAAAACTTACTTC |
| | | TATTCTTTTTCCTACTGGGGACAGGGGACTCT |
| | | CGTTACAGTCAGTAGCGCCTCCACCAAGGGT |
| | | CCTAGTGTCTTTCCCCTGGCCCCCTCATCCAA |
| | | GTCCACGTCAGGAGGCACCGCGGCTCTGGGC |
| | | TGTCTGGTCAAAGACTACTTTCCTGAGCCAG |
| | | TCACCGTGTCCTGGAATTCCGGCGCGCTTAC |
| | | TTCTGGCGTGCACACTTTCCCCGCCGTCCTCC |
| | | AGAGCAGTGGGCTGTATTCCCTGTCTTCCGT |
| | | AGTCACTGTGCCAAGCTCCAGTCTGGGAACC |
| | | CAGACCTATATTTGTAATGTGAATCATAAGC |
| | | CGAGCAACACCAAGGTGGACAAGAAGGTGG |
| | | AACCGAAGTCATGTGACAAAACCCACACTGC |
| | | TAAGCCCACCACGACGCCAGCGCCGCGACC |
| | | ACCAACACCGGCGCCCACCATCGCGTCGCAG |
| | | CCCCTGTCCCTGCGCCCAGAGGCGTGCCGGC |
| | | CAGCGGCGGGGGGCGCAGTGCACACGAGGG |
| | | GGCTGGACTTCGCCCCTAGGAAAATTGAAGT |
| | | TATGTATCCTCCTCCTTACCTAGACAATGAG |
| | | AAGAGCAATGGAACCATTATCCATGTGAAAG |
| | | GGAAACACCTTTGTCCAAGTCCCCTATTTCC |
| | | CGGACCTTCTAAGCCCTTTTGGGTGCTGGTG |
| | | GTGGTTGGTGGAGTCCTGGCTTGCTATAGCT |
| | | TGCTAGTAACAGTGGCCTTTATTATTTTCTGG |
| | | GTGAGGAGTAAGAGGAGCAGGCTCCTGCAC |
| | | AGTGACTACATGAACATGACTCCCCGCCGCC |
| | | CCGGGCCCACCCGCAAGCATTACCAGCCCTA |
| | | TGCCCCACCACGCGACTTCGCAGCCTATCGC |
| | | TCCAGAGTGAAGTTCAGCAGGAGCGCAGAC |
| | | GCCCCCGCGTACCAGCAGGGCCAGAACCAG |
| | | CTCTATAACGAGCTCAATCTAGGACGAAGAG |
| | | AGGAGTACGATGTTTTGGACAAGAGACGTGG |
| | | CCGGGACCCTGAGATGGGGGGAAAGCCGAG |
| | | AAGGAAGAACCCTCAGGAAGGCCTGTACAA |
| | | TGAACTGCAGAAAGATAAGATGGCGGAGGC |
| | | CTACAGTGAGATTGGGATGAAAGGCGAGCG |
| | | CCGGAGGGGCAAGGGGCACGATGGCCTTTA |
| | | CCAGGGTCTCAGTACAGCCACCAAGGACACC |
| | | TACGACGCCCTTCACATGCAGGCCCTGCCCC |
| | | CTCGCTAA |
| 49 | Anti-PSMA | QVQLVQSGGGLVQPGGSLRLSCAASGFTFSSY |
| | Clone All | WMSWVRQAPGKGLEWVANIKQDGSEKYYVD |
| | Heavy | SVKGRFTISRDNAKNSLYLQMNSLRAEDTAVY |
| | Chain | YCARVWDYYYDSSGDAFDIWGQGTMVTVSS |
| | Variable | |
| | Domain | |
| 50 | Anti-PSMA | VIWMTQSPSSVSASVGDRVTITCRASQGISSWL |
| | Clone All | AWYQQKPGKAPKLLIYAASNLQSGVPSRFSGS |
| | Light | GSGTDFTLTISSLQPEDFATYYCQQANSFPLTFG |
| | Chain | GGTKVDIK |
| | Variable | |
| | Domain | |
| 51 | IL13R | MAFVCLAIGCLYTFLISTTFGCTSSSDTEIKVNP |
| | amino acid | PQDFEIVDPGYLGYLYLQWQPPLSLDHFKECTV |
| | sequence | EYELKYRNIGSETWKTIITKNLHYKDGFDLNKG |
| | | IEAKIHTLLPWQCTNGSEVQSSWAETTYWISPQ |
| | | GIPETKVQDMDCVYYNWQYLLCSWKPGIGVL |
| | | LDTNYNLFYWYEGLDHALQCVDYIKADGQNI |
| | | GCRFPYLEASDYKDFYICVNGSSENKPIRSSYFT |
| | | FQLQNIVKPLPPVYLTFTRESSCEIKLKWSIPLGP |
| | | IPARCFDYEIEIREDDTTLVTATVENETYTLKTT |
| | | NETRQLCFVVRSKVNIYCSDDGIWSEWSDKQC |
| | | WEGEDLSKKTLLRFWLPFGFILILVIFVTGLLLR |
| | | KPNTYPKMIPEFFCDT |
| 52 | Anti- | ETTLTQSPATLSVSPGERATVSCRASQSLGSNL |
| | VEGFR2 | GWFQQKPGQAPRLLIYGASTRATGIPARFSGSG |
| | VK-B8 | SGTEFTLTISSLQSEDFAVYFCQQYNDWPITFGQ |
| | Light | GTRLEIK |
| | Chain | |
| | Variable | |
| | Domain | |
| 53 | Anti- | MAQVQLVQSGAEVKKPGSSVKVSCKAYGGTF |
| | VEGFR2 | GSYGVSWVRRAPGQGLEWMGRLIPIFGTRDYA |
| | VK-B8 | QKFQGRVTLTADESTNTAYMELSSLRSEDTAV |
| | Heavy | YYCARDGDYYGSGSYYGMDVWGQGTLVTVS |
| | Chain | S |
| | Variable | |
| | Domain | |
| 54 | PolyGlySer | GGGSGGGGSGGGSGGGGSGGGSGGGGSGGGG |
| | linker | SGGGSGGGGS |
| 55 | Hybrid | AKPTTTPAPRPPTPAPTIASQPLSLRPEACRPAA |
| | CD8 and | GGAVHTRGLDFAPRKIEVMYPPPYLDNEKSNG |
| | CD28 | TIIHVKGKHLCPSPLFPGPSKP |
| | hinge | |
| | amino acid | |
| | sequence | |
| 56 | CD8 | AKPTTTPAPRPPTPAPTIASQPLSLRPEACRPAA |
| | portion | GGAVHTRGLDFA |
| | of hybrid | |
| | CD8 and | |
| | CD28 | |
| | hinge | |
| | amino acid | |
| | sequence | |
| 57 | Hinge | PR |
| | linker | |
| | amino acid | |
| | sequence | |
| 58 | CD28 | KIEVMYPPPYLDNEKSNGTIIHVKGKHLCPSPLF |
| | portion | PGPSKP |
| | of hybrid | |
| | CD8 and | |
| | CD28 | |
| | hinge | |
| | amino acid | |
| | sequence | |
| 59 | CD3ζ in- | RVKFSRSADAPAYQQGQNQLYNELNLGRREEY |
| | tracellular | DVLDKRRGRDPEMGGKPRRKNPQEGLYNELQ |
| | domain | KDKMAEAYSEIGMKGERRRGKGHDGLYQGLS |
| | amino acid | TATKDTYDALHMQALPPR |
| | sequence | |
| 60 | Hybrid | GCTAAGCCCACCACGACGCCAGCGCCGCGA |
| | CD8 and | CCACCAACACCGGCGCCCACCATCGCGTCGC |
| | CD28 | AGCCCCTGTCCCTGCGCCCAGAGGCGTGCCG |
| | hinge | GCCAGCGGCGGGGGGCGCAGTGCACACGAG |
| | nucleic | GGGGCTGGACTTCGCCCCTAGGAAAATTGAA |
| | acid | GTTATGTATCCTCCTCCTTACCTAGACAATGA |
| | sequence | GAAGAGCAATGGAACCATTATCCATGTGAAA |
| | | GGGAAACACCTTTGTCCAAGTCCCCTATTTC |
| | | CCGGACCTTCTAAGCCC |
| 61 | CD28 | TTTTGGGTGCTGGTGGTGGTTGGTGGAGTCC |
| | trans- | TGGCTTGCTATAGCTTGCTAGTAACAGTGGC |
| | membrane | CTTTATTATTTTCTGGGTG |
| | domain | |
| | nucleic | |
| | acid | |
| | sequence | |
| 62 | CD3 in- | AGAGTGAAGTTCAGCAGGAGCGCAGACGCC |
| | tracellular | CCCGCGTACCAGCAGGGCCAGAACCAGCTCT |
| | domain | ATAACGAGCTCAATCTAGGACGAAGAGAGG |
| | nucleic | AGTACGATGTTTTGGACAAGAGACGTGGCCG |
| | acid | GGACCCTGAGATGGGGGGAAAGCCGAGAAG |
| | sequence | GAAGAACCCTCAGGAAGGCCTGTACAATGA |
| | | ACTGCAGAAAGATAAGATGGCGGAGGCCTA |
| | | CAGTGAGATTGGGATGAAAGGCGAGCGCCG |
| | | GAGGGGCAAGGGGCACGATGGCCTTTACCA |
| | | GGGTCTCAGTACAGCCACCAAGGACACCTAC |
| | | GACGCCCTTCACATGCAGGCCCTGCCCCCTC |
| | | GCTAA |
| 63 | Myc-tag | GAGCAGAAGCTGATTAGCGAAGAGGACCTG |
| | nucleic | |
| | acid | |
| | sequence | |
| 64 | SS-14 | Ala-Gly-cyclo(Cys-Lys-Asn-Phe- |
| | (somatostatin | Phe-Trp-Lys-Thr-Phe-Thr-Ser-Cys) |
| | analog) | |
| 65 | OC | D-Phe1-cyclo(Cys2-Phe3-D-Trp4- |
| | (somatostatin | Lys5-Thr6-Cys7)Thr(ol)8 |
| | analog) | |
| 66 | TOC | D-Phe1-cyclo(Cys2-Tyr3-D-Trp4- |
| | (somatostatin | Lys5-Thr6-Cys7)Thr(ol)8 |
| | analog) | |
| 67 | TATE | D-Phe1-cyclo(Cys2-Tyr3-D-Trp4- |
| | (somatostatin | Lys5-Thr6-Cys7)Thr8 |
| | analog) | |
| 68 | NOC | D-Phe1-cyclo(Cys2-1-NaI3-D-Trp4- |
| | (somatostatin | Lys5-Thr6-Cys7)Thr(ol)8 |
| | analog) | |
| 69 | NOC-ATE | D-Phe1-cyclo(Cys2-1-NaI3-D-Trp4- |
| | (somatostatin | Lys5-Thr6-Cys7)Thr8 |
| | analog) | |
| 70 | BOC | D-Phe1-cyclo(Cys2-BzThi3-D-Trp4- |
| | (somatostatin | Lys5-Thr6-Cys7)Thr(ol)8 |
| | analog) | |
| 71 | BOC-ATE | D-Phe1-cyclo(Cys2-BzThi3-D-Trp4- |
| | (somatostatin | Lys5-Thr6-Cys7)Thr8 |
| | analog) | |
| 72 | KE108 | Tyr-cyclo(DAB-Arg-Phe-Phe-D-Trp- |
| | (somatostatin | Lys-Thr-Phe) |
| | analog) | |
| 73 | LM3 | p-Cl-Phe-cyclo(D-Cys-Tyr-D- |
| | (somatostatin | Aph(Cbm)-Lys-Thr-Cys)D-Tyr-NH2 |
| | analog) | |
| 74 | BN (bombesin | pGlu1-Gln2-Arg3-Leu4-Gly5-Asn6- |
| | analog) | Gln7-Trp8-Ala9-Val10-Gly11- |
| | | His12-Leu13-Met14-NH2 |
| 75 | RP527 | N3S-Gly-5-Ava-[Gln7-Trp8-Ala9- |
| | (bombesin | Val10-Gly11-His12-Leu13-Met14- |
| | analog) | NH2] |
| 76 | Demobesin 1 | N40-1-bzlg0[D-Phe6-Gln7-Trp8- |
| | (bombesin | Ala9-Val10-Gly11-His12-Leu- |
| | analog) | NHEt13] |
| 77 | Demobesin 4 | N4-[Pro1-Gln2-Arg3-Tyr4-Gly5- |
| | (bombesin | Asn6-Gln7-Trp8-Ala9-Val10- |
| | analog) | Gly11-His12-Leu13-Nle14-NH2] |
| 78 | BBS-38 | (NαHis)Ac-β-Ala-β-Ala-[Gln7- |
| | (bombesin | Trp8-Ala9-Val10-Gly11-His12- |
| | analog) | Cha13-Nle14-NH2] |
| 79 | BAY 86-4367 | 3-cyano-4-trimethylammonium- |
| | (bombesin | benzoyl-Ala(SO3H)-Ala(SO3H)- |
| | analog) | Ava[Gln7-Trp8-Ala9-Val10- |
| | | NMeGly11-His12-Sta13-Leu14- |
| | | NH2] |
| 80 | MG | Leu1-Glu2-Glu3-Glu4-Glu5-Glu6- |
| | (minigastrin | Ala7-Tyr8-Gly9-Trp10-Met11- |
| | analog) | Asp12-Phe13-NH2 |
| 81 | MGO | D-Glu1-Glu2-Glu3-Glu4-Glu5- |
| | (minigastrin | Glu6-Ala7-Tyr8-Gly9-Trp10- |
| | analog) | Met11-Asp12-Phe13-NH2 |
| 82 | MG11 | D-Glu-Ala-Tyr-Gly-Trp-Met- |
| | (minigastrin | Asp-Phe-NH2 |
| | analog) | |
| 83 | H2-Met | His-His-Glu-Ala-Tyr-Gly- |
| | (minigastrin | Trp-Met-Asp-Phe-NH2 |
| | analog) | |
| 84 | H2-Nle | His-His-Glu-Ala-Tyr-Gly- |
| | (minigastrin | Trp-Nle-Asp-Phe-NH2 |
| | analog) | |
| 85 | Demogastrin | N4-D-Glu-(Glu)5-Ala-Tyr- |
| | (minigastrin | Gly-Trp-Met-Asp-Phe-NH2 |
| | analog) | |
| 86 | Cyclo-MG1 | c(γ-D-Glu-Ala-Tyr-D-Lys)-Trp- |
| | (minigastrin | Met-Asp-Phe-NH2 |
| | analog) | |
| 87 | MGD5 | Gly-Ser-Cys(succinimido- |
| | (minigastrin | propionyl-Glu-Ala-Tyr-Gly- |
| | analog) | Trp-Nle-Asp-Phe-NH2)-Glu-Ala- |
| | | Tyr-Gly-Trp-Nle-Asp-Phe-NH2 |
| 88 | Buserelin | pGlu1-His2-Trp3-Ser4-Tyr5-D- |
| | (GnRH | Ser(tBu)6-Leu7-Arg8-Pro9- |
| | analog) | NHC2H5 |
| 89 | Goserelin | pGlu1-His2-Trp3-Ser4-Tyr5-D- |
| | (GnRH | Ser(tBu)6-Leu7-Arg8-Pro9- |
| | analog) | AzGly10-NH2 |
| 90 | Leuprolide | pGlu1-His2-Trp3-Ser4-Tyr5-D- |
| | (GnRH | Leu6-Leu7-Arg8-Pro9-NHC2H5 |
| | analog) | |
| 91 | Nafarelin | pGlu1-His2-Trp3-Ser4-Tyr5-D- |
| | (GnRH | Nal(2)6-Leu7-Arg8-Pro9- |
| | analog) | NHC2H5 |
| 92 | Triptorelin | pGlu1-His2-Trp3-Ser4-Tyr5-D- |
| | (GnRH | Trp6-Leu7-Arg8-Pro9-Gly10- |
| | analog) | NH2 |
| 93 | Abarelix | Ac-D-Ala1-D-Cpa2-D-Ala3- |
| | (GnRH | Ser4-Tyr5-D-Asp6-Leu7- |
| | analog) | Ilys8-Pro9-D-Ala10-NH2 |
| 94 | Acyline | Ac-D-Nal1-D-Cpa2-D-Pal3- |
| | (GnRH | Ser4-Aph(Ac)5-D-Aph(Ac)6- |
| | analog) | Leu7-Ilys8-Pro9-D-Ala10-NH2 |
| 95 | Antarelix | Ac-D-Nal1-D-Cpa2-D-Pal3- |
| | (GnRH | Ser4-Tyr5-D-Hci6-Leu7- |
| | analog) | Ilys8-Pro9-D-Ala10-NH2 |
| 96 | Antide | Ac-D-Nal1-D-Cpa2-D-Pal3- |
| | (GnRH | Ser4-Lys(Nic)5-D-Lys(Nic)6- |
| | analog) | Leu7-Ilys8-Pro9-D-Ala10-NH2 |
| 97 | Azaline B | Ac-D-Nal1-D-Cpa2-D-Pal3-Ser4- |
| | (GnRH | Aph(Atz)5-D-Aph(Atz)6-Leu7- |
| | analog) | Ilys8-Pro9-D-Ala10-NH2 |
| 98 | Cetrorelix | Ac-D-Nal1-D-Cpa2-D-Pal3-Ser4- |
| | (GnRH | Tyr5-D-Cit6-Leu7-Arg8-Pro9-D- |
| | analog) | Ala10-NH2 |
| 99 | Degarelix | Ac-D-Nal1-D-Cpa2-D-Pal3-Ser4- |
| | (GnRH | Aph(L-hydroorotyl)5-D- |
| | analog) | Aph(carbamoyl)6-Leu7-Ilys8- |
| | | Pro9-D-Ala10-NH2 |
| 100 | Ganirelix | Ac-D-Nal1-D-Cpa2-D-Pa13-Ser4- |
| | (GnRH | Tyr5-D-hArg(Et2)6-Leu7- |
| | analog) | hArg(Et2)8-Pro9-D-Ala10-NH2 |
| 101 | Ozarelix | Ac-D-Nal1-D-Cpa2-D-Pal3-Ser4- |
| | (GnRH | N-MeTyr5-D-hCit6-Nle7-Arg8- |
| | analog) | Pro9-D-Ala10-NH2 |
| 102 | Murine | DVVMTQTPLSLPVRLGDQASISCRSSQSLLHTY |
| | PUCR with | GSPYLNWYLQKPGQSPKLLIYKVSNRFSGVPD |
| | Myc-tag | RFSGSGSGTDFTLRISRVEAEDLGVYFCSQGTH |
| | without | LPYTFGGGTKLEIKGGGGSGGGGSGGGGSEVK |
| | signal | LVESGGGLVQPGGTMKLSCEISGLTFRNYWMS |
| | peptide | WVRQSPEKGLEWVAEIRLRSDNYATHYAESV |
| | amino acid | KGKFTISRDDSKSRLYLQMNSLRTEDTGIYYCK |
| | sequence | TYFYSFSYWGQGTLVTVSAEQKLISEEDLAKPT |
| | | TTPAPRPPTPAPTIASQPLSLRPEACRPAAGGAV |
| | | HTRGLDFALDPKLCYLLDGILFIYGVILTALFLR |
| | | VKRSKRSRLLHSDYMNMTPRRPGPTRKHYQP |
| | | YAPPRDFAAYRSRVKFSRSADAPAYQQGQNQL |
| | | YNELNLGRREEYDVLDKRRGRDPEMGGKPQR |
| | | RKNPQEGLYNELQKDKMAEAYSEIGMKGERR |
| | | RGKGHDGLYQGLSTATKDTYDALHMQALPPR |
| 103 | Humanized | ELQMTQSPSSLSASVGDRVTITCRSSQSLLHTY |
| | PUCR with | GSPYLNWYLQKPGQSPKLLIYKVSNRFSGVPSR |
| | Myc-tag | FSGSGSGTDFTLTISSLQPEDFAVYFCSQGTHLP |
| | without | YTFGGGTKVEIKGGGGSGGGGSGGGGSEVQLV |
| | signal | ESGGGLVQPGGSLRLSCAASGFTFSNYWMSW |
| | peptide | VRQSPEKGLEWVSEIRLRSDNYATHYAESVKG |
| | amino acid | RFTISRDNSKNTLYLQMNSLRAEDTGIYYCKTY |
| | sequence | FYSFSYWGQGTLVTVSSEQKLISEEDLAKPTTT |
| | | PAPRPPTPAPTIASQPLSLRPEACRPAAGGAVHT |
| | | RGLDFALDPKLCYLLDGILFIYGVILTALFLRVK |
| | | RSKRSRLLHSDYMNMTPRRPGPTRKHYQPYAP |
| | | PRDFAAYRSRVKFSRSADAPAYQQGQNQLYN |
| | | ELNLGRREEYDVLDKRRGRDPEMGGKPQRRK |
| | | NPQEGLYNELQKDKMAEAYSEIGMKGERRRG |
| | | KGHDGLYQGLSTATKDTYDALHMQALPPR |
| 104 | Full | ELQMTQSPSSLSASVGDRVTITCRSSQSLLHTY |
| | length | GSPYLNWYLQKPGQSPKLLIYKVSNRFSGVPSR |
| | humanized | FSGSGSGTDFTLTISSLQPEDFAVYFCSQGTHLP |
| | 38C2 scFab | YTFGGGTKVEIKRTVAAPSVFIFPPSDEQLKSGT |
| | without | ASVVCLLNNFYPREAKVQWKVDNALQSGNSQ |
| | signal | ESVTEQDSKDSTYSLSSTLTLSKADYEKHKVYA |
| | peptide | CEVTHQGLSSPVTKSFNRGECGGGSGGGGSGG |
| | amino acid | GSGGGGSGGGSGGGGSGGGGSGGGSGGGGSE |
| | sequence | VQLVESGGGLVQPGGSLRLSCAASGFTFSNYW |
| | | MSWVRQSPEKGLEWVSEIRLRSDNYATHYAES |
| | | VKGRFTISRDNSKNTLYLQMNSLRAEDTGIYYC |
| | | KTYFYSFSYWGQGTLVTVSSASTKGPSVFPLAP |
| | | SSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHT |
| 105 | Full | ELQMTQSPSSLSASVGDRVTITCRSSQSLLHTY |
| | length | GSPYLNWYLQKPGQSPKLLIYKVSNRFSGVPSR |
| | PUCR com- | FSGSGSGTDFTLTISSLQPEDFAVYFCSQGTHLP |
| | prising | YTFGGGTKVEIKRTVAAPSVFIFPPSDEQLKSGT |
| | 38C2 scFab | ASVVCLLNNFYPREAKVQWKVDNALQSGNSQ |
| | without | ESVTEQDSKDSTYSLSSTLTLSKADYEKHKVYA |
| | signal | CEVTHQGLSSPVTKSFNRGECGGGSGGGGSGG |
| | peptide | GSGGGGSGGGSGGGGSGGGGSGGGSGGGGSE |
| | amino acid | VQLVESGGGLVQPGGSLRLSCAASGFTFSNYW |
| | sequence | MSWVRQSPEKGLEWVSEIRLRSDNYATHYAES |
| | | VKGRFTISRDNSKNTLYLQMNSLRAEDTGIYYC |
| | | KTYFYSFSYWGQGTLVTVSSASTKGPSVFPLAP |
| | | SSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKKVEPKSCDKTHTAKP |
| | | TTTPAPRPPTPAPTIASQPLSLRPEACRPAAGGA |
| | | VHTRGLDFAPRKIEVMYPPPYLDNEKSNGTIIH |
| | | VKGKHLCPSPLFPGPSKPFWVLVVVGGVLACY |
| | | SLLVTVAFIIFWVRSKRSRLLHSDYMNMTPRRP |
| | | GPTRKHYQPYAPPRDFAAYRSRVKFSRSADAP |
| | | AYQQGQNQLYNELNLGRREEYDVLDKRRGRD |
| | | PEMGGKPRRKNPQEGLYNELQKDKMAEAYSEI |
| | | GMKGERRRGKGHDGLYQGLSTATKDTYDALH |
| | | MQALPPR |
| 106 | Full | AGCGAACTGCAGATGACCCAGTCCCCATCCA |
| | length | GTCTGAGCGCTAGCGTTGGTGACAGAGTTAC |
| | PUCR com- | TATCACCTGCCGCTCTTCACAGAGCCTGTTG |
| | prising | CACACTTACGGCTCTCCTTACCTGAACTGGT |
| | 38C2 scFab | ATCTTCAGAAGCCTGGCCAAAGCCCTAAGCT |
| | without | GCTCATCTACAAGGTGTCTAACAGGTTCTCC |
| | signal | GGGGTTCCGTCCCGCTTTTCAGGGAGCGGGT |
| | peptide | CAGGAACAGACTTCACCTTGACAATCTCAAG |
| | nucleic | CCTCCAGCCCGAGGATTTTGCCGTCTATTTCT |
| | acid | GCTCACAAGGCACACATCTGCCGTATACCTT |
| | sequence | TGGGGGCGGGACAAAAGTCGAGATCAAAAG |
| | | GACCGTCGCTGCACCATCCGTGTTTATCTTCC |
| | | CACCAAGTGACGAACAGCTCAAGAGCGGTA |
| | | CTGCCTCCGTTGTTTGTCTGCTGAACAACTTC |
| | | TATCCAAGGGAAGCAAAGGTGCAATGGAAA |
| | | GTAGACAACGCTCTGCAGTCAGGCAACTCCC |
| | | AGGAGTCAGTGACCGAGCAGGATAGCAAAG |
| | | ATTCAACATACAGCCTGAGCAGCACCCTCAC |
| | | CCTGAGTAAGGCCGATTACGAGAAGCACAA |
| | | GGTTTACGCCTGCGAGGTGACCCACCAGGGC |
| | | CTTTCATCCCCAGTCACCAAATCTTTTAACCG |
| | | CGGCGAATGCGGGGGAGGCTCTGGTGGAGG |
| | | CGGTTCTGGAGGGGGCTCAGGAGGAGGCGG |
| | | TAGCGGCGGTGGTAGTGGGGGTGGCGGATCT |
| | | GGCGGAGGTGGCTCAGGAGGAGGTAGCGGC |
| | | GGCGGGGGCAGCGAGGTCCAGCTGGTAGAG |
| | | TCAGGTGGAGGATTGGTGCAGCCCGGCGGCA |
| | | GTCTTAGACTCAGCTGTGCGGCCAGCGGATT |
| | | TACTTTCTCAAATTATTGGATGTCTTGGGTCA |
| | | GGCAGAGCCCAGAGAAAGGCCTGGAATGGG |
| | | TGTCAGAGATCCGACTGAGAAGCGATAATTA |
| | | CGCGACTCATTATGCGGAAAGCGTTAAAGGT |
| | | CGGTTCACTATTTCACGAGATAATTCTAAGA |
| | | ATACCCTTTATCTGCAGATGAACAGCTTGCG |
| | | CGCCGAGGACACAGGCATCTACTACTGTAAA |
| | | ACTTACTTCTATTCTTTTTCCTACTGGGGACA |
| | | GGGGACTCTCGTTACAGTCAGTAGCGCCTCC |
| | | ACCAAGGGTCCTAGTGTCTTTCCCCTGGCCC |
| | | CCTCATCCAAGTCCACGTCAGGAGGCACCGC |
| | | GGCTCTGGGCTGTCTGGTCAAAGACTACTTT |
| | | CCTGAGCCAGTCACCGTGTCCTGGAATTCCG |
| | | GCGCGCTTACTTCTGGCGTGCACACTTTCCCC |
| | | GCCGTCCTCCAGAGCAGTGGGCTGTATTCCC |
| | | TGTCTTCCGTAGTCACTGTGCCAAGCTCCAGT |
| | | CTGGGAACCCAGACCTATATTTGTAATGTGA |
| | | ATCATAAGCCGAGCAACACCAAGGTGGACA |
| | | AGAAGGTGGAACCGAAGTCATGTGACAAAA |
| | | CCCACACTGCTAAGCCCACCACGACGCCAGC |
| | | GCCGCGACCACCAACACCGGCGCCCACCATC |
| | | GCGTCGCAGCCCCTGTCCCTGCGCCCAGAGG |
| | | CGTGCCGGCCAGCGGCGGGGGGCGCAGTGC |
| | | ACACGAGGGGGCTGGACTTCGCCCCTAGGAA |
| | | AATTGAAGTTATGTATCCTCCTCCTTACCTAG |
| | | ACAATGAGAAGAGCAATGGAACCATTATCC |
| | | ATGTGAAAGGGAAACACCTTTGTCCAAGTCC |
| | | CCTATTTCCCGGACCTTCTAAGCCCTTTTGGG |
| | | TGCTGGTGGTGGTTGGTGGAGTCCTGGCTTG |
| | | CTATAGCTTGCTAGTAACAGTGGCCTTTATTA |
| | | TTTTCTGGGTGAGGAGTAAGAGGAGCAGGCT |
| | | CCTGCACAGTGACTACATGAACATGACTCCC |
| | | CGCCGCCCCGGGCCCACCCGCAAGCATTACC |
| | | AGCCCTATGCCCCACCACGCGACTTCGCAGC |
| | | CTATCGCTCCAGAGTGAAGTTCAGCAGGAGC |
| | | GCAGACGCCCCCGCGTACCAGCAGGGCCAG |
| | | AACCAGCTCTATAACGAGCTCAATCTAGGAC |
| | | GAAGAGAGGAGTACGATGTTTTGGACAAGA |
| | | GACGTGGCCGGGACCCTGAGATGGGGGGAA |
| | | AGCCGAGAAGGAAGAACCCTCAGGAAGGCC |
| | | TGTACAATGAACTGCAGAAAGATAAGATGG |
| | | CGGAGGCCTACAGTGAGATTGGGATGAAAG |
| | | GCGAGCGCCGGAGGGGCAAGGGGCACGATG |
| | | GCCTTTACCAGGGTCTCAGTACAGCCACCAA |
| | | GGACACCTACGACGCCCTTCACATGCAGGCC |
| | | CTGCCCCCTCGCTAA |

1. An isolated nucleic acid sequence encoding a programmable universal cell receptor, wherein said programmable universal cell receptor comprises:
a. a catalytic antibody, or a catalytic portion thereof, comprising a reactive amino acid residue;
b. a transmembrane domain; and
c. an intracellular domain.

2. The isolated nucleic acid sequence of claim 1, wherein the catalytic antibody, or a catalytic portion thereof, is selected from the group consisting of an aldolase catalytic antibody, a beta lactamase catalytic antibody, an amidase catalytic antibody, a thioesterase catalytic antibody, and catalytic portions thereof.

3. (canceled)

4. The isolated nucleic acid sequence of claim 1, wherein the reactive amino acid residue is selected from the group consisting of a reactive cysteine residue, a reactive tyrosine residue, a reactive lysine residue, and a reactive tyrosine residue.

5. (canceled)

6. The isolated nucleic acid sequence of claim 1,
wherein the catalytic antibody, or a catalytic portion thereof, is selected from the group consisting of a humanized monoclonal antibody 38C2, or a catalytic portion thereof a humanized monoclonal antibody 33F12, or a catalytic portion thereof; a murine monoclonal antibody 38C2 or a catalytic portion thereof; or a murine monoclonal antibody 33F12, or a catalytic portion thereof.

7. The isolated nucleic acid sequence of claim 1, wherein the catalytic antibody, or a catalytic portion thereof, comprises the amino acid sequence of SEQ ID NO: 4, or a catalytic portion thereof.

8.-11. (canceled)

12. The isolated nucleic acid sequence of claim 1, wherein the catalytic portion is selected from the group consisting of a single chain variable fragment (scFv), a scFab, a diabody, a F(ab′)₂ fragment, a Fd fragment consisting of the VH and CH1 domains, and a dAb fragment.

13. The isolated nucleic acid sequence of claim 1, wherein the intracellular domain comprises a signaling domain, wherein the signaling domain is a CD3-ζ signaling domain or a CD28 signaling domain.

14.-15. (canceled)

16. The isolated nucleic acid sequence of claim 1, wherein the intracellular domain comprises a co-stimulatory signaling domain.

17. The isolated nucleic acid sequence of claim 16, wherein the co-stimulatory signaling domain comprises an intracellular domain of a protein selected from the group consisting of CD27, CD28, 4-1BB, OX40, CD30, CD40, ICOS, lymphocyte function-associated antigen-1 (LFA-1), CD2, CD7, LIGHT, NKG2C, a CD83 ligand, and any combination thereof.

18. The isolated nucleic acid sequence of claim 1, wherein the transmembrane domain comprises the transmembrane domain of a protein selected from the group consisting of: the alpha chain of the T-cell receptor, the beta chain of the T-cell receptor, the zeta chain of the T-cell receptor, CD28, CD3 epsilon, CD45, CD4, CD5, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and fragments thereof.

19. The isolated nucleic acid sequence of claim 1, wherein the programmable universal cell receptor further comprises a hinge region.

20. The isolated nucleic acid sequence of claim 19, wherein the hinge region is a CD8 hinge region.

21. The isolated nucleic acid sequence of claim 1, wherein the programmable universal cell receptor further comprises a detectable moiety.

22.-23. (canceled)

24. An isolated nucleic acid sequence encoding a programmable universal cell receptor, wherein the programmable universal cell receptor comprises an amino acid sequence as set forth in SEQ ID NO: 10.

25. A vector comprising the nucleic acid sequence of claim 1.

26.-27. (canceled)

28. An isolated host cell comprising the isolated nucleic acid of claim 1.

29. The host cell of claim 28, wherein the programmable universal cell receptor is conjugated to a specificity agent via a reactive moiety, wherein the reactive moiety is bound to the reactive amino acid residue of the catalytic antibody, or catalytic portion thereof.

30. (canceled)

31. The host cell of claim 29, wherein the reactive moiety is selected from the group consisting of a diketone, a N-sulfonyl-beta-lactam, and an azetidinone.

32. The host cell of claim 29, wherein the specificity agent comprises a reactive moiety that is conjugated via a linker.

33. (canceled)

34. The host cell of claim 29, wherein the specificity agent binds to a protein associated with cancer, a viral protein, or a protein expressed by a disease-causing organism.

35. The host cell of claim 34, wherein the protein associated with cancer is selected from the group consisting of CD19, an integrin, VEGFR2, PSMA, CEA, GM2, GD2, GD3, EGFR, EGFRvIII, HER2, IL13R, and MUC-1.

36. (canceled)

37. The host cell of claim 34, wherein the viral protein is an HIV protein, a hepatitis virus protein, an influenza virus protein, a herpes virus protein, a rotavirus protein, a respiratory syncytial virus protein, a poliovirus protein, a rhinovirus protein, a cytomegalovirus protein, a simian immunodeficiency virus protein, an encephalitis virus protein, a varicella zoster virus protein, and an Epstein-Barr virus protein.

38.-40. (canceled)

41. The host cell of claim 34, wherein the disease-causing organism is selected from the group consisting of a virus, a prion, a bacterium, a fungus, a protozoan, and a parasite.

42. The host cell of claim 29, wherein the specificity agent comprises a binding protein, small molecule, a peptide, a peptidomimetic, a therapeutic agent, a targeting agent, a protein agonist, a protein antagonist, a metabolic regulator, a hormone, a toxin, or a growth factor.

43. The host cell of claim 42, wherein the small molecule is folic acid or DUPA.

44. The host cell of claim 42, wherein the binding protein is an antibody, an antigen-binding portion of an antibody, a ligand, a cytokine, or a receptor.

45. The host cell of claim 29, wherein the host cell comprises
a programmable universal cell receptor which is conjugated to a specificity agent specific for
a first antigen, and
a programmable universal cell receptor which is conjugated to a specificity agent specific for
a second antigen, which is different than the first antigen.

46. The host cell of claim 29, wherein the host cell is an immune cell selected from the group consisting of a dendritic cell, a monocyte, a mast cell, an eosinophil, a T cell, a B cell, a cytotoxic T lymphocyte, a macrophage, a Natural Killer cell, a monocyte, and a Natural Killer T (NKT) cell.

47.-50. (canceled)

51. A population of host cells of claim 29, wherein the population of comprises:
a. a subpopulation of host cells comprising a programmable universal cell receptor linked to a specificity agent that binds to a first antigen, and
b. a subpopulation of host cells comprising a programmable universal cell receptor linked to a specificity agent that binds to a second antigen, which is different than the first antigen.

52. A method for treating a cancer or a medical condition caused by a disease-causing organism, or inhibiting tumor growth, in a subject in need thereof, the method comprising administering to the subject the host cell of claim 29, thereby treating the cancer, the medical condition caused by the disease-causing organism, or inhibiting tumor growth in the subject.

53. (canceled)

54. A method of making a customized therapeutic host cell for use in the treatment of cancer in a subject in need thereof, the method comprising:
contacting an immune cell with a specificity agent that binds to a programmable universal cell receptor that is expressed on the cell membrane of the immune cell, wherein the specificity agent binds to a cancer-associated antigen corresponding to a cancer antigen profile of the subject in need thereof.

55.-59. (canceled)

60. The method of claim 54, wherein the specificity agent comprises a binding protein, small molecule, a peptide, a peptidomimetic, a therapeutic agent, a targeting agent, a protein agonist, a protein antagonist, a metabolic regulator, a hormone, a toxin, or a growth factor.

61. The method of claim 60, wherein the binding protein is an antibody or an antigen binding fragment thereof.

62. (canceled)

63. The method of claim 61, wherein the antibody or antibody binding fragment thereof comprises a variable kappa light chain.

64. A method for treating a cancer in a subject in need thereof, said method comprising:
(a) determining a cancer antigen profile of the subject;
(b) selecting a specificity agent that binds to the antigen identified in (a); and
(c) administering an immune cell comprising a programmable universal cell receptor bound to the specificity agent identified in (b),
thereby treating the cancer in the subject in need thereof.

65. A kit comprising a container comprising a population of host cells comprising a programmable universal cell receptor,
wherein the programmable universal cell receptor comprises a catalytic antibody, or a catalytic portion thereof, comprising a reactive amino acid residue,
wherein the reactive amino acid residue is not bound to a substrate;
a transmembrane domain; and
an intracellular domain.

66.-71. (canceled)

72. A method for treating a cancer or a medical condition caused by a disease-causing organism, or inhibiting tumor growth, in a subject in need thereof, the method comprising administering to the subject the population of host cells of claim 51, thereby treating the cancer, the medical condition caused by the disease-causing organism, or inhibiting tumor growth in the subject.
